(19) United States
(12) Patent Application Publication
(10) Pub. No.: US 2026/0002925 A1
(43) Pub. Date: Jan. 1, 2026

(54) NANOPORE SYSTEMS AND METHODS FOR SINGLE-MOLECULE POLYMER PROFILING

(71) Applicant: Rijksuniversiteit Groningen, Groningen (NL)

(72) Inventors: Giovanni MAGLIA, Glimmen (NL); Adina SAUCIUC, Groningen (NL)

(21) Appl. No.: 19/190,340

(22) Filed: Apr. 25, 2025

Related U.S. Application Data
(63) Continuation of application No. PCT/NL2023/050568, filed on Oct. 30, 2023.

(30) Foreign Application Priority Data
Oct. 28, 2022 (EP) 22204589.0

(51) Int. Cl. G01N 33/487; G01N 33/68

(52) U.S. Cl. CPC G01N 33/48721; G01N 33/68

(57) ABSTRACT

The invention relates to means and methods for analysis of target analytes using nanopore-based sensors, more in particular to methods, nanopore systems and devices for single-molecule profiling of polymers, e.g. polypeptide or polysaccharides. Provided is a method for translocating a non-nucleic acid based polymer analyte through a nanopore, the nanopore being comprised in a membrane separating a fluidic chamber of a nanopore system into a cis side and a trans side, comprising adding the analyte to the cis side of and allowing for translocation, wherein the nanopore system has a cis to trans electro-osmotic force (EOF) resulting from a net ionic current flow cis to trans, preferably wherein the cis to trans EOF results from a net ionic current flow cis to trans over total ionic current flow of greater than 0.2 or less than −0.2.

### CROSS-REFERENCE

This application is a continuation of International Application No. PCT/NL2023/050568, filed Oct. 30, 2023, which claims benefit of European Application No. EP22204589.0, filed Oct. 28, 2022, each of which is herein incorporated by reference in its entirety.

### SEQUENCE LISTING

The instant application contains a Sequence Listing which has been submitted electronically in XML file format and is hereby incorporated by reference in its entirety. Said XML copy, created on Jul. 2, 2025, is named 64828-706_301_SL.xml and is 32,239 bytes in size.

### BACKGROUND

Characterizing and identifying analytes is an important aspect of scientific studies. These scientific studies can have important impacts on clinical and scientific endeavors.

### SUMMARY

In an aspect, the present disclosure provides a method comprising: providing: a nanopore system, wherein the nanopore system comprises a fluidic chamber and; a membrane comprising a nanopore, wherein the membrane separates the fluidic chamber into a cis side and a trans side; and a non-nucleic acid based polymer analyte, wherein the non-nucleic acid based polymer analyte comprises a linear length greater than a channel length of the nanopore; translocating the non-nucleic acid based polymer analyte from the cis side to the trans side of the fluidic chamber, wherein the non-nucleic acid based polymer analyte comprises an elongated structure, wherein the nanopore system has a cis to trans electro-osmotic force resulting from a cis to trans net ionic current flow, wherein the cis to trans electro-osmotic force translocates the non-nucleic acid based polymer analyte through the nanopore against an electrophoretic force acting in a direction opposite the cis to trans electro-osmotic force.

In some embodiments, the electro-osmotic force is at least 10% greater than the electrophoretic force. In some embodiments, the electro-osmotic force is at least 50% greater than the electrophoretic force. In some embodiments, the electro-osmotic force is at least 100% greater than the electrophoretic force. In some embodiments, the cis side of the fluidic chamber comprises a first solution and the trans side of the fluidic chamber comprises a second solution. In some embodiments, the first solution comprises a first concentration of a solute and the second solution comprises a second concentration of a solute. In some embodiments, the solute comprises an ion or an osmolyte. In some embodiments, a difference between the first concentration of the solute and the second concentration of the solute is configured to generate the cis to trans electro-osmotic force in a presence of an applied potential.

In some embodiments, the non-nucleic acid based polymer analyte is an unmodified (label-free) non-nucleic acid based polymer analyte. In some embodiments, an analyte is an unmodified analyte. In some cases, an unmodified analyte can be a wild-type version of the analyte. In some cases, the unmodified analyte may not comprise any additional molecules coupled to the unmodified analyte. In some embodiments, an analyte is a label-free or tag-free analyte. In some cases, a label-free analyte can comprise analyte that is not coupled to any peptide labels, any protein labels, any nucleic acid labels, any saccharide labels, any lipids labels, or any combination thereof. In some cases, a tag-free analyte can comprise analyte that is not coupled to any peptide tags, any protein tags, any nucleic acid tags, any saccharide tags, any tags labels, or any combination thereof. In some embodiments, termini of the non-nucleic acid based polymer analyte lack a three-dimensional structure. In some embodiments, at least a portion of the non-nucleic acid based polymer analyte is denatured. In some embodiments, the linear length of the non-nucleic acid based polymer analyte is greater than the channel length of the nanopore transversing the membrane when the non-nucleic acid based polymer analyte is elongated. In some embodiments, the non-nucleic acid based polymer analyte comprises at least about 25 repeating units. In some embodiments, the non-nucleic acid based polymer analyte comprises peptide units, saccharide units, water-soluble plastic monomers, or any combination thereof. In some embodiments, the non-nucleic acid based polymer analyte comprises a polypeptide, a polysaccharide, or a water-soluble plastic.

In some embodiments, the non-nucleic acid based polymer analyte comprises a polypeptide of at least 30 peptide units. In some embodiments, the at least 30 peptide units comprise positively or negatively charged peptide units. In some embodiments, the polypeptide is in a denatured state. In some embodiments, the polypeptide is provided in a folded state.

In some embodiments, the method further comprises measuring a signal generated by the translocating the non-nucleic acid based polymer analyte through the nanopore.

In some embodiments, the measuring comprises: measuring a signal for a state of (a) an open channel of the nanopore; (b) capture of the non-nucleic acid based polymer analyte by the nanopore; or (c) passage of the non-nucleic acid based polymer analyte through the nanopore. In some embodiments, the measuring comprises detecting differences between states (a), (b) and (c). In some embodiments, the signal comprises an ionic current, a change in ionic current, or derivations thereof.

In some embodiments, the linear length of the non-nucleic acid based polymer analyte is at least 1 kDa. In some embodiments, the linear length of the non-nucleic acid based polymer analyte is at most 4,000 kDa. In some embodiments, the linear length of the non-nucleic acid based polymer analyte is at least 2 times greater than the channel length of the nanopore. In some embodiments, the linear length of the non-nucleic acid based polymer analyte is at most 2 times greater than the channel length of the nanopore. In some embodiments, the linear length of the non-nucleic acid based polymer analyte is at least 3 nanometers.

In some embodiments, the cis to trans electro-osmotic force comprises a net ionic current flow cis-to-trans. In some embodiments, the cis to trans electro-osmotic force is modulated by a pH, a type of a salt, a concentration of a salt, an osmotic pressure across the membrane of the system, a modification of the nanopore, or any combinations thereof. In some embodiments, the cis to trans electro-osmotic force is modulated by a modification of a charge of the nanopore. In some embodiments, the cis to trans electro-osmotic force is modulated by an asymmetric salt distribution between the cis side of the membrane and the trans side of the membrane. In some embodiments, the nanopore has an ion-selectivity P(+)/P(−) of greater than 2.0. In some embodiments, the nanopore has an ion-selectivity P(+)/P(−) of less than 0.50.

In some embodiments, the nanopore system further comprises a pair of electrodes. In some embodiments, the pair of electrodes is configured to provide an applied voltage to generate the electrophoretic force. In some embodiments, the applied voltage is a negative voltage on the trans side. In some embodiments, the applied voltage is a positive voltage on the trans side. In some embodiments, a magnitude of the applied voltage is less than 300 mV. In some embodiments, a magnitude of the applied voltage is greater than 20 mV. In some embodiments, an absolute relative net electro-osmotic current over the applied voltage is greater than about 0.10 pA/mV. In some embodiments, the nanopore comprises an inner pore constriction from about 0.5 nanometers to about 2 nanometers (nm).

In some embodiments, the nanopore comprises an alpha-helical oligomeric pore structure. In some embodiments, the nanopore comprises a beta-barrel oligomeric pore structure. In some embodiments, the nanopore comprises a recombinant nanopore. In some embodiments, the nanopore comprises a protein of Aerolysin (Aer), Cytolysin K (CytK), MspA, alpha-hemolysin (aHL), CsgG, Fragaceatoxin C (FraC), Lysenin, OmpF, OmpG, FhuA, phage derived portal proteins, modified variants thereof, or ion-selective mutants thereof.

In some embodiments, the nanopore comprises a biological nanopore. In some embodiments, the biological nanopore is modified to limit passage of one or more ions through the channel of the nanopore. In some embodiments, the biological nanopore limits passage of one or more ions through the channel of the nanopore by modifying a charge of the channel of the nanopore. In some embodiments, a net charge of the channel is negative. In some embodiments, a net charge of the channel is positive.

In some embodiments, the nanopore comprises a mutant CytK nanopore. In some embodiments, the mutant CytK nanopore comprises one or more amino acid substitutions. In some embodiments, the one or more amino acid substitutions comprises K128D, K128F, K115D, S120D, Q122D, S151D, or any combination thereof. In some embodiments, the one or more amino acid substitutions comprises K128D, K155Q, T116D, S120D, Q122D, S126D, T143D, Q145D, T147D, S151D, or any combination thereof. In some embodiments, the mutant CytK nanopore comprises one of the following combinations of amino acid substitutions: (a) K128D and K155D; (b) K128D, K155D and T116D; (c) T147D or S151D; (d) K128D, K155D, and S120D; (e) Q122D, T147D or S155D; (f) K128D, K155D, Q145D and S151D; and (g) a combination thereof. In some embodiments, the mutant CytK nanopore comprises one or more of the following combinations of amino acid substitutions: (a) S120D, G122D, or K155D; (b) S120D in combination with K128F/K128D; (c) Q122D or S151D; (d) K128D or K128F; (e) S120D, K115D and Q122D; (f) K128F, S120D and G122D; (g) K128F, S120D G122D, and K155D; and (h) a combination thereof.

In another aspect, the present disclosure provides a system comprising: a fluidic chamber; a membrane comprising a nanopore, wherein the membrane separates the fluidic chamber into a cis side comprising a first solution and a trans side comprising a second solution, wherein the first solution and the second solution are configured to translocate a non-nucleic acid based polymer analyte across the nanopore using an electro-osmotic flow, wherein the non-nucleic acid based polymer analyte comprises an elongated structure, wherein the non-nucleic acid based polymer analyte comprises a linear length greater than a channel length of the nanopore; a pair of electrodes comprising a first electrode and a second electrode, wherein the first electrode is disposed on the cis side of the fluidic chamber and the second electrode is disposed on the trans side of the fluidic chamber, wherein the pair of electrodes are configured to generate an electrophoretic force acting in an opposite direction to the electro-osmotic flow.

In another aspect, the present disclosure provides a system comprising: a fluidic chamber; a membrane comprising a nanopore, wherein the membrane separates the fluidic chamber into a cis side comprising a first solution and a trans side comprising a second solution, wherein the first solution and the second solution are configured to translocate a non-nucleic acid based polymer analyte using an electro-osmotic flow; a pair of electrodes comprising a first electrode and a second electrode; and a controller operatively coupled to the fluidic chamber, said nanopore, and the pair of electrodes, wherein the controller: uses the pair of electrodes to generate an electrophoretic force acting in an opposite direction to the electro-osmotic flow that translocates the non-nucleic acid based polymer analyte through the nanopore, and detects one or more signals associated with at least one characteristic of the non-nucleic acid based polymer analyte during or subsequent to translocation of the non-nucleic acid based polymer analyte through the nanopore, wherein the non-nucleic acid based polymer analyte comprises a linear length greater than a channel length of the nanopore.

In some embodiments, the controller uses said pair of electrodes to detect the one or more signals associated with the at least one characteristic of the non-nucleic acid based polymer analyte. In some embodiments, the electro-osmotic flow is greater than the electrophoretic force.

In some embodiments, the electro-osmotic flow is at least 10% greater than the electrophoretic force. In some embodiments, the electro-osmotic flow is at least 50% greater than the electrophoretic force. In some embodiments, the electro-osmotic flow is at least 100% greater than the electrophoretic force. In some embodiments, the first solution comprises a first concentration of a solute and the second solution comprises a second concentration of a solute.

In some embodiments, the solute comprises an ion or an osmolyte. In some embodiments, a difference between the first concentration of the solute and the second concentration of the solute is configured to generate the electro-osmotic flow in a presence of an applied potential.

In some embodiments, the electro-osmotic flow comprises a net ionic current flow cis-to-trans. In some embodiments, the electro-osmotic flow is modulated by a pH, a type of a salt, a concentration of a salt, an osmotic pressure across the membrane of the system, a modification of the nanopore, or any combinations thereof. In some embodiments, the electro-osmotic flow is modulated by a modification of a charge of the nanopore. In some embodiments, the electro-osmotic flow is modulated by an asymmetric salt distribution between the cis side of the membrane and the trans side of the membrane. In some embodiments, the nanopore has an ion-selectivity P(+)/P(−) of greater than 2.0. In some embodiments, the nanopore has an ion-selectivity P(+)/P(−) of less than 0.50.

In some embodiments a pair of electrodes can be configured to provide an applied voltage. The applied voltage can be across a membrane. The applied voltage can result in an electrophoretic force. In some embodiments a pair of electrodes can be configured to provide an electrophoretic force across a membrane. The pair of electrodes can be configured to measure a signal.

In some embodiments, the nanopore comprises an alpha-helical oligomeric pore structure. In some embodiments, the nanopore comprises a beta-barrel oligomeric pore structure. In some embodiments, the nanopore comprises a recombinant nanopore. In some embodiments, the nanopore comprises a protein of Aerolysin (Aer), Cytolysin K (CytK), MspA, alpha-hemolysin (aHL), CsgG, Fragaceatoxin C (FraC), Lysenin, OmpF, OmpG, FhuA, phage derived portal proteins, modified variants thereof, or ion-selective mutants thereof.

In some embodiments, the nanopore comprises a biological nanopore. In some embodiments, the biological nanopore is modified to limit passage of one or more ions through a channel of the nanopore. In some embodiments, the biological nanopore limits passage of one or more ions through the channel of the nanopore by modifying a charge of the channel of the nanopore. In some embodiments, a net charge of the channel is negative. In some embodiments, a net charge of the channel is positive.

In some embodiments, the nanopore comprises a mutant CytK nanopore. In some embodiments, the mutant CytK nanopore comprises one or more amino acid substitutions. In some embodiments, the one or more amino acid substitutions comprises K128D, K128F, K115D, S120D, Q122D, S151D, or any combination thereof. In some embodiments, the one or more amino acid substitutions comprises K128D, K155Q, T116D, S120D, Q122D, S126D, T143D, Q145D, T147D, S151D, or any combination thereof. In some embodiments, the mutant CytK nanopore comprises one of the following combinations of amino acid substitutions: (a) K128D and K155D; (b) K128D, K155D and T116D; (c) T147D or S151D; (d) K128D, K155D, and S120D; (e) Q122D, T147D or S155D; (f) K128D, K155D, Q145D and S151D; and (g) a combination thereof. In some embodiments, the mutant CytK nanopore comprises one or more of the following combinations of amino acid substitutions: (a) S120D, G122D, or K155D; (b) S120D in combination with K128F/K128D; (c) Q122D or S151D; (d) K128D or K128F; (e) S120D, K115D and Q122D; (f) K128F, S120D and G122D; (g) K128F, S120D G122D, and K155D; and (h) a combination thereof.

In some embodiments, the non-nucleic acid based polymer analyte is an unmodified (label-free) non-nucleic acid based polymer analyte. In some embodiments, termini of the non-nucleic acid based polymer analyte lack a three-dimensional structure. In some embodiments, at least a portion of the non-nucleic acid based polymer analyte is denatured. In some embodiments, the linear length of the non-nucleic acid based polymer analyte is greater than the channel length of the nanopore transversing the membrane when the non-nucleic acid based polymer analyte is elongated. In some embodiments, the non-nucleic acid based polymer analyte comprises at least about 25 repeating units.

In some embodiments, the non-nucleic acid based polymer analyte comprises peptide units, saccharide units, water-soluble plastic monomers, or any combination thereof. In some embodiments, the non-nucleic acid based polymer analyte comprises a polypeptide, a polysaccharide, or a water-soluble plastic. In some embodiments, the non-nucleic acid based polymer analyte comprises a polypeptide of at least 30 peptide units. In some embodiments, the at least 30 peptide units comprise positively or negatively charged peptide units. In some embodiments, the polypeptide is in a denatured state. In some embodiments, the polypeptide is provided in a folded state. In some embodiments, the linear length of the non-nucleic acid based polymer analyte is at least 1 kDa. In some embodiments, the linear length of the non-nucleic acid based polymer analyte is at most 4,000 kDa. In some embodiments, the linear length of the non-nucleic acid based polymer analyte is at least 2 times greater than the channel length of the nanopore. In some embodiments, the linear length of the non-nucleic acid based polymer analyte is at most 2 times greater than the channel length of the nanopore.

In another aspect, the present disclosure provides a device comprising an array of a system comprising any of the systems disclosed herein.

In another aspect, the present disclosure provides a use of any of the methods, kits, or devices disclosed herein for characterizing at least one feature of the non-nucleic acid based polymer analyte.

In another aspect, the present disclosure provides a use of any of the systems disclosed herein for characterizing at least one feature of the non-nucleic acid based polymer analyte.

In another aspect, the present disclosure provides a use of any of the methods, kits, or devices disclosed herein for detection and analysis of one or more non-nucleic acid based polymer analytes at a single molecule level.

In another aspect, the present disclosure provides a use of any of the systems disclosed herein for detection and analysis of one or more non-nucleic acid based polymer analytes at a single molecule level.

In another aspect, the present disclosure provides a use of any of the methods, kits, or devices disclosed herein for detection and analysis of one or more polypeptides.

In another aspect, the present disclosure provides a use of any of the systems disclosed herein for detection and analysis of one or more polypeptides.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Another aspect of the present disclosure provides a method for translocating a non-nucleic acid based polymer analyte through a nanopore, the nanopore being comprised in a membrane separating a fluidic chamber of a nanopore system into a cis side and a trans side, comprising adding the polymer analyte to the cis side of and allowing for polymer analyte translocation, wherein the length of the elongated polymer analyte is larger than the longitudinal axis of the central channel of the nanopore in the direction perpendicular to the membrane,

and wherein the nanopore system has a cis to trans electro-osmotic force (EOF) resulting from a net ionic current flow cis to trans, and wherein the cis to trans EOF overcomes a trans to cis electrophoretic force (EPF) acting on the polymer analyte.

In some embodiments, the polymer analyte is an unmodified (label-free) analyte.

In some embodiments of any one of the preceding embodiments, the termini of the polymer are unstructured, preferably wherein the polymer is denatured or partially denatured.

In some embodiments of any one of the preceding embodiments, the polymer analyte comprises at least 25 repeating units, preferably at least 35 repeating units, more preferably at least 45 repeating units.

In some embodiments of any one of the preceding embodiments, the polymer analyte is of synthetic, semi-synthetic or biological origin, such as a biopolymer, preferably comprising or consisting of peptide units, saccharide units and water-soluble plastic monomers, and any combination thereof. In some embodiments, the polymer analyte is a polypeptide, polysaccharide, or a water-soluble plastic, such as PEG, or a PEGylated polypeptide. In some embodiments, the polymer analyte is a polypeptide of at least 30 peptide units and comprising positively and negatively charged residues. In some embodiments, the polypeptide is in a denatured/unfolded state, preferably wherein the polypeptide is added in a pre-denatured state.

In some embodiments of any one of the preceding embodiments, further comprising (c) measuring ionic current changes caused by translocation of the target polymer through the nanopore, preferably wherein operation (c) comprises measuring current changes for states of (i) open channel, (ii) capture of the polymer by the nanopore, and (iii) passage of a polymer from (ii) through the nanopore, more preferably wherein the measuring comprises detecting differences between states (i), (ii) and (iii).

Another aspect of the present disclosure provides a nanopore system for translocating a polymer analyte through a nanopore, the system comprising a nanopore comprised in a membrane separating a fluidic chamber of the nanopore system into a cis side and a trans side and wherein the analyte is to be added to the cis side, nanopore system has a cis to trans electro-osmotic force (EOF) resulting from a net ionic current flow cis to trans, and wherein the cis to trans EOF overcomes a trans to cis electrophoretic force (EPF) acting on the polymer analyte.

In some embodiments of any one of the preceding embodiments, the nanopore system has a cis to trans EOF resulting from a net ionic current flow cis-to-trans over total ionic current flow of greater than 0.2 or less than −0.2, preferably greater than 0.3 or less than −0.3, more preferably greater than 0.35 or less than −0.35.

In some embodiments of any one of the preceding embodiments, the cis to trans EOF is arranged by modulating the pH, type and/or concentration of a salt and/or osmotic pressure across the membrane of the nanopore system, by modification (e.g. genetic engineering) of the nanopore charge, or any combination thereof.

In some embodiments of any one of the preceding embodiments, the cis to trans EOF is arranged by modification of the nanopore and/or asymmetric salt distribution between the cis and trans side of the chamber.

In some embodiments of any one of the preceding embodiments, the nanopore system has an ion-selectivity P₍₊₎/P₍₋₎ of greater than 2.0 or less than 0.5, preferably greater than 2.5 or less than 0.4, most preferably greater than 3.0 or less than 0.33.

In some embodiments of any one of the preceding embodiments, the system has an ion-selectivity P(+)/P(−) of greater than 2.0 preferably greater than 2.5, more preferably greater than 3.0 and wherein there is a negative applied voltage at the trans side, preferably wherein the system comprises a cation-selective (mutant) nanopore.

In some embodiments of any one of the preceding embodiments, the nanopore is a biological nanopore, preferably having an inner pore constriction in the range of 0.5-2 nm.

In some embodiments of any one of the preceding embodiments, the nanopore is an alpha-helical or beta-barrel oligomeric pore forming toxin or porin, preferably wherein the nanopore is selected from the group consisting of Aerolysin (Aer), Cytolysin K (CytK), MspA, alpha-hemolysin (aHL), CsgG, Fragaceatoxin C (FraC), Lysenin, phage derived portal proteins, and modified variants thereof, or an ion-selective mutant thereof.

In some embodiments of any one of the preceding embodiments, the nanopore comprises a biological nanopore that is modified, e.g. by genetic engineering, to provide the desired ion selectivity, preferably wherein the ion-selective nanopore is modified to have a net charge in the lumen facing regions of >21, preferably >28, more preferably >35, most preferably wherein said net charge is negative.

In some embodiments of any one of the preceding embodiments, the nanopore is a mutant CytK nanopore comprising one or more of the amino acid substitutions selected from the group consisting of K128D, K155Q, T116D, S120D, Q122D, S126D, T143D, Q145D, T147D and S151D, wherein the numbering corresponds to the CytK amino acid available under accession number A0A2S1A9G3_9BACI in UniProt, preferably wherein the CytK mutant nanopore comprises one of the following combinations of amino acid substitutions: K128D and K155D; K128D, K155D and T116D, optionally further comprising T147D and/or S151D; K128D, K155D and S120D, optionally further comprising Q122D, T147D and/or S155D; K128D, K155D, Q145D and S151D.

Another aspect of the present disclosure provides an analytical device comprising an array of nanopore systems according to any one of the preceding embodiments,

Another aspect of the present disclosure provides the use of a method, nanopore system, or device according to any one of the preceding embodiments for characterizing at least one feature of a target polymer, preferably for detection and analysis of one or more target polymer(s) at the single molecule level, more preferably for detection and analysis of one or more a target polypeptide(s).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

In some embodiments, methods are provided relating to the analysis of an analyte.

One aspect of the present disclosure provides a method comprising providing a nanopore system. The nanopore system can comprise a fluidic chamber. The fluidic chamber can be separated into a cis side and a trans side. The method can further comprise providing a non-nucleic acid based polymer analyte. In some cases, the non-nucleic acid based polymer analyte can comprise a linear length greater than a channel length of the nanopore. The method can further comprise translocating the non-nucleic acid based polymer analyte. In some cases, the non-nucleic acid based polymer analyte can be translocated from the cis side to the trans side of the fluidic chamber. The non-nucleic acid based polymer analyte can comprise an elongated structure. In some cases, the nanopore system can have a cis to trans electro-osmotic force. The electro-osmotic force can comprise a cis to trans net ionic current flow. In some cases, the cis to trans electro-osmotic force can translocate the non-nucleic acid based polymer analyte through the nanopore against an electrophoretic force. In some cases, the electrophoretic case can act in a direction opposite of the cis to trans electro-osmotic force.

One aspect of the present disclosure provides a system comprising a nanopore system. The nanopore system can comprise a fluidic chamber. The system can also comprise a membrane. In some cases, the membrane can comprise a nanopore. In some cases, the membrane can separate the fluidic chamber into a cis side and a trans side. The fluidic chamber can be separated into a cis side and a trans side. In some cases, the cis side can comprise a first solution. In some cases, the trans side can comprise a second solution. In some cases, the cis side can comprise a first solution and the trans side can comprise a second solution. The system can further comprise a non-nucleic acid based polymer analyte. The first solution and the second solution can be configured to translocate the non-nucleic acid based polymer analyte across the nanopore using an electro-osmotic flow. In some cases, the non-nucleic acid based polymer analyte can have an elongated structure. In some cases, the non-nucleic acid based polymer analyte can have a linear length greater than a channel length of the nanopore. The system can further comprise a pair of electrodes. In some cases, the pair of electrodes can comprise a first electrode and a second electrode. The first electrode can be disposed on the cis side of the fluidic chamber. The second electrode can be disposed on the trans side of the fluidic chamber. The first electrode can be disposed on the cis side of the fluidic chamber and the second electrode can be disposed on the trans side of the fluidic chamber. In some cases, the pair of electrodes can be configured to generate an electrophoretic force. In some cases, the electrophoretic force can act in an opposite direction to the electro-osmotic flow.

In some embodiments, a change in ionic current can be measured while the analyte translocates through the nanopore. In some cases, the change in ionic current can be measured by a voltage based chip. In some cases, the voltage based chip can measure the voltage and/or change in current across the nanopore. In some cases, the voltage based chip can be a trans electrode.

The characterisation methods may involve measuring the ion current flow through the pore, typically by measurement of a current. Alternatively, the ion flow through the pore may be measured optically, such as disclosed by Heron etal: J. Am. Chem. Soc. 9 Vol. 131, No. 5, 2009. Therefore the apparatus may also comprise an electrical circuit capable of applying a potential and measuring an electrical signal across the membrane and pore. The characterisation methods may be carried out using a patch clamp or a voltage clamp. The characterization methods can involve the use of a voltage clamp. In some embodiments, a spacer can be attached to the analyte.

In some embodiments, an analyte comprises a polymer analyte. The analyte can comprise a nucleic acid based polymer analyte or a non-nucleic acid based polymer analyte. The analyte can be of synthetic, semi-synthetic, or biological origin. For example, a synthetic analyte may comprise an analyte constructed by a non-biological chemical process, such as polyethylene glycol (PEG), synthetically constructed peptides of proteins, or a synthetically constructed DNA molecule. A biological analyte can comprise an analyte produced by a biological process, such as a protein produced by a cell or by systems employing cellular (or cellular derived) components (e.g. enzymatic in vitro translation systems). A semi-synthetic analyte can comprise portions created by biological and non-biological origins, for example, a biologically-produced protein conjugated to a PEG molecule. Possible electrical measurements can include current measurements, impedance measurements, tunneling, electron tunneling measurements (Ivanov A P et al., Nano Lett. 2011 Jan. 12; 11(1):279-85), FET measurements (International Application WO 2005/124888), voltage FET measurements, or any combination thereof. In some embodiments, the signal may be electron tunneling across a solid state nanopore or a voltage FET measurement across a solid state nanopore.

The characterisation methods may involve measuring the ion current flow through the pore, by measurement of a current. Alternatively, the ion flow through the pore may be measured optically, such as disclosed by Heron et al: J. Am. Chem. Soc. 9 Vol. 131, No. 5, 2009. Therefore the apparatus may also comprise an electrical circuit capable of applying a potential and measuring an electrical signal across the membrane and pore. The characterisation methods may be carried out using a patch clamp or a voltage clamp. The characterisation methods preferably involve the use of a voltage clamp.

The characterisation methods may be carried out on an array of wells or nanopore channels where each array comprises 128, 256, 512, 1024, 2000, 3000, 4000, 6000, 10000, 12000, 15000 or more wells or nanopore channels.

The characterisation methods may involve the measuring of a current flowing through the pore. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, 20 mV, +50 mV, +100 mV, +150 mV, +200 mV, +300 mV and +400 mV. The voltage used is more preferably in the range mV to 240 mV and most preferably in the range of 120 mV to 220 mV. It is possible to increase discrimination between different nucleotides by a pore by using an increased applied potential.

In some embodiments, an analyte comprises a protein or peptide. A protein or peptide can comprise a folded state, an unfolded state, or intermediate states thereof. A folded state comprises a state of a protein or peptide in which the polymer is at a low-energy state such that the protein or peptide maintains a two or three dimensional structure. This low-energy state can be based on the interactions of the amino acids of the peptide or protein with each other. An unfolded state can comprise a state of a protein or peptide in which the polymer is at a high-energy state such that the protein or peptide does not maintain a two or three dimensional structure. An intermediate state between a folded and unfolded state can be an energy state at which a portion or portions of the peptide or protein may maintain a two or three dimensional structure, and other portions of the peptide or protein do not maintain a two or three dimensional structure.

In some embodiments, the analyte can comprise a non-nucleic acid based polymer analyte. In some embodiments, a portion of non-nucleic acid based polymer analyte can comprise a nucleic acid molecule. In some cases, the portion of the non-nucleic acid polymer analyte can be from 0% to about 100% of the non-nucleic acid polymer analyte. In some cases, the portion of the non-nucleic acid polymer analyte can be at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of the non-nucleic acid polymer analyte. In some cases, the portion of the non-nucleic acid polymer analyte can be at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, or at most about 0% of the non-nucleic acid polymer analyte. In some cases, the portion of the non-nucleic acid polymer analyte can be about 0%, about 5%, about 10%, about 15%, about 20%, about 25%, about 30%, about 35%, about 40%, about 45%, about 50%, about 55%, about 60%, about 65%, about 75%, about 80%, about 85%, about 90%, about 95%, or about 100% of the non-nucleic acid polymer analyte.

In some embodiments, a portion of non-nucleic acid based polymer analyte can comprise an oligosaccharide molecule. In some cases, the portion of the non-nucleic acid polymer analyte can be from 0% to about 100% of the non-nucleic acid polymer analyte. In some cases, the portion of the non-nucleic acid polymer analyte can be at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of the non-nucleic acid polymer analyte. In some cases, the portion of the non-nucleic acid polymer analyte can be at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, or at most about 0% of the non-nucleic acid polymer analyte. In some cases, the portion of the non-nucleic acid polymer analyte can be about 0%, about 5%, about 10%, about 15%, about 20%, about 25%, about 30%, about 35%, about 40%, about 45%, about 50%, about 55%, about 60%, about 65%, about 75%, about 80%, about 85%, about 90%, about 95%, or about 100% of the non-nucleic acid polymer analyte.

The analyte can comprise a contour length. In some embodiments, the contour length can comprise the length of the analyte when the analyte is not completely unfolded. In some cases, the analyte can be between 1% to about 100% unfolded. In some cases, the analyte can be at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% unfolded. In some cases, the analyte can be at most about 100%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, or less than 1% unfolded. In some cases, the analyte can be about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% unfolded. In some cases, the linear length of the analyte can be when the analyte is 100% unfolded. The analyte can comprise a linear length. In some embodiments, the contour length of the analyte can be the linear length of the analyte. The linear length can be the length of an analyte in an unfolded state. In some embodiments, the linear length of the analyte can be from about 3 nanometers (nm) to about 5,000 nm. In some embodiments, the linear length of the analyte can be from about 3 to about 5 nm, from about 5 nm to about 10 nm, from about 10 nm to about 15 nm, from about 15 nm to about 20 nm, from about 20 nm to about 25 nm, from about 25 nm to about 30 nm, from about 30 nm to about 35 nm, from about 35 nm to about 40 nm, from about 40 nm to about 45 nm, from about 45 nm to about 50 nm, from about 50 nm to about 55 nm, from about 55 nm to about 60 nm, from about 60 nm to about 65 nm, from about 65 nm to about 70 nm, from about 70 nm to about 75 nm, from about 75 nm to about 80 nm, from about 80 nm to about 85 nm, from about 85 nm to about 90 nm, from about 90 nm to about 95 nm, from about 95 nm to about 100 nm, from about 100 nm to about 150 nm, from about 150 nm to about 200 nm, from about 200 nm to about 250 nm, from about 250 nm to about 300 nm, from about 300 nm to about 350 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to about 700 nm, from about 700 nm to about 750 nm, from about 750 nm to about 800 nm, from about 800 nm to about 850 nm, from about 850 nm to about 900 nm, from about 900 nm to about 950 nm, from about 950 nm to about 1,000 nm, from about 1,000 nm to about 1,100 nm, from about 1,100 nm to about 1,200 nm, from about 1,200 nm to about 1,300 nm, from about 1,300 nm to about 1,400 nm, from about 1,400 nm to about 1,500 nm, from about 1,500 nm to about 1,600 nm, from about 1,600 nm to about 1,700 nm, from about 1,700 nm to about 1,800 nm, from about 1,800 nm to about 1,900 nm, from about 1,900 nm to about 2,000 nm, from about 2,000 nm to about 2,100 nm, from about 2,100 nm to about 2,200 nm, from about 2,200 nm to about 2,300 nm, from about 2,300 nm to about 2,400 nm, from about 2,400 to about 2,500 nm, from about 2,500 nm to about 2,600 nm, from about 2,600 nm to about 2,700 nm, from about 2,700 nm to about 2,800 nm, from about 2,800 nm to about 2,900 nm, from about 2,900 nm to about 3,000 nm, from about 3,000 nm to about 3,100 nm, from about 3,200 nm to about 3,300 nm, from about 3,300 nm to about 3,400 nm, from about 3,400 nm to about 3,500 nm, from about 3,500 to about 3,600 nm, from about 3,600 nm to about 3,700 nm, from about 3,700 nm to about 3,800 nm, from about 3,800 nm to about 3,900 nm, from about 3,900 nm to about 4,000 nm, from about 4,000 nm to about 4,100 nm, from about 4,100 to about 4,200 nm, from about 4,200 nm to about 4,300 nm, from about 4,300 nm to about 4,400 nm, from about 4,400 nm to about 4,500 nm, from about 4,500 nm to about 4,600 nm, from about 4,600 nm to about 4,700 nm, from about 4,700 nm to about 4,800 nm, from about 4,800 nm to about 4,900 nm, or from about 4,900 nm to about 5,000 nm.

In some embodiments, the linear length of the analyte can be at least about 3 nm, at least about 4 nm, at least about 5 nm, at least about 10 nm, at least about 15 nm, at least about 20 nm, at least about 25 nm, at least about 30 nm, at least about 35 nm, at least about 40 nm, at least about 45 nm, at least about 50 nm, at least about 55, at least about 60 nm, at least about 65 nm, at least about 70 nm, at least about 75 nm, at least about 80 nm, at least about 85 nm, at least about 90 nm, at least about 95 nm, at least about 100 nm, at least about 110 nm, at least about 120 nm, at least about 130 nm, at least about 140 nm, at least about 150 nm, at least about 160 nm, at least about 170 nm, at least about 180 nm, at least about 190 nm, at least about 200 nm, at least about 210 nm, at least about 220 nm, at least about 230 nm, at least about 240 nm, at least about 250 nm, at least about 260 nm, at least about 270 nm, at least about 280 nm, at least about 290, at least about 300 nm, at least about 310 nm, at least about 320 nm, at least about 330 nm, at least about 340 nm, at least about 350 nm, at least about 360 nm, at least about 370 nm, at least about 380 nm, at least about 390 nm, at least about 400 nm, at least about 410 nm, at least about 420 nm, at least about 430 nm, at least about 440 nm, at least about 450 nm, at least about 460 nm, at least about 470 nm, at least about 480 nm, at least about 490 nm, at least about 500 nm, at least about 510 nm, at least about 520 nm, at least about 530 nm, at least about 540 nm, at least about 550 nm, at least about 560 nm, at least about 570 nm, at least about 580 nm, at least about 590 nm, at least about 600 nm, at least about 610 nm, at least about 620 nm, at least about 630 nm, at least about 640 nm, at least about 650 nm, at least about 660 nm, at least about 670 nm, at least about 680 nm, at least about 690 nm, at least about 700 nm, at least about 710 nm, at least about 720 nm, at least about 730 nm, at least about 740 nm, at least about 750 nm, at least about 760 nm, at least about 770 nm, at least about 780 nm, at least about 790 nm, at least about 800 nm, at least about 810 nm, at least about 820 nm, at least about 830 nm, at least about 840 nm, at least about 850 nm, at least about 860 nm, at least about 870 nm, at least about 880 nm, at least about 890 nm, at least about 900 nm, at least about 910 nm, at least about 920 nm, at least about 930 nm, at least about 940 nm, at least about 950 nm, at least about 960 nm, at least about 970 nm, at least about 980 nm, at least about 990 nm, at least about 1,000 nm, at least about 1,100 nm, at least about 1,200 nm, at least about 1,300 nm, at least about 1,400 nm, at least about 1,500 nm, at least about 1,600 nm, at least about 1,700 nm, at least about 1,800 nm, at least about 1,900 nm, at least about 2,000 nm, at least about 2,100 nm, at least about 2,200 nm, at least about 2,300 nm, at least about 2,400 nm, at least about 2,500 nm, at least about 2,600 nm, at least about 2,700 nm, at least about 2,800 nm, at least about 2,900 nm, at least about 3,000 nm, at least about 3,100 nm, at least about 3,200 nm, at least about 3,300 nm, at least about 3,400 nm, at least about 3,500 nm, at least about 3,600 nm, at least about 3,700 nm, at least about 3,800 nm, at least about 3,900 nm, at least about 4,000 nm, at least about 4,100 nm, at least about 4,200 nm, at least about 4,300 nm, at least about 4,400 nm, at least about 4,500 nm, at least about 4,600 nm, at least about 4,700 nm, at least about 4,800 nm, at least about 4,900 nm, at least about 5,000 nm, or greater than about 5,000 nm.

In some embodiments, the linear length of the analyte can be at most about 5,000 nm, at most about 4,900 nm, at most about 4,800 nm, at most about 4,700 nm, at most about 4,600 nm, at most about 4,500 nm, at most about 4,400 nm, at most about 4,300 nm, at most about 4,200 nm, at most about 4,100 nm, at most about 4,000 nm, at most about 3,900 nm, at most about 3,800 nm, at most about 3,700 nm, at most about 3,600 nm, at most about 3,500 nm, at most about 3,400 nm, at most about 3,300 nm, at most about 3,200 nm, at most about 3,100 nm, at most about 3,000 nm, at most about 2,900 nm, at most about 2,800 nm, at most about 2,700 nm, at most about 2,600 nm, at most about 2,500 nm, at most about 2,400 nm, at most about 2,300 nm, at most about 2,200 nm, at most about 2,100 nm, at most about 2,000 nm, at most about 1,900 nm, at most about 1,800 nm, at most about 1,700 nm, at most about 1,600 nm, at most about 1,500 nm, at most about 1,400 nm, at most about 1,300 nm, at most about 1,200, at most about 1,100, at most about 1,000 nm, at most about 990, at most about 980 nm, at most about 970 nm, at most about 960 nm, at most about 950 nm, at most about 940 nm, at most about 930 nm, at most about 920 nm, at most about 910 nm, at most about 900 nm, at most about 890 nm, at most about 880 nm, at most about 870 nm, at most about 860 nm, at most about 850 nm, at most about 840 nm, at most about 830 nm, at most about 820 nm, at most about 810 nm, at most about 800 nm, at most about 790 nm, at most about 780 nm, at most about 770 nm, at most about 760 nm, at most about 750 nm, at most about 740 nm, at most about 730 nm, at most about 720 nm, at most about 710 nm, at most about 700 nm, at most about 690 nm, at most about 680 nm, at most about 670 nm, at most about 660 nm, at most about 650 nm, at most about 640 nm, at most about 630 nm, at most about 620 nm, at most about 610 nm, at most about 600 nm, at most about 590 nm, at most about 580 nm, at most about 570 nm, at most about 560 nm, at most about 550 nm, at most about 540 nm, at most about 530 nm, at most about 520 nm, at most about 510 nm, at most about 500 nm, at most about 490 nm, at most about 480 nm, at most about 470 nm, at most about 460 nm, at most about 450 nm, at most about 440 nm, at most about 430 nm, at most about 420 nm, at most about 410 nm, at most about 400 nm, at most about 390 nm, at most about 380 nm, at most about 370 nm, at most about 360 nm, at most about 350 nm, at most about 340 nm, at most about 330 nm, at most about 320 nm, at most about 310 nm, at most about 300 nm, at most about 290 nm, at most about 280 nm, at most about 270 nm, at most about 260 nm, at most about 250 nm, at most about 240 nm, at most about 230 nm, at most about 220 nm, at most about 210 nm, at most about 200 nm, at most about 190 nm, at most about 180 nm, at most about 170 nm, at most about 160 nm, at most about 150 nm, at most about 140 nm, at most about 130 nm, at most about 120 nm, at most about 110 nm, at most about 100 nm, at most about 95 nm, at most about 90 nm, at most about 85 nm, at most about 80 nm, at most about 75 nm, at most about 70 nm, at most about 65 nm, at most about 60 nm, at most about 55 nm, at most about 50 nm, at most about 45 nm, at most about 40 nm, at most about 35 nm, at most about 30 nm, at most about 25 nm, at most about 20 nm, at most about 15 nm, at most about 10 nm, at most about 5 nm, at most about 3 nm, or less than about 3 nm.

In some embodiments, the linear length of the analyte can be about 3 nm, about 4 nm, about 5 nm, about 10 nm, about 15 nm, about 20 nm, about 25 nm, about 30 nm, about 35 nm, about 40 nm, about 45 nm, about 50 nm, about 55, about 60 nm, about 65 nm, about 70 nm, about 75 nm, about 80 nm, about 85 nm, about 90 nm, about 95 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, about 650 nm, about 660 nm, about 670 nm, about 680 nm, about 690 nm, about 700 nm, about 710 nm, about 720 nm, about 730 nm, about 740 nm, about 750 nm, about 760 nm, about 770 nm, about 780 nm, about 790 nm, about 800 nm, about 810 nm, about 820 nm, about 830 nm, about 840 nm, about 850 nm, about 860 nm, about 870 nm, about 880 nm, about 890 nm, about 900 nm, about 910 nm, about 920 nm, about 930 nm, about 940 nm, about 950 nm, about 960 nm, about 970 nm, about 980 nm, about 990 nm, about 1,000 nm, about 1,100 nm, about 1,200 nm, about 1,300 nm, about 1,400 nm, about 1,500 nm, about 1,600 nm, about 1,700 nm, about 1,800 nm, about 1,900 nm, about 2,000 nm, about 2,100 nm, about 2,200 nm, about 2,300 nm, about 2,400 nm, about 2,500 nm, about 2,600 nm, about 2,700 nm, about 2,800 nm, about 2,900 nm, about 3,000 nm, about 3,100 nm, about 3,200 nm, about 3,300 nm, about 3,400 nm, about 3,500 nm, about 3,600 nm, about 3,700 nm, about 3,800 nm, about 3,900 nm, about 4,000 nm, about 4,100 nm, about 4,200 nm, about 4,300 nm, about 4,400 nm, about 4,500 nm, about 4,600 nm, about 4,700 nm, about 4,800 nm, about 4,900 nm, or about 5,000 nm.

An analyte in an unfolded state may or may not comprise secondary structure elements. Secondary structure elements may include α-helices, β-helices, coils, or β-sheets. Helices may be left handed or right handed. An analyte may in an unfolded state may be fully or partially unfolded. The contour length can be the length of a polymer analyte when two termini of the polymer analyte are fully extended from each other. Alternatively, in some cases, the contour length of an analyte when two termini of the analyte are not fully extended from each other. In some embodiments, an analyte can comprise a structured portion, an unstructured portion, a denatured portion, a partially denatured portion, or combinations thereof. In some embodiments, an analyte comprises at least about 1, at least about 2, at least about 3, at least about 4, at least about 5, or at least about 6 termini. In some embodiments, a terminus of an analyte can comprise a structured portion, an unstructured portion, a denatured portion, a partially denatured portion, or combinations thereof.

The analyte can comprise repeating units. In some embodiments, the analyte can comprise from about 2 to about 100 repeating units. In some cases, the analytes can comprise from about 2 to about 5 repeating units, from about 5 to about 10 repeating units, from about 10 to about 15 repeating units, from about 15 to about 20 repeating units, from about 20 to about 25 repeating units, from about 25 to about 30 repeating units, from about 30 to about 35 repeating units, from about 35 to about 40 repeating units, from about 40 to about 45 repeating units, from about 45 to about 50 repeating units, from about 50 to about 55 repeating units, from about 55 to about 60 repeating units, from about 60 to about 65 repeating units, from about 65 to about 70 repeating units, from about 70 to about 75 repeating units, from about 75 to about 80 repeating units, from about 80 to about 85 repeating units, from about 85 to about 90 repeating units, from about 90 to about 95 repeating units, or from about 95 to about 100 repeating units.

In some embodiments, the analyte can comprise at least about 2 repeating units, at least about 3 repeating units, at least about 4 repeating units, at least about 5 repeating units, at least about 10 repeating units, at least about 15 repeating units, at least about 20 repeating units, at least about 25 repeating units, at least about 30 repeating units, at least about 35 repeating units, at least about 40 repeating units, at least about 45 repeating units, at least about 50 repeating units, at least about 55 repeating units, at least about 60 repeating units, at least about 65 repeating units, at least about 70 repeating units, at least about 75 repeating units, at least about 80 repeating units, at least about 85 repeating units, at least about 90 repeating units, at least about 95 repeating units, at least about 100 repeating units, or more than 100 repeating units. In some embodiments, the analyte can comprise at most about 100 repeating units, at most about 95 repeating units, at most about 90 repeating units, at most about 85 repeating units, at most about 80 repeating units, at most about 75 repeating units, at most about 70 repeating units, at most about 65 repeating units, at most about 60 repeating units, at most about 55 repeating units, at most about 50 repeating units, at most about 45 repeating units, at most about 40 repeating units, at most about 35 repeating units, at most about 30 repeating units, at most about 25 repeating units, at most about 20 repeating units, at most about 15 repeating units, at most about 10 repeating units, at most about 5 repeating units, at most about 4 repeating units, at most about 3 repeating units, at most about 2 repeating units, or less than 2 repeating units. In some embodiments, the analyte can comprise about 2 repeating units, about 3 repeating units, about 4 repeating units, about 5 repeating units, about 10 repeating units, about 15 repeating units, about 20 repeating units, about 25 repeating units, about 30 repeating units, about 35 repeating units, about 40 repeating units, about 45 repeating units, about 50 repeating units, about 55 repeating units, about 60 repeating units, about 65 repeating units, about 70 repeating units, at least about 75 repeating units, at least about 80 repeating units, about 85 repeating units, about 90 repeating units, about 95 repeating units, or about 100 repeating units.

The units can comprise peptide units, saccharide units, lipid units, nucleotides, water-soluble plastic monomers, or combinations thereof. The analyte can comprise a polypeptide, a polysaccharide, a lipid, a nucleic acid, a water-soluble plastic, or combinations thereof. In some embodiments, the analyte can comprise a charge. The charge can be positive or negative. The charge can be distributed evenly or unevenly across the analyte. In some embodiments, the charge can be the result of an amino acid residue. The amino acid residue can be a natural or mutated residue. In some cases, a mutated residue can be a point mutation in the analyte. In some cases, the mutated residue can be a residue that differs from a wild-type sequence of the analyte. In some embodiments the analyte can comprise a peptide. A peptide can comprise a polypeptide or protein. A protein can be a full length protein or a truncated protein. A truncated protein (e.g., a peptide) can be a protein that is shorter in length than when the protein was first made. For example, the protein can be shorter due to cleavage (e.g., by a peptidase) or degradation (e.g., due to acidic or basic conditions). A protein can comprise a sequence that is a native protein sequence or a modified protein sequence. The sequence can be modified by mutation, deletion, or insertion of a sequence. A sequence can be a combination of sequences. For example, first native sequence can be appended to or inserted into a second native sequence to form a third sequence that is a combination of the first and second sequences.

In another aspect, the present disclosure provides systems for determining one or more characteristics of an analyte. In some embodiments, the system comprises a fluidic chamber. In some embodiments, the system comprises a membrane. The membrane can divide the fluidic chamber into two or more sides. The membrane can divide the fluidic chamber into a cis side and a trans side. The cis side can comprise a fluidic solution. The trans side can comprise a fluidic solution. The fluidic solutions can be configured to provide an electro-osmotic flow, also termed an electro-osmotic force. The electro-osmotic force can act across the membrane. In some embodiments, the membrane comprises a nanopore. In some embodiments a pair of electrodes is provided. The pair of electrodes can be disposed with one electrode on a cis side of the fluidic chamber, and the other electrode on the trans side of the fluidic chamber. In some embodiments, an electrophoretic force is provided.

In another aspect, the present disclosure provides methods for determining one or more characteristics of an analyte. In some embodiments, the method comprises translocating an analyte through the nanopore. The translocation can be assisted by the electro-osmotic force, the electrophoretic force, or combinations thereof. The translocation can be opposed by the electro-osmotic force, the electrophoretic force, or combinations thereof. In some embodiments, the analyte is in a pre-denatured state prior to translocation. In some embodiments, the analyte can be translocated through the nanopore in an elongated form. In some cases, the elongated form of the analyte may not comprise a three-dimensional structure. In some cases, the elongated form of the analyte can be in a completely linear structure. In some cases, the elongated form of the analyte can be in a linear structure. In some cases, the analyte can be translocated through the nanopore in a folded structure. In some cases, an analyte in a folded structure can comprise a non-elongated analyte. In some cases, the non-elongated structure may not comprise any three-dimensional structures in the analyte. In some cases, an analyte with a folded structure can comprise a three-dimensional structure. In some embodiments, the method comprises measuring a signal. The signal can be caused or influenced by the translocation of the analyte. In some embodiments, one or more analytes are translocated. The signals of one or more translocated analytes may be measured.

In some embodiments, the translocation of the analyte through the nanopore occurs in a cis to trans direction. In some embodiments, the translocation of the analyte through the nanopore occurs in a trans to cis direction. In some embodiments, the translocation of the analyte through the nanopore occurs in the direction of the electro-osmotic force (EOF). In some embodiments, the translocation of the analyte through the nanopore occurs in the opposite direction of the electrophoretic force (EPF). In some embodiments, the translocation of the analyte through the nanopore occurs in the direction of the EOF or opposite the direction of the EPF.

In some embodiments, the EOF can be greater than the EPF. In some cases, the EOF is from about 0.1% to about 500% greater than the EPF. In some cases, the EOF is from about 0.1% to about 0.5%, from about 0.5% to about 1%, from about 1% to about 5%, from about 5% to about 10%, from about 10% to about 20%, from about 20% to about 30%, from about 30% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, from about 95% to about 100%, from about 100% to about 110%, from about 110% to about 120%, from about 120% to about 130%, from about 130% to about 140%, from about 140% to about 150%, from about 150% to about 160%, from about 160% to about 170%, from about 170% to about 180%, from about 180% to about 190%, from about 190% to about 200%, from about 200% to about 210%, from about 210% to about 220%, from about 220% to about 230%, from about 230% to about 240%, from about 240% to about 250%, from about 250% to about 260%, from about 260% to about 270%, from about 270% to about 280%, from about 280% to about 290%, from about 290% to about 300%, from about 300% to about 310%, from about 310% to about 320%, from about 320% to about 330%, from about 330% to about 340%, from about 340% to about 350%, from about 350% to about 360%, from about 360% to about 370%, from about 370% to about 380%, from about 380% to about 390%, from about 390% to about 400%, from about 400% to about 410%, from about 410% to about 420%, from about 420% to about 430%, from about 430% to about 440%, from about 440% to about 450%, from about 450% to about 460%, from about 460% to about 470%, from about 470% to about 480%, from about 480% to about 490%, or from about 490% to about 500% longer greater than the EPF.

In some cases, the EOF can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% greater than the EPF.

In some cases, the EOF can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% greater than the EPF.

In some cases, the EOF can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% greater than the EPF.

In some embodiments, the translocation of the analyte through the nanopore occurs in the direction of the EOF. In some embodiments, the translocation of the analyte through the nanopore occurs in the direction of the EPF. In some embodiments, the translocation of the analyte through the nanopore occurs in the direction of the EOF or the direction of the EPF.

Alternatively, in some embodiments, the translocation of the analyte through the nanopore occurs in the direction of the EPF. In some embodiments, the translocation of the analyte through the nanopore occurs in the opposite direction of the EOF. In some embodiments, the translocation of the analyte through the nanopore occurs in the direction of the EPF or opposite the direction of the EOF.

Alternatively, in some embodiments, the EPF can be greater than the EOF. In some embodiments, the EPF can be greater than the EOF. In some cases, the EPF is from about 0.1% to about 500% greater than the EOF. In some cases, the EPF is from about 0.1% to about 0.5%, from about 0.5% to about 1%, from about 1% to about 5%, from about 5% to about 10%, from about 10% to about 20%, from about 20% to about 30%, from about 30% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, from about 95% to about 100%, from about 100% to about 110%, from about 110% to about 120%, from about 120% to about 130%, from about 130% to about 140%, from about 140% to about 150%, from about 150% to about 160%, from about 160% to about 170%, from about 170% to about 180%, from about 180% to about 190%, from about 190% to about 200%, from about 200% to about 210%, from about 210% to about 220%, from about 220% to about 230%, from about 230% to about 240%, from about 240% to about 250%, from about 250% to about 260%, from about 260% to about 270%, from about 270% to about 280%, from about 280% to about 290%, from about 290% to about 300%, from about 300% to about 310%, from about 310% to about 320%, from about 320% to about 330%, from about 330% to about 340%, from about 340% to about 350%, from about 350% to about 360%, from about 360% to about 370%, from about 370% to about 380%, from about 380% to about 390%, from about 390% to about 400%, from about 400% to about 410%, from about 410% to about 420%, from about 420% to about 430%, from about 430% to about 440%, from about 440% to about 450%, from about 450% to about 460%, from about 460% to about 470%, from about 470% to about 480%, from about 480% to about 490%, or from about 490% to about 500% longer greater than the EOF.

In some cases, the EPF can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% greater than the EOF.

In some cases, the EPF can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% greater than the EOF.

In some cases, the EPF can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% greater than the EOF.

In some embodiments, the translocation of the analyte through the nanopore can occur in the absence of one or more assisting proteins. In some embodiments, the translocation of the analyte through the nanopore can occur in the presence of the EOF. In some cases, the translocation of the analyte can occur in the presence of the EOF and in the absence of the one or more assisting proteins. In some cases, the one or more assisting proteins may be capable of moving an analyte through the nanopore. In some cases, the one or more assisting proteins can comprise translocases, helicases, unfoldases, DNA polymerases, RNA polymerases, topoisomerases, or any combinations thereof.

In some embodiments, a nanopore comprises a biological nanopore or a solid state nanopore. A biological nanopore can comprise a mutation to a portion of the biological nanopore. The mutation can comprise an insert, a substitution, a deletion, or combinations thereof. In some embodiments, the nanopore can comprise a recombinant nanopore. In some cases, the recombinant nanopore can comprise components from one or more different types of nanopores. In some embodiments, the nanopore can be modified to limit passage of one or more ions through the channel of the nanopore. In some cases, the nanopore can limit the passage of one or more ions through the channel of the nanopore by modifying a charge of the channel of the nanopore. In some cases, the nanopore can be modified to have a net negative charged channel. In some cases, a net negative charged channel can limit the passage of one or more anions through the channel of the nanopore. In some cases, the nanopore can be modified to have a net positive charged channel. In some cases, a net positive charged channel can limit the passage of one or more cations through the channel of the nanopore. In some cases, the charge of the nanopore can be modified at the cis entrance of the channel. In some cases, the charge of the nanopore can be modified at the trans entrance of the channel. In some cases, the charge of the nanopore can be modified in a central channel of the nanopore.

A nanopore can comprise a geometry. The geometry can comprise a toroidal shape, comprising a ring or a channel. The toroidal shape may comprise a toroidal polyhedral shape comprising a ring or a channel. The ring may comprise the protein or proteins that form the nanopore. The ring may comprise a cross sectional geometry similar to the protein or proteins that form the nanopore. The ring may be wider at the cis side than the trans side, or wider at the trans side than the cis side. The ring can comprise a portion comprising a conical geometry, a cylindrical geometry, an amorphous geometry, or combinations thereof. The channel can comprise the central portion of the nanopore geometry that does not comprise the proteins or peptides of the nanopore. The channel may allow molecules to pass through the nanopore (e.g., through the channel). A channel may restrict molecules from passing through the nanopore. The restriction may be based on a width of the channel or a charge of the channel. The channel can comprise a channel length. The channel length can be the length of the channel as measured along a longitudinal axis of the channel, perpendicular to the ring of the toroidal shape of the geometry of the nanopore. The channel length can be measured as the distance along the longitudinal axis of the channel between the most distant points of the nanopore along the longitudinal axis of the channel. In some embodiment, a channel may have a start point on a cis side of a nanopore, and an end point on a trans side of a nanopore, or a start point on a trans side of a nanopore, and an end point on a cis side of a nanopore. In some embodiments a channel length is less than a linear length or a contour length of an analyte. In some embodiments a channel length is greater than a linear length or a contour length of an analyte. In some embodiments, a channel comprises a channel length of from about 2-40 nm. In some embodiments, the channel comprises a channel length of from about 2-5 nm, from about 5-10 nm, from about 10-15 nm, from about 15-20 nm, from about 20-25 nm, from about 25-30 nm, from about 30-35 nm, or from about 35-40 nm. In some cases, the channel comprises a channel length of at least about 2 nm, at least about 3 nm, at least about 4 nm, at least about 5 nm, at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, at least about 20 nm, at least about 21 nm, at least about 22 nm, at least about 23 nm, at least about 24 nm, at least about 25 nm, at least about 26 nm, at least about 27 nm, at least about 28 nm, at least about 29 nm, at least about 30 nm, at least about 31 nm, at least about 32 nm, at least about 33 nm, at least about 34 nm, at least about 35 nm, at least about 36 nm, at least about 37 nm, at least about 38 nm, at least about 39 nm, at least about 40 nm, or more than 40 nm. In some cases, the channel comprises a channel length of at most about 40 nm, at most about 39 nm, at most about 38 nm, at most about 37 nm, at most about 36 nm, at most about 35 nm, at most about 34 nm, at most about 33 nm, at most about 32 nm, at most about 31 nm, at most about 30 nm, at most about 29 nm, at most about 28 nm, at most about 27 nm, at most about 26 nm, at most about 25 nm, at most about 24 nm, at most about 23 nm, at most about 22 nm, at most about 21 nm, at most about 20 nm, at most about 19 nm, at most about 18 nm, at most about 17, at most about 16 nm, at most about 15 nm, at most about 14 nm, at most about 13 nm, at most about 12 nm, at most about 11 nm, at most about 10 nm, at most about 9 nm, at most about 8 nm, at most about 7 nm, at most about 6 nm, at most about 5 nm, at most about 4 nm, at most about 3 nm, at most about 2 nm, or less than 2 nm. In some cases, the channel comprises a channel of about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 39 nm, or about 40 nm.

In some embodiments, the inner nanopore channel can comprise a lumen. In some cases, the lumen of the nanopore channel can be from about 0.5 nm to about 10 nm. In some cases, the lumen of the nanopore channel can be at least about 0.5 nm, at least about 1.0 nm, at least about 1.5 nm, at least about 2.0 nm, at least about 2.5 nm, at least about 3.0 nm, at least about 3.5 nm, at least about 4.0 nm, at least about 4.5 nm, at least about 5.0 nm, at least about 5.5 nm, at least about 6.0 nm, at least about 6.5 nm, at least about 7.0 nm, at least about 7.5 nm, at least about 8.0 nm, at least about 8.5 nm, at least about 9.0 nm, at least about 9.5 nm, at least about 10.0 nm, or greater than 10.0 nm. In some cases, the lumen of the nanopore channel can be at most about 10.0 nm, at most about 9.5 nm, at most about 9.0 nm, at most about 8.5 nm, at most about 8.0 nm, at most about 7.5 nm, at most about 7.0 nm, at most about 6.5 nm, at most about 6.0 nm, at most about 5.5 nm, at most about 5.0 nm, at most about 4.5 nm, at most about 4.0 nm, at most about 3.5 nm, at most about 3.0 nm, at most about 2.5 nm, at most about 2.0 nm, at most about 1.5 nm, at most about 1.0 nm, at most about 0.5 nm, or less than 0.5 nm. In some cases, the lumen of the nanopore channel can be about 0.5 nm, about 1.0 nm, about 1.5 nm, about 2.0 nm, about 2.5 nm, about 3.0 nm, about 3.5 nm, about 4.0 nm, about 4.5 nm, about 5.0 nm, about 5.5 nm, about 6.0 nm, about 6.5 nm, about 7.0 nm, about 7.5 nm, about 8.0 nm, about 8.5, about 9.0 nm, about 9.5 nm, or about 10.0 nm.

In some embodiments, the inner nanopore channel can comprise one or more constrictions. In some cases, the inner nanopore channel can comprise from about one constriction to about 50 constrictions. In some cases, the inner nanopore channel can comprise at least about one constriction, at least about five constrictions, at least about 10 constrictions, at least about 15 constrictions, at least about 20 constrictions, at least about 25 constrictions, at least about 30 constrictions, at least about 25 constrictions, at least about 30 constrictions, at least about 35 constrictions, at least about 40 constrictions, at least about 45 constrictions, at least about 50 constrictions, or more than 50 constrictions. In some cases, the inner nanopore channel can comprise at most about 50 constrictions, at most about 45 constrictions, at most about 40 constrictions, at most about 35 constrictions, at most about 30 constrictions, at most about 25 constrictions, at most about 20 constrictions, at most about 15 constrictions, at most about 10 constrictions, at most about 5 constrictions, at most about one constriction, or less than one constriction. In some cases, the inner nanopore channel can comprise about one constriction, about five constrictions, about 10 constrictions, about 15 constrictions, about 20 constrictions, about 25 constrictions, about 30 constrictions, about 35 constrictions, about 40 constrictions, about 45 constrictions, or about 50 constrictions.

In some embodiments, the one or more constrictions can be from about 0.2 nm to about 2 nm in size. In some cases, the one or more constrictions can be at least about 0.2 nm at least about 0.3 nm, at least about 0.4 nm, at least about 0.5 nm, at least about 0.6 nm, at least about 0.7 nm, at least about 0.8 nm, at least about 0.9 nm, at least about 1.0 nm, at least about 1.1 nm, at least about 1.2 nm, at least about 1.3 nm, at least about 1.4 nm, at least about 1.5 nm, at least about 1.6 nm, at least about 1.7 nm, at least about 1.8 nm, at least about 1.9 nm, at least about 2.0 nm, or greater than 2.0 nm in size. In some cases, the one or more constrictions can be at most about 2.0 nm, at most about 1.9 nm, at most about 1.8 nm, at most about 1.7 nm, at most about 1.6 nm, at most about 1.5 nm, at most about 1.4 nm, at most about 1.3 nm, at most about 1.2 nm, at most about 1.1 nm, at most about 1.0 nm, at most about 0.9 nm, at most about 0.8 nm, at most about 0.7 nm, at most about 0.6 nm, at most about 0.5 nm, at most about 0.4 nm, at most about 0.3 nm, at most about 0.2 nm, or less than 0.2 nm in size. In some cases, the one or more constrictions can be about 0.2 nm, about 0.3 nm, about 0.4 nm, about 0.5 nm, about 0.6 nm, about 0.7 nm, about 0.8 nm, about 0.9 nm, about 1.0 nm, about 1.1 nm, about 1.2 nm, about 1.3 nm, about 1.4 nm, about 1.5 nm, about 1.6 nm, about 1.7 nm, about 1.8 nm, about 1.9 nm, or about 2.0 nm in size.

In some embodiments, the analyte is longer than the length of a channel of the nanopore. In some embodiments, the analyte can be at least about 2 times, at least about 3 times, at least about 4 times, at least about 5 times, at least about 6 times, at least about 7 times, at least about 8 times, at least about 9 times, at least about 10 times, at least about 12 times, at least about 13 times, at least about 14 times, at least about 15 times, at least about 16 times, at least about 17 times, at least about 18 times, at least about 19 times, at least about 20 times, at least about 25 times, at least about 30 times, at least about 35 times, at least about 40 times, at least about 45 times, at least about 50 times, at least about 55 times, at least about 60 times, at least about 65 times, at least about 70 times, at least about 75 times, at least about 80 times, at least about 85 times, at least about 90 times, at least about 95 times, at least about 100 times, or greater than about 100 times the channel length of the nanopore. In some embodiments, the analyte can be at most about 100 times, at most about 95 times, at most about 90 times, at most about 80 times, at most about 75 times, at most about 70 times, at most about 65 times, at most about 60 times, at most about 55 times, at most about 50 times, at most about 45 times, at most about 40 times, at most about 35 times, at most about 30 times, at most about 25 times, at most about 20 times, at most about 19 times, at most about 18 times, at most about 17 times, at most about 16 times, at most about 15 times, at most about 14 times, at most about 13 times, at most about 12 times, at most about 11 times, at most about 10 times, at most about 9 times, at most about 8 times, at most about 7 times, at most about 6 times, at most about 5 times, at most about 4 times, at most about 3 times, at most about 2 times, or less than about 2 times the channel length of the nanopore.

In some embodiments, the analyte can be from about 2 times to about 100 times the channel length of the nanopore. In some embodiments, the analyte can be from about 2 times to about 5 times, about 2 times to about 10 times, about 2 times to about 20 times, about 2 times to about 30 times, about 2 times to about 40 times, about 2 times to about 50 times, about 2 times to about 60 times, about 2 times to about 70 times, about 2 times to about 80 times, about 2 times to about 90 times, about 2 times to about 100 times, about 5 times to about 10 times, about 5 times to about 20 times, about 5 times to about 30 times, about 5 times to about 40 times, about 5 times to about 50 times, about 5 times to about 60 times, about 5 times to about 70 times, about 5 times to about 80 times, about 5 times to about 90 times, about 5 times to about 100 times, about 10 times to about 20 times, about 10 times to about 30 times, about 10 times to about 40 times, about 10 times to about 50 times, about 10 times to about 60 times, about 10 times to about 70 times, about 10 times to about 80 times, about 10 times to about 90 times, about 10 times to about 100 times, about 20 times to about 30 times, about 20 times to about 40 times, about 20 times to about 50 times, about 20 times to about 60 times, about 20 times to about 70 times, about 20 times to about 80 times, about 20 times to about 90 times, about 20 times to about 100 times, about 30 times to about 40 times, about 30 times to about 50 times, about 30 times to about 60 times, about 30 times to about 70 times, about 30 times to about 80 times, about 30 times to about 90 times, about 30 times to about 100 times, about 40 times to about 50 times, about 40 times to about 60 times, about 40 times to about 70 times, about 40 times to about 80 times, about 40 times to about 90 times, about 40 times to about 100 times, about 50 times to about 60 times, about 50 times to about 70 times, about 50 times to about 80 times, about 50 times to about 90 times, about 50 times to about 100 times, about 60 times to about 70 times, about 60 times to about 80 times, about 60 times to about 90 times, about 60 times to about 100 times, about 70 times to about 80 times, about 70 times to about 90 times, about 70 times to about 100 times, about 80 times to about 90 times, about 80 times to about 100 times, or about 90 times to about 100 times the channel length of the nanopore.

In some embodiments, the analyte can be at least about 2 times, about 3 times, about 4 times, about 5 times, about 6 times, about 7 times, about 8 times, about 9 times, about 10 times, about 12 times, about 13 times, about 14 times, about 15 times, about 16 times, about 17 times, about 18 times, about 19 times, about 20 times, about 25 times, about 30 times, about 35 times, about 40 times, about 45 times, about 50 times, about 55 times, about 60 times, about 65 times, about 70 times, about 75 times, about 80 times, about 85 times, about 90 times, about 95 times, or about 100 times the channel length of the nanopore.

In some embodiments, the linear length of the analyte is greater than the channel length of the nanopore. In some cases, the linear length of the analyte is from about 0.1% to about 500% longer than the channel length of the nanopore. In some embodiments, the linear length of the analyte is from about 0.10% to about 0.5%, from about 0.5% to about 10%, from about 10% to about 5%, from about 5% to about 10%, from about 10% to about 20%, from about 20% to about 30%, from about 30% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, from about 95% to about 100%, from about 100% to about 110%, from about 110% to about 120%, from about 120% to about 130%, from about 130% to about 140%, from about 140% to about 150%, from about 150% to about 160%, from about 160% to about 170%, from about 170% to about 180%, from about 180% to about 190%, from about 190% to about 200%, from about 200% to about 210%, from about 210% to about 220%, from about 220% to about 230%, from about 230% to about 240%, from about 240% to about 250%, from about 250% to about 260%, from about 260% to about 270%, from about 270% to about 280%, from about 280% to about 290%, from about 290% to about 300%, from about 300% to about 310%, from about 310% to about 320%, from about 320% to about 330%, from about 330% to about 340%, from about 340% to about 350%, from about 350% to about 360%, from about 360% to about 370%, from about 370% to about 380%, from about 380% to about 390%, from about 390% to about 400%, from about 400% to about 410%, from about 410% to about 420%, from about 420% to about 430%, from about 430% to about 440%, from about 440% to about 450%, from about 450% to about 460%, from about 460% to about 470%, from about 470% to about 480%, from about 480% to about 490%, or from about 490% to about 500% longer than the channel length of the nanopore.

In some cases, the linear length of the analyte is at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% longer than the channel length of the nanopore.

In some cases, the linear length of the analyte is at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% longer than the channel length of the nanopore.

In some cases, the linear length of the analyte is about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% longer than the channel length of the nanopore.

In some embodiments, the linear length of the analyte is from about 1 nm to about 5,000 nm longer than the channel length of the nanopore. In some embodiments, the linear length of the analyte can be from about 0.1 nm to about 0.5 nm, from about 0.5 nm to about 1 nm, from about 1 nm to about 3 nm, from about 3 to about 5 nm, from about 5 nm to about 10 nm, from about 10 nm to about 15 nm, from about 15 nm to about 20 nm, from about 20 nm to about 25 nm, from about 25 nm to about 30 nm, from about 30 nm to about 35 nm, from about 35 nm to about 40 nm, from about 40 nm to about 45 nm, from about 45 nm to about 50 nm, from about 50 nm to about 55 nm, from about 55 nm to about 60 nm, from about 60 nm to about 65 nm, from about 65 nm to about 70 nm, from about 70 nm to about 75 nm, from about 75 nm to about 80 nm, from about 80 nm to about 85 nm, from about 85 nm to about 90 nm, from about 90 nm to about 95 nm, from about 95 nm to about 100 nm, from about 100 nm to about 150 nm, from about 150 nm to about 200 nm, from about 200 nm to about 250 nm, from about 250 nm to about 300 nm, from about 300 nm to about 350 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to about 700 nm, from about 700 nm to about 750 nm, from about 750 nm to about 800 nm, from about 800 nm to about 850 nm, from about 850 nm to about 900 nm, from about 900 nm to about 950 nm, from about 950 nm to about 1,000 nm, from about 1,000 nm to about 1,100 nm, from about 1,100 nm to about 1,200 nm, from about 1,200 nm to about 1,300 nm, from about 1,300 nm to about 1,400 nm, from about 1,400 nm to about 1,500 nm, from about 1,500 nm to about 1,600 nm, from about 1,600 nm to about 1,700 nm, from about 1,700 nm to about 1,800 nm, from about 1,800 nm to about 1,900 nm, from about 1,900 nm to about 2,000 nm, from about 2,000 nm to about 2,100 nm, from about 2,100 nm to about 2,200 nm, from about 2,200 nm to about 2,300 nm, from about 2,300 nm to about 2,400 nm, from about 2,400 to about 2,500 nm, from about 2,500 nm to about 2,600 nm, from about 2,600 nm to about 2,700 nm, from about 2,700 nm to about 2,800 nm, from about 2,800 nm to about 2,900 nm, from about 2,900 nm to about 3,000 nm, from about 3,000 nm to about 3,100 nm, from about 3,200 nm to about 3,300 nm, from about 3,300 nm to about 3,400 nm, from about 3,400 nm to about 3,500 nm, from about 3,500 to about 3,600 nm, from about 3,600 nm to about 3,700 nm, from about 3,700 nm to about 3,800 nm, from about 3,800 nm to about 3,900 nm, from about 3,900 nm to about 4,000 nm, from about 4,000 nm to about 4,100 nm, from about 4,100 to about 4,200 nm, from about 4,200 nm to about 4,300 nm, from about 4,300 nm to about 4,400 nm, from about 4,400 nm to about 4,500 nm, from about 4,500 nm to about 4,600 nm, from about 4,600 nm to about 4,700 nm, from about 4,700 nm to about 4,800 nm, from about 4,800 nm to about 4,900 nm, or from about 4,900 nm to about 5,000 nm longer than the channel length of the nanopore.

In some embodiments, the analyte can be present in the cis side of the nanopore system. In some embodiments, the analyte can be present in the trans side of the nanopore system. In some embodiments, the analyte can be present in the channel of the nanopore. In some embodiments, the analyte can be present in the cis side of the nanopore system and present in the channel of the nanopore at the same time. In some embodiments, the analyte can be present in trans side of the nanopore system and present in the channel of the nanopore at the same time. In some embodiments, the analyte can be present in the cis side of the nanopore system, present in the channel of the nanopore, and present in the trans side of the nanopore system at the same time.

In some embodiments, the linear length of the analyte can be at least about 0.1 nm, at least about 0.5 nm, at least about 1 nm, at least about 2 nm, at least about 3 nm, at least about 4 nm, at least about 5 nm, at least about 10 nm, at least about 15 nm, at least about 20 nm, at least about 25 nm, at least about 30 nm, at least about 35 nm, at least about 40 nm, at least about 45 nm, at least about 50 nm, at least about 55, at least about 60 nm, at least about 65 nm, at least about 70 nm, at least about 75 nm, at least about 80 nm, at least about 85 nm, at least about 90 nm, at least about 95 nm, at least about 100 nm, at least about 110 nm, at least about 120 nm, at least about 130 nm, at least about 140 nm, at least about 150 nm, at least about 160 nm, at least about 170 nm, at least about 180 nm, at least about 190 nm, at least about 200 nm, at least about 210 nm, at least about 220 nm, at least about 230 nm, at least about 240 nm, at least about 250 nm, at least about 260 nm, at least about 270 nm, at least about 280 nm, at least about 290, at least about 300 nm, at least about 310 nm, at least about 320 nm, at least about 330 nm, at least about 340 nm, at least about 350 nm, at least about 360 nm, at least about 370 nm, at least about 380 nm, at least about 390 nm, at least about 400 nm, at least about 410 nm, at least about 420 nm, at least about 430 nm, at least about 440 nm, at least about 450 nm, at least about 460 nm, at least about 470 nm, at least about 480 nm, at least about 490 nm, at least about 500 nm, at least about 510 nm, at least about 520 nm, at least about 530 nm, at least about 540 nm, at least about 550 nm, at least about 560 nm, at least about 570 nm, at least about 580 nm, at least about 590 nm, at least about 600 nm, at least about 610 nm, at least about 620 nm, at least about 630 nm, at least about 640 nm, at least about 650 nm, at least about 660 nm, at least about 670 nm, at least about 680 nm, at least about 690 nm, at least about 700 nm, at least about 710 nm, at least about 720 nm, at least about 730 nm, at least about 740 nm, at least about 750 nm, at least about 760 nm, at least about 770 nm, at least about 780 nm, at least about 790 nm, at least about 800 nm, at least about 810 nm, at least about 820 nm, at least about 830 nm, at least about 840 nm, at least about 850 nm, at least about 860 nm, at least about 870 nm, at least about 880 nm, at least about 890 nm, at least about 900 nm, at least about 910 nm, at least about 920 nm, at least about 930 nm, at least about 940 nm, at least about 950 nm, at least about 960 nm, at least about 970 nm, at least about 980 nm, at least about 990 nm, at least about 1,000 nm, at least about 1,100 nm, at least about 1,200 nm, at least about 1,300 nm, at least about 1,400 nm, at least about 1,500 nm, at least about 1,600 nm, at least about 1,700 nm, at least about 1,800 nm, at least about 1,900 nm, at least about 2,000 nm, at least about 2,100 nm, at least about 2,200 nm, at least about 2,300 nm, at least about 2,400 nm, at least about 2,500 nm, at least about 2,600 nm, at least about 2,700 nm, at least about 2,800 nm, at least about 2,900 nm, at least about 3,000 nm, at least about 3,100 nm, at least about 3,200 nm, at least about 3,300 nm, at least about 3,400 nm, at least about 3,500 nm, at least about 3,600 nm, at least about 3,700 nm, at least about 3,800 nm, at least about 3,900 nm, at least about 4,000 nm, at least about 4,100 nm, at least about 4,200 nm, at least about 4,300 nm, at least about 4,400 nm, at least about 4,500 nm, at least about 4,600 nm, at least about 4,700 nm, at least about 4,800 nm, at least about 4,900 nm, at least about 5,000 nm, or more than 5,000 nm longer than the channel length of the nanopore.

In some embodiments, the linear length of the analyte can be at most about 5,000 nm, at most about 4,900 nm, at most about 4,800 nm, at most about 4,700 nm, at most about 4,600 nm, at most about 4,500 nm, at most about 4,400 nm, at most about 4,300 nm, at most about 4,200 nm, at most about 4,100 nm, at most about 4,000 nm, at most about 3,900 nm, at most about 3,800 nm, at most about 3,700 nm, at most about 3,600 nm, at most about 3,500 nm, at most about 3,400 nm, at most about 3,300 nm, at most about 3,200 nm, at most about 3,100 nm, at most about 3,000 nm, at most about 2,900 nm, at most about 2,800 nm, at most about 2,700 nm, at most about 2,600 nm, at most about 2,500 nm, at most about 2,400 nm, at most about 2,300 nm, at most about 2,200 nm, at most about 2,100 nm, at most about 2,000 nm, at most about 1,900 nm, at most about 1,800 nm, at most about 1,700 nm, at most about 1,600 nm, at most about 1,500 nm, at most about 1,400 nm, at most about 1,300 nm, at most about 1,200, at most about 1,100, at most about 1,000 nm, at most about 990, at most about 980 nm, at most about 970 nm, at most about 960 nm, at most about 950 nm, at most about 940 nm, at most about 930 nm, at most about 920 nm, at most about 910 nm, at most about 900 nm, at most about 890 nm, at most about 880 nm, at most about 870 nm, at most about 860 nm, at most about 850 nm, at most about 840 nm, at most about 830 nm, at most about 820 nm, at most about 810 nm, at most about 800 nm, at most about 790 nm, at most about 780 nm, at most about 770 nm, at most about 760 nm, at most about 750 nm, at most about 740 nm, at most about 730 nm, at most about 720 nm, at most about 710 nm, at most about 700 nm, at most about 690 nm, at most about 680 nm, at most about 670 nm, at most about 660 nm, at most about 650 nm, at most about 640 nm, at most about 630 nm, at most about 620 nm, at most about 610 nm, at most about 600 nm, at most about 590 nm, at most about 580 nm, at most about 570 nm, at most about 560 nm, at most about 550 nm, at most about 540 nm, at most about 530 nm, at most about 520 nm, at most about 510 nm, at most about 500 nm, at most about 490 nm, at most about 480 nm, at most about 470 nm, at most about 460 nm, at most about 450 nm, at most about 440 nm, at most about 430 nm, at most about 420 nm, at most about 410 nm, at most about 400 nm, at most about 390 nm, at most about 380 nm, at most about 370 nm, at most about 360 nm, at most about 350 nm, at most about 340 nm, at most about 330 nm, at most about 320 nm, at most about 310 nm, at most about 300 nm, at most about 290 nm, at most about 280 nm, at most about 270 nm, at most about 260 nm, at most about 250 nm, at most about 240 nm, at most about 230 nm, at most about 220 nm, at most about 210 nm, at most about 200 nm, at most about 190 nm, at most about 180 nm, at most about 170 nm, at most about 160 nm, at most about 150 nm, at most about 140 nm, at most about 130 nm, at most about 120 nm, at most about 110 nm, at most about 100 nm, at most about 95 nm, at most about 90 nm, at most about 85 nm, at most about 80 nm, at most about 75 nm, at most about 70 nm, at most about 65 nm, at most about 60 nm, at most about 55 nm, at most about 50 nm, at most about 45 nm, at most about 40 nm, at most about 35 nm, at most about 30 nm, at most about 25 nm, at most about 20 nm, at most about 15 nm, at most about 10 nm, at most about 5 nm, at most about 3 nm, at most about 2 nm, at most about 1 nm, at most about 0.5 nm, at most about 0.1 nm, or less than 0.1 nm longer than the channel length of the nanopore.

In some embodiments, the linear length of the analyte can be about 0.1 nm, about 0.5 nm, about 1 nm, about 2 nm, about 3 nm, about 4 nm, about 5 nm, about 10 nm, about 15 nm, about 20 nm, about 25 nm, about 30 nm, about 35 nm, about 40 nm, about 45 nm, about 50 nm, about 55, about 60 nm, about 65 nm, about 70 nm, about 75 nm, about 80 nm, about 85 nm, about 90 nm, about 95 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, about 650 nm, about 660 nm, about 670 nm, about 680 nm, about 690 nm, about 700 nm, about 710 nm, about 720 nm, about 730 nm, about 740 nm, about 750 nm, about 760 nm, about 770 nm, about 780 nm, about 790 nm, about 800 nm, about 810 nm, about 820 nm, about 830 nm, about 840 nm, about 850 nm, about 860 nm, about 870 nm, about 880 nm, about 890 nm, about 900 nm, about 910 nm, about 920 nm, about 930 nm, about 940 nm, about 950 nm, about 960 nm, about 970 nm, about 980 nm, about 990 nm, about 1,000 nm, about 1,100 nm, about 1,200 nm, about 1,300 nm, about 1,400 nm, about 1,500 nm, about 1,600 nm, about 1,700 nm, about 1,800 nm, about 1,900 nm, about 2,000 nm, about 2,100 nm, about 2,200 nm, about 2,300 nm, about 2,400 nm, about 2,500 nm, about 2,600 nm, about 2,700 nm, about 2,800 nm, about 2,900 nm, about 3,000 nm, about 3,100 nm, about 3,200 nm, about 3,300 nm, about 3,400 nm, about 3,500 nm, about 3,600 nm, about 3,700 nm, about 3,800 nm, about 3,900 nm, about 4,000 nm, about 4,100 nm, about 4,200 nm, about 4,300 nm, about 4,400 nm, about 4,500 nm, about 4,600 nm, about 4,700 nm, about 4,800 nm, about 4,900 nm, or about 5,000 nm longer than the channel length of the nanopore.

In some embodiments the nanopore comprises a lumen. The lumen can be the surface of the nanopore that faces the channel. The lumen can comprise the surfaces of the nanopore's components that face the channel, including proteins, peptides, or amino acid residues that face the channel. These components can comprise a charge. The charge of these components can provide a net charge of the lumen. These components can have a shape. The shape of these components can provide a geometry of the lumen. The net charge of the lumen, the geometry of the lumen, or combinations thereof, can influence a flow of molecules through the lumen (e.g., through the nanopore).

In some embodiments, a pore comprises a lumen. In some embodiments, a nanopore lumen comprises a net charge of at least about 2, at least about 3, at least about 4, at least about 5, at least about 10, at least about 15, at least about 20, at least about 25, at least about 30, at least about 35, at least about 40, at least about 45, at least about 50, at least about 55, at least about 60, at least about 70, at least about 80, at least about 90, at least about 100, at least about 150, at least about 200, or greater than about 200. In some embodiments, a nanopore lumen comprises a net charge of at most about 200, at most about 150, at most about 100, at most about 90, at most about 80, at most about 70, at most about 60, at most about 55, at most about 50, at most about 45, at most about 40, at most about 35, at most about 30, at most about 25, at most about 20, at most about 15, at most about 10, at most about 5, at most about 4, at most about 3, at most about 2, or less than about 2.

In some embodiments, a nanopore lumen comprises a net charge from about 2 to about 200. In some embodiments, a nanopore lumen comprises a net charge from at most about 200. In some embodiments, a nanopore lumen comprises a net charge from about 2 to about 5, about 2 to about 10, about 2 to about 20, about 2 to about 30, about 2 to about 40, about 2 to about 50, about 2 to about 75, about 2 to about 100, about 2 to about 125, about 2 to about 150, about 2 to about 200, about 5 to about 10, about 5 to about 20, about 5 to about 30, about 5 to about 40, about 5 to about 50, about 5 to about 75, about 5 to about 100, about 5 to about 125, about 5 to about 150, about 5 to about 200, about 10 to about 20, about 10 to about 30, about 10 to about 40, about 10 to about 50, about 10 to about 75, about 10 to about 100, about 10 to about 125, about 10 to about 150, about 10 to about 200, about 20 to about 30, about 20 to about 40, about 20 to about 50, about 20 to about 75, about 20 to about 100, about 20 to about 125, about 20 to about 150, about 20 to about 200, about 30 to about 40, about 30 to about 50, about 30 to about 75, about 30 to about 100, about 30 to about 125, about 30 to about 150, about 30 to about 200, about 40 to about 50, about 40 to about 75, about 40 to about 100, about 40 to about 125, about 40 to about 150, about 40 to about 200, about 50 to about 75, about 50 to about 100, about 50 to about 125, about 50 to about 150, about 50 to about 200, about 75 to about 100, about 75 to about 125, about 75 to about 150, about 75 to about 200, about 100 to about 125, about 100 to about 150, about 100 to about 200, about 125 to about 150, about 125 to about 200, or about 150 to about 200.

In some embodiments, a nanopore lumen comprises a net charge of about 2, about 3, about 4, about 5, about 10, about 15, about 20, about 25, about 30, about 35, about 40, about 45, about 50, about 55, about 60, about 70, about 80, about 90, about 100, about 150, about 200. In some embodiments, a pore lumen comprises a net positive charge. In some embodiments, a pore lumen comprises a net negative charge.

In some embodiments, the analyte can lack a three-dimensional structure. In some cases, an analyte lacking three-dimensional structure can be a denatured analyte. In some embodiments, one or more portions of the analyte can lack a three-dimensional structure. In some cases, the one or more portions can comprise one or more termini of the analyte. In some cases, one of the analyte's termini can lack a three-dimensional structure. In some cases, two of the analyte's terminus can lack a three-dimensional structure. In some cases, at least two of the analyte's terminus can lack a three-dimensional structure. In some embodiments, the internal portion (e.g., portion of analyte that is not termini) can lack a three-dimensional structure. In some embodiments, the analyte can comprise a three-dimensional structure. In some cases, an analyte with a three-dimensional structure can be a folded analyte.

The flowing molecules can be analytes, ions, water, or other molecules on a cis or a trans side of a nanopore. The flowing molecules can generate an ionic current from a flow of ions. As an analyte passes through a pore, other molecules, such as ions, can be obstructed from passing through the pore. This can change the ionic current by changing the rate of flow of ions. This change can be measured, for example, by a pair of electrodes configured to measure a current from cis to trans across the nanopore, or the membrane the nanopore may be disposed. A narrow geometry of the lumen can slow a progression of an analyte through a pore. A change to a net charge or a geometry of a lumen can change the flow of molecules through a pore. For example, changing a lumen to have a more positive net charge can reduce a flow of a positively charged molecule (e.g., a sodium ion). For example, changing a lumen to have a wider geometry can increase a flow of a larger molecule (e.g., a glucose molecule or a peptide analyte). For example, changing a lumen to have a more negative net charge and a narrower geometry can reduce a flow of a large, negatively charged molecule (e.g., a glutamate ion). The net charge of the lumen can influence the flow of charged molecules through the nanopore. The net charge can make some charged molecules pass through more easily, or more difficultly.

A channel can comprise a constriction zone. A constriction zone can be a portion of the channel that is narrower than the surrounding section. A channel can comprise multiple constriction zones. The net charge of the lumen, the geometry of the lumen, or combinations thereof, can result in constriction zones. Modifications to the net charge of the lumen, the geometry of the lumen, or combinations thereof, can modify a characteristic of a constriction zone. A characteristic of a constriction zone may be a placement, a location, a width, a charge, or combinations thereof. For example, a change to a geometry of the lumen can change the width of a constriction zone, or a change to a net charge of the lumen can change the charge of a constriction zone.

A nanopore can comprise a permeability to an ion. A permeability to an ion can be the ability or likelihood of an ion to flow or diffuse through the channel of the nanopore. A permeability to an ion (P) may be different for different ions. By comparing different permeabilities, a relative ion selectivity can be generated. A relative ion selectivity can be the permeability of a given cation (P₍₊₎) (e.g., a potassium ion) divided by the permeability of a given anion (P₍₋₎) (e.g., a chlorine ion), to provide the relative ion selectivity (P₍₊₎/P₍₋₎). The net charge and the geometry of the lumen can each influence a permeability of an ion. For example, a positive net charge can reduce a permeability of a cation, or a negative net charge can reduce a permeability of an anion. A geometry of the lumen can influence a permeability of an ion based on the size of an ion. For example a smaller geometry can decrease the permeability of a larger ion (e.g. a potassium ion) relative to a smaller ion (e.g. a lithium ion).

The net charge and the geometry of the lumen can influence a relative ion flux. A relative ion flux can be the net flow of ions through the nanopore. In some embodiments, a nanopore can comprise a relative ion selectivity P₍₊₎/P₍₋₎ of greater than about 0.1, greater than about 0.2, greater than about 0.3, greater than about 0.4, greater than about 0.5, greater than about 0.6, greater than about 0.7, greater than about 0.8, greater than about 0.9, greater than about 1.0, greater than about 1.2, greater than about 1.4, greater than about 1.6, greater than about 1.8, greater than about 2.0, greater than about 2.5, greater than about 3, greater than about 3.2, greater than about 3.4, greater than about 3.6, greater than about 3.8, greater than about 4.0, greater than about 4.1, greater than about 4.2, greater than about 4.3, greater than about 4.4, greater than about 4.5, greater than about 4.6, greater than about 4.7, greater than about 4.8, greater than about 4.9, or greater than about 5.0. In some embodiments, a pore can comprise a relative ion selectivity P₍₊₎/P₍₋₎ of less than about 0.1, less than about 0.2, less than about 0.3, less than about 0.4, less than about 0.5, less than about 0.6, less than about 0.7, less than about 0.8, less than about 0.9, less than about 1.0, less than about 1.2, less than about 1.4, less than about 1.6, less than about 1.8, less than about 2.0, less than about 2.5, less than about 3, less than about 3.2, less than about 3.4, less than about 3.6, less than about 3.8, less than about 4.0, less than about 4.1, less than about 4.2, less than about 4.3, less than about 4.4, less than about 4.5, less than about 4.6, less than about 4.7, less than about 4.8, less than about 4.9, or less than about 5.0.

A nanopore can comprise at least one inner pore constriction. In some embodiments, the inner pore constriction is at least about 0.2, at least about 0.3, at least about 0.4, at least about 0.5, at least about 0.6, at least about 0.7, at least about 0.8, at least about 0.9, at least about 1.0, at least about 1.1, at least about 1.2, at least about 1.3, at least about 1.4, at least about 1.5, at least about 1.6, at least about 1.7, at least about 1.8, at least about 1.9, at least about 2.0, at least about 2.1, at least about 2.2, at least about 2.3, at least about 2.4, at least about 2.5, at least about 2.6, at least about 2.7, at least about 2.8, at least about 2.9, at least about 3.0, at least about 3.1, at least about 3.2, at least about 3.3, at least about 3.4, at least about 3.5, at least about 3.6, at least about 3.7, at least about 3.8, at least about 3.9, or at least about 4.0 nm. In some embodiments, the inner pore constriction is at most about 0.2, at most about 0.3, at most about 0.4, at most about 0.5, at most about 0.6, at most about 0.7, at most about 0.8, at most about 0.9, at most about 1.0, at most about 1.1, at most about 1.2, at most about 1.3, at most about 1.4, at most about 1.5, at most about 1.6, at most about 1.7, at most about 1.8, at most about 1.9, at most about 2.0, at most about 2.1, at most about 2.2, at most about 2.3, at most about 2.4, at most about 2.5, at most about 2.6, at most about 2.7, at most about 2.8, at most about 2.9, at most about 3.0, at most about 3.1, at most about 3.2, at most about 3.3, at most about 3.4, at most about 3.5, at most about 3.6, at most about 3.7, at most about 3.8, at most about 3.9, or at most about 4.0 nm.

In some embodiments, a nanopore channel comprises a lumen. In some embodiments, a nanopore lumen comprises a net charge of at least about 2 coulombs, at least about 3 coulombs, at least about 4 coulombs, at least about 5 coulombs, at least about 10 coulombs, at least about 15 coulombs, at least about 20 coulombs, at least about 25 coulombs, at least about 30 coulombs, at least about 35 coulombs, at least about 40 coulombs, at least about 45 coulombs, at least about 50 coulombs, at least about 55 coulombs, at least about 60 coulombs, at least about 70 coulombs, at least about 80 coulombs, at least about 90 coulombs, at least about 100 coulombs, at least about 150 coulombs, at least about 200 coulombs, or greater than about 200 coulombs. In some embodiments coulombs, a nanopore lumen comprises a net charge of at most about 200 coulombs, at most about 150 coulombs, at most about 100 coulombs, at most about 90 coulombs, at most about 80 coulombs, at most about 70 coulombs, at most about 60 coulombs, at most about 55 coulombs, at most about 50 coulombs, at most about 45 coulombs, at most about 40 coulombs, at most about 35 coulombs, at most about 30 coulombs, at most about 25 coulombs, at most about 20 coulombs, at most about 15 coulombs, at most about 10 coulombs, at most about 5 coulombs, at most about 4 coulombs, at most about 3 coulombs, at most about 2 coulombs, or less than about 2 coulombs.

In some embodiments, a nanopore lumen comprises a net charge from about 2 to about 200 coulombs. In some embodiments, a nanopore lumen comprises a net charge from at most about 200. In some embodiments, a nanopore lumen comprises a net charge from about 2 to about 5, about 2 to about 10, about 2 to about 20, about 2 to about 30, about 2 to about 40, about 2 to about 50, about 2 to about 75, about 2 to about 100, about 2 to about 125, about 2 to about 150, about 2 to about 200, about 5 to about 10, about 5 to about 20, about 5 to about 30, about 5 to about 40, about 5 to about 50, about 5 to about 75, about 5 to about 100, about 5 to about 125, about 5 to about 150, about 5 to about 200, about 10 to about 20, about 10 to about 30, about 10 to about 40, about 10 to about 50, about 10 to about 75, about 10 to about 100, about 10 to about 125, about 10 to about 150, about 10 to about 200, about 20 to about 30, about 20 to about 40, about 20 to about 50, about 20 to about 75, about 20 to about 100, about 20 to about 125, about 20 to about 150, about 20 to about 200, about 30 to about 40, about 30 to about 50, about 30 to about 75, about 30 to about 100, about 30 to about 125, about 30 to about 150, about 30 to about 200, about 40 to about 50, about 40 to about 75, about 40 to about 100, about 40 to about 125, about 40 to about 150, about 40 to about 200, about 50 to about 75, about 50 to about 100, about 50 to about 125, about 50 to about 150, about 50 to about 200, about 75 to about 100, about 75 to about 125, about 75 to about 150, about 75 to about 200, about 100 to about 125, about 100 to about 150, about 100 to about 200, about 125 to about 150, about 125 to about 200, or about 150 to about 200 coulombs.

In some embodiments, a nanopore lumen comprises a net charge of about 2 coulombs, about 3 coulombs, about 4 coulombs, about 5 coulombs, about 10 coulombs, about 15 coulombs, about 20 coulombs, about 25 coulombs, about 30 coulombs, about 35 coulombs, about 40 coulombs, about 45 coulombs, about 50 coulombs, about 55 coulombs, about 60 coulombs, about 70 coulombs, about 80 coulombs, about 90 coulombs, about 100 coulombs, about 150 coulombs, about 200 coulombs. In some embodiments, a nanopore lumen comprises a net positive charge. In some embodiments, a nanopore lumen comprises a net negative charge.

In some embodiments, the nanopore lumen can comprise a net charge from about −20 to about +20. In some cases, the nanopore lumen can comprise a net charge of at least about −20, at least about −19, at least about −18, at least about −17, at least about −16, at least about −15, at least about −14, at least about −13, at least about −12, at least about −11, at least about −10, at least about −9, at least about −8, at least about −7, at least about −6, at least about −5, at least about −4, at least about −3, at least about −2, at least about −1, at least about 0, at least about +1, at least about +2, at least about +3, at least about +4, at least about +5, at least about +6, at least about +7, at least about +8, at least about +9, at least about +10, at least about +11, at least about +12, at least about +13, at least about +14, at least about +15, at least about +16, at least about +17, at least about +18, at least about +19, at least about +20, or more than +20. In some cases, the nanopore lumen can comprise a net charge of at most about +20, at most about +19, at most about +18, at most about +17, at most about +16, at most about +15, at most about +14, at most about +13, at most about +12, at most about +11, at most about +10, at most about +9, at most about +8, at most about +7, at most about +6, at most about +5, at most about +4, at most about +3, at most about +2, at most about +1, at most about 0, at most about −1, at most about −2, at most about −3, at most about −4, at most about −5, at most about −6, at most about −7, at most about −8, at most about −9, at most about −10, at most about −11, at most about −12, at most about −13, at most about −14, at most about −15, at most about −16, at most about −17, at most about −18, at most about −19, at most about −20, or less than −20. In some cases, the nanopore lumen can comprise a net charge of about −20, about −19, about −18, about −17, about −16, about −15, about −14, about −13, about −12, about −11, about −10, about −9, about −8, about −7, about −6, about −5, about −4, about −3, about −2, about −1, about 0, about +1, about +2, about +3, about +4, about +5, about +6, about +7, about +8, about +9, about +10, about +11, about +12, about +13, about +14, about +15, about +16, about +17, about +18, about +19, or about +20.

In some embodiments, the nanopore can comprise one or more subunits. In some cases, each subunit of the one or more subunits can comprise from one to 20 charged amino acids. In some embodiments, from about 1 to 20 charged repeating units can be distributed within the lumen. In some cases, the charged repeating units can be negatively charged. In some cases, the charged repeating units can be positively charged. In some cases, the charged repeating units can be positively charged and negatively charged. In some cases, the lumen can comprise at least about 1 charged repeating unit, at least about 2 charged repeating units, at least about 3 charged repeating units, at least about 4 charged repeating units, at least about 5 charged repeating units, at least about 6 charged repeating units, at least about 7 charged repeating units, at least about 8 charged repeating units, at least about 9 charged repeating units, at least about 10 charged repeating units, at least about 11 charged repeating units, at least about 12 charged repeating units, at least about 13 charged repeating units, at least about 14 charged repeating units, at least about 15 charged repeating units, at least about 16 charged repeating units, at least about 17 charged repeating units, at least about 18 charged repeating units, at least about 19 charged repeating units, at least about 20 charged repeating units, or more than 20 charged repeating units. In some cases, the lumen can comprise at most about 20 charged repeating units, at most about 19 charged repeating units, at most about 18 charged repeating units, at most about 17 charged repeating units, at most about 16 charged repeating units, at most about 15 charged repeating units, at most about 14 charged repeating units, at most about 13 charged repeating units, at most about 12 charged repeating units, at most about 11 charged repeating units, at most about 10 charged repeating units, at most about 9 charged repeating units, at most about 8 charged repeating units, at most about 7 charged repeating units, at most about 6 charged repeating units, at most about 5 charged repeating units, at most about 4 charged repeating units, at most about 3 charged repeating units, at most about 2 charged repeating units, at most about 1 charged repeating unit, or less. In some cases, the lumen can comprise about 1 charged repeating unit, about 2 charged repeating units, about 3 charged repeating units, about 4 charged repeating units, about 5 charged repeating units, about 6 charged repeating units, about 7 charged repeating units, about 8 charged repeating units, about 9 charged repeating units, about 10 charged repeating units, about 11 charged repeating units, about 12 charged repeating units, about 13 charged repeating units, about 14 charged repeating units, about 15 charged repeating units, about 16 charged repeating units, about 17 charged repeating units, about 18 charged repeating units, about 19 charged repeating units, or about 20 charged repeating units. In some embodiments, the charged repeating units can be evenly distributed in the lumen. In some cases, the charged repeating units can be distributed every from about 2 repeating units to about 50 repeating units. In some cases, the charged repeating units can be distributed at least about every 2 repeating units, at least about every 5 repeating units, at least about every 10 repeating units, at least about every 15 repeating units, at least about every 20 repeating units, at least about every 25 repeating units, at least about every 30 repeating units, at least about every 40 repeating units, at least about every 45 repeating units, at least about every 50 repeating units, or more than every 50 repeating units. In some cases, the charged repeating units can be distributed at most about every 50 repeating units, at most about every 45 repeating units, at most about every 40 repeating units, at most about every 35 repeating units, at most about every 30 repeating units, at most about every 25 repeating units, at most about every 20 repeating units, at most about every 15 repeating units, at most about every 10 repeating units, at most about every 5 repeating units, at most about every 2 repeating units, or less than every 2 repeating units. In some cases, the charged repeating units can be distributed about every two repeating units, about every 5 repeating units, about every 10 repeating units, about every 15 repeating units, about every 20 repeating units, about every 25 repeating units, about every 30 repeating units, about every 35 repeating units, about every 30 repeating units, about every 35 repeating units, about every 40 repeating units, about every 45 repeating units, or about every 50 repeating units. In some embodiments, the charged repeating units can be unevenly distributed in the lumen.

The repeating units can be positively or negatively charged. Charged repeating units Asp, Glu, or Asp, can be readily introduced by single amino acid substitution(s). In some embodiments, the number of negatively charged amino acids that may be distributed evenly within the lumen can be at least about 1 amino acid, at least about 2 amino acids, at least about 3 amino acids, at least about 4 amino acids, at least about 5 amino acids, at least about 6 amino acids, at least about 7 amino acids, at least about 8 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, at least about 20 amino acids, or greater than about 20 charged amino acids. In some embodiments, the number of negatively charged amino acids that may be distributed evenly within the lumen can be at most about 20 amino acids, at most about 19 amino acids, at most about 18 amino acids, at most about 17 amino acids, at most about 16 amino acids, at most about 15 amino acids, at most about 14 amino acids, at most about 13 amino acids, at most about 12 amino acids, at most about 11 amino acids, at most about 10 amino acids, at most about 9 amino acids, at most about 8 amino acids, at most about 7 amino acids, at most about 6 amino acids, at most about 5 amino acids, at most about 4 amino acids, at most about 3 amino acids, at most about 2 amino acids, at most about 1 amino acid, or less than about 1 charged amino acid.

In some embodiments, the number of negatively charged amino acids that may be distributed evenly within the lumen can be from about 1 to about 20 charged amino acids. In some embodiments, the number of amino acids that may be distributed evenly within the lumen can be from about 1 to about 2, about 1 to about 3, about 1 to about 4, about 1 to about 5, about 1 to about 8, about 1 to about 9, about 1 to about 10, about 1 to about 12, about 1 to about 15, about 1 to about 18, about 1 to about 20, about 2 to about 3, about 2 to about 4, about 2 to about 5, about 2 to about 8, about 2 to about 9, about 2 to about 10, about 2 to about 12, about 2 to about 15, about 2 to about 18, about 2 to about 20, about 3 to about 4, about 3 to about 5, about 3 to about 8, about 3 to about 9, about 3 to about 10, about 3 to about 12, about 3 to about 15, about 3 to about 18, about 3 to about 20, about 4 to about 5, about 4 to about 8, about 4 to about 9, about 4 to about 10, about 4 to about 12, about 4 to about 15, about 4 to about 18, about 4 to about 20, about 5 to about 8, about 5 to about 9, about 5 to about 10, about 5 to about 12, about 5 to about 15, about 5 to about 18, about 5 to about 20, about 8 to about 9, about 8 to about 10, about 8 to about 12, about 8 to about 15, about 8 to about 18, about 8 to about 20, about 9 to about 10, about 9 to about 12, about 9 to about 15, about 9 to about 18, about 9 to about 20, about 10 to about 12, about 10 to about 15, about 10 to about 18, about 10 to about 20, about 12 to about 15, about 12 to about 18, about 12 to about 20, about 15 to about 18, about 15 to about 20, or about 18 to about 20 charged amino acids.

In some embodiments, the number of negatively charged amino acids that may be distributed evenly within the lumen can be about 1 amino acid, about 2 amino acids, about 3 amino acids, about 4 amino acids, about 5 amino acids, about 6 amino acids, about 7 amino acids, about 8 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, or about 20 charged amino acids. In some cases, the charged repeating units can be distributed every from about 2 repeating units to about 50 repeating units. In some cases, the charged repeating units can be distributed at least about every 2 repeating units, at least about every 5 repeating units, at least about every 10 repeating units, at least about every 15 repeating units, at least about every 20 repeating units, at least about every 25 repeating units, at least about every 30 repeating units, at least about every 40 repeating units, at least about every 45 repeating units, at least about every 50 repeating units, or more than every 50 repeating units. In some cases, the charged repeating units can be distributed at most about every 50 repeating units, at most about every 45 repeating units, at most about every 40 repeating units, at most about every 35 repeating units, at most about every 30 repeating units, at most about every 25 repeating units, at most about every 20 repeating units, at most about every 15 repeating units, at most about every 10 repeating units, at most about every 5 repeating units, at most about every 2 repeating units, or less than every 2 repeating units. In some cases, the charged repeating units can be distributed about every two repeating units, about every 5 repeating units, about every 10 repeating units, about every 15 repeating units, about every 20 repeating units, about every 25 repeating units, about every 30 repeating units, about every 35 repeating units, about every 30 repeating units, about every 35 repeating units, about every 40 repeating units, about every 45 repeating units, or about every 50 repeating units.

In some cases, the nanopore can comprise one or more subunits. In some cases, each subunit of the one or more subunits can have about 1 to about 20 charged amino acids. In some embodiments, each subunit of the one or more subunits can have from about 1 to about 2, about 1 to about 3, about 1 to about 4, about 1 to about 5, about 1 to about 8, about 1 to about 9, about 1 to about 10, about 1 to about 12, about 1 to about 15, about 1 to about 18, about 1 to about 20, about 2 to about 3, about 2 to about 4, about 2 to about 5, about 2 to about 8, about 2 to about 9, about 2 to about 10, about 2 to about 12, about 2 to about 15, about 2 to about 18, about 2 to about 20, about 3 to about 4, about 3 to about 5, about 3 to about 8, about 3 to about 9, about 3 to about 10, about 3 to about 12, about 3 to about 15, about 3 to about 18, about 3 to about 20, about 4 to about 5, about 4 to about 8, about 4 to about 9, about 4 to about 10, about 4 to about 12, about 4 to about 15, about 4 to about 18, about 4 to about 20, about 5 to about 8, about 5 to about 9, about 5 to about 10, about 5 to about 12, about 5 to about 15, about 5 to about 18, about 5 to about 20, about 8 to about 9, about 8 to about 10, about 8 to about 12, about 8 to about 15, about 8 to about 18, about 8 to about 20, about 9 to about 10, about 9 to about 12, about 9 to about 15, about 9 to about 18, about 9 to about 20, about 10 to about 12, about 10 to about 15, about 10 to about 18, about 10 to about 20, about 12 to about 15, about 12 to about 18, about 12 to about 20, about 15 to about 18, about 15 to about 20, or about 18 to about 20 charged amino acids.

In some embodiments, each subunit of the one or more subunits can have about 1 amino acid, about 2 amino acids, about 3 amino acids, about 4 amino acids, about 5 amino acids, about 6 amino acids, about 7 amino acids, about 8 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, or about 20 charged amino acids. In some cases, the charged repeating units can be distributed every from about 2 repeating units to about 50 repeating units. In some cases, the charged repeating units can be distributed at least about every 2 repeating units, at least about every 5 repeating units, at least about every 10 repeating units, at least about every 15 repeating units, at least about every 20 repeating units, at least about every 25 repeating units, at least about every 30 repeating units, at least about every 40 repeating units, at least about every 45 repeating units, at least about every 50 repeating units, or more than every 50 repeating units. In some cases, the charged repeating units can be distributed at most about every 50 repeating units, at most about every 45 repeating units, at most about every 40 repeating units, at most about every 35 repeating units, at most about every 30 repeating units, at most about every 25 repeating units, at most about every 20 repeating units, at most about every 15 repeating units, at most about every 10 repeating units, at most about every 5 repeating units, at most about every 2 repeating units, or less than every 2 repeating units. In some cases, the charged repeating units can be distributed about every two repeating units, about every 5 repeating units, about every 10 repeating units, about every 15 repeating units, about every 20 repeating units, about every 25 repeating units, about every 30 repeating units, about every 35 repeating units, about every 30 repeating units, about every 35 repeating units, about every 40 repeating units, about every 45 repeating units, or about every 50 repeating units.

At least 1 negatively charged “flanking” residue may be positioned or introduced at pore entry or at least 1 negatively charged residue may be positioned or introduced at pore exit. In some embodiments, the spacing between the Ca atom of the at least 1 internal negatively charged amino acid and the Ca atoms of the negatively charged flanking amino acid can be at least about 1 Å, at least about 2 Å, at least about 3 Å, at least about 4 Å, at least about 5 Å, at least about 6 Å, at least about 7 Å, at least about 8 Å, at least about 9 Å, at least about 10 Å, at least about 11 Å, at least about 12 Å, at least about 13 Å, at least about 14 Å, at least about 15 Å, at least about 16 Å, at least about 17 Å, at least about 18 Å, at least about 20 Å, at least about 21 Å, at least about 22 Å, at least about 23 Å, at least about 24 Å, at least about 25 Å, 26 Å, at least about 27 Å, 28 Å, at least about 29 Å, at least about 30 Å, or greater than about 30 Å.

In some embodiments, the spacing between the Cα atom of the at least 1 internal negatively charged amino acid and the Cα atoms of the negatively charged flanking amino acid can be from about 1 Å to about 30 Å. In some embodiments, the spacing between the Cα atom of the at least 1 internal negatively charged amino acid and the Cα atoms of the negatively charged flanking amino acid can be from about 1 Å to about 2 Å, about 1 Å to about 3 Å, about 1 Å to about 4 Å, about 1 Å to about 5 Å, about 1 Å to about 6 Å, about 1 Å to about 8 Å, about 1 Å to about 10 Å, about 1 Å to about 15 Å, about 1 Å to about 20 Å, about 1 Å to about 25 Å, about 1 Å to about 30 Å, about 2 Å to about 31 Å, about 2 Å to about 4 Å, about 2 Å to about 5 Å, about 2 Å to about 6 Å, about 2 Å to about 8 Å, about 2 Å to about 10 Å, about 2 Å to about 15 Å, about 2 Å to about 20 Å, about 2 Å to about 25 Å, about 2 Å to about 30 Å, about 3 Å to about 4 Å, about 3 Å to about 5 Å, about 3 Å to about 6 Å, about 3 Å to about 8 Å, about 3 Å to about 10 Å, about 3 Å to about 15 Å, about 3 Å to about 20 Å, about 3 Å to about 25 Å, about 3 Å to about 30 Å, about 4 Å to about 5 Å, about 4 Å to about 6 Å, about 4 Å to about 8 Å, about 4 Å to about 10 Å, about 4 Å to about 15 Å, about 4 Å to about 20 Å, about 4 Å to about 25 Å, about 4 Å to about 30 Å, about 5 Å to about 6 Å, about 5 Å to about 8 Å, about 5 Å to about 10 Å, about 5 Å to about 15 Å, about 5 Å to about 20 Å, about 5 Å to about 25 Å, about 5 Å to about 30 Å, about 6 Å to about 8 Å, about 6 Å to about 10 Å, about 6 Å to about 15 Å, about 6 Å to about 20 Å, about 6 Å to about 25 Å, about 6 Å to about 30 Å, about 8 Å to about 10 Å, about 8 Å to about 15 Å, about 8 Å to about 20 Å, about 8 Å to about 25 Å, about 8 Å to about 30 Å, about 10 Å to about 15 Å, about 10 Å to about 20 Å, about 10 Å to about 25 Å, about 10 Å to about 30 Å, about 15 Å to about 20 Å, about 15 Å to about 25 Å, about 15 Å to about 30 Å, about 20 Å to about 25 Å, about 20 Å to about 30 Å, or about 25 Å to about 30 Å.

In some embodiments, the spacing between the Cα atom of the at least 1 internal negatively charged amino acid and the Cα atoms of the negatively charged flanking amino acid can be about 1 Å, about 2 Å, about 3 Å, about 4 Å, about 5 Å, about 6 Å, about 7 Å, about 8 Å, about 9 Å, about 10 Å, about 11 Å, about 12 Å, about 13 Å, about 14 Å, about 15 Å, about 16 Å, about 17 Å, about 18 Å, about 20 Å, about 21 Å, about 22 Å, about 23 Å, about 24 Å, about 25 Å, 26 Å, about 27 Å, 28 Å, about 29 Å, or about 30 Å.

A nanopore lumen can comprise separate sets of charges oriented along the ring of the toroidal geometry of the nanopore. These sets of charges may be arranged along the longitudinal length of the channel. In some embodiments, a nanopore may comprise at least about 1, at least about 2, at least about 3, at least about 4, at least about 5, at least about 6, at least about 7, at least about 8, at least about 10, at least about 11, at least about 12, at least about 13, at least about 14, at least about 15, at least about 16, at least about 17, at least about 18, at least about 19, at least about 20, or greater than about 20 separate sets of charges. In some embodiments, a nanopore may comprise at most about 20, at most about 19, at most about 18, at most about 17, at most about 16, at most about 15, at most about 14, at most about 13, at most about 12, at most about 11, at most about 10, at most about 9, at most about 8, at most about 7, at most about 6, at most about 5, at most about 4, at most about 3, at most about 2, at most about 1, or less than about 1 separate set of charges.

In some embodiments, a nanopore may comprise from about 1 to about 20 separate sets of charges. In some embodiments, a nanopore may comprise from about 1 to about 2, about 1 to about 3, about 1 to about 4, about 1 to about 5, about 1 to about 8, about 1 to about 9, about 1 to about 10, about 1 to about 12, about 1 to about 15, about 1 to about 18, about 1 to about 20, about 2 to about 3, about 2 to about 4, about 2 to about 5, about 2 to about 8, about 2 to about 9, about 2 to about 10, about 2 to about 12, about 2 to about 15, about 2 to about 18, about 2 to about 20, about 3 to about 4, about 3 to about 5, about 3 to about 8, about 3 to about 9, about 3 to about 10, about 3 to about 12, about 3 to about 15, about 3 to about 18, about 3 to about 20, about 4 to about 5, about 4 to about 8, about 4 to about 9, about 4 to about 10, about 4 to about 12, about 4 to about 15, about 4 to about 18, about 4 to about 20, about 5 to about 8, about 5 to about 9, about 5 to about 10, about 5 to about 12, about 5 to about 15, about 5 to about 18, about 5 to about 20, about 8 to about 9, about 8 to about 10, about 8 to about 12, about 8 to about 15, about 8 to about 18, about 8 to about 20, about 9 to about 10, about 9 to about 12, about 9 to about 15, about 9 to about 18, about 9 to about 20, about 10 to about 12, about 10 to about 15, about 10 to about 18, about 10 to about 20, about 12 to about 15, about 12 to about 18, about 12 to about 20, about 15 to about 18, about 15 to about 20, or about 18 to about 20 separate sets of charges.

In some embodiments, a nanopore may comprise about 1, about 2, about 3, about 4, about 5, about 6, about 7, about 8, about 10, about 11, about 12, about 13, about 14, about 15, about 16, about 17, about 18, about 19, or about 20 separate sets of charges.

In some embodiments, the sets of charges may each be spaced at least about 0.1 nanometers, at least about 0.2 nanometers, at least about 0.3 nanometers, at least about 0.4 nanometers, at least about 0.5 nanometers, at least about 0.6 nanometers, at least about 0.7 nanometers, at least about 0.8 nanometers, at least about 0.9 nanometers, at least about 1 nanometers, at least about 2 nanometers, at least about 3 nanometers, at least about 4 nanometers, at least about 5 nanometers, at least about 6 nanometers, at least about 7 nanometers, at least about 8 nanometers, at least about 9 nanometers, at least about 10 nanometers, or greater than about 10 nanometers apart from each other along the longitudinal length of the channel. In some embodiments, the sets of charges may each be spaced at most about 10 nanometers, at most about 9 nanometers, at most about 8 nanometers, at most about 7 nanometers, at most about 6 nanometers, at most about 5 nanometers, at most about 4 nanometers, at most about 3 nanometers, at most about 2 nanometers, at most about 1 nanometers, at most about 0.9 nanometers, at most about 0.8 nanometers, at most about 0.7 nanometers, at most about 0.6 nanometers, at most about 0.5 nanometers, at most about 0.4 nanometers, at most about 0.3 nanometers, at most about 0.2 nanometers, at most about 0.1 nanometers, or less than about 0.1 nanometer apart from each other along the longitudinal length of the channel.

In some embodiments, the sets of charges may each be spaced from about 0.1 nanometers to about 5 nanometers apart from each other along the longitudinal length of the channel. In some embodiments, the sets of charges may each be spaced from about 0.1 nanometers to about 0.2 nanometers, about 0.1 nanometers to about 0.3 nanometers, about 0.1 nanometers to about 0.4 nanometers, about 0.1 nanometers to about 0.5 nanometers, about 0.1 nanometers to about 1 nanometer, about 0.1 nanometers to about 1.5 nanometers, about 0.1 nanometers to about 2 nanometers, about 0.1 nanometers to about 2.5 nanometers, about 0.1 nanometers to about 3 nanometers, about 0.1 nanometers to about 4 nanometers, about 0.1 nanometers to about 5 nanometers, about 0.2 nanometers to about 0.3 nanometers, about 0.2 nanometers to about 0.4 nanometers, about 0.2 nanometers to about 0.5 nanometers, about 0.2 nanometers to about 1 nanometer, about 0.2 nanometers to about 1.5 nanometers, about 0.2 nanometers to about 2 nanometers, about 0.2 nanometers to about 2.5 nanometers, about 0.2 nanometers to about 3 nanometers, about 0.2 nanometers to about 4 nanometers, about 0.2 nanometers to about 5 nanometers, about 0.3 nanometers to about 0.4 nanometers, about 0.3 nanometers to about 0.5 nanometers, about 0.3 nanometers to about 1 nanometer, about 0.3 nanometers to about 1.5 nanometers, about 0.3 nanometers to about 2 nanometers, about 0.3 nanometers to about 2.5 nanometers, about 0.3 nanometers to about 3 nanometers, about 0.3 nanometers to about 4 nanometers, about 0.3 nanometers to about 5 nanometers, about 0.4 nanometers to about 0.5 nanometers, about 0.4 nanometers to about 1 nanometer, about 0.4 nanometers to about 1.5 nanometers, about 0.4 nanometers to about 2 nanometers, about 0.4 nanometers to about 2.5 nanometers, about 0.4 nanometers to about 3 nanometers, about 0.4 nanometers to about 4 nanometers, about 0.4 nanometers to about 5 nanometers, about 0.5 nanometers to about 1 nanometer, about 0.5 nanometers to about 1.5 nanometers, about 0.5 nanometers to about 2 nanometers, about 0.5 nanometers to about 2.5 nanometers, about 0.5 nanometers to about 3 nanometers, about 0.5 nanometers to about 4 nanometers, about 0.5 nanometers to about 5 nanometers, about 1 nanometer to about 1.5 nanometers, about 1 nanometer to about 2 nanometers, about 1 nanometer to about 2.5 nanometers, about 1 nanometer to about 3 nanometers, about 1 nanometer to about 4 nanometers, about 1 nanometer to about 5 nanometers, about 1.5 nanometers to about 2 nanometers, about 1.5 nanometers to about 2.5 nanometers, about 1.5 nanometers to about 3 nanometers, about 1.5 nanometers to about 4 nanometers, about 1.5 nanometers to about 5 nanometers, about 2 nanometers to about 2.5 nanometers, about 2 nanometers to about 3 nanometers, about 2 nanometers to about 4 nanometers, about 2 nanometers to about 5 nanometers, about 2.5 nanometers to about 3 nanometers, about 2.5 nanometers to about 4 nanometers, about 2.5 nanometers to about 5 nanometers, about 3 nanometers to about 4 nanometers, about 3 nanometers to about 5 nanometers, about 4 nanometers to about 5 nanometers, about 5 nanometers to about 6 nanometers, about 6 nanometers to about 7 nanometers, about 7 nanometers to about 8 nanometers, about 8 nanometers to about 9 nanometers, or about 9 nanometers to about 10 nanometers apart from each other along the longitudinal length of the channel.

In some embodiments, the sets of charges may each be spaced about 0.1 nanometers, about 0.2 nanometers, about 0.3 nanometers, about 0.4 nanometers, about 0.5 nanometers, about 0.6 nanometers, about 0.7 nanometers, about 0.8 nanometers, about 0.9 nanometers, about 1 nanometers, about 2 nanometers, about 3 nanometers, about 4 nanometers, about 5 nanometers, about 6 nanometers, about 7 nanometers, about 8 nanometers, about 9 nanometers, or about 10 nanometers apart from each other along the longitudinal length of the channel. In some embodiments, one set of charges can be present at the cis entrance of the nanopore. In some embodiments, one set of charges can be present at the trans entrance of the nanopore. In some embodiments, one set of charges can be present at the cis entrance of the nanopore and one set of charges can be present at the trans entrance of the nanopore.

In some embodiments, a solution or solutions on either the cis side or the trans side of the fluidic chamber may be configured to have a set pH. The solution or solutions may have a pH of at least about 1, at least about 2, at least about 3, at least about 3.8, at least about 4, at least about 4.5, at least about 6, at least about 7, at least about 8, at least about 9, at least about 10, at least about 10.5 at least about 11, at least about 12, at least about 13, or greater than about 13 that can be employed. The solution or solutions may have a pH of at most about 13, at most about 12, at most about 11, at most about 10.5, at most about 10, at most about 9, at most about 8, at most about 7, at most about 6, at most about 4.5, at most about 4, at most about 3.8, at most about 3, at most about 2, at most about 1, or less than about 1 that can be employed.

The solution or solutions may have a pH from about 1 to about 13 that can be employed. The solution or solutions may have a pH from about 1 to about 2, about 1 to about 3, about 1 to about 4, about 1 to about 6, about 1 to about 7, about 1 to about 8, about 1 to about 9, about 1 to about 10, about 1 to about 11, about 1 to about 12, about 1 to about 13, about 2 to about 3, about 2 to about 4, about 2 to about 6, about 2 to about 7, about 2 to about 8, about 2 to about 9, about 2 to about 10, about 2 to about 11, about 2 to about 12, about 2 to about 13, about 3 to about 4, about 3 to about 6, about 3 to about 7, about 3 to about 8, about 3 to about 9, about 3 to about 10, about 3 to about 11, about 3 to about 12, about 3 to about 13, about 4 to about 6, about 4 to about 7, about 4 to about 8, about 4 to about 9, about 4 to about 10, about 4 to about 11, about 4 to about 12, about 4 to about 13, about 6 to about 7, about 6 to about 8, about 6 to about 9, about 6 to about 10, about 6 to about 11, about 6 to about 12, about 6 to about 13, about 7 to about 8, about 7 to about 9, about 7 to about 10, about 7 to about 11, about 7 to about 12, about 7 to about 13, about 8 to about 9, about 8 to about 10, about 8 to about 11, about 8 to about 12, about 8 to about 13, about 9 to about 10, about 9 to about 11, about 9 to about 12, about 9 to about 13, about 10 to about 11, about 10 to about 12, about 10 to about 13, about 11 to about 12, about 11 to about 13, or about 12 to about 13 that can be employed.

The solution or solutions may have a pH of about 1, about 2, about 3, about 3.8, about 4, about 4.5, about 6, about 7, about 8, about 9, about 10, about 10.5 about 11, about 12, or about 13 that can be employed.

In some embodiments, an electro-osmotic flow (also termed an electro-osmotic force) acts across the membrane in a cis to trans direction or a trans to cis direction. An electro-osmotic flow can be the flow that results from a net flow of a mobile layer of ions along a surface as induced by an applied potential. For example, a charged surface may form a static layer of oppositely charged mobile ions. Under an applied potential the charged mobile ions may be induced to move in the direction of higher potential if negative, or in the direction of lower potential if positive. The flow of charged ions can create a drag on the surrounding solvent (e.g., water) molecules, which in turn can result in a net flow that exerts a force acting on the surrounding molecules, both charged and neutral. For example, in a negatively charged nanopore lumen, an electroosmotic flow can result from a net flow of positive ions in a cis to trans direction (e.g. due to a lower potential on the trans side) causing the surrounding water to flow cis to trans and exert a force on surrounding molecules. The amount of ion flow and the corresponding magnitude of the electroosmotic flow can be influenced by parameters including an ion concentration difference across the membrane, a difference in potential, a net charge of a nanopore lumen, a geometry of a nanopore lumen, or combinations thereof. In some embodiments, an electro-osmotic flow can be the flow that results from one or more constrictions present in a nanopore channel. In some embodiments, an electro-osmotic flow can be the flow that results from a net flow of mobile ions along a surface as induced by an applied potential and one or more constrictions present in a nanopore channel.

In some embodiments, an electro-osmotic flow can be created or modified by a difference between a solution on a cis side of a membrane and a solution on a trans side of a membrane. The difference can be a difference in concentration of a molecule, including an ion, an electrolyte or an osmolyte.

In some embodiments, a difference between solutions can be a salt asymmetry or an ion asymmetry, wherein one side of a membrane (e.g. a cis side) comprises a different concentration of an ion than the other side (e.g. a trans side). An ion asymmetry can influence an ionic current across a membrane, as described by the Goldman-Hodgkin-Katz equation.

$${I}_{( s )} = {P}_{( S )} {z}_{s}^{2} * \frac{{V}_{m} {F}^{2}}{RT} \frac{{[ S ]}_{trans} - {[ S ]}_{cis} * {e}^{- {z}_{s} \frac{{V}_{m} F}{RT}}}{1 - {e}^{- {z}_{s} \frac{{V}_{m} F}{RT}}}$$

Where the ionic current (I(S)) ion species S across the membrane as a function of the applied potential (Vm): where P_{(S)} is the membrane permeability of ion species S, zₛ the valency of the ion, F the Faraday constant, R the gas constant, T the temperature and [S]_{cis} and [S]ₜᵣₐₙₛ the cis and trans concentrations of an ion species S, respectively. As the difference in concentration of the ions on the cis and trans sides impacts the ionic flux, the combined ionic flux of different species can thus influence an electro-osmotic force as ions flow in different directions across the membrane. This can be used to strengthen or weaken an electro-osmotic force by having a difference in ion concentration between the cis and trans sides that minimizes or maximizes a contribution of the ionic current of the species S to the net ionic flux.

A difference in a concentration of a molecule between two sides of a membrane can modify an electro-osmotic flux by providing a competing or assisting osmotic flux. A difference in concentration across a membrane can create an osmotic gradient, wherein a solvent (e.g. water) may diffuse across a membrane in the direction of a higher concentration of the molecule so as to minimize the difference in concentration between the sides of the membrane. The osmotic gradient can be oriented so as to drive a water flow in the same direction as the electro-osmotic force, or in a different direction. For example, a high ion concentration on a cis side relative to a trans side can create an osmotic gradient that competes with a cis to trans electro-osmotic force, as the osmotic gradient can drive water flow in a trans to cis direction. The ion concentrations may support a cis to trans electro-osmotic flow even if they also provide an osmotic gradient.

In some embodiments an electro-osmotic force can act in the same direction as an electrophoretic force or in an opposing direction to an electrophoretic force. In some embodiments, the electro-osmotic force can be greater than the electrophoretic force. In some embodiments, the electro-osmotic force can be less than the electrophoretic force.

In some embodiments, a cis to trans EOF can comprise a net ionic current flow from the cis side of the membrane to the trans side of the membrane. In some embodiments, a trans to cis EOF can comprise a net ionic current flow from the trans side of the membrane to the cis side of the membrane. In some cases, the nanopore system can comprise a total ionic current flow. In some instances, the net ionic current flow can comprise the flow of less than all of the total ions in the nanopore system. In some cases, the net ionic current flow can comprise the flow of less than all of the total ions in the nanopore system in a specific direction. In some cases, the specific direction can from the cis side of the membrane to the trans side of the membrane. In some cases, the specific direction can from the trans side of the membrane to the cis side of the membrane. In some cases, the total ionic current flow can comprise the total flow of all ions in the nanopore system. In some cases, the total ionic current flow can comprise the total flow of all ions in the nanopore system in a specific direction. In some cases, the specific direction can from the cis side of the membrane to the trans side of the membrane. In some cases, the specific direction can from the trans side of the membrane to the cis side of the membrane.

In some embodiments, the net ionic current flow can comprise from about 0.001% to about 100% of the total ionic current flow. In some cases, the net ionic current flow can comprise from about 0.001% to about 0.01%, from about 0.01% to about 0.1%, from about 0.1% to about 1%, from about 1% to about 10%, or from about 10% to about 100% of the total ionic current flow. In some cases, the net ionic current flow can comprise at least about 0.001%, at least about 0.005%, at least about 0.01%, at least about 0.05%, at least about 0.1%, at least about 0.5%, at least about 1%, at least about 5%, at least about 10%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 99.5%, or at least about 100% of the total ionic current flow. In some instances, the net ionic current flow can comprise at most about 100%, at most about 99.5%, at most about 99%, at most about 98%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, at most about 0.05%, at most about 0.01%, at most about 0.005%, at most about 0.001%, or less than 0.001% of the total ionic current flow. In some cases, the net ionic current flow can comprise about 0.001%, about 0.005%, about 0.01%, about 0.05%, about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, about 99%, about 99.5%, or about 100% of the total ionic current flow.

In some embodiments, the rate of translocation can be from about 0.1 amino acids per second (aa/s) to about 1,000 aa/s. In some cases, the rate of translocation can be at least about 0.1 aa/s, at least about 0.5 aa/s, at least about 1 aa/s, at least about 5 aa/s, at least about 10 aa/s, at least about 50 aa/s, at least about 100 aa/s, at least about 500 aa/s, at least about 1,000 aa/s, or more than 1,000 aa/s. In some cases, the rate of translocation can at least most about 1,000 aa/s, at most about 500 aa/s, at most about 100 aa/s, at most about 50 aa/s, at most about 10 aa/s, at most about 5 aa/s, at most about 1 aa/s, at most about 0.5 aa/s, at most about 0.1 aa/s, or less than 0.1 aa/s. In some cases, the rate of translocation can be about 0.1 aa/s, about 0.5 aa/s, about 1 aa/s, about 5 aa/s, about 10 aa/s, about 50 aa/s, about 100 aa/s, about 500 aa/s, or about 1,000 aa/s.

In some embodiments, a cis to trans EOF results from a net ionic current flow cis to trans over a total ionic current flow, also referred to as a relative net current flow cis to trans, of greater than about 0.0, greater than about 0.1, greater than about 0.2, greater than about 0.3, greater than about 0.4, greater than about 0.5, greater than about 0.6, greater than about 0.7, greater than about 0.8, greater than about 0.9, greater than about 0.95, or greater than about 0.99. In some embodiments, a cis to trans EOF results from a net ionic current flow trans to cis over a total ionic current flow, also referred to as a relative net current flow cis to trans, of less than about 0.0, less than about −0.1, less than about −0.2, less than about −0.3, less than about −0.4, less than about −0.5, less than about −0.6, less than about −0.7, less than about −0.8, less than about −0.9, less than about −0.95, or less than about −0.99.

In some embodiments, a cis to trans EOF results from a net ionic current flow cis to trans over a total ionic current flow, also referred to as a relative net current flow cis to trans, of at least about −0.99, at least about −0.95, at least about −0.9, at least about −0.8, at least about −0.7, at least about −0.6, at least about −0.5, at least about −0.4, at least about −0.3, at least about −0.2, at least about −0.1, at least about 0.0, at least about 0.1, at least about 0.2, at least about 0.3, at least about 0.4, at least about 0.5, at least about 0.6, at least about 0.7, at least about 0.8, at least about 0.9, at least about 0.95, about 0.99, or greater than about 0.99. In some embodiments, a cis to trans EOF results from a net ionic current flow cis to trans over a total ionic current flow, also referred to as a relative net current flow cis to trans, of at most about 0.99, at most about 0.9, at most about 0.8, at most about 0.7, at most about 0.6, at most about 0.5, at most about 0.4, at most about 0.3, at most about 0.2, at most about 0.1, at most about 0.0, at most about −0.1, at most about −0.2, at most about −0.3, at most about −0.4, at most about −0.5, at most about −0.6, at most about −0.7, at most about −0.8, at most about −0.9, −0.95, at most about −0.99, or less than about −0.99 In some embodiments, a cis to trans EOF results from a net ionic current flow cis to trans over a total ionic current flow, also referred to as a relative net current flow cis to trans, from about −0.99 to about 0.99. In some embodiments, a cis to trans EOF results from a net ionic current flow cis to trans over a total ionic current flow, also referred to as a relative net current flow cis to trans, from about −0.99 to about −0.9, about −0.99 to about −0.8, about −0.99 to about −0.6, about −0.99 to about −0.4, about −0.99 to about −0.2, about −0.99 to about 0, about −0.99 to about 0.2, about −0.99 to about 0.4, about −0.99 to about 0.6, about −0.99 to about 0.8, about −0.99 to about 0.99, about −0.9 to about −0.8, about −0.9 to about −0.6, about −0.9 to about −0.4, about −0.9 to about −0.2, about −0.9 to about 0, about −0.9 to about 0.2, about −0.9 to about 0.4, about −0.9 to about 0.6, about −0.9 to about 0.8, about −0.9 to about 0.99, about −0.8 to about −0.6, about −0.8 to about −0.4, about −0.8 to about −0.2, about −0.8 to about 0, about −0.8 to about 0.2, about −0.8 to about 0.4, about −0.8 to about 0.6, about −0.8 to about 0.8, about −0.8 to about 0.99, about −0.6 to about −0.4, about −0.6 to about −0.2, about −0.6 to about 0, about −0.6 to about 0.2, about −0.6 to about 0.4, about −0.6 to about 0.6, about −0.6 to about 0.8, about −0.6 to about 0.99, about −0.4 to about −0.2, about −0.4 to about 0, about −0.4 to about 0.2, about −0.4 to about 0.4, about −0.4 to about 0.6, about −0.4 to about 0.8, about −0.4 to about 0.99, about −0.2 to about 0, about −0.2 to about 0.2, about −0.2 to about 0.4, about −0.2 to about 0.6, about −0.2 to about 0.8, about −0.2 to about 0.99, about 0 to about 0.2, about 0 to about 0.4, about 0 to about 0.6, about 0 to about 0.8, about 0 to about 0.99, about 0.2 to about 0.4, about 0.2 to about 0.6, about 0.2 to about 0.8, about 0.2 to about 0.99, about 0.4 to about 0.6, about 0.4 to about 0.8, about 0.4 to about 0.99, about 0.6 to about 0.8, about 0.6 to about 0.99, or about 0.8 to about 0.99.

In some embodiments, a cis to trans EOF results from a net ionic current flow cis to trans over a total ionic current flow, also referred to as a relative net current flow cis to trans, of about −0.99, about −0.95, about −0.9, about −0.8, about −0.7, about −0.6, about −0.5, about −0.4, about −0.3, about −0.2, about −0.1, about 0.0, about 0.1, about 0.2, about 0.3, about 0.4, about 0.5, about 0.6, about 0.7, about 0.8, about 0.9, about 0.95, or about 0.99.

In some embodiments, the absolute relative net electro-osmotic current over applied voltage (I_{reIV}), can be at least about 0.01 pA/mV, at least about 0.02 pA/mV, at least about 0.03 pA/mV, at least about 0.04 pA/mV, at least about 0.05 pA/mV, at least about 0.06 pA/mV, at least about 0.07 pA/mV, at least about 0.08 pA/mV, at least about 0.09 pA/mV, at least about 0.10 pA/mV, at least about 0.15 pA/mV, at least about 0.2 pA/mV, at least about 0.3 pA/mV, at least about 0.4 pA/mV, at least about 0.5 pA/mV, at least about 0.6 pA/mV, at least about 0.7 pA/mV, at least about 0.8 pA/mV, at least about 0.9 pA/mV, at least about 1 pA/mV, or greater than about 1 pA/mV. In some embodiments, the absolute relative net electro-osmotic current over applied voltage (I_{reIV}), can be at most about 1 pA/mV, at most about 0.9, at most about 0.8, at most about 0.7, at most about 0.6, at most about 0.5, at most about 0.4, at most about 0.3, at most about 0.2, at most about 0.15, at most about 0.10, at most about 0.09, at most about 0.08, at most about 0.07, at most about 0.06, at most about 0.05, at most about 0.04, at most about 0.03, at most about 0.02, at most about 0.01, or less than about 0.1 pA/mV.

In some embodiments, the absolute relative net electro-osmotic current over applied voltage (I_{reIV}), can be from about 0.01 pA/mV to about 1 pA/mV. In some embodiments, the absolute relative net electro-osmotic current over applied voltage (I_{reIV}), can be from about 0.01 pA/mV to about 0.02 pA/mV, about 0.01 pA/mV to about 0.04 pA/mV, about 0.01 pA/mV to about 0.06 pA/mV, about 0.01 pA/mV to about 0.08 pA/mV, about 0.01 pA/mV to about 0.1 pA/mV, about 0.01 pA/mV to about 0.15 pA/mV, about 0.01 pA/mV to about 0.2 pA/mV, about 0.01 pA/mV to about 0.4 pA/mV, about 0.01 pA/mV to about 0.6 pA/mV, about 0.01 pA/mV to about 0.8 pA/mV, about 0.01 pA/mV to about 1 pA/mV, about 0.02 pA/mV to about 0.04 pA/mV, about 0.02 pA/mV to about 0.06 pA/mV, about 0.02 pA/mV to about 0.08 pA/mV, about 0.02 pA/mV to about 0.1 pA/mV, about 0.02 pA/mV to about 0.15 pA/mV, about 0.02 pA/mV to about 0.2 pA/mV, about 0.02 pA/mV to about 0.4 pA/mV, about 0.02 pA/mV to about 0.6 pA/mV, about 0.02 pA/mV to about 0.8 pA/mV, about 0.02 pA/mV to about 1 pA/mV, about 0.04 pA/mV to about 0.06 pA/mV, about 0.04 pA/mV to about 0.08 pA/mV, about 0.04 pA/mV to about 0.1 pA/mV, about 0.04 pA/mV to about 0.15 pA/mV, about 0.04 pA/mV to about 0.2 pA/mV, about 0.04 pA/mV to about 0.4 pA/mV, about 0.04 pA/mV to about 0.6 pA/mV, about 0.04 pA/mV to about 0.8 pA/mV, about 0.04 pA/mV to about 1 pA/mV, about 0.06 pA/mV to about 0.08 pA/mV, about 0.06 pA/mV to about 0.1 pA/mV, about 0.06 pA/mV to about 0.15 pA/mV, about 0.06 pA/mV to about 0.2 pA/mV, about 0.06 pA/mV to about 0.4 pA/mV, about 0.06 pA/mV to about 0.6 pA/mV, about 0.06 pA/mV to about 0.8 pA/mV, about 0.06 pA/mV to about 1 pA/mV, about 0.08 pA/mV to about 0.1 pA/mV, about 0.08 pA/mV to about 0.15 pA/mV, about 0.08 pA/mV to about 0.2 pA/mV, about 0.08 pA/mV to about 0.4 pA/mV, about 0.08 pA/mV to about 0.6 pA/mV, about 0.08 pA/mV to about 0.8 pA/mV, about 0.08 pA/mV to about 1 pA/mV, about 0.1 pA/mV to about 0.15 pA/mV, about 0.1 pA/mV to about 0.2 pA/mV, about 0.1 pA/mV to about 0.4 pA/mV, about 0.1 pA/mV to about 0.6 pA/mV, about 0.1 pA/mV to about 0.8 pA/mV, about 0.1 pA/mV to about 1 pA/mV, about 0.15 pA/mV to about 0.2 pA/mV, about 0.15 pA/mV to about 0.4 pA/mV, about 0.15 pA/mV to about 0.6 pA/mV, about 0.15 pA/mV to about 0.8 pA/mV, about 0.15 pA/mV to about 1 pA/mV, about 0.2 pA/mV to about 0.4 pA/mV, about 0.2 pA/mV to about 0.6 pA/mV, about 0.2 pA/mV to about 0.8 pA/mV, about 0.2 pA/mV to about 1 pA/mV, about 0.4 pA/mV to about 0.6 pA/mV, about 0.4 pA/mV to about 0.8 pA/mV, about 0.4 pA/mV to about 1 pA/mV, about 0.6 pA/mV to about 0.8 pA/mV, about 0.6 pA/mV to about 1 pA/mV, or about 0.8 pA/mV to about 1 pA/mV.

In some embodiments, the absolute relative net electro-osmotic current over applied voltage (I_{reIV}), can be about 0.01 pA/mV, about 0.02 pA/mV, about 0.03 pA/mV, about 0.04 pA/mV, about 0.05 pA/mV, about 0.06 pA/mV, about 0.07 pA/mV, about 0.08 pA/mV, about 0.09 pA/mV, about 0.10 pA/mV, about 0.15 pA/mV, about 0.2 pA/mV, about 0.3 pA/mV, about 0.4 pA/mV, about 0.5 pA/mV, about 0.6 pA/mV, about 0.7 pA/mV, about 0.8 pA/mV, about 0.9 pA/mV, or about 1 pA/mV. In some cases, the absolute relative net electo-osmotic current over applied voltage can comprise a value that is calculated by dividing the net electro-osmotic flow by the applied voltage. In some cases, the net electro-osmotic flow can comprise the total flow of a subset of the ions or salts in the nanopore system.

In some embodiments, a pore can comprise a relative ion selectivity P(+)/P(−) of at least about 0.1, at least about 0.2, at least about 0.3, at least about 0.4, at least about 0.5, at least about 0.6, at least about 0.7, at least about 0.8, at least about 0.9, at least about 1, at least about 2, at least about 3, at least about 4, at least about 5, or greater than about 5 under an applied voltage difference across the membrane. In some embodiments, a pore can comprise a relative ion selectivity P(+)/P(−) of at most about 5, at most about 4, at most about 3, at most about 2, at most about 1, at most about 0.9, at most about 0.8, at most about 0.7, at most about 0.6, at most about 0.5, at most about 0.4, at most about 0.3, at most about 0.2, at most about 0.1, or less than about 0.1 under an applied voltage difference across the membrane.

In some embodiments, a pore can comprise a relative ion selectivity P(+)/P(−) from about 0.1 to about 5 under an applied voltage difference across the membrane. In some embodiments, a pore can comprise a relative ion selectivity P(+)/P(−) from about 0.1 to about 0.2, about 0.1 to about 0.3, about 0.1 to about 0.4, about 0.1 to about 0.5, about 0.1 to about 1, about 0.1 to about 1.5, about 0.1 to about 2, about 0.1 to about 2.5, about 0.1 to about 3, about 0.1 to about 4, about 0.1 to about 5, about 0.2 to about 0.3, about 0.2 to about 0.4, about 0.2 to about 0.5, about 0.2 to about 1, about 0.2 to about 1.5, about 0.2 to about 2, about 0.2 to about 2.5, about 0.2 to about 3, about 0.2 to about 4, about 0.2 to about 5, about 0.3 to about 0.4, about 0.3 to about 0.5, about 0.3 to about 1, about 0.3 to about 1.5, about 0.3 to about 2, about 0.3 to about 2.5, about 0.3 to about 3, about 0.3 to about 4, about 0.3 to about 5, about 0.4 to about 0.5, about 0.4 to about 1, about 0.4 to about 1.5, about 0.4 to about 2, about 0.4 to about 2.5, about 0.4 to about 3, about 0.4 to about 4, about 0.4 to about 5, about 0.5 to about 1, about 0.5 to about 1.5, about 0.5 to about 2, about 0.5 to about 2.5, about 0.5 to about 3, about 0.5 to about 4, about 0.5 to about 5, about 1 to about 1.5, about 1 to about 2, about 1 to about 2.5, about 1 to about 3, about 1 to about 4, about 1 to about 5, about 1.5 to about 2, about 1.5 to about 2.5, about 1.5 to about 3, about 1.5 to about 4, about 1.5 to about 5, about 2 to about 2.5, about 2 to about 3, about 2 to about 4, about 2 to about 5, about 2.5 to about 3, about 2.5 to about 4, about 2.5 to about 5, about 3 to about 4, about 3 to about 5, or about 4 to about 5 under an applied voltage difference across the membrane.

In some embodiments, a pore can comprise a relative ion selectivity P(+)/P(−) of about 0.1, about 0.2, about 0.3, about 0.4, about 0.5, about 0.6, about 0.7, about 0.8, about 0.9, about 1, about 2, about 3, about 4, or about 5 under an applied voltage difference across the membrane.

In some embodiments, the solutions on the cis side and trans side of the fluidic chamber are configured to generate an electro-osmic force. The electro-osmic force can be generated due to a difference in concentration of a solute between the solution on the cis side and the solution on the cis side. The solute can be one or more ions or one or more osmolytes. In some cases, the one or more ions can comprise chloride, carbonate, chlorite, chlorate, phosphate, bicarbonate, bromide, ammonium sulfate, ammonium, sulfate, sulfide, calcium, fluoride, hydroxide, aluminum, barium, bismuth, cadmium, cesium, chromium, cobalt, copper, hydrogen, iron, lead, lithium, magnesium, mercury, nickel, potassium, rubidium, silver, sodium, strontium, tin, zinc, iodide, nitride, oxide, glutamate, acetate, formats, acetates, butyrates, benzoates, carboxylates, alkoxides, penolates, oxalates, amlonates, tartrates, malates, citrates, gluconates, maleates, sorbates, stearates, lactages, glycerates, urates, diazonium salts, iminium salts, phosphinates, organophosphates, mesylates, bechgaard salts, picolinates, salts of cocaine, salts of morphine, nonsodium glutamate, trolamine salicylate, triphenylmethyl hexafluorophosphate, choline chloride, copper ibuprofenate, homatropine methylbromide, mellite, tetrpropylammonium perruthenate, collidinium p-toluenesulfonate, pyridinium chloride, tetrasodium EDTA, lithium diisopropylamide, lithium bis(trimethylsiyl)amide, potassium trispyrazolylborate, redox salts, ferrocyanide, ferricyanide, or any combinations thereof. In some embodiments, the one or more osmolytes can be one or more types of salt. In some cases, the one or more types of salt can comprise sodium chloride, sodium carbonate, ammonium chloride, sodium acetate, potassium cyanide, zinc chloride hydroxide, potassium chlorate, calcium phosphate, sodium nitrate, potassium cerium fluoride, Mohr's salt, sodium potassium sulphate, potassium permanganate, tetra amino cupric sulphate, zinc chloride hydroxide monohydrate, monosodium glutamate, copper sulfate, calcium chloride, potassium chloride, magnesium sulfate, magnesium chloride, sodium acetate, magnesium nitrate, or any combination thereof. These ions or osmolytes can flow across the membrane through the nanopore. These ions can be high mobility ions or low mobility ions.

In some embodiments, the EOF can be generated by an asymmetric salt distribution between the cis side of the membrane and the trans side of the membrane. In some cases, the concentration of one or more salts on the cis side of the membrane can be different from the concentration of the one or more salts on the trans side of the membrane. In some cases, the concentration of one or more salts on the cis side of the membrane can be higher than the concentration of the one or more salts on the trans side of the membrane. In some cases, the concentration of one or more salts on the cis side of the membrane can be lower than the concentration of one or more salts on the trans side of the membrane. In some cases, the concentration of one or more salts on the trans side of the membrane can be higher than the concentration of the one or more salts on the cis side of the membrane. In some cases, the concentration of one or more salts on the trans side of the membrane can be lower than the concentration of the one or more salts on the cis side of the membrane.

In some cases, the concentration of one or more salts on the cis side of the membrane can be between about 1 nanomolar (nM) to about 1,000 nM. In some instances, the concentration of one or more salts on the cis side of the membrane can be between about 1 nM to about 10 nM, between about 10 nM to about 100 nM, or between about 100 nM to about 1,000 nM. In some cases, the concentration of one or more salts on the cis side of the membrane can be at least about 1 nM, at least about 5 nM, at least about 10 nM, at least about 15 nM, at least about 20 nM, at least about 25 nM, at least about 30 nM, at least about 35 nM, at least about 40 nM, at least about 45 nM, at least about 50 nM, at least about 55 nM, at least about 60 nM, at least about 65 nM, at least about 70 nM, at least about 75 nM, at least about 80 nM, at least about 85 nM, at least about 90 nM, at least about 95 nM, at least about 100 nM, at least about 150 nM, at least about 200 nM, at least about 250 nM, at least about 300 nM, at least about 350 nM, at least about 400 nM, at least about 450 nM, at least about 500 nM, at least about 550 nM, at least about 600 nM, at least about 650 nM, at least about 700 nM, at least about 750 nM, at least about 800 nM, at least about 850 nM, at least about 900 nM, at least about 950 nM, at least about 1,000 nM, or more than 1,000 nM. In some cases, the concentration of one or more salts on the cis side of the membrane can at most about 1,000 nM, at most about 950 nM, at most about 900 nM, at most about 850 nM, at most about 800 nM, at most about 750 nM, at most about 700 nM, at most about 650 nM, at most about 600 nM, at most about 550 nM, at most about 500 nM, at most about 450 nM, at most about 400 nM, at most about 350 nM, at most about 300 nM, at most about 250 nM, at most about 200 nM, at most about 150 nM, at most about 100 nM, at most about 95 nM, at most about 90 nM, at most about 85 nM, at most about 80 nM, at most about 75 nM, at most about 70 nM, at most about 65 nM, at most about 60 nM, at most about 55 nM, at most about 45 nM, at most about 40 nM, at most about 35 nM, at most about 30 nM, at most about 25 nM, at most about 20 nM, at most about 15 nM, at most about 10 nM, at most about 5 nM, at most about 1 nM, or less than 1 nM. In some cases, the concentration of one or more salts on the cis side of the membrane can about 1 nM, about 5 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, about 100 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, or about 1,000 nM.

In some embodiments, a salt, ion, osmolyte, or electrolyte concentration on the cis side can be at least about 0.01 M, at least about 0.05 M, at least about 0.10 M, at least about 0.20 M, at least about 0.30 M, at least about 0.40 M, at least about 0.50 M, at least about 0.60 M, at least about 0.70 M, at least about 0.80 M, at least about 0.90 M, at least about 1.00 M, at least about 1.10 M, at least about 1.25 M, at least about 1.50 M, at least about 1.75 M, at least about 2 M, at least about 2.5 M, at least about 3 M, at least about 3.5 M, at least about 4 M, at least about 4.5 M, at least about 5 M, or greater than about 5 M. In some embodiments, a salt, ion, osmolyte, or electrolyte concentration on the cis side can be at most about 5 M, at most about 4.5 M, at most about 4 M, at most about 3.5 M, at most about 3 M, at most about 2.5 M, at most about 2 M, at most about 1.75 M, at most about 1.50 M, at most about 1.25 M, at most about 1 M, at most about 0.90 M, at most about 0.80 M, at most about 0.70 M, at most about 0.60 M, at most about 0.50 M, at most about 0.40 M, at most about 0.30 M, at most about 0.20 M, at most about 0.10 M, at most about 0.05 M, at most about 0.01 M, or less than about 0.01 M.

In some embodiments, a salt, ion, osmolyte, or electrolyte concentration on the cis side can be from about 0.01 M to about 5 M. In some embodiments, a salt, ion, osmolyte, or electrolyte concentration on the cis side can be from about 0.01 M to about 0.1 M, about 0.01 M to about 0.5 M, about 0.01 M to about 1 M, about 0.01 M to about 1.5 M, about 0.01 M to about 2 M, about 0.01 M to about 2.5 M, about 0.01 M to about 3 M, about 0.01 M to about 3.5 M, about 0.01 M to about 4 M, about 0.01 M to about 4.5 M, about 0.01 M to about 5 M, about 0.1 M to about 0.5 M, about 0.1 M to about 1 M, about 0.1 M to about 1.5 M, about 0.1 M to about 2 M, about 0.1 M to about 2.5 M, about 0.1 M to about 3 M, about 0.1 M to about 3.5 M, about 0.1 M to about 4 M, about 0.1 M to about 4.5 M, about 0.1 M to about 5 M, about 0.5 M to about 1 M, about 0.5 M to about 1.5 M, about 0.5 M to about 2 M, about 0.5 M to about 2.5 M, about 0.5 M to about 3 M, about 0.5 M to about 3.5 M, about 0.5 M to about 4 M, about 0.5 M to about 4.5 M, about 0.5 M to about 5 M, about 1 M to about 1.5 M, about 1 M to about 2 M, about 1 M to about 2.5 M, about 1 M to about 3 M, about 1 M to about 3.5 M, about 1 M to about 4 M, about 1 M to about 4.5 M, about 1 M to about 5 M, about 1.5 M to about 2 M, about 1.5 M to about 2.5 M, about 1.5 M to about 3 M, about 1.5 M to about 3.5 M, about 1.5 M to about 4 M, about 1.5 M to about 4.5 M, about 1.5 M to about 5 M, about 2 M to about 2.5 M, about 2 M to about 3 M, about 2 M to about 3.5 M, about 2 M to about 4 M, about 2 M to about 4.5 M, about 2 M to about 5 M, about 2.5 M to about 3 M, about 2.5 M to about 3.5 M, about 2.5 M to about 4 M, about 2.5 M to about 4.5 M, about 2.5 M to about 5 M, about 3 M to about 3.5 M, about 3 M to about 4 M, about 3 M to about 4.5 M, about 3 M to about 5 M, about 3.5 M to about 4 M, about 3.5 M to about 4.5 M, about 3.5 M to about 5 M, about 4 M to about 4.5 M, about 4 M to about 5 M, or about 4.5 M to about 5 M.

In some embodiments, a salt, ion, osmolyte, or electrolyte concentration on the cis side can be about 0.01 M, about 0.05 M, about 0.10 M, about 0.20 M, about 0.30 M, about 0.40 M, about 0.50 M, about 0.60 M, about 0.70 M, about 0.80 M, about 0.90 M, about 1.00 M, about 1.10 M, about 1.25 M, about 1.50 M, about 1.75 M, about 2 M, about 2.5 M, about 3 M, about 3.5 M, about 4 M, about 4.5 M, or about 5 M.

In some cases, the concentration of one or more salts on the trans side of the membrane can be between about 1 nanomolar (nM) to about 1,000 nM. In some instances, the concentration of one or more salts on the trans side of the membrane can be between about 1 nM to about 10 nM, between about 10 nM to about 100 nM, or between about 100 nM to about 1,000 nM. In some cases, the concentration of one or more salts on the trans side of the membrane can be at least about 1 nM, at least about 5 nM, at least about 10 nM, at least about 15 nM, at least about 20 nM, at least about 25 nM, at least about 30 nM, at least about 35 nM, at least about 40 nM, at least about 45 nM, at least about 50 nM, at least about 55 nM, at least about 60 nM, at least about 65 nM, at least about 70 nM, at least about 75 nM, at least about 80 nM, at least about 85 nM, at least about 90 nM, at least about 95 nM, at least about 100 nM, at least about 150 nM, at least about 200 nM, at least about 250 nM, at least about 300 nM, at least about 350 nM, at least about 400 nM, at least about 450 nM, at least about 500 nM, at least about 550 nM, at least about 600 nM, at least about 650 nM, at least about 700 nM, at least about 750 nM, at least about 800 nM, at least about 850 nM, at least about 900 nM, at least about 950 nM, at least about 1,000 nM, or more than about 1,000 nM. In some cases, the concentration of one or more salts on the trans side of the membrane can at most about 1,000 nM, at most about 950 nM, at most about 900 nM, at most about 850 nM, at most about 800 nM, at most about 750 nM, at most about 700 nM, at most about 650 nM, at most about 600 nM, at most about 550 nM, at most about 500 nM, at most about 450 nM, at most about 400 nM, at most about 350 nM, at most about 300 nM, at most about 250 nM, at most about 200 nM, at most about 150 nM, at most about 100 nM, at most about 95 nM, at most about 90 nM, at most about 85 nM, at most about 80 nM, at most about 75 nM, at most about 70 nM, at most about 65 nM, at most about 60 nM, at most about 55 nM, at most about 45 nM, at most about 40 nM, at most about 35 nM, at most about 30 nM, at most about 25 nM, at most about 20 nM, at most about 15 nM, at most about 10 nM, at most about 5 nM, at most about 1 nM, or less than 1 nM. In some cases, the concentration of one or more salts on the trans side of the membrane can about 1 nM, about 5 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, about 100 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, or about 1,000 nM.

In some embodiments, a salt, ion, osmolyte, or electrolyte concentration on the trans side can be at least about 0.01 M, at least about 0.05 M, at least about 0.10 M, at least about 0.20 M, at least about 0.30 M, at least about 0.40 M, at least about 0.50 M, at least about 0.60 M, at least about 0.70 M, at least about 0.80 M, at least about 0.90 M, at least about 1.00 M, at least about 1.10 M, at least about 1.25 M, at least about 1.50 M, at least about 1.75 M, at least about 2 M, at least about 2.5 M, at least about 3 M, at least about 3.5 M, at least about 4 M, at least about 4.5 M, at least about 5 M, or greater than about 5 M. In some embodiments, a salt, ion, osmolyte, or electrolyte concentration on the trans side can be at most about 5 M, at most about 4.5 M, at most about 4 M, at most about 3.5 M, at most about 3 M, at most about 2.5 M, at most about 2 M, at most about 1.75 M, at most about 1.50 M, at most about 1.25 M, at most about 1 M, at most about 0.90 M, at most about 0.80 M, at most about 0.70 M, at most about 0.60 M, at most about 0.50 M, at most about 0.40 M, at most about 0.30 M, at most about 0.20 M, at most about 0.10 M, at most about 0.05 M, at most about 0.01 M, or less than about 0.01 M.

In some embodiments, a salt, ion, osmolyte, or electrolyte concentration on the trans side can be from about 0.01 M to about 5 M. In some embodiments, a salt, ion, osmolyte, or electrolyte concentration on the cis side can be from about 0.01 M to about 0.1 M, about 0.01 M to about 0.5 M, about 0.01 M to about 1 M, about 0.01 M to about 1.5 M, about 0.01 M to about 2 M, about 0.01 M to about 2.5 M, about 0.01 M to about 3 M, about 0.01 M to about 3.5 M, about 0.01 M to about 4 M, about 0.01 M to about 4.5 M, about 0.01 M to about 5 M, about 0.1 M to about 0.5 M, about 0.1 M to about 1 M, about 0.1 M to about 1.5 M, about 0.1 M to about 2 M, about 0.1 M to about 2.5 M, about 0.1 M to about 3 M, about 0.1 M to about 3.5 M, about 0.1 M to about 4 M, about 0.1 M to about 4.5 M, about 0.1 M to about 5 M, about 0.5 M to about 1 M, about 0.5 M to about 1.5 M, about 0.5 M to about 2 M, about 0.5 M to about 2.5 M, about 0.5 M to about 3 M, about 0.5 M to about 3.5 M, about 0.5 M to about 4 M, about 0.5 M to about 4.5 M, about 0.5 M to about 5 M, about 1 M to about 1.5 M, about 1 M to about 2 M, about 1 M to about 2.5 M, about 1 M to about 3 M, about 1 M to about 3.5 M, about 1 M to about 4 M, about 1 M to about 4.5 M, about 1 M to about 5 M, about 1.5 M to about 2 M, about 1.5 M to about 2.5 M, about 1.5 M to about 3 M, about 1.5 M to about 3.5 M, about 1.5 M to about 4 M, about 1.5 M to about 4.5 M, about 1.5 M to about 5 M, about 2 M to about 2.5 M, about 2 M to about 3 M, about 2 M to about 3.5 M, about 2 M to about 4 M, about 2 M to about 4.5 M, about 2 M to about 5 M, about 2.5 M to about 3 M, about 2.5 M to about 3.5 M, about 2.5 M to about 4 M, about 2.5 M to about 4.5 M, about 2.5 M to about 5 M, about 3 M to about 3.5 M, about 3 M to about 4 M, about 3 M to about 4.5 M, about 3 M to about 5 M, about 3.5 M to about 4 M, about 3.5 M to about 4.5 M, about 3.5 M to about 5 M, about 4 M to about 4.5 M, about 4 M to about 5 M, or about 4.5 M to about 5 M.

In some embodiments, a salt, ion, osmolyte, or electrolyte concentration on the trans side can be about 0.01 M, about 0.05 M, about 0.10 M, about 0.20 M, about 0.30 M, about 0.40 M, about 0.50 M, about 0.60 M, about 0.70 M, about 0.80 M, about 0.90 M, about 1.00 M, about 1.10 M, about 1.25 M, about 1.50 M, about 1.75 M, about 2 M, about 2.5 M, about 3 M, about 3.5 M, about 4 M, about 4.5 M, or about 5 M.

In some embodiments, a difference in salt, ion, or electrolyte concentrations between the cis and trans sides can be at least about 0.01 M, at least about 0.05, at least about 0.10, at least about 0.20, at least about 0.30, at least about 0.40, at least about 0.50, at least about 0.60, at least about 0.70, at least about 0.80, at least about 0.90, at least about 1.00, at least about 1.10, at least about 1.25, at least about 1.50, at least about 1.75, at least about 2, at least about 2.5, at least about 3, at least about 3.5, at least about 4, at least about 4.5, at least about 5 M, or greater than about 5 M. In some embodiments, a difference in salt, ion, or electrolyte concentrations between the cis and trans sides can be at most about 5 M, at most about 4.5 M, at most about 4 M, at most about 3.5 M, at most about 3 M, at most about 2.5 M, at most about 2 M, at most about 1.75 M, at most about 1.50 M, at most about 1.25 M, at most about 1 M, at most about 0.90 M, at most about 0.80 M, at most about 0.70 M, at most about 0.60 M, at most about 0.50 M, at most about 0.40 M, at most about 0.30 M, at most about 0.20 M, at most about 0.10 M, at most about 0.05 M, at most about 0.01 M, or less than about 0.01 M.

In some embodiments, a difference in salt, ion, or electrolyte concentrations between the cis and trans sides can be from about 0.01 M to about 5 M. In some embodiments, a difference in salt, ion, or electrolyte concentrations between the cis and trans sides can be from about 0.01 M to about 0.1 M, about 0.01 M to about 0.5 M, about 0.01 M to about 1 M, about 0.01 M to about 1.5 M, about 0.01 M to about 2 M, about 0.01 M to about 2.5 M, about 0.01 M to about 3 M, about 0.01 M to about 3.5 M, about 0.01 M to about 4 M, about 0.01 M to about 4.5 M, about 0.01 M to about 5 M, about 0.1 M to about 0.5 M, about 0.1 M to about 1 M, about 0.1 M to about 1.5 M, about 0.1 M to about 2 M, about 0.1 M to about 2.5 M, about 0.1 M to about 3 M, about 0.1 M to about 3.5 M, about 0.1 M to about 4 M, about 0.1 M to about 4.5 M, about 0.1 M to about 5 M, about 0.5 M to about 1 M, about 0.5 M to about 1.5 M, about 0.5 M to about 2 M, about 0.5 M to about 2.5 M, about 0.5 M to about 3 M, about 0.5 M to about 3.5 M, about 0.5 M to about 4 M, about 0.5 M to about 4.5 M, about 0.5 M to about 5 M, about 1 M to about 1.5 M, about 1 M to about 2 M, about 1 M to about 2.5 M, about 1 M to about 3 M, about 1 M to about 3.5 M, about 1 M to about 4 M, about 1 M to about 4.5 M, about 1 M to about 5 M, about 1.5 M to about 2 M, about 1.5 M to about 2.5 M, about 1.5 M to about 3 M, about 1.5 M to about 3.5 M, about 1.5 M to about 4 M, about 1.5 M to about 4.5 M, about 1.5 M to about 5 M, about 2 M to about 2.5 M, about 2 M to about 3 M, about 2 M to about 3.5 M, about 2 M to about 4 M, about 2 M to about 4.5 M, about 2 M to about 5 M, about 2.5 M to about 3 M, about 2.5 M to about 3.5 M, about 2.5 M to about 4 M, about 2.5 M to about 4.5 M, about 2.5 M to about 5 M, about 3 M to about 3.5 M, about 3 M to about 4 M, about 3 M to about 4.5 M, about 3 M to about 5 M, about 3.5 M to about 4 M, about 3.5 M to about 4.5 M, about 3.5 M to about 5 M, about 4 M to about 4.5 M, about 4 M to about 5 M, or about 4.5 M to about 5 M.

In some embodiments, a difference in salt, ion, or electrolyte concentrations between the cis and trans sides can be about 0.01 M, about 0.05 M, about 0.10 M, about 0.20 M, about 0.30 M, about 0.40 M, about 0.50 M, about 0.60 M, about 0.70 M, about 0.80 M, about 0.90 M, about 1.00 M, about 1.10 M, about 1.25 M, about 1.50 M, about 1.75 M, about 2 M, about 2.5 M, about 3 M, about 3.5 M, about 4 M, about 4.5 M, or about 5 M.

In some embodiments, the one or more salts can comprise sodium chloride, sodium carbonate, ammonium chloride, sodium acetate, potassium cyanide, zinc chloride hydroxide, potassium chlorate, calcium phosphate, sodium nitrate, potassium cerium fluoride, Mohr's salt, sodium potassium sulphate, potassium permanganate, tetra amino cupric sulphate, zinc chloride hydroxide monohydrate, monosodium glutamate, copper sulfate, calcium chloride, potassium chloride, magnesium sulfate, magnesium chloride, sodium acetate, magnesium nitrate, potassium glutamate, sodium ferricyanide, sodium ferrocyanide, potassium ferricyanide, potassium ferrocyanide, or any combination thereof.

In some embodiments, the one or more salts on the cis side of the membrane can comprise sodium chloride, sodium carbonate, ammonium chloride, sodium acetate, potassium cyanide, zinc chloride hydroxide, potassium chlorate, calcium phosphate, sodium nitrate, potassium cerium fluoride, Mohr's salt, sodium potassium sulphate, potassium permanganate, tetra amino cupric sulphate, zinc chloride hydroxide monohydrate, monosodium glutamate, copper sulfate, calcium chloride, potassium chloride, magnesium sulfate, magnesium chloride, sodium acetate, magnesium nitrate, or any combination thereof. In some embodiments, the one or more salts on the trans side of the membrane can comprise sodium chloride, sodium carbonate, ammonium chloride, sodium acetate, potassium cyanide, zinc chloride hydroxide, potassium chlorate, calcium phosphate, sodium nitrate, potassium cerium fluoride, Mohr's salt, sodium potassium sulphate, potassium permanganate, tetra amino cupric sulphate, zinc chloride hydroxide monohydrate, monosodium glutamate, copper sulfate, calcium chloride, potassium chloride, magnesium sulfate, magnesium chloride, sodium acetate, magnesium nitrate, or any combination thereof.

In some embodiments, the one or more salts on the cis side of the membrane can be the same as the one or more salts on the trans side of the membrane. In some cases, the one or more salts on the cis side of the membrane can be the same types of salt on the trans side of the membrane. In some embodiments, one or more salts on the cis side of the membrane can be different from the one or more salts on the trans side of the membrane. In some cases, the one or more types of salts on the cis side of the membrane can be different types of salts than the one or more salts on the trans side of the membrane.

In some embodiments, the one or more salts can comprise between about one type of salt to about ten types of salts. In some cases, the one or more salts can comprise at least about one type of salt, at least about two types of salts, at least about three types of salts, at least about four types of salts, at least about five types of salts, at least about six types of salts, at least about seven types of salts, at least about eight types of salts, at least about nine types of salts, at least about ten types of salts, or more than ten types of salt. In some cases, the one or more salts can comprise at most about ten types of salts, at most about nine types of salts, at most about eight types of salts, at most about seven types of salts, at most about six types of salts, at most about five types of salts, at most about four types of salts, at most about three types of salts, at most about two types of salts, at most about one type of salt, or less than one type of salt. In some cases, the one or more salts can comprise one type of salt, about two types of salts, about three types of salts, about four types of salts, about five types of salts, about six types of salts, about seven types of salts, about eight types of salts, about nine types of salts, or about ten types of salts.

In some embodiments, the one or more salts on the cis side membrane can be the same types of salts as the one or more salts on the trans side of the membrane. In some cases, the same types of salts present on the cis side and the trans side of the membrane can be present in the same concentrations. In some cases, the same type of salts present on the cis side and the trans side of the membrane can be present in different concentrations.

In some embodiments, the one or more salts on the cis side membrane can be different salt types than the one or more salts on the trans side of the membrane. In some embodiments, the different types of salts present on the cis side and the trans side of the membrane can be present in the same concentrations. In some cases, the different types of salt present on the cis side and the trans side of the membrane can be present in different concentrations.

In some embodiments, the concentration of one or more salts on the cis side of the membrane can be between about 0.1% to about 500% higher than the concentration of one or more salts on the trans side of the membrane. In some cases, the concentration of one or more salts on the cis side of the membrane can be between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% higher than the concentration of one or more salts on the trans side of the membrane.

In some cases, the concentration of one or more salts on the cis side of the membrane can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% higher than the concentration of one or more salts on the trans side of the membrane.

In some cases, the concentration of one or more salts on the cis side of the membrane can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% higher than the concentration of one or more salts on the trans side of the membrane.

In some cases, the concentration of one or more salts on the cis side of the membrane can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% higher than the concentration of one or more salts on the trans side of the membrane.

In some embodiments, the concentration of one or more salts on the cis side of the membrane can be between about 0.1% to about 500% lower than the concentration of one or more salts on the trans side of the membrane. In some cases, the concentration of one or more salts on the cis side of the membrane can be between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% lower than the concentration of one or more salts on the trans side of the membrane.

In some cases, the concentration of one or more salts on the cis side of the membrane can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% lower than the concentration of one or more salts on the trans side of the membrane.

In some cases, the concentration of one or more salts on the cis side of the membrane can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% lower than the concentration of one or more salts on the trans side of the membrane.

In some cases, the concentration of one or more salts on the cis side of the membrane can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% lower than the concentration of one or more salts on the trans side of the membrane.

In some embodiments, the concentration of one or more salts on the trans side of the membrane can be between about 0.1% to about 500% higher than the concentration of one or more salts on the cis side of the membrane. In some cases, the concentration of one or more salts on the trans side of the membrane can be between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% higher than the concentration of one or more salts on the cis side of the membrane.

In some cases, the concentration of one or more salts on the trans side of the membrane can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% higher than the concentration of one or more salts on the cis side of the membrane.

In some cases, the concentration of one or more salts on the trans side of the membrane can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% higher than the concentration of one or more salts on the cis side of the membrane.

In some cases, the concentration of one or more salts on the trans side of the membrane can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% higher than the concentration of one or more salts on the cis side of the membrane.

In some embodiments, the concentration of one or more salts on the trans side of the membrane can be between about 0.1% to about 500% lower than the concentration of one or more salts on the cis side of the membrane. In some cases, the concentration of one or more salts on the trans side of the membrane can be between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% lower than the concentration of one or more salts on the cis side of the membrane.

In some cases, the concentration of one or more salts on the trans side of the membrane can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% lower than the concentration of one or more salts on the cis side of the membrane.

In some cases, the concentration of one or more salts on the trans side of the membrane can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% lower than the concentration of one or more salts on the cis side of the membrane.

In some cases, the concentration of one or more salts on the trans side of the membrane can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% lower than the concentration of one or more salts on the cis side of the membrane.

In some embodiments, the EOF can be generated by asymmetric salt distribution. An asymmetric salt distribution may be when the concentration of the one or more salts on the cis side of the membrane is greater or less than the concentration of the one or more salts on the trans side of the membrane.

Alternatively, the EOF can be generated by a symmetric salt distribution between the cis side of the membrane and the trans side of the membrane. Symmetric salt distribution may be when the concentration of the one or more salts on the cis side of the membrane is the same as the concentration of the one or more salts on the trans side of the membrane. In some embodiments, the concentration of one or more salts on the cis side of the membrane can be the same as the concentration of the one or more salts on the trans side of the membrane.

In some embodiments, the EOF can be generated by an asymmetric ion distribution between the cis side of the membrane and the trans side of the membrane. Asymmetric ion distribution may be when the concentration of the one or more ions on the cis side of the membrane is greater or less than the concentration of the one or more ions on the trans side of the membrane. In some cases, the concentration of one or more ions on the cis side of the membrane can be greater or less than the concentration of the one or more ions on the trans side of the membrane. In some cases, the concentration of one or more ions on the cis side of the membrane can be higher than the concentration of the one or more ions on the trans side of the membrane. In some cases, the concentration of one or more ions on the cis side of the membrane can be lower than the concentration of one or more ions on the trans side of the membrane. In some cases, the concentration of one or more ions on the trans side of the membrane can be higher than the concentration of the one or more ions on the cis side of the membrane. In some cases, the concentration of one or more ions on the trans side of the membrane can be lower than the concentration of one or more ions on the cis side of the membrane.

In some cases, the concentration of one or more ions on the cis side of the membrane can be between about 1 nanomolar (nM) to about 1,000 nM. In some instances, the concentration of one or more ions on the cis side of the membrane can be between about 1 nM to about 10 nM, between about 10 nM to about 100 nM, or between about 100 nM to about 1,000 nM. In some cases, the concentration of one or more ions on the cis side of the membrane can be at least about 1 nM, at least about 5 nM, at least about 10 nM, at least about 15 nM, at least about 20 nM, at least about 25 nM, at least about 30 nM, at least about 35 nM, at least about 40 nM, at least about 45 nM, at least about 50 nM, at least about 55 nM, at least about 60 nM, at least about 65 nM, at least about 70 nM, at least about 75 nM, at least about 80 nM, at least about 85 nM, at least about 90 nM, at least about 95 nM, at least about 100 nM, at least about 150 nM, at least about 200 nM, at least about 250 nM, at least about 300 nM, at least about 350 nM, at least about 400 nM, at least about 450 nM, at least about 500 nM, at least about 550 nM, at least about 600 nM, at least about 650 nM, at least about 700 nM, at least about 750 nM, at least about 800 nM, at least about 850 nM, at least about 900 nM, at least about 950 nM, at least about 1,000 nM, or more than 1,000 nM. In some cases, the concentration of one or more ions on the cis side of the membrane can at most about 1,000 nM, at most about 950 nM, at most about 900 nM, at most about 850 nM, at most about 800 nM, at most about 750 nM, at most about 700 nM, at most about 650 nM, at most about 600 nM, at most about 550 nM, at most about 500 nM, at most about 450 nM, at most about 400 nM, at most about 350 nM, at most about 300 nM, at most about 250 nM, at most about 200 nM, at most about 150 nM, at most about 100 nM, at most about 95 nM, at most about 90 nM, at most about 85 nM, at most about 80 nM, at most about 75 nM, at most about 70 nM, at most about 65 nM, at most about 60 nM, at most about 55 nM, at most about 45 nM, at most about 40 nM, at most about 35 nM, at most about 30 nM, at most about 25 nM, at most about 20 nM, at most about 15 nM, at most about 10 nM, at most about 5 nM, at most about 1 nM, or less than 1 nM. In some cases, the concentration of salt on the cis side of the membrane can about 1 nM, about 5 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, about 100 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, or about 1,000 nM.

In some cases, the concentration of one or more ions on the trans side of the membrane can be between about 1 nanomolar (nM) to about 1,000 nM. In some instances, the concentration of one or more ions on the trans side of the membrane can be between about 1 nM to about 10 nM, between about 10 nM to about 100 nM, or between about 100 nM to about 1,000 nM. In some cases, the concentration of one or more ions on the trans side of the membrane can be at least about 1 nM, at least about 5 nM, at least about 10 nM, at least about 15 nM, at least about 20 nM, at least about 25 nM, at least about 30 nM, at least about 35 nM, at least about 40 nM, at least about 45 nM, at least about 50 nM, at least about 55 nM, at least about 60 nM, at least about 65 nM, at least about 70 nM, at least about 75 nM, at least about 80 nM, at least about 85 nM, at least about 90 nM, at least about 95 nM, at least about 100 nM, at least about 150 nM, at least about 200 nM, at least about 250 nM, at least about 300 nM, at least about 350 nM, at least about 400 nM, at least about 450 nM, at least about 500 nM, at least about 550 nM, at least about 600 nM, at least about 650 nM, at least about 700 nM, at least about 750 nM, at least about 800 nM, at least about 850 nM, at least about 900 nM, at least about 950 nM, at least about 1,000 nM, or more than 1,000 nM. In some cases, the concentration of one or more ions on the trans side of the membrane can at most about 1,000 nM, at most about 950 nM, at most about 900 nM, at most about 850 nM, at most about 800 nM, at most about 750 nM, at most about 700 nM, at most about 650 nM, at most about 600 nM, at most about 550 nM, at most about 500 nM, at most about 450 nM, at most about 400 nM, at most about 350 nM, at most about 300 nM, at most about 250 nM, at most about 200 nM, at most about 150 nM, at most about 100 nM, at most about 95 nM, at most about 90 nM, at most about 85 nM, at most about 80 nM, at most about 75 nM, at most about 70 nM, at most about 65 nM, at most about 60 nM, at most about 55 nM, at most about 45 nM, at most about 40 nM, at most about 35 nM, at most about 30 nM, at most about 25 nM, at most about 20 nM, at most about 15 nM, at most about 10 nM, at most about 5 nM, at most about 1 nM, or less than 1 nM. In some cases, the concentration of one or more ions on the trans side of the membrane can about 1 nM, about 5 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, about 100 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, or about 1,000 nM.

In some embodiments, the one or more ions can comprise chloride, carbonate, chlorite, chlorate, phosphate, bicarbonate, bromide, ammonium sulfate, ammonium, sulfate, sulfide, calcium, fluoride, hydroxide, aluminum, barium, bismuth, cadmium, cesium, chromium, cobalt, copper, hydrogen, iron, lead, lithium, magnesium, mercury, nickel, potassium, rubidium, silver, sodium, strontium, tin, zinc, iodide, nitride, oxide, or any combinations thereof.

In some embodiments, the one or more ions on the cis side of the membrane can comprise chloride, carbonate, chlorite, chlorate, phosphate, bicarbonate, bromide, ammonium sulfate, ammonium, sulfate, sulfide, calcium, fluoride, hydroxide, aluminum, barium, bismuth, cadmium, cesium, chromium, cobalt, copper, hydrogen, iron, lead, lithium, magnesium, mercury, nickel, potassium, rubidium, silver, sodium, strontium, tin, zinc, iodide, nitride, oxide, or any combinations thereof.

In some embodiments, the one or more ions on the trans side of the membrane can comprise chloride, carbonate, chlorite, chlorate, phosphate, bicarbonate, bromide, ammonium sulfate, ammonium, sulfate, sulfide, calcium, fluoride, hydroxide, aluminum, barium, bismuth, cadmium, cesium, chromium, cobalt, copper, hydrogen, iron, lead, lithium, magnesium, mercury, nickel, potassium, rubidium, silver, sodium, strontium, tin, zinc, iodide, nitride, oxide, or any combinations thereof.

In some embodiments, the one or more ions on the cis side of the membrane can be the same types of ions as the one or more ions on the trans side of the membrane. In some embodiments, one or more ions on the cis side of the membrane can be different types of ions from the one or more ions on the trans side of the membrane.

In some embodiments, the one or more ions can comprise between about one ion to about ten ions. In some cases, the one or more ions can comprise at least about one ion, at least about two ions, at least about three ions, at least about four ions, at least about five ions, at least about six ions, at least about seven ions, at least about eight ions, at least about nine ions, at least about ten ions, or more than ten ions. In some cases, the one or more ions can comprise at most about ten ions, at most about nine ions, at most about eight ions, at most about seven ions, at most about six ions, at most about five ions, at most about four ions, at most about three ions, at most about two ions, at most about one ion, or less than one ion. In some cases, the one or more ions can comprise about one ion, about two ions, about three ions, about four ions, about five ions, about six ions, about seven ions, about eight ions, about nine ions, or about ten ions.

In some embodiments, the one or more ions on the cis side of the membrane can be present in the same concentration as the one or more ions on the trans side of the membrane. In some cases, the one or more ions on the cis side of the membrane can be present in different concentrations as the one or more ions on the trans side of the membrane.

In some embodiments, the concentration of one or more ions on the cis side of the membrane can be between about 0.1% to about 500% higher than the concentration of one or more ions on the trans side of the membrane. In some cases, the concentration of one or more ions on the cis side of the membrane can be between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% higher than the concentration of one or more ions on the trans side of the membrane.

In some cases, the concentration of one or more ions on the cis side of the membrane can beat least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% higher than the concentration of one or more ions on the trans side of the membrane.

In some cases, the concentration of one or more ions on the cis side of the membrane can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% higher than the concentration of one or more ions on the trans side of the membrane.

In some cases, the concentration of one or more ions on the cis side of the membrane can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% higher than the concentration of one or more ions on the trans side of the membrane.

In some embodiments, the concentration of one or more ions on the cis side of the membrane can be between about 0.1% to about 500% lower than the concentration of one or more ions on the trans side of the membrane. In some cases, the concentration of one or more ions on the cis side of the membrane can be between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% lower than the concentration of one or more ions on the trans side of the membrane.

In some cases, the concentration of one or more ions on the cis side of the membrane can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% lower than the concentration of one or more ions on the trans side of the membrane.

In some cases, the concentration of one or more ions on the cis side of the membrane can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% lower than the concentration of one or more ions on the trans side of the membrane.

In some cases, the concentration of one or more ions on the cis side of the membrane can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% lower than the concentration of one or more ions on the trans side of the membrane.

In some embodiments, the concentration of one or more ions on the trans side of the membrane can be between about 0.1% to about 500% higher than the concentration of salt on the cis side of the membrane. In some cases, the concentration of salt on the trans side of the membrane can be between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% higher than the concentration of one or more ions on the cis side of the membrane.

In some cases, the concentration of one or more ions on the trans side of the membrane can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% higher than the concentration of one or more ions on the cis side of the membrane.

In some cases, the concentration of one or more ions on the trans side of the membrane can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% higher than the concentration of one or more ions on the cis side of the membrane.

In some cases, the concentration of one or more ions on the trans side of the membrane can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% higher than the concentration of one or more ions on the cis side of the membrane.

In some embodiments, the concentration of one or more ions on the trans side of the membrane can be between about 0.1% to about 500% lower than the concentration of one or more ions on the cis side of the membrane. In some cases, the concentration of one or more ions on the trans side of the membrane can be between about 0.1% to about 0.5%, between about 0.5% to about 1%, between about 1% to about 5%, between about 5% to about 10%, between about 10% to about 20%, between about 20% to about 30%, between about 30% to about 40%, between about 40% to about 45%, between about 45% to about 50%, between about 50% to about 55%, between about 55% to about 60%, between about 60% to about 65%, between about 65% to about 70%, between about 70% to about 75%, between about 75% to about 80%, between about 80% to about 85%, between about 85% to about 90%, between about 90% to about 95%, between about 95% to about 100%, between about 100% to about 110%, between about 110% to about 120%, between about 120% to about 130%, between about 130% to about 140%, between about 140% to about 150%, between about 150% to about 160%, between about 160% to about 170%, between about 170% to about 180%, between about 180% to about 190%, between about 190% to about 200%, between about 200% to about 210%, between about 210% to about 220%, between about 220% to about 230%, between about 230% to about 240%, between about 240% to about 250%, between about 250% to about 260%, between about 260% to about 270%, between about 270% to about 280%, between about 280% to about 290%, between about 290% to about 300%, between about 300% to about 310%, between about 310% to about 320%, between about 320% to about 330%, between about 330% to about 340%, between about 340% to about 350%, between about 350% to about 360%, between about 360% to about 370%, between about 370% to about 380%, between about 380% to about 390%, between about 390% to about 400%, between about 400% to about 410%, between about 410% to about 420%, between about 420% to about 430%, between about 430% to about 440%, between about 440% to about 450%, between about 450% to about 460%, between about 460% to about 470%, between about 470% to about 480%, between about 480% to about 490%, or between about 490% to about 500% lower than the concentration of one or more ions on the cis side of the membrane.

In some cases, the concentration of one or more ions on the trans side of the membrane can be at least about 0.1%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more than 500% lower than the concentration of one or more ions on the cis side of the membrane.

In some cases, the concentration of one or more ions on the trans side of the membrane can be at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, at most about 0.5%, at most about 0.1%, or less than 0.1% lower than the concentration of one or more ions on the cis side of the membrane.

In some cases, the concentration of one or more ions on the trans side of the membrane can be about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% lower than the concentration of one or more ions on the cis side of the membrane.

Alternatively, the EOF can be generated by a symmetric ion distribution between the cis side of the membrane and the trans side of the membrane. A symmetric ion distribution may be when the concentration of the one or more ions on the cis side of the membrane can be the same concentration of the one or more ions on the trans side of the membrane. In some cases, the concentration of the one or more ions on the cis side of the membrane is the same as the concentration of the one or more ions on the trans side of the membrane.

In some embodiments, the EOF can be generated by an asymmetric concentration of one or more salts and an asymmetric concentration of one or more ions between the cis side of the membrane and the trans side of the membrane.

In some embodiments an electro-osmotic force can act in the same direction as an electrophoretic force or in an opposing direction to an electrophoretic force. In some embodiments, the electro-osmotic force can be greater than the electrophoretic force. In some embodiments, the electro-osmotic force can be less than the electrophoretic force.

The invention relates to systems and methods for analysis of target analytes using nanopore-based sensors. More in particular, it relates to methods, nanopore systems and devices for the single-molecule profiling of polymers, e.g. polypeptides or polysaccharides.

Various studies have demonstrated both freely translocating and motor controlled movement of polypeptides (proteins that are unfolded during or before translocation through narrow nanopores) through narrow nanopores (typically <2 nm in diameter). However, unlike polynucleotides having a fixed negative charge that can be electrophoretically drawn into nanopores by an electric field from an applied voltage, it remains a challenge to capture and control the movement of peptides of diverse composition. This is because the diverse composition leads to a range of electrical and/or structural properties (e.g. a mix of positive, negative, neutral, hydrophilic, hydrophobic, aromatic) that prevent simple capture under electrophoretic conditions and translocation in an unfolded state.

It was previously not thought possible to push/feed analytes (e.g., proteins, peptides, polypeptides) into pores from the cis side in their native form (e.g. without attaching to DNA leaders or adding other (e.g. polyanion) tags to create electrophoretic capture motifs) due to their complex composition. The diverse charge can result in the unfolded peptides are sometimes attracted and sometimes repelled from a nanopore depending on charge and/or applied voltage, so it is not possible to translocate a diverse repertoire of complex peptides through nanopores by electrophoretic means alone. Indeed, previous studies have only demonstrated translocation of either very short peptides with a contour length shorter than the length of the nanopore channel or of very carefully selected (model) protein substrates, whose charge, structure or added electrophoretic tags favor capture and translocation through nanopores by electrophoresis. See for example Cressiot et al., ACS Nano 2015, 9 (9), 9050-9061; Oukhaled et al., Phys. Rev. Lett. 2007, 98 (15);

Merstorf et al., ACS Chem Biol 2012, 7 (4), 652-658; Pastoriza-Gallego et al., ACS Nano 2014, 8 (11), 11350-11360; Rosen et al., Nat. Biotechnol. 2014, 32 (2), 179-181; Yu et al., bioRxiv 2021, 2021.09.28.462155.

However, in no way is this representative for the broad amino acid composition of proteins that are found in nature. See for example Motone et al. (iScience 24, Sep. 24, 2021) reviewing recent approaches that use a range of techniques aimed at driving protein strands and peptides through nanopores. It is stated therein that nanopore protein sequencing is a challenging frontier that has yet to be realized.

Bayat et al. (Nature Comm. 2022 Vol. 13, 5113) reported on the label-free detection and analysis of highly anionic linear polysaccharides by a protein nanopore. It was found that wild-type aerolysin nanopore can detect and characterize glycosaminoglycan oligosaccharides with various sulfate patterns, osidic bonds and epimers of uronic acid residues.

Robertson et al. (BBA—Biomembranes, Vol. 1863, Issue 9, 2021) focussed on the physical chemistry of nanopore sensing and reviewed what types of analytes can be detected. Among others, reference was made to the size discrimination of PEG up to a length of about 48 repeating units by a nanopore system based on a modified alpha-hemolysin (aHL) or aerolysin.

The present disclosure provides a novel approach that is simple and provides robust means of feeding long non-nucleic acid based polymers through nanopores for e.g. the purpose of sequencing or characterizing them. In some cases, the present disclosure can result in polymers being translocated against the direction of the prevailing EPF acting on them to prevent translocation, and not requiring tagging of the polymer analyte.

It was found that these goals can be achieved by using a large and/or dominant cis-to-trans electro-osmotic flow (EOF), generated by a large cis-to-trans excess of ions flowing through the nanopore, that can feed and pass a wide range of elongated, complex polymeric substrates from cis to trans through the nanopore, even against the direction of the electro-phoretic force (EPF) acting on the polymers. In some embodiments, the cis-to-trans osmotic flow can be generated by the flow of ions and/or solvents from the cis side of the nanopore system to the trans side of the nanopore system.

The present disclosure provides a system can utilize strong electro-osmotic forces to capture and feed polymer analytes from the cis side of a nanopore. In some embodiments, the strong electro-osmotic forces pull on the polymer as it translocates through the pore, and enables the structure and/or composition dependent changes in current to be measured and/or characterized.

Accordingly, in one embodiment the invention relates a method for translocating a non-nucleic acid based polymer analyte through a nanopore, the nanopore being comprised in a membrane separating a fluidic chamber of a nanopore system into a cis side and a trans side, comprising adding the analyte to the cis side of and allowing for translocation, wherein the nanopore system has a cis to trans electro-osmotic force (EOF) resulting from a net ionic current flow cis-to-trans. Provided is a method for translocating a non-nucleic acid based polymer analyte through a nanopore, the nanopore being comprised in a membrane separating a fluidic chamber of a nanopore system into a cis side and a trans side, comprising adding the polymer analyte to the cis side of and allowing for polymer analyte translocation to the trans side of the pore, wherein the length of the elongated polymer analyte is larger than the longitudinal axis of the central channel of the nanopore in the direction perpendicular to the membrane, and wherein the nanopore system has a cis to trans EOF resulting from a net ionic current flow cis to trans, and wherein the cis to trans EOF overcomes a trans to cis EPF acting on the polymer analyte.

For example, the nanopore system has a cis to trans EOF resulting from a net ionic current flow cis to trans over total ionic current flow (herein also referred to as Iᵣₑₗ; see below) of greater than 0.2 or less than −0.2, preferably greater than 0.3 or less than −0.3, most preferably greater than 0.35 or less than −0.35.

In some embodiments, the nanopore system has an ion-selectivity P₍₊₎/P₍₋₎ of greater than 2.0 or less than 0.5, preferably greater than 2.5 or less than 0.4, most preferably greater than 3.0 or less than 0.33. The cis to trans EOF is against the trans to cis electrophoretic force (EPF) acting on the analyte. In one aspect, the nanopore system has an ion selectivity P₍₊₎/P₍₋₎ of greater than 3.0 or less than 0.3 under an applied voltage across the membrane.

In some embodiments, the ion-selectivity P₍₊₎/P₍₋₎ can be at least about 2.0, at least about 2.2, at least about 2.4, at least about 2.5, at least about 2.6, at least about 2.7, at least about 2.8, at least about 2.9, at least about 3.0, at least about 3.1, at least about 3.2, at least about 3.3, at least about 3.4, at least about 3.5, at least about 3.6, at least about 3.7, at least about 3.8, at least about 3.9, at least about 4.0, at least about 4.1, at least about 4.2, at least about 4.3, at least about 4.4, at least about 4.5, at least about 4.6, at least about 4.8, at least about 5.0 or greater than about 5.0 in magnitude under an applied voltage across the membrane.

In some embodiments, the ion-selectivity P₍₊₎/P₍₋₎ can be at most about 0.5, at most about 0.45, at most about 0.42, at most about 0.40 mV, at most about 0.38, at most about 0.36, at most about 0.35, at most about 0.34, at most about 0.33, at most about 0.31, at most about 0.30, at most about 0.29, at most about 0.28, at most about 0.27, at most about 0.26, at most about 0.25, at most about 0.24, at most about 0.23, at most about 0.22, at most about 0.21, at most about 0.20 or less than 0.20 in magnitude under an applied voltage across the membrane.

In some embodiments, the ion-selectivity P₍₊₎/P₍₋₎ can be from about 2.0 to about 5.0 in magnitude. In some embodiments, the ion-selectivity P₍₊₎/P₍₋₎ can be from about 2.0 to about 2.2, about 2.0 to about 2.4, about 2.0 to about 2.6, about 2.0 to about 2.8, about 2.0 to about 3.0, about 2.0 to about 3.3, about 2.0 to about 3.6, about 2.0 to about 3.8, about 2.0 to about 4.0, about 2.0 to about 4.3, about 2.0 to about 4.5, about 2.0 to about 4.7, about 2.0 to about 5.0, about 2.5 to about 2.6, about 2.5 to about 2.8, about 2.5 to about 3.0, about 2.5 to about 3.3, about 2.5 to about 3.6, about 2.5 to about 3.8, about 2.5 to about 4.0, about 2.5 to about 4.3, about 2.5 to about 4.5, about 2.5 to about 4.7, about 2.5 to about 5.0, about 3.0 to about 3.2, about 3.0 to about 3.3, about 3.0 to about 3.4, about 3.0 to about 3.5, about 3.0 to about 3.6, about 3.0 to about 3.7, about 3.0 to about 3.8, about 3.0 to about 4.0, about 3.0 to about 4.3, about 3.0 to about 4.5, about 3.0 to about 4.7, about 3.0 to about 5.0, about 3.3 to about 3.4, about 3.3 to about 3.5, about 3.3 to about 3.6, about 3.3 to about 3.7, about 3.3 to about 3.8, about 3.3 to about 4.0, about 3.3 to about 4.3, about 3.3 to about 4.5, about 3.3 to about 4.7, about 3.3 to about 5.0, about 3.5 to about 3.7, about 3.5 to about 3.8, about 3.5 to about 4.0, about 3.5 to about 4.3, about 3.5 to about 4.5, about 3.5 to about 4.7, about 3.5 to about 5.0.

In some embodiments, the ion-selectivity P₍₊₎/P₍₋₎ can be from about 0.20 to about 0.5 in magnitude. In some embodiments, the ion-selectivity P₍₊₎/P₍₋₎ can be from about 0.2 to about 0.22, about 0.2 to about 0.24, about 0.2 to about 0.26, about 0.20 to about 0.28, about 0.20 to about 0.3, about 0.2 to about 0.33, about 0.2 to about 0.36, about 0.20 to about 0.38, about 0.2 to about 0.4, about 0.2 to about 0.43, about 0.20 to about 0.45, about 0.20 to about 0.47, about 0.2 to about 0.48, about 0.25 to about 0.27, about 0.25 to about 0.28, about 0.25 to about 0.30, about 0.25 to about 0.33, about 0.25 to about 0.36, about 0.25 to about 0.38, about 0.25 to about 0.40, about 0.25 to about 0.43, about 0.25 to about 0.45, about 0.25 to about 0.47, about 0.25 to about 5.0, about 0.30 to about 0.32, about 0.30 to about 0.33, about 0.30 to about 0.34, about 0.30 to about 0.35, about 0.30 to about 0.36, about 0.30 to about 0.37, about 0.30 to about 0.38, about 0.30 to about 0.40, about 0.30 to about 0.43, about 0.30 to about 0.45, about 0.30 to about 0.47, about 0.30 to about 0.5.

In some embodiments, the ion-selectivity P₍₊₎/P₍₋₎ can be about 2.0, about 2.2, about 2.4, about 2.6, about 2.8, about 3.0, about 3.3, about 3.5, about 3.6, about 3.8, about 4.0, about 4.3, about 4.6, about 4.8, about 5.0, about 0.5, about 0.45, about 0.40, about 0.38, about 0.35, about 0.33, about 0.30, about 0.28, about 0.25, about 0.23 or about 0.20 in magnitude.

In some embodiments, the applied voltage across the membrane can be at least about 1 mV, at least about 5 mV, at least about 10 mV, at least about 20 mV, at least about 30 mV, at least about 40 mV, at least about 50 mV, at least about 60 mV, at least about 70 mV, at least about 80 mV, at least about 90 mV, at least about 100 mV, at least about 150 mV, at least about 200 mV, at least about 250 mV, at least about 300 mV, at least about 350 mV, at least about 400 mV, at least about 450 mV, at least about 500 mV, at least about 600 mV, at least about 700 mV, at least about 800 mV, at least about 900 mV, at least about 1000 mV, or greater than about 1000 mV in magnitude. In some embodiments, the applied voltage across the membrane can be at least about 1000 mV, at most about 900 mV, at most about 800 mV, at most about 700 mV, at most about 600 mV, at most about 500 mV, at most about 450 mV, at most about 400 mV, at most about 350 mV, at most about 300 mV, at most about 250 mV, at most about 200 mV, at most about 150 mV, at most about 100 mV, at most about 90 mV, at most about 80 mV, at most about 70 mV, at most about 60 mV, at most about 50 mV, at most about 40 mV, at most about 30 mV, at most about 20 mV, at most about 10 mV, at most about 5 mV, at most about 1 mV, or less than about 1 mV in magnitude.

In some embodiments, the applied voltage across the membrane can be from about 1 mV to about 100 mV in magnitude. In some embodiments, the applied voltage across the membrane can be from about 1 mV to about 5 mV, about 1 mV to about 10 mV, about 1 mV to about 20 mV, about 1 mV to about 30 mV, about 1 mV to about 40 mV, about 1 mV to about 50 mV, about 1 mV to about 60 mV, about 1 mV to about 70 mV, about 1 mV to about 80 mV, about 1 mV to about 90 mV, about 1 mV to about 100 mV, about 5 mV to about 10 mV, about 5 mV to about 20 mV, about 5 mV to about 30 mV, about 5 mV to about 40 mV, about 5 mV to about 50 mV, about 5 mV to about 60 mV, about 5 mV to about 70 mV, about 5 mV to about 80 mV, about 5 mV to about 90 mV, about 5 mV to about 100 mV, about 10 mV to about 20 mV, about 10 mV to about 30 mV, about 10 mV to about 40 mV, about 10 mV to about 50 mV, about 10 mV to about 60 mV, about 10 mV to about 70 mV, about 10 mV to about 80 mV, about 10 mV to about 90 mV, about 10 mV to about 100 mV, about 20 mV to about 30 mV, about 20 mV to about 40 mV, about 20 mV to about 50 mV, about 20 mV to about 60 mV, about 20 mV to about 70 mV, about 20 mV to about 80 mV, about 20 mV to about 90 mV, about 20 mV to about 100 mV, about 30 mV to about 40 mV, about 30 mV to about 50 mV, about 30 mV to about 60 mV, about 30 mV to about 70 mV, about 30 mV to about 80 mV, about 30 mV to about 90 mV, about 30 mV to about 100 mV, about 40 mV to about 50 mV, about 40 mV to about 60 mV, about 40 mV to about 70 mV, about 40 mV to about 80 mV, about 40 mV to about 90 mV, about 40 mV to about 100 mV, about 50 mV to about 60 mV, about 50 mV to about 70 mV, about 50 mV to about 80 mV, about 50 mV to about 90 mV, about 50 mV to about 100 mV, about 60 mV to about 70 mV, about 60 mV to about 80 mV, about 60 mV to about 90 mV, about 60 mV to about 100 mV, about 70 mV to about 80 mV, about 70 mV to about 90 mV, about 70 mV to about 100 mV, about 80 mV to about 90 mV, about 80 mV to about 100 mV, or about 90 mV to about 100 mV in magnitude.

In some embodiments, the applied voltage across the membrane can be from about 100 mV to about 1,000 mV in magnitude. In some embodiments, the applied voltage across the membrane can be from about 100 mV to about 150 mV, about 100 mV to about 200 mV, about 100 mV to about 250 mV, about 100 mV to about 300 mV, about 100 mV to about 400 mV, about 100 mV to about 500 mV, about 100 mV to about 600 mV, about 100 mV to about 700 mV, about 100 mV to about 800 mV, about 100 mV to about 900 mV, about 100 mV to about 1,000 mV, about 150 mV to about 200 mV, about 150 mV to about 250 mV, about 150 mV to about 300 mV, about 150 mV to about 400 mV, about 150 mV to about 500 mV, about 150 mV to about 600 mV, about 150 mV to about 700 mV, about 150 mV to about 800 mV, about 150 mV to about 900 mV, about 150 mV to about 1,000 mV, about 200 mV to about 250 mV, about 200 mV to about 300 mV, about 200 mV to about 400 mV, about 200 mV to about 500 mV, about 200 mV to about 600 mV, about 200 mV to about 700 mV, about 200 mV to about 800 mV, about 200 mV to about 900 mV, about 200 mV to about 1,000 mV, about 250 mV to about 300 mV, about 250 mV to about 400 mV, about 250 mV to about 500 mV, about 250 mV to about 600 mV, about 250 mV to about 700 mV, about 250 mV to about 800 mV, about 250 mV to about 900 mV, about 250 mV to about 1,000 mV, about 300 mV to about 400 mV, about 300 mV to about 500 mV, about 300 mV to about 600 mV, about 300 mV to about 700 mV, about 300 mV to about 800 mV, about 300 mV to about 900 mV, about 300 mV to about 1,000 mV, about 400 mV to about 500 mV, about 400 mV to about 600 mV, about 400 mV to about 700 mV, about 400 mV to about 800 mV, about 400 mV to about 900 mV, about 400 mV to about 1,000 mV, about 500 mV to about 600 mV, about 500 mV to about 700 mV, about 500 mV to about 800 mV, about 500 mV to about 900 mV, about 500 mV to about 1,000 mV, about 600 mV to about 700 mV, about 600 mV to about 800 mV, about 600 mV to about 900 mV, about 600 mV to about 1,000 mV, about 700 mV to about 800 mV, about 700 mV to about 900 mV, about 700 mV to about 1,000 mV, about 800 mV to about 900 mV, about 800 mV to about 1,000 mV, or about 900 mV to about 1,000 mV in magnitude.

In some embodiments, the applied voltage across the membrane can be about 1 mV, about 5 mV, about 10 mV, about 20 mV, about 30 mV, about 40 mV, about 50 mV, about 60 mV, about 70 mV, about 80 mV, about 90 mV, about 100 mV, about 150 mV, about 200 mV, about 250 mV, about 300 mV, about 350 mV, about 400 mV, about 450 mV, about 500 mV, about 600 mV, about 700 mV, about 800 mV, about 900 mV, or about 1000 mV in magnitude. In some embodiments the voltage is negative cis to trans. In some embodiments the voltage is positive cis to trans.

In some embodiments a signal is measured. The signal can comprise an electrical signal. The signal can be related to or caused by the translocation of an analyte. The signal can comprise an ionic current, or a change in ionic current. The signal can comprise a voltage, or a change in voltage across the membrane and/or nanopore. The signal can comprise a measurement of a current change between states of a nanopore. The states of a nanopore can comprise an open channel, a capture of an analyte by the nanopore, or a passage of a polymer from a captured state through the nanopore. In some embodiments, measuring the signal can comprise comparing the signal during different states of the nanopore.

In some embodiments, the pair of electrodes are configured to provide an applied voltage to generate the electrophoretic force. In some embodiments, the applied voltage is a negative voltage on the trans side. In some embodiments, the applied voltage is a positive voltage on the trans side. In some embodiments, a magnitude of the applied voltage is less than 300 mV. In some embodiments, a magnitude of the applied voltage is greater than 20 mV. In some embodiments, an absolute relative net electro-osmotic current over the applied voltage is greater than about 0.10 pA/mV. In some embodiments, the nanopore comprises an inner pore constriction from about 0.5 nanometers to about 2 nanometers (nm).

Also provided is a nanopore system for translocating a non-nucleic acid based polymer analyte (e.g., an analyte) through a nanopore, the system comprising a nanopore comprised in a membrane separating a fluidic chamber of the nanopore system into a cis side and a trans side and wherein the analyte is to be added to the cis side, wherein the nanopore system has a cis to trans electro-osmotic force (EOF) resulting from a net ionic current flow cis-to-trans, so that the target polymer is captured in the nanopore. The dominant cis to trans EOF results for example from a net ionic current flow cis-to-trans over total ionic current flow of greater than 0.2 or less than −0.2, preferably greater than 0.3 or less than −0.3, most preferably greater than 0.35 or less than −0.35.

In a specific aspect, a nanopore system of the invention has an ion-selectivity P₍₊₎/P₍₋₎ of greater than 2.0 or less than 0.5, preferably greater than 2.5 or less than 0.4, most preferably greater than 3.0 or less than 0.33 such as >3.5 or <0.28.

A method or nanopore system of the present invention relying on a dominant cis-to-trans EOF resulting from a net ionic current flow cis-to-trans is not taught or suggested in the art.

In some cases, EPF may be the dominant process driving capture and/or translocation in nanopore systems. Hence, all previous demonstrations have either used chosen model polymers (having a net charge that aids EPF), or modified the polymers with highly charged tags (e.g. adding polyanion tags), so that the EPF forces acting on the polymers are in the cis-to-trans direction to drive translocation. Where EOF has previously been employed in nanopore systems, it has most often been either in the trans-to-cis direction acting against a cis-to-trans EPF (slowing down the EPF driven translocation, or trapping the molecule in the nanopore), or in the cis-to-trans direction in combination with a cis-to-trans EPF to aid translocation. While some previous studies have shown the capture of neutral or weakly charged small molecules or small polymers in nanopores via weak electro-osmotic forces (https://doi.org/10.1073/pnas.2531778100; https://pubs.acs.org/doi/full/10.1021/ja4026193; https://doi.org/10.1063/1.2723088) there are no enabling disclosures that it is possible to capture and/or translocate long and/or complex polymers (with a contour length greater than the length of pore) using cis-to-trans EOF that is capable of overcoming trans-to-cis EPF acting in the opposite direction.

See also US2022/0283140 Å1 disclosing a method and system for performing single molecule proteomics utilizing a nanopore sensor to measure an electronic signature of protein or peptide being transported through the nanopore utilizing an agent, such as guanidinium chloride, to bind to the nanopore's interior and/or provide an electroosmotic force within the nanopore. In this system, EOF is used to aid in the translocation, but it is setup where EPF is in the same direction. The present disclosure provides methods and systems in which a cis to trans EOF may overcome a reverse EPF.

While some previous studies have shown the capture of neutral or weakly charged small molecules or small polymers in nanopores via weak electro-osmotic forces, there are no enabling disclosures that it is possible to capture and/or translocate long and/or complex polymers (with a contour length greater than the length of pore) using cis-to-trans EOF that is capable of overcoming trans-to-cis EPF acting in the opposite direction.

In some embodiments, the novel system relies on arranging specific strong electro-osmotic means in the direction of translocation. This could not have been predicted, as the EOF to the system acting on the polymer analyte could have been repelled the analyte, thus preventing its capture and/or translocation. Furthermore, the long polymer analyte could have become clogged in the nanopore. In fact, this is how EOF has been used most in prior art nanopore systems, to act to create a trap to keep the analyte in the pore. In some cases, the nanopore captures a free end of the polymer as there are no tags on the end to create strong EPF.

The polymer analyte can be of synthetic, semi-synthetic or biological origin. For example, it is a biopolymer other than DNA. It may comprise or consist of peptide units, saccharide units or water-soluble plastic monomers, and any combination thereof. Preferably, the polymer analyte is a polypeptide, polysaccharide, or a water-soluble plastic, such as PEG, or a PEGylated polypeptide.

In one embodiment, the polymer analyte is an unmodified (label-free) analyte. Suitably, in a method of the invention, the termini of the polymer are unstructured, preferably wherein the polymer is denatured or partially denatured. In one aspect, the length of the elongated polymer is larger than the than the longitudinal axis of the central channel of the nanopore in direction perpendicular to the membrane, preferably wherein the length of the polymer is >50 monomer units, such as >50 peptide units.

In a preferred embodiment, the invention provides a method for translocating according a non-nucleic acid based polymer analyte (e.g., analyte, polypeptide) of at least 30 peptide units and/or comprising positively and/or negatively charged residues. The polypeptide may be in a denatured/unfolded state, preferably the polypeptide is added in a pre-denatured state.

The method may further comprise (c) measuring ionic current changes caused by translocation of the target polymer through the nanopore, preferably wherein operation (c) comprises measuring current changes for states of (i) open channel, (ii) capture of the polymer by the nanopore, and/or (iii) passage of a polymer from (ii) through the nanopore, more preferably wherein the measuring comprises detecting differences between states (i), (ii) and/or (iii). In one embodiment, the measuring comprises measuring differences during state (iii) caused by the composition and/or structure of the polymer passing through the nanopore.)

The cis to trans EOF can be achieved by various means. For example, it is arranged by modulating the pH, type and/or concentration of a salt and/or osmotic pressure across the membrane of the nanopore system, by modification (e.g. genetic engineering) of the nanopore charge, or any combination thereof. Preferably, the dominant EOF is achieved by modification of the nanopore and/or asymmetric salt distribution between the cis and trans side of the chamber.

In a specific aspect, the system has an ion-selectivity P₍₊₎/P₍₋₎ of greater than 2.0, preferably greater than 2.5, most preferably greater than 3.0, preferably wherein the system comprises a cation-selective (mutant) nanopore.

In one embodiment, the nanopore is a solid state nanopore or a biological nanopore, preferably having an inner pore constriction with a diameter in the range of 0.5-2 nm.

In some embodiments, the nanopore can be a biological nanopore, more preferably an alpha-helical or beta-barrel oligomeric pore forming toxin or porin. The nanopore is suitably selected from the group consisting of Aerolysin (Aer), Cytolysin K (CytK), MspA, alpha-hemolysin (aHL), CsgG, Fragaceatoxin C (FraC), Lysenin, phage derived portal proteins (Phi29, G20c, etc) or a mutant thereof. In certain aspects, the nanopore is selected from the mutant CytK nanopores listed in Table 1. A person of skill will understand the nanopore can also be one that is built from elements of existing nanopores (see e.g. WO2021/101378) or developed de novo using predictive protein engineering software (see e.g. Shimizu et al. 2022, Nature Nanotechnology volume 17, pg. 67-75).

A method according to invention may further comprise operation (c) of measuring ionic current changes caused by translocation of the polymer analyte through the nanopore. operation (c) preferably comprises measuring current changes for states of (i) open channel, (ii) capture of the polymer by the nanopore, and/or (iii) passage of a polymer from (ii) through the nanopore. For example, it comprises detecting differences between states (i), (ii) and/or (iii). In a specific aspect, the measuring comprises measuring differences during state (iii) caused by the composition and/or structure of the non-nucleic acid based polymer analyte (e.g., analyte, protein) passing through the nanopore. The method suitably comprises taking one or more measurements characteristic of the target polymer. The one or more measurements may be characteristic of one, two, three, four or five or more characteristics of the polymer analyte. One or more characteristics are preferably selected from (i) length of the polymer; (ii) polymer identity; (iii) polymer sequence; (iv) secondary or tertiary structures of the polymer; and/or (v) whether the polymer was (post-translationally) modified or not. Any combination of (i) to (v) may be measured in accordance with the invention.

A further embodiment of the invention relates to a nanopore system for translocating a polymer analyte through a nanopore, comprising:

(a) a membrane having nanopore therein, said membrane separating a chamber into a cis side and a trans side, wherein the polymer analyte is to be added to the cis side and translocated through the nanopore to the trans side; (b) on the cis side of said chamber a polymer analyte captured by a protein translocase, which can bind and translocate the polymer analyte through the nanopore in a sequential order; and (c) means for providing a voltage difference between the cis side and the trans side of the membrane.

In some embodiments, the nanopore system is further characterized by a cis to trans electro-osmotic force (EOF) resulting from a net ionic current flow cis-to-trans, so that the polymer analyte is captured in the nanopore. Preferably, the nanopore system has a cis to trans EOF resulting from a net ionic current flow cis-to-trans (Iᵣₑₗ) over total ionic current flow of greater than 0.2 or less than −0.2, more preferably greater than 0.3 or less than −0.3, most preferably greater than 0.35 or less than −0.35.

In a specific aspect, the nanopore system has an ion-selectivity P₍₊₎/P₍₋₎ of greater than 2.0 or less than 0.5, preferably greater than 2.5 or less than 0.4, more preferably greater than 3.0 or less than 0.33, or even greater than 3.5 or less than 0.2.

A voltage difference can be provided in various ways, for example one circuit can both apply the voltage and/or measure the current; or the system contains a first circuit to apply a voltage and/or a second circuit to measure the current. It is also possible to create a voltage difference with an asymmetric salt across the membrane. For example, the device comprises a circuit for providing a voltage between the cis side and the trans side and for measuring ionic current flowing through the nanopore. See FIG. 1. Preferably, a negative voltage is applied on the trans side.

The system may further comprise means for measuring a signal based on ionic current flowing through the nanopore during a period of time of translocation. These measuring means are set up to detect changes in the signal that reflect characteristics of the analyte (e.g., protein) as it is translocated.

The system may employ alternative means of measuring the voltage-current properties of the nanopore system, such as those that employ fluorescence probes of ionic flux or field effect transistor systems than measure changes in voltage. However, there are also other suitable detection methods, such as tunneling, surface enhanced raman, plasmonics, and/or other spectroscopic methods that do not measure the ionic current and instead measure the properties of the target analyte in the nanopore directly.

Also provided is an analytical device comprising one or more nanopore systems as herein disclosed, e.g. in the form of an array.

A further embodiment relates the use of a method, nanopore system or device according to the invention for characterizing at least one feature of a polymer analyte, preferably for detection and/or analysis of one or more polymer analyte(s) at the single molecule level. The method does not rely on the charge of the polymer analyte to be analyzed so, in principle, any type of polymer can be analyzed. The method and corresponding system provides a highly desired single molecule polymer-sequencing approach. Embodiments of the invention may be applied to protein or glycan sequencing, single-molecule protein or glycan sequencing, proteomics, detection of post-translational modifications in single cells, glyco-peptide analysis, detection of protein or glycan biomarkers and/or their post-translational modifications, and/or detection of disease biomarkers. One preferred application for this method is single-molecule protein sequencing, and/or the discovery and/or quantification of post-translational modifications in proteins.

### Definitions

### Electro-Osmotic Force

According to the invention, the nanopore system has a cis-to-trans electro-osmotic flow, or vice versa, which creates a drag on the particles dispersed in the solution (independent of their charge) that is often termed an electro-osmotic force (EOF). The EOF arises from a net flow of ions (e.g. cis to trans) that creates a strong force on the solvent itself (water) sufficient to move the fluid (Chinappi et al., 2020, ACS Nano, 14, 11, pg. 15816-15828), which imposes a significant force on any molecules within the flux. Electroosmosis can either compete or cooperate with electrophoresis (EPF).

According to the invention, the nanopore system has a cis-to-trans electro-osmotic flow with an EOF that dominates over EPF. Surprisingly, a sufficiently dominant cis-to-trans EOF enables capture and/or translocation of complex and/or charged polymers against EPF acting trans-to-cis. This selected high and/or dominant EOF is believed to capture and/or retain the target analyte in the nanopore. The EOF pulls on the polymer directly, pulling the polymer through the nanopore.

In some embodiments, the cis side is meant to indicate the compartment of the sensor system to which analyte(s) is added and/or the nanopore is added in the case of a biologically derived nanopore (and assuming vectorial insertion as most nanopores have a selective insertion orientation based on which compartment they are inserted from). However, it is to be noted that the terms “trans” and “cis” are used herein as the common convention determined by electronics/voltage polarity at the trans electrode. For example, without wishing to be bound to any one type of electrical circuit as many options are possible, the cis chamber is at ground and the applied transmembrane potential is given as the potential on the trans side i.e. the trans potential minus the cis potential. A positive current is one in which positive charge (e.g. K⁺ ions) moves through the nanopore from the trans to the cis side, or negative charge (e.g. Cl⁻ ions) from the cis to the trans side (see e.g. Maglia et al. Methods Enzymol. 2010; 475: 591-623).

In some embodiments, the present invention teaches that the direction of the EOF is dependent on the polarity of the applied voltage and/or the relative conditions in the cis and trans compartments in combination with any ion selectivity of the nanopore. Further, the invention teaches that the direction of the EOF (be it cis-to-trans or trans-to-cis) dictates the direction of the net forces acting to translocate the polymer analyte across the nanopore, and thus dictates to which side the polymer analyte is added within the context of the methods described herein. Thus, for example, it is also possible within the context of the invention to add the analyte to the trans side of the membrane to enable trans-to-cis threading for a system where the EOF is created trans-to-cis. A person of skill will also understand that while it is conventional to insert biological nanopores from the cis compartment, it will also be possible to insert them from the trans compartment, and that both orientations of the nanopore relative to the EOF can be employed.

In some embodiments, a core principle underlying the present invention is the generation of a net ion flux in one direction across the nanopore to create a dominant EOF that enables capture and/or transport of polymer analytes. Ion selective ion flux across membranes can be described by the well-known Goldman-Hodgkin-Katz (GHK) flux equation (Bertil Hille, 2001, Ion channels of excitable membranes, 3rd ed.), which is used to determine the ionic current (I_{(S)}) ion species S across the membrane as a function of the applied potential (Vₘ):

$${I}_{( s )} = {P}_{( s )} {z}_{s}^{2} * \frac{{V}_{m} {F}^{2}}{RT} \frac{{[ S ]}_{trans} - {[ S ]}_{cis} * {e}^{- {z}_{s} \frac{{V}_{m} F}{RT}}}{1 - {e}^{- {z}_{s} \frac{{V}_{m} F}{RT}}}$$

where P_{(S)} is the membrane permeability of ion species S, zₛ the valency of the ion, F the Faraday constant, R the gas constant, T the temperature and [S]_{cis} and [S]ₜᵣₐₙₛ the cis and trans concentrations of ion species S, respectively. The GHK flux equation can therefore be used to determine the separate current flow contributions (e.g. I_{(S1)}, I_{(S2)}, I_{(S3)}, etc) of all the ion species (e.g. S1, S2, S3, etc) in the system, flowing either cis-to-trans or trans-to-cis.

The separate ionic current contributions can be combined to determine the measured current (I) (which accounts for the direction of the ionic flows relative to the polarity of the applied voltage):

$$I = {I}_{( s 1 )} + {I}_{( s 2 )} + {I}_{( s 3 )}$$

which will approximately match the ionic current that is measured experimentally across the nanopore system under an applied voltage (ignoring any access resistance from the bulk solution).

In some embodiments, the total absolute ionic current (Iₜₒₜₐₗ) flowing through the nanopore regardless of direction is given by the sum of absolute component currents:

$${I}_{total} = \vert {I}_{( s 1 )} \vert + \vert {I}_{( s 2 )} \vert + \vert {I}_{( s 3 )} \vert$$

In some cases, when accounting for direction of flow, the separate ionic currents (e.g. I_{(S1)}, I_{(S2)}, I_{(S3)}, etc) can also be combined to determine the separate components of the net ionic current flowing cis-to-trans (I_{c→t}) and net ionic current flowing trans-to-cis (I_{t→c}).

$${I}_{c \to t} = - 1 \times {\sum }_{{I}_{( Sn )} < 1} {I}_{( s 1 )} + {I}_{( s 2 )} + {I}_{( s 3 )}$$

$${I}_{t \to c} = {\sum }_{{I}_{( Sn )} > 1} {I}_{( s 1 )} + {I}_{( s 2 )} + {I}_{( s 3 )}$$

These in turn can be used to determine the net ion current flow cis-to-trans (I_{Δc→t}):

$${I}_{Δ c \to t} = {I}_{c \to t} - {I}_{t \to c}$$

In some cases, to understand the relative magnitude of the net current flow cis-to-trans as a proportion of the total current flowing through the nanopore, the net cis-to-trans is divided by the total amount of current flowing to obtain:

$${I}_{rel} = \frac{{I}_{c \to t} - {I}_{t \to c}}{{I}_{c \to t} + {I}_{t \to c}} = \frac{{I}_{Δ c \to t}}{{I}_{total}}$$

where Iᵣₑₗ is the relative net current flow cis-to-trans.

In some cases, in a balanced nanopore system, under an applied voltage the cis-to-trans current (I_{c→t}) is typically balanced by an equal trans-to-cis current (I_{t→c}), so that I_{Δc→t} ≈0 and Iᵣₑₗ≈0. In a nanopore system where the net current flowing is cis-to-trans then Iᵣₑₗ>0 up to a maximum of Iᵣₑₗ=1 when all the current is flowing cis-to-trans. Vice versa, in a nanopore system where the net current flowing is trans-to-cis then Iᵣₑₗ<0 to a maximum of Iᵣₑₗ=−1. Therefore, Iᵣₑₗ varies between −1 and 1, and the further away from 0 the stronger the net current flowing through the nanopore in one direction is, and hence the stronger the resulting EOF is in that direction.

According to the invention, polymer analyte capture and/or translocation is enabled in a nanopore system with a large net cis-to-trans current I_{Δc→t}>>0 arising from a large relative difference between the cis-to-trans current and the trans-to-cis current (I_{Δt→c}), or vice versa (I_{Δt→c}>>0 from Iᵣₑₗ<1). Suitable Iᵣₑₗ are greater than 0.2 or less than −0.2, preferably greater than 0.3 or less than −0.3, most preferably greater than 0.4 or less than −0.4.

In some embodiments, the GHK flux equation indicates how to create a large net cis-to-trans current by altering one or more of the nanopore system variables, including the system ion-selectivity P₍₊₎/P₍₋₎, the mixtures of salts used, the salt concentrations and/or salt asymmetries, and/or the applied voltage. At least three methods can generate a net total ion flux across the membrane: 1) an asymmetry in electrolyte concentration (e.g. 1 M KCl buffer in cis and 0.1 M KCl buffer in trans), 2) an asymmetry in electrolyte compositions with different permeabilities (e.g. 1 M KCl in cis and 1 M KGlutamate in trans), 3) the use of ion-selective membrane channels. These methods can be used as such or in any combination.

In some embodiments, a salt, ion, osmolyte, or electrolyte concentration on the cis side can be at least about 0.01 M, at least about 0.05 M, at least about 0.10 M, at least about 0.20 M, at least about 0.30 M, at least about 0.40 M, at least about 0.50 M, at least about 0.60 M, at least about 0.70 M, at least about 0.80 M, at least about 0.90 M, at least about 1.00 M, at least about 1.10 M, at least about 1.25 M, at least about 1.50 M, at least about 1.75 M, at least about 2 M, at least about 2.5 M, at least about 3 M, at least about 3.5 M, at least about 4 M, at least about 4.5 M, at least about 5 M, or greater than about 5 M. In some embodiments, a salt, ion, osmolyte, or electrolyte concentration on the cis side can be at most about 5 M, at most about 4.5 M, at most about 4 M, at most about 3.5 M, at most about 3 M, at most about 2.5 M, at most about 2 M, at most about 1.75 M, at most about 1.50 M, at most about 1.25 M, at most about 1 M, at most about 0.90 M, at most about 0.80 M, at most about 0.70 M, at most about 0.60 M, at most about 0.50 M, at most about 0.40 M, at most about 0.30 M, at most about 0.20 M, at most about 0.10 M, at most about 0.05 M, at most about 0.01 M, or less than about 0.01 M.

In some embodiments, a salt, ion, osmolyte, or electrolyte concentration on the cis side can be from about 0.01 M to about 5 M. In some embodiments, a salt, ion, osmolyte, or electrolyte concentration on the cis side can be from about 0.01 M to about 0.1 M, about 0.01 M to about 0.5 M, about 0.01 M to about 1 M, about 0.01 M to about 1.5 M, about 0.01 M to about 2 M, about 0.01 M to about 2.5 M, about 0.01 M to about 3 M, about 0.01 M to about 3.5 M, about 0.01 M to about 4 M, about 0.01 M to about 4.5 M, about 0.01 M to about 5 M, about 0.1 M to about 0.5 M, about 0.1 M to about 1 M, about 0.1 M to about 1.5 M, about 0.1 M to about 2 M, about 0.1 M to about 2.5 M, about 0.1 M to about 3 M, about 0.1 M to about 3.5 M, about 0.1 M to about 4 M, about 0.1 M to about 4.5 M, about 0.1 M to about 5 M, about 0.5 M to about 1 M, about 0.5 M to about 1.5 M, about 0.5 M to about 2 M, about 0.5 M to about 2.5 M, about 0.5 M to about 3 M, about 0.5 M to about 3.5 M, about 0.5 M to about 4 M, about 0.5 M to about 4.5 M, about 0.5 M to about 5 M, about 1 M to about 1.5 M, about 1 M to about 2 M, about 1 M to about 2.5 M, about 1 M to about 3 M, about 1 M to about 3.5 M, about 1 M to about 4 M, about 1 M to about 4.5 M, about 1 M to about 5 M, about 1.5 M to about 2 M, about 1.5 M to about 2.5 M, about 1.5 M to about 3 M, about 1.5 M to about 3.5 M, about 1.5 M to about 4 M, about 1.5 M to about 4.5 M, about 1.5 M to about 5 M, about 2 M to about 2.5 M, about 2 M to about 3 M, about 2 M to about 3.5 M, about 2 M to about 4 M, about 2 M to about 4.5 M, about 2 M to about 5 M, about 2.5 M to about 3 M, about 2.5 M to about 3.5 M, about 2.5 M to about 4 M, about 2.5 M to about 4.5 M, about 2.5 M to about 5 M, about 3 M to about 3.5 M, about 3 M to about 4 M, about 3 M to about 4.5 M, about 3 M to about 5 M, about 3.5 M to about 4 M, about 3.5 M to about 4.5 M, about 3.5 M to about 5 M, about 4 M to about 4.5 M, about 4 M to about 5 M, or about 4.5 M to about 5 M.

In some embodiments, a salt, ion, osmolyte, or electrolyte concentration on the cis side can be about 0.01 M, about 0.05 M, about 0.10 M, about 0.20 M, about 0.30 M, about 0.40 M, about 0.50 M, about 0.60 M, about 0.70 M, about 0.80 M, about 0.90 M, about 1.00 M, about 1.10 M, about 1.25 M, about 1.50 M, about 1.75 M, about 2 M, about 2.5 M, about 3 M, about 3.5 M, about 4 M, about 4.5 M, or about 5 M.

In some embodiments, a salt, ion, osmolyte, or electrolyte concentration on the trans side can be at least about 0.01 M, at least about 0.05 M, at least about 0.10 M, at least about 0.20 M, at least about 0.30 M, at least about 0.40 M, at least about 0.50 M, at least about 0.60 M, at least about 0.70 M, at least about 0.80 M, at least about 0.90 M, at least about 1.00 M, at least about 1.10 M, at least about 1.25 M, at least about 1.50 M, at least about 1.75 M, at least about 2 M, at least about 2.5 M, at least about 3 M, at least about 3.5 M, at least about 4 M, at least about 4.5 M, at least about 5 M, or greater than about 5 M. In some embodiments, a salt, ion, osmolyte, or electrolyte concentration on the trans side can be at most about 5 M, at most about 4.5 M, at most about 4 M, at most about 3.5 M, at most about 3 M, at most about 2.5 M, at most about 2 M, at most about 1.75 M, at most about 1.50 M, at most about 1.25 M, at most about 1 M, at most about 0.90 M, at most about 0.80 M, at most about 0.70 M, at most about 0.60 M, at most about 0.50 M, at most about 0.40 M, at most about 0.30 M, at most about 0.20 M, at most about 0.10 M, at most about 0.05 M, at most about 0.01 M, or less than about 0.01 M.

In some embodiments, a salt, ion, osmolyte, or electrolyte concentration on the trans side can be from about 0.01 M to about 5 M. In some embodiments, a salt, ion, osmolyte, or electrolyte concentration on the trans side can be from about 0.01 M to about 0.1 M, about 0.01 M to about 0.5 M, about 0.01 M to about 1 M, about 0.01 M to about 1.5 M, about 0.01 M to about 2 M, about 0.01 M to about 2.5 M, about 0.01 M to about 3 M, about 0.01 M to about 3.5 M, about 0.01 M to about 4 M, about 0.01 M to about 4.5 M, about 0.01 M to about 5 M, about 0.1 M to about 0.5 M, about 0.1 M to about 1 M, about 0.1 M to about 1.5 M, about 0.1 M to about 2 M, about 0.1 M to about 2.5 M, about 0.1 M to about 3 M, about 0.1 M to about 3.5 M, about 0.1 M to about 4 M, about 0.1 M to about 4.5 M, about 0.1 M to about 5 M, about 0.5 M to about 1 M, about 0.5 M to about 1.5 M, about 0.5 M to about 2 M, about 0.5 M to about 2.5 M, about 0.5 M to about 3 M, about 0.5 M to about 3.5 M, about 0.5 M to about 4 M, about 0.5 M to about 4.5 M, about 0.5 M to about 5 M, about 1 M to about 1.5 M, about 1 M to about 2 M, about 1 M to about 2.5 M, about 1 M to about 3 M, about 1 M to about 3.5 M, about 1 M to about 4 M, about 1 M to about 4.5 M, about 1 M to about 5 M, about 1.5 M to about 2 M, about 1.5 M to about 2.5 M, about 1.5 M to about 3 M, about 1.5 M to about 3.5 M, about 1.5 M to about 4 M, about 1.5 M to about 4.5 M, about 1.5 M to about 5 M, about 2 M to about 2.5 M, about 2 M to about 3 M, about 2 M to about 3.5 M, about 2 M to about 4 M, about 2 M to about 4.5 M, about 2 M to about 5 M, about 2.5 M to about 3 M, about 2.5 M to about 3.5 M, about 2.5 M to about 4 M, about 2.5 M to about 4.5 M, about 2.5 M to about 5 M, about 3 M to about 3.5 M, about 3 M to about 4 M, about 3 M to about 4.5 M, about 3 M to about 5 M, about 3.5 M to about 4 M, about 3.5 M to about 4.5 M, about 3.5 M to about 5 M, about 4 M to about 4.5 M, about 4 M to about 5 M, or about 4.5 M to about 5 M.

In some embodiments, a salt, ion, osmolyte, or electrolyte concentration on the trans side can be about 0.01 M, about 0.05 M, about 0.10 M, about 0.20 M, about 0.30 M, about 0.40 M, about 0.50 M, about 0.60 M, about 0.70 M, about 0.80 M, about 0.90 M, about 1.00 M, about 1.10 M, about 1.25 M, about 1.50 M, about 1.75 M, about 2 M, about 2.5 M, about 3 M, about 3.5 M, about 4 M, about 4.5 M, or about 5 M.

In some embodiments, a difference in salt, ion, or electrolyte concentrations between the cis and trans sides can be at least about 0.01 M, at least about 0.05, at least about 0.10, at least about 0.20, at least about 0.30, at least about 0.40, at least about 0.50, at least about 0.60, at least about 0.70, at least about 0.80, at least about 0.90, at least about 1.00, at least about 1.10, at least about 1.25, at least about 1.50, at least about 1.75, at least about 2, at least about 2.5, at least about 3, at least about 3.5, at least about 4, at least about 4.5, at least about 5 M, or greater than about 5 M. In some embodiments, a difference in salt, ion, or electrolyte concentrations between the cis and trans sides can be at most about 5 M, at most about 4.5 M, at most about 4 M, at most about 3.5 M, at most about 3 M, at most about 2.5 M, at most about 2 M, at most about 1.75 M, at most about 1.50 M, at most about 1.25 M, at most about 1 M, at most about 0.90 M, at most about 0.80 M, at most about 0.70 M, at most about 0.60 M, at most about 0.50 M, at most about 0.40 M, at most about 0.30 M, at most about 0.20 M, at most about 0.10 M, at most about 0.05 M, at most about 0.01 M, or less than about 0.01 M.

In some embodiments, a difference in salt, ion, or electrolyte concentrations between the cis and trans sides can be from about 0.01 M to about 5 M. In some embodiments, a difference in salt, ion, or electrolyte concentrations between the cis and trans sides can be from about 0.01 M to about 0.1 M, about 0.01 M to about 0.5 M, about 0.01 M to about 1 M, about 0.01 M to about 1.5 M, about 0.01 M to about 2 M, about 0.01 M to about 2.5 M, about 0.01 M to about 3 M, about 0.01 M to about 3.5 M, about 0.01 M to about 4 M, about 0.01 M to about 4.5 M, about 0.01 M to about 5 M, about 0.1 M to about 0.5 M, about 0.1 M to about 1 M, about 0.1 M to about 1.5 M, about 0.1 M to about 2 M, about 0.1 M to about 2.5 M, about 0.1 M to about 3 M, about 0.1 M to about 3.5 M, about 0.1 M to about 4 M, about 0.1 M to about 4.5 M, about 0.1 M to about 5 M, about 0.5 M to about 1 M, about 0.5 M to about 1.5 M, about 0.5 M to about 2 M, about 0.5 M to about 2.5 M, about 0.5 M to about 3 M, about 0.5 M to about 3.5 M, about 0.5 M to about 4 M, about 0.5 M to about 4.5 M, about 0.5 M to about 5 M, about 1 M to about 1.5 M, about 1 M to about 2 M, about 1 M to about 2.5 M, about 1 M to about 3 M, about 1 M to about 3.5 M, about 1 M to about 4 M, about 1 M to about 4.5 M, about 1 M to about 5 M, about 1.5 M to about 2 M, about 1.5 M to about 2.5 M, about 1.5 M to about 3 M, about 1.5 M to about 3.5 M, about 1.5 M to about 4 M, about 1.5 M to about 4.5 M, about 1.5 M to about 5 M, about 2 M to about 2.5 M, about 2 M to about 3 M, about 2 M to about 3.5 M, about 2 M to about 4 M, about 2 M to about 4.5 M, about 2 M to about 5 M, about 2.5 M to about 3 M, about 2.5 M to about 3.5 M, about 2.5 M to about 4 M, about 2.5 M to about 4.5 M, about 2.5 M to about 5 M, about 3 M to about 3.5 M, about 3 M to about 4 M, about 3 M to about 4.5 M, about 3 M to about 5 M, about 3.5 M to about 4 M, about 3.5 M to about 4.5 M, about 3.5 M to about 5 M, about 4 M to about 4.5 M, about 4 M to about 5 M, or about 4.5 M to about 5 M.

In some embodiments, a difference in salt, ion, or electrolyte concentrations between the cis and trans sides can be about 0.01 M, about 0.05 M, about 0.10 M, about 0.20 M, about 0.30 M, about 0.40 M, about 0.50 M, about 0.60 M, about 0.70 M, about 0.80 M, about 0.90 M, about 1.00 M, about 1.10 M, about 1.25 M, about 1.50 M, about 1.75 M, about 2 M, about 2.5 M, about 3 M, about 3.5 M, about 4 M, about 4.5 M, or about 5 M.

In some embodiments, the concentration of the solute is greater on the cis side than the trans side. In some embodiments, the concentration of the solute is greater on the trans side than the cis side.

According to the invention, creating a highly ion-selective nanopore system is one means of creating a directional net flow of water (the EOF) across a membrane, even in a system where there is no salt asymmetry. Nanopores can be ion selective due to the electrostatic effect that charge (in particular charge in the inner surface of the central water-filled channel) has on the nearby ions flowing through the constrained dimensions of the central channel. The ion-selectivity (the preference for translocating one ion species over another) is reflected in the GHK flux equation by the ion-permeability of each ion species.

The ion selectivity of a nanopore system can be quantified by well-known means of measuring the current-voltage (I-V) relationship under asymmetric electrolyte conditions. Under asymmetric electrolyte conditions, a net flow of ions will occur when no voltage is applied (Vₘ=0 mV). However, when a specific reversal potential (Vᵣ) is applied, the flux of positive and negative ions is equal in magnitude and direction and no net current is measured across the system, enabling the GHK flux equation to be solved at 0 pA for both species of ions to discover the ion-selectivity ratio:

$$\frac{{P}_{( X + )}}{{P}_{( Y - )}} = \frac{{[ {a}_{{Y}^{-}} ]}_{trans} - {[ {a}_{{Y}^{-}} ]}_{cis} * {e}^{\frac{{V}_{r} E}{RT}}}{{[ {a}_{{X}^{+}} ]}_{trans} * {e}^{\frac{{V}_{r} E}{RT}} - {[ {a}_{{X}^{+}} ]}_{cis}}$$

wherein P_{(X+)} and P_{(Y-)} denote the permeability of the nanopore system for cation species X and anion species Y respectively. [α_{γ}−] and [αₓ−] are the activity of ion Y and X respectively in the indicated compartment, and can be calculated by multiplying the concentration with the mean ion activity coefficient (known and tabulated for most electrolytes (Lide, D. R., 2003, CRC handbook of chemistry and physics, 84th edition, Handb. Chem. Phys. 53, 2616)). The latter is to correct for the presence of other ions in concentrated electrolyte solutions. The empirical ion-selectivity ratio (P_{(X+)}/P_{(Y-)}) can inserted back into the GHK flux equations in combination with experimental measurements of ionic current versus applied voltage (I-V curves) for a nanopore system containing the XY salts on both cis and trans to determine the absolute values of P_{(X+)} and P_{(Y-)}. Thus, permeability P_{(S)} can be determined for any ion species S employed in the nanopore system of the invention, and then used in the GHK flux equations to determine the underlying ionic current flows for nanopore systems containing a mixtures of two or more ion species (e.g. asymmetric salts types).

In some embodiments, under symmetric salt conditions, in a system comprised primarily of two ions X+ and Y− in both compartments, the ion-selectivity ratio (P_{(X+)}/P_{(Y-)}) determines the relative ion flux that will flow across the membrane cis-to-trans and trans-to-cis. Thus, if P_{(X+)}/P_{(Y-)}>1, the cation species dominate the ion flux and the EOF is directed towards the negative electrode, whereas the EOF is directed towards the positive electrode when P_{(X+)}/P_{(Y-)}<1. Pores with larger (P_{(X+)}/P_{(Y-)}) ratios will have a larger net ion flux and hence a larger EOF.

In some cases, when mixtures of salts are employed, to a first approximation the ion-selectivity ratio is given by:

$$\frac{\overline{{P}_{( + )}}}{\overline{{P}_{( - )}}} = \frac{\overline{{P}_{( + )}} cis}{\overline{{P}_{( - )}} trans} for V ≪ 0 ; \frac{\overline{{P}_{( + )}}}{\overline{{P}_{( - )}}} = \frac{\overline{{P}_{( + )}} trans}{\overline{{P}_{( - )}} cis} for V ≫ 0$$

where P̅_̅{̅(̅ )̅}̅ is the average permeability of the indicated polarity ions in the indicated compartment, where average permeability is calculated using P̅_̅{̅(̅ )̅}̅=(P₍₁₎[P₍₁₎]+P₍₂₎[P₍₂₎]+ . . . )/[total] for the indicated polarity ions in the indicated compartment, where P₍ₛ₎ and [P₍ₛ₎] are the permeability and concentration respectively for species S=1, 2, . . . etc in given compartment, and [total] is the total concentration of the same ions.

According to the invention, it has been discovered that an ion-selectivity ratio P̅₍̅₊̅₎̅/P̅₍̅₋̅₎̅>2.0 or <0.5, preferably >2.5 or <0.4, preferably >3.0 or <0.33, most preferably >3.5 or <0.29, in combination with a symmetrical salt system is sufficient to drive capture and/or translocation of complex non-nucleic acid based polymer analytes (e.g., polypeptides) against any prevailing EPF under an applied voltage across the membrane of 20 mV to 1 V, preferably 50 mV to 300 mV, most preferably 75 mV to 200 mV.

In some embodiments, methods of controlling the EOF by inducing a strong net flow of ions in one direction across the nanopore are known in the art. These include modification of the nanopore, applying specific electrolyte asymmetries and/or concentrations, or any combination thereof.

For example, means of controlling or arranging the EOF including the genetic engineering (e.g. mutating) of the inner channel of the nanopore to alter the steric and/or the electrostatic conditions, to in turn adjust the preference for translocating one ion over another. For example, the net charge of the inner channel of the nanopore can be increased so as to electrostatically limit the flux of one of the ions from one direction across the nanopore, while retaining/enhancing the flux of the oppositely charged ion flowing in the opposite direction under an applied voltage. Creating a strong EOF is enhanced by creating a strong overlap between the Debye layers (alternatively termed the Stern layer, the Gouy-Chapman diffuse layer or the electric double layer) from adjacent walls within a nanopore (Chinappi et al., 2020, ACS Nano, 14, 11, pg. 15816-15828). Thus, a person of skill in the art would understand that the EOF can be enhanced by either adding more charges to the residues lining the walls of the channel, or narrowing the channel dimensions, or a combination thereof.

In some embodiments, mutating the pore suitably comprises one or more amino acid substitution(s), preferably mutating one or more lumen-facing residues. Mutations may also employ non-naturally occurring amino-acids to further control the magnitude and/or position of the charge relative to the amino-acid backbone of the nanopore. Preferably, mutating the pore involves mutating the narrowest regions of the inner channel of the nanopore (the constriction(s)) to create the highest overlap between Debye double-layers and thus the strongest energy barriers to limit the flow of a specific ion. A person of skill will understand that high salt is advantageous in nanopore systems for creating a large EOF and/or for creating more signal for characterization of the target analyte. However, higher salt concentrations screen the surface charge of the nanopores, reducing the Debye length, and/or reducing the effective ion-selectivity properties. Therefore, according to the invention, preferably multiple charge mutations are made along the longitudinal axis of the channel cis-to-trans to create multiple sequential energy barriers to limit the flow of a specific ion under high salt conditions (e.g. >0.1 M, most preferably in >1 M).

For example, a nanopore may be engineered to contain regions of at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, or greater than about 20 separate rings of charges along the longitudinal length of the channel. A nanopore may be engineered to contain regions of at most about 20, at most about 19, at most about 18, at most about 17, at most about 16, at most about 15, at most about 14, at most about 13, at most about 12, at most about 11, at most about 10, at most about 9, at most about 8, at most about 7, at most about 6, at most about 5, at most about 4, at most about 3, at most about 2, or less than about 2 separate rings of charges along the longitudinal length of the channel.

A nanopore may be engineered to contain regions from about 2 to about 20 separate rings of charges along the longitudinal length of the channel. A nanopore may be engineered to contain regions from about 2 to about 3, about 2 to about 4, about 2 to about 5, about 2 to about 6, about 2 to about 7, about 2 to about 8, about 2 to about 9, about 2 to about 10, about 2 to about 15, about 2 to about 20, about 3 to about 4, about 3 to about 5, about 3 to about 6, about 3 to about 7, about 3 to about 8, about 3 to about 9, about 3 to about 10, about 3 to about 15, about 3 to about 20, about 4 to about 5, about 4 to about 6, about 4 to about 7, about 4 to about 8, about 4 to about 9, about 4 to about 10, about 4 to about 15, about 4 to about 20, about 5 to about 6, about 5 to about 7, about 5 to about 8, about 5 to about 9, about 5 to about 10, about 5 to about 15, about 5 to about 20, about 6 to about 7, about 6 to about 8, about 6 to about 9, about 6 to about 10, about 6 to about 15, about 6 to about 20, about 7 to about 8, about 7 to about 9, about 7 to about 10, about 7 to about 15, about 7 to about 20, about 8 to about 9, about 8 to about 10, about 8 to about 15, about 8 to about 20, about 9 to about 10, about 9 to about 15, about 9 to about 20, about 10 to about 15, about 10 to about 20, or about 15 to about 20 separate rings of charges along the longitudinal length of the channel.

A nanopore may be engineered to contain regions of about 2, about 3, about 4, about 5, about 6, about 7, about 8, about 9, about 10, about 11, about 12, about 13, about 14, about 15, about 16, about 17, about 18, about 19, or about 20 separate rings of charges along the longitudinal length of the channel. In some embodiments, the rings of charges may each be spaced at least about 0.1 nanometers, at least about 0.2 nanometers, at least about 0.3 nanometers, at least about 0.4 nanometers, at least about 0.5 nanometers, at least about 0.6 nanometers, at least about 0.7 nanometers, at least about 0.8 nanometers, at least about 0.9 nanometers, at least about 1 nanometers, at least about 2 nanometers, at least about 3 nanometers, at least about 4 nanometers, at least about 5 nanometers, at least about 6 nanometers, at least about 7 nanometers, at least about 8 nanometers, at least about 9 nanometers, or greater than about 10 nanometers apart from each other along the longitudinal length of the channel. In some embodiments, the rings of charges may each be spaced at most about 10 nanometers, at most about 9 nanometers, at most about 8 nanometers, at most about 7 nanometers, at most about 6 nanometers, at most about 5 nanometers, at most about 4 nanometers, at most about 3 nanometers, at most about 2 nanometers, at most about 1 nanometers, at most about 0.9 nanometers, at most about 0.8 nanometers, at most about 0.7 nanometers, at most about 0.6 nanometers, at most about 0.5 nanometers, at most about 0.4 nanometers, at most about 0.3 nanometers, at most about 0.2 nanometers, at most about 0.1 nanometers, or less than about 0.1 nanometer apart from each other along the longitudinal length of the channel.

In some embodiments, the rings of charges may each be spaced from about 0.1 nanometers to about 10 nanometers apart from each other along the longitudinal length of the channel. In some embodiments, the rings of charges may each be spaced from about 0.1 nanometers to about 0.2 nanometers, about 0.1 nanometers to about 0.3 nanometers, about 0.1 nanometers to about 0.4 nanometers, about 0.1 nanometers to about 0.5 nanometers, about 0.1 nanometers to about 1 nanometer, about 0.1 nanometers to about 1.5 nanometers, about 0.1 nanometers to about 2 nanometers, about 0.1 nanometers to about 2.5 nanometers, about 0.1 nanometers to about 3 nanometers, about 0.1 nanometers to about 4 nanometers, about 0.1 nanometers to about 5 nanometers, about 0.2 nanometers to about 0.3 nanometers, about 0.2 nanometers to about 0.4 nanometers, about 0.2 nanometers to about 0.5 nanometers, about 0.2 nanometers to about 1 nanometer, about 0.2 nanometers to about 1.5 nanometers, about 0.2 nanometers to about 2 nanometers, about 0.2 nanometers to about 2.5 nanometers, about 0.2 nanometers to about 3 nanometers, about 0.2 nanometers to about 4 nanometers, about 0.2 nanometers to about 5 nanometers, about 0.3 nanometers to about 0.4 nanometers, about 0.3 nanometers to about 0.5 nanometers, about 0.3 nanometers to about 1 nanometer, about 0.3 nanometers to about 1.5 nanometers, about 0.3 nanometers to about 2 nanometers, about 0.3 nanometers to about 2.5 nanometers, about 0.3 nanometers to about 3 nanometers, about 0.3 nanometers to about 4 nanometers, about 0.3 nanometers to about 5 nanometers, about 0.4 nanometers to about 0.5 nanometers, about 0.4 nanometers to about 1 nanometer, about 0.4 nanometers to about 1.5 nanometers, about 0.4 nanometers to about 2 nanometers, about 0.4 nanometers to about 2.5 nanometers, about 0.4 nanometers to about 3 nanometers, about 0.4 nanometers to about 4 nanometers, about 0.4 nanometers to about 5 nanometers, about 0.5 nanometers to about 1 nanometer, about 0.5 nanometers to about 1.5 nanometers, about 0.5 nanometers to about 2 nanometers, about 0.5 nanometers to about 2.5 nanometers, about 0.5 nanometers to about 3 nanometers, about 0.5 nanometers to about 4 nanometers, about 0.5 nanometers to about 5 nanometers, about 1 nanometer to about 1.5 nanometers, about 1 nanometer to about 2 nanometers, about 1 nanometer to about 2.5 nanometers, about 1 nanometer to about 3 nanometers, about 1 nanometer to about 4 nanometers, about 1 nanometer to about 5 nanometers, about 1.5 nanometers to about 2 nanometers, about 1.5 nanometers to about 2.5 nanometers, about 1.5 nanometers to about 3 nanometers, about 1.5 nanometers to about 4 nanometers, about 1.5 nanometers to about 5 nanometers, about 2 nanometers to about 2.5 nanometers, about 2 nanometers to about 3 nanometers, about 2 nanometers to about 4 nanometers, about 2 nanometers to about 5 nanometers, about 2.5 nanometers to about 3 nanometers, about 2.5 nanometers to about 4 nanometers, about 2.5 nanometers to about 5 nanometers, about 3 nanometers to about 4 nanometers, about 3 nanometers to about 5 nanometers, about 4 nanometers to about 5 nanometers, about 5 nanometers to about 6 nanometers, about 6 nanometers to about 7 nanometers, about 7 nanometers to about 8 nanometers, about 8 nanometers to about 9 nanometers, or about 9 nanometers to about 10 nanometers apart from each other along the longitudinal length of the channel.

In some embodiments, the rings of charges may each be spaced about 0.1 nanometers, about 0.2 nanometers, about 0.3 nanometers, about 0.4 nanometers, about 0.5 nanometers, about 0.6 nanometers, about 0.7 nanometers, about 0.8 nanometers, about 0.9 nanometers, about 1 nanometers, about 2 nanometers, about 3 nanometers, about 4 nanometers, about 5 nanometers, about 6 nanometers, about 7 nanometers, about 8 nanometers, about 9 nanometers, or about 10 nanometers apart from each other along the longitudinal length of the channel. In some cases, one ring of charges can be located at the cis entrance of the nanopore. In some cases, one ring of charges can be located at the trans entrance of the nanopore. In some cases, one ring of charges can be located at the cis entrance of the nanopore and one ring of charges can be located at the trans entrance of the nanopore. Exemplary high EOF mutant nanopore systems include those listed in table 1.

Charge and/or steric barriers to the flow of specific ions can also be created by chemical modification of the inner lining of a nanopore. For example, cysteine residues can be reacted with derivates of maleimide or iodoacetate. A person of skill in the art would understand that a wide array of chemical modifications and/or reaction types are available for use in the invention to improve ion selectivity, including but not limited to modification of cysteines, modification of lysines, incorporation of unnatural amino acids, modification of unnatural amino acids with click chemistry groups, and/or the like.

Charge and/or steric barriers to the flow of specific ions can also be created by use of proteinaceous or chemical adapters inside the nanopore channel. For example, circular chemical adapters such as cyclodextrins or cucurbiturils can be incorporated into the nanopore (Gu et al. Biophys J. 2000 October; 79(4): 1967-1975). Alternatively, protein based adapters can be employed, such as the CsgF subunit of the CsgG nanopore (Van der Verren et al. 2020 Nature Biotechnology volume 38, pg. 1415-1420), which can separately be mutated and/or engineered to create steric and/or electrostatic barriers. The protein or chemical adapters might be attached either by non-covalent docking or by covalent means.

Charge and/or steric barriers might be engineered into a nanopore channel by the addition of amino acids into the sequence in and/or around the regions that comprise the channel (e.g. into the beta-barrel transmembrane region of a beta-barrel nanopore such as alpha-hemolysin) to create a loop, turn, constriction or other extrusion that reduces the diameter of the nanopore. Alternatively, charge and/or steric barriers can be created at either the cis or trans entrance to the nanopore channel, and away from the narrowest parts of the nanopore where analyte discrimination is strongest, to create a locally depleted regions of charge that alter the ion-selectivity through the nanopore.

Ion-selectivity biases that create a strong EOF can also be created by altering the system conditions or adding additives that change the properties of the water-facing residues in the channel of the nanopore. For example, the pH of the system can be adjusted, either on both side of the membrane or just one side of the membrane, to change the protonation state of the nanopore. For example, low pH can be employed (e.g. preferably <6.0, most preferably <4.0) to increase the net positive charge inside the nanopore, to increase the bias towards anion flow. Alternatively high pH can be used (e.g. preferably >8.0, most preferably >10.5) to increase the net negative charge inside the nanopore to increase the bias towards cation flow. Alternatively, additives that interact with the water-facing residues can be added to the solution to change the ionic or steric properties of the water-facing residues inside the nanopore. The solution or solutions may have a pH of at least about 1, at least about 2, at least about 3, at least about 3.8, at least about 4, at least about 4.5, at least about 6, at least about 7, at least about 8, at least about 9, at least about 10, at least about 10.5 at least about 11, at least about 12, at least about 13, or greater than about 13 that can be employed. The solution or solutions may have a pH of at most about 13, at most about 12, at most about 11, at most about 10.5, at most about 10, at most about 9, at most about 8, at most about 7, at most about 6, at most about 4.5, at most about 4, at most about 3.8, at most about 3, at most about 2, at most about 1, or less than about 1 that can be employed.

The solution or solutions may have a pH from about 1 to about 13 that can be employed. The solution or solutions may have a pH from about 1 to about 2, about 1 to about 3, about 1 to about 4, about 1 to about 6, about 1 to about 7, about 1 to about 8, about 1 to about 9, about 1 to about 10, about 1 to about 11, about 1 to about 12, about 1 to about 13, about 2 to about 3, about 2 to about 4, about 2 to about 6, about 2 to about 7, about 2 to about 8, about 2 to about 9, about 2 to about 10, about 2 to about 11, about 2 to about 12, about 2 to about 13, about 3 to about 4, about 3 to about 6, about 3 to about 7, about 3 to about 8, about 3 to about 9, about 3 to about 10, about 3 to about 11, about 3 to about 12, about 3 to about 13, about 4 to about 6, about 4 to about 7, about 4 to about 8, about 4 to about 9, about 4 to about 10, about 4 to about 11, about 4 to about 12, about 4 to about 13, about 6 to about 7, about 6 to about 8, about 6 to about 9, about 6 to about 10, about 6 to about 11, about 6 to about 12, about 6 to about 13, about 7 to about 8, about 7 to about 9, about 7 to about 10, about 7 to about 11, about 7 to about 12, about 7 to about 13, about 8 to about 9, about 8 to about 10, about 8 to about 11, about 8 to about 12, about 8 to about 13, about 9 to about 10, about 9 to about 11, about 9 to about 12, about 9 to about 13, about 10 to about 11, about 10 to about 12, about 10 to about 13, about 11 to about 12, about 11 to about 13, or about 12 to about 13 that can be employed.

The solution or solutions may have a pH of about 1, about 2, about 3, about 3.8, about 4, about 4.5, about 6, about 7, about 8, about 9, about 10, about 10.5 about 11, about 12, or about 13 that can be employed.

In some embodiments, mechanisms for creating and/or enhancing EOF include the use of selected salt type(s), salt asymmetries, pH, additives such as Guanidinium chloride or guanidine hydrochloride (abbreviated GdmCl and/or sometimes GdnHCl or GuHCl, GuCl) and/or osmotics.

In one embodiment, EOF is arranged through type of salts used and/or how they are distributed on each side of the membrane of the nanopore system. Means of altering the EOF by adjusting ion-selectivity through choice and/or concentration of salts are known in the art.

In some embodiments, according to the GHK flux equations, one means of creating a strong asymmetric ion flow to create a net EOF employs asymmetric salt concentrations on either side of the membrane. Low salt concentration conditions can be used in the compartment from which it is desired to have low ionic transfer, relative to higher salt concentration in the compartment from which high ionic flux is desired. For example, for setting up a system with a strong cis-to-trans EOF, a low concentration of salt can be employed in the trans compartment to limit the flow of ions from trans to cis. For example, a nanopore system can be set up with 1 M KGlu (potassium glutamate) in the cis compartment and 0.2 M KGlu in the trans compartment. The salt gradient between the compartments is preferably greater than 0.1 M, 0.2 M, preferably greater than 0.5 M. Under a cis>trans salt concentration asymmetry both cations and anions will flow cis-to-trans at moderate to low applied voltages. Thus, salt applied electro-osmosis can be highly advantageous for creating or enhancing EOF under lower voltages where repulsive EPF effects on the polymer analyte are reduced. High asymmetry salt conditions may be used in combination with nanopores that are engineered with enhanced ion-selectivity.

Salt imbalances across nanopore systems can create strong osmotic gradients, which can either enhance or compete with EOF depending on the relative direction of the fluid flow. For example, for a high-salt-cis low-salt-trans system that is set up to create a net cis-to-trans EOF, the osmotic gradient competes with the EOF. For systems where the osmotic gradient competes with EOF, the low salt compartment preferably also contains an osmolyte fully or partially balance the osmotic imbalance created by salt concentration asymmetry. Many common osmolytes are suitable for the invention, including but not limited to non-ionic or zwitterionic solutes such as glycine betaine, glucose, sucrose, glycerol, PEGs, dextrans, etc. For example, a salt imbalance of 0.5 M KCl can be balanced with about 1 M Glycine betaine. Means for measuring osmolarity of specific osmolytes and/or balancing with the correct concentration are known in the art. Furthermore, osmolytes can be added either to symmetrical salt concentration or asymmetric salt concentration systems to create an osmotic gradient that acts in the same direction as the EOF (i.e. the fluid flow from both effects moves in the same direction) to enhance the capture and/or translocation of the target polymer. For example, osmolyte (e.g. 1 M glycine betaine) can be added to the trans compartment of an ion-selective nanopore system (e.g. 1 M K Glu cis and 1 M KGlu trans) to enhance the cis-to-trans EOF.

Altering the EOF by means adjusting the salt asymmetry between the cis and trans compartment can be used in combination with an ion-selective nanopore. In one embodiment, high mobility ions can be used on one side of the membrane and low mobility and/or sterically inhibited counterions on the other side of the membrane. Ionic mobility properties well known in the art can be used to help select for appropriate high mobility and low mobility ions for use in the invention. For example, a salt with a high mobility ion can be used on the cis side of the membrane, and a salt with a low mobility (counter) ion used on the trans side of the membrane to create a stronger cis-to-trans ion-selectivity under the appropriate applied voltage. The low mobility ions might comprise all or part of their respective ionic content in the system. Suitably, the low mobility ions for altering the EOF comprise >50% of the salt content, more preferably >90% of the salt content on the side of the membrane from which they flow across the nanopore. The high mobility ions might comprise all or part of their respective ionic content in the system. Suitably, the high mobility ions for altering the EOF comprise >50% of the salt content, more preferably >90% of the salt content on the side of the membrane from which they flow across the nanopore. Preferably, for a system where the target analyte (e.g., protein) is added to the cis and translocated cis-to-trans via a strong cis-to-trans EOF, the nanopore system is set up with a highly mobile cation salt on the cis (eg. K+, Na+, NH₄+) and a low mobility anion salt on the trans (eg. glutamate, acetate, etc), wherein a negative voltage is applied to the trans. For example, a system can be set up with 1 M KCl in the cis compartment and 1 M KGlu in trans compartment, so that a greater EOF is achieved cis-to-trans under negative applied voltage to trans than the EOF generated trans-to-cis when a positive voltage is applied to the trans, due to relative lower mobility of glutamate anions versus chloride anions.

In some embodiments, the high mobility ions may comprise at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or greater than about 95% of the salt content on the side of the membrane from which they flow through the nanopore. In some embodiments, the high mobility ions may comprise at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 9%, at most about 8%, at most about 7%, at most about 6%, at most about 5%, at most about 4%, at most about 3%, at most about 2%, at most about 1%, or less than about 1% of the salt content on the side of the membrane from which they flow through the nanopore.

In some embodiments, the high mobility ions may comprise from about 1% to about 95% of the salt content on the side of the membrane from which they flow through the nanopore. In some embodiments, the high mobility ions may comprise from about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 95%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 95%, about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 20% to about 30%, about 20% to about 40%, about 20% to about 50%, about 20% to about 60%, about 20% to about 70%, about 20% to about 80%, about 20% to about 90%, about 20% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95% of the salt content on the side of the membrane from which they flow through the nanopore.

In some embodiments, the high mobility ions may comprise about 1%, about 2%, about 3%, about 4%, about 5%, about 6%, about 7%, about 8%, about 9%, about 10%, about 15%, about 20%, about 25%, about 35%, about 40%, about 45%, about 50%, about 55%, about 60%, about 65%, about 70%, about 75%, about 80%, about 85%, about 90%, or about 95% of the salt content on the side of the membrane from which they flow through the nanopore. In some embodiments, the low mobility ions may comprise at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or greater than about 95% of the salt content on the side of the membrane from which they flow through the nanopore. In some embodiments, the low mobility ions may comprise at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 9%, at most about 8%, at most about 7%, at most about 6%, at most about 5%, at most about 4%, at most about 3%, at most about 2%, at most about 1%, or less than about 1% of the salt content on the side of the membrane from which they flow through the nanopore.

In some embodiments, the low mobility ions may comprise from about 1% to about 95% of the salt content on the side of the membrane from which they flow through the nanopore. In some embodiments, the low mobility ions may comprise from about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 95%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 95%, about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 20% to about 30%, about 20% to about 40%, about 20% to about 50%, about 20% to about 60%, about 20% to about 70%, about 20% to about 80%, about 20% to about 90%, about 20% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95% of the salt content on the side of the membrane from which they flow through the nanopore.

In some embodiments, the low mobility ions may comprise about 1%, about 2%, about 3%, about 4%, about 5%, about 6%, about 7%, about 8%, about 9%, about 10%, about 15%, about 20%, about 25%, about 35%, about 40%, about 45%, about 50%, about 55%, about 60%, about 65%, about 70%, about 75%, about 80%, about 85%, about 90%, or about 95% of the salt content on the side of the membrane from which they flow through the nanopore.

According to the invention, preferably immobile and/or sterically hindered ions on one side of the membrane are combined with ion selective nanopores to further limit the flux of one or more of the ions. Due to highly constrained dimensions of a nanopore channel, particularly when filled with analyte, the mobility of a specific ion through the nanopore can differ significantly from that in bulk solution, and this can be enhanced by further mutations that increase the net charge or reduce the diameter of channel. Hence, according to the invention, the ion permeability of large and/or immobile salts in ion-selective nanopore systems (e.g. based on mutated nanopores) can be determined experimentally using the GHK equation under asymmetric salt conditions, to create a system with a sufficiently large net ion-flux to capture and/or translocate complex polymer analytes. Preferably, the system is engineered so that the flux of immobile or sterically hindered ions is effectively zero under an applied voltage, either for the open-pore state and/or the analyte filled state, so that all ionic flux is in one direction (e.g. cis to trans).

According to the invention, preferably small and/or highly mobile ions on one side of the membrane are combined with ion selective nanopores to further increase flux of given ions. For example, small and highly mobile cations (e.g. K+, Na+, NH4+, etc) can be combined with ion-selective nanopores with high internal net negative charge. In such systems the net negative charge inside the pore interacts favorably with the positive cations, acting to increase the absolute flux of the cations relative to the same nanopore with less negative charge. Thus, the increased cation flux not only increases the relative proportion of net electro-osmotic flux in one direction (e.g cis-to-trans), it also increases the absolute net electro-osmotic flux at a given voltage, which is advantageous for creating a stronger EOF versus EPF.

In some embodiments, the EOF is based on cation biased flux through the nanopore. A cation-biased EOF (P₍₊₎>>P₍₋₎) can be created or enhanced by the choice of salts in both the cis and trans compartments. Most preferably the flux employs high mobility monovalent cations such as K+=NH4+>Na+>Li+>etc. High cation biased EOF can be further enhanced by exploiting salts with large anions that are relatively immobile or otherwise restricted from translocating through the nanopore. Suitable anions are well known in the art, and may include for example high molecular mass inorganic anions (Br, Phosphate, sulphate, FeCN₆, etc) or organic anions (Acetate, Glutamate, succinate, maleate, butyrate dextrans, etc) or ionic liquid anions (e.g. tetrafluoroborate (BF4), hexafluorophosphate (PF6), bis-trifluoromethanesulfonimide (NTf2), trifluoromethanesulfonate (OTf), dicyanamide (N(CN)2), hydrogen sulphate (HSO4), and/or ethyl sulphate (EtOSO3)). For a system where the polymer analyte is added to the cis and translocated cis-to-trans via a strong cis-to-trans cation biased EOF, the system is advantageously set up with a highly mobile cation salt on the cis (eg. K+, Na+, NH4+) and a low mobility anion salt on the trans (eg. glutamate, acetate, FeCN₆, etc), wherein a negative voltage is applied to the trans. For example, the cis and trans compartments contain >0.3 M, >0.5 M, >1.0 M or >2.0 M K Glu under and applied voltage of −40 mV to −200 mV. Alternatively the trans compartment contains >0.3 M, >0.5 M, >1.0 M or >2.0 M potassium salts of butyrate, tetrafluoroborate, or ferri- or ferro-cyanide. Suitably, the polymer analyte(s) may contain polycationic tag(s) so that EPF aids in the initial nanopore capture under these conditions.

In some cases, ion selectivity of the unoccupied nanopore system will closely but not exactly match the state when the nanopore is translocating the target polymer (which will be variable). This is in part due to the additional volume the analyte occupies in the nanopore channel (thus creating a further barrier to the passage of larger ions), and partly due to any charges on the polymer, e.g. a polypeptide, that alter the ion-selectivity, and thus varies by its (amino acid) composition. The invention teaches suitable conditions and/or ions that can be selected to create strong selective conditions. Based on further experimentation, particularly when employing larger ions that are likely to experience a greater effect from an analyte-filled nanopore, the skilled artisan will be able to easily determine if the selectivity is enhanced when carrying out translocation measurements.

According to the invention, the large and/or dominant net EOF can be created either cis-to-trans or trans-to-cis relative to the convention of stating the polarity of the applied voltage at the trans electrode, and can be either cation biased or anion biased. A person of skill in the art will appreciate that the direction of the net EOF determines to which compartment the polymer analyte is added according to the methods described herein. According to the invention, the absolute net electro-osmotic flow across the nanopore in any one direction (and hence directly correlated to the magnitude of the force applied to the translocated molecule) is dependent on the applied voltage, varying to a first approximation according to the GHK flux equations as described herein. Since EPF is also directly correlated to applied voltage (increasing in magnitude as the voltage is increased), a person of skill will understand that the it is preferable to create a nanopore system with sufficiently large absolute net electro-osmotic flow at relatively low voltages to overcome repulsive EPF forces acting on the polymer analyte. According to the invention, the absolute relative net electro-osmotic current over applied voltage (I_{reIV}), given by:

$${I}_{relV} = \vert \frac{{I}_{Δ c \to t}}{{V}_{m}} \vert$$

is preferably greater than 0.1 pA/mV, greater than 0.2 pA/mV, most preferably greater than 0.3 pA/mV. Therefore, for example, at −80 mV a system according to the invention has >8 pA, >16 pA, most preferably greater than 24 pA of net electro-osmotic flow in one direction.

### Nanopore

Any suitable nanopore can be used in the disclosed methods, systems and devices. The nanopore can be a solid state artificial nanopore or a biological nanopore. In one embodiment, the nanopore is a hybrid solid state-biological nanopore such as those described in Cressiot et al. (Nature Comm. Vol. 9, 4652 (2018) or WO2009/020682. For example, many of the biological nanopores discussed herein are suitable candidates for imbedding in a solid state membrane to form a hybrid solid state-biological nanopore. Further, many non-transmembrane toroidal proteins are suitable candidates for imbedding in a solid state membrane to form a hybrid solid state-biological nanopore, for example proteins derived from Phage portal complexes, cellular transmembrane transport complexes, etc.

In some embodiments, the nanopore suitably has an inner pore constriction with a diameter in the range of 0.2 to 10 nm, such as 0.5 to 5 or 0.5-2 nm. The constriction is typically a narrowing in the channel which runs through the nanopore which may determine or control the signal obtained when the target substrate moves with respect to the nanopore. As used herein, both biological and solid state nanopores typically comprise a “constriction”.

In one embodiment the nanopore is a solid state nanopore such as those described in Xue et al., 2020, Nature Reviews Materials, Vol 5, pg. 931-951. In some cases, a solid state nanopore can comprise a nanopore made from synthetic materials. In a further embodiment, the system comprises multiple solid state nanopores in sequence that act in concert to control the capture and/or translocation of the target polymer via a strong EOF (e.g. systems such as those described in Liu et al. 2019, Small, 15, 30). Suitable solid state nanopores included coated and/or multilayered nanopores, where the coating enhances the surface charge and/or ion-selectivity properties of the nanopores (e.g. Tsutsui et al., 2022, Cell Reports Physical Science, Vol 3, Issue 10, pg. 101065).

In some cases, the nanopore is a biological nanopore. In some cases, the biological nanopore can be a nanopore that originates in nature. In one embodiment, the biological nanopore is partially or wholly comprised on non-proteinaceous organic structure. For example, the nanopore comprised wholly or partly of DNA. In one embodiment the nanopore is formed from DNA origami structures, such as those described in Bell et al., 2014, FEBS Lett., 588, 19, pg. 3564-70. According to the invention, DNA based nanopores are advantageous as they carry a large surface charge that can be used to create strongly ion-selective electro-osmotic gradients. In an alternative embodiment the nanopores are formed from assemblies of cell penetrating molecules, for example cyclic peptides (such as those described in Rodriguez-Vázquez et al., 2014, Curr Top Med Chem., 14, 23, pg. 2647-61) or cell penetrating peptides (such as those described in Krishnan R et al., 2019, J Am Chem Soc, 141, 7, pg. 2949-2959).

In some cases, the nanopore is a transmembrane protein pore which is a monomer or an oligomer. The pore is preferably made up of several repeating subunits, such as at least 6, at least 7, at least 8, at least 9, at least 10, at least 11, at least 12, at least 13, at least 14, at least 15, or at least 16 subunits. The pore is preferably a hexameric, heptameric, octameric or nonameric pore. The pore may be a homo-oligomer or a heterooligomer. In one embodiment, the transmembrane protein pore comprises a barrel or channel through which the ions may flow. The subunits of the pore typically surround a central axis and/or contribute strands to a transmembrane beta-barrel or channel or a transmembrane alpha-helix bundle or channel.

In one embodiment, the nanopore is a transmembrane protein pore derived from beta-barrel pores or alpha-helix bundle pores, beta-barrel pores comprise a barrel or channel that is formed from beta-strands. Suitable beta-barrel pores include, beta-toxins, such as alpha-hemolysin, anthrax toxin and leukocidins, and/or outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A, outer membrane protein FhuA, outer membrane protein A (OmpA) and Neisseria autotransporter lipoprotein (NalP) and/or other pores, such as lysenin, bacterial nucleoside transporter Tsx. Alpha-helix bundle pores comprise a barrel or channel that is formed from alpha-helices. Suitable alpa-helix bundle pores include inner membrane proteins and/or outer membrane proteins, such as WZA and FraC. In a specific embodiment, the nanopore is selected from the group consisting of Aer, CytK, MspA, aHL, CsgG, FraC or an engineered mutant thereof. In one embodiment the nanopore is a transmembrane pore derived from or based on Msp, e.g. MspA, a-hemolysin (a-HL), lysenin, CsgG, ClyA, Spl or haemolytic protein fragaceatoxin C (FraC).

Also encompassed are engineered nanopores as disclosed for example in WO2021/101378, and variants thereof comprising oligomeric accessory protein coupled to transmembrane domains derived from naturally existing nanopores. Engineered nanopores also includes hybrid systems, such as protein nanopores templated by DNA structures (eg. Spruit et al. Nature Nanotechnology, Vol 13, pg. 739-745). Still further, the nanopore is a de novo nanopores based on de novo alpha-helical or beta-barrel transmembrane regions (see e.g. Shimizu et al. 2022, Nature Nanotechnology volume 17, pg. 67-75; or Scott et al. 2021, Nature Chemistry volume, 13, pg. 643-650; or Vorobieva et al. 2021, Science, Vol 371, Issue 6531)).

In some cases, a (mutated) biological nanopore is suitably can be used “as such” i.e. without added structures or components. However, in a method, a device or system of the invention, the nanopore may be coupled or fused to an accessory partner protein that aids the binding and/or functioning of the protein translocase. In one embodiment, the nanopore is coupled to inactive ClpP. It is well known that the protease activity of ClpP can be disabled by mutation while retaining its ability to bind ClpX (e.g. Ortega et al., 2000, Mol Cell, 6, pg. 1515-21).

In some cases, a nanopore confers or is (genetically) modified to confer the strong and/or dominant EOF in the direction cis to trans across the membrane of a nanopore system as herein disclosed. These nanopores are herein also referred to as “strong EOF” nanopores. In one aspect, the nanopore is modified to show a relative ion selectivity P₍₊₎/P₍₋₎ of greater than 2.0 or less than 0.5, preferably greater than 3.5 or less than 0.2, under an applied voltage difference across a membrane wherein it is comprised.

In one embodiment, the nanopore lumen is engineered to have a net charge of >21, preferably >28, more preferably >35, preferably wherein said net charge is negative. In some embodiments, the nanopore lumen can comprise one or more constrictions. In some cases, the one or more constrictions can create a narrow portion of the lumen. In some cases, the narrow portion of the lumen can be the narrowest portion of the nanopore lumen. In some cases, the net charge of the nanopore channel can originate from the narrow portion of the lumen. In some cases, the rest of the lumen (e.g., the nanopore lumen minus the narrow portion) can have no net charge. In some cases, the rest of the lumen can have minimal net charge. In some cases, the narrow portion of the lumen can generate the electro-osmotic force. In some embodiments, the nanopore is selected from the group consisting of Aerolysin (Aer), Cytolysin K (CytK), MspA, alpha-hemolysin (aHL), CsgG, Fragaceatoxin C (FraC), Lysenin, phage derived portal proteins, and modified variants thereof, wherein the nanopore is modified to have a net charge in the lumen facing regions of >21, >28, >35, wherein said net charge is positive. In some embodiments, any amino acid residue in a lumen facing region of the nanopore can be mutated. In some cases, the mutated amino acid residue can be mutated to a negatively charged amino acid. In some cases, the mutated amino acid residue can be mutated to a positively charged amino acid. In some cases, the mutated amino acid residue can be mutated to a neutrally charged amino acid.

In some embodiments, the nanopore can be an oligomer. In some cases, the oligomer nanopore can comprise one or more subunits. In some cases, each subunit of the one or more subunits can comprise from about 20 to about 40 charges in the lumen facing regions of the subunit. In some cases, each subunit of the one or more subunits can comprise at least about 20 charges, at least about 21 charges, at least about 22 charges, at least about 23 charges, at least about 24 charges, at least about 25 charges, at least about 26 charges, at least about 27 charges, at least about 28 charges, at least about 29 charges, at least about 30 charges, at least about 31 charges, at least about 32 charges, at least about 33 charges, at least about 34 charges, at least about 35 charges, at least about 36 charges, at least about 37 charges, at least about 38 charges, at least about 39 charges, at least about 40 charges, or more than 40 charges in the lumen facing regions of the subunit. In some cases, each subunit of the one or more subunits can comprise at most about 40 charges, at most about 39 charges, at most about 38 charges, at most about 37 charges, at most about 36 charges, at most about 35 charges, at most about 34 charges, at most about 33 charges, at most about 32 charges, at most about 31 charges, at most about 30 charges, at most about 29 charges, at most about 28 charges, at most about 27 charges, at most about 26 charges, at most about 25 charges, at most about 24 charges, at most about 23 charges, at most about 22 charges, at most about 21 charges, at most about 20 charges, or less than 20 charges in the lumen facing regions of the subunit. In some cases, each subunit of the one or more subunits can comprise about 20 charges, about 21 charges, about 22 charges, about 23 charges, about 24 charges, about 25 charges, about 26 charges, about 27 charges, about 28 charges, about 29 charges, about 30 charges, about 31 charges, about 32 charges, about 33 charges, about 34 charges, about 35 charges, about 36 charges, about 37 charges, about 38 charges, about 39 charges, or about 40 charges in the lumen facing regions of the subunit.

In another embodiment, the nanopore is modified to increase the net positive charge of the pore lumen. In one aspect, the pore comprises at least 3, more preferably at least 4, negatively charged amino acids pointing towards the lumen of the pore per protomer for an oligomeric nanopore consisting of at least 4 protomers, where the protomers assemble to create the oligomeric nanopore and each protomer contributes to the channel region of the nanopore. Protomers may be separate entities or fused by chemical or genetic means as known in the art (Hammerstein et al., 2011, J Biol Chem., 286, 16, pg. 14324-34 or Pavelenok et al., 2022, Biophys J., 121, 5, pg. 742-754). The protomers that comprise the oligomeric nanopore may be identical or of different sequence (Miles et al., 2006, J Biol Chem., 281, 4: pg. 2205-14). In mixed protomers systems the mutations may be made to one or all of the protomer types that comprise the oligomeric assembly.

For nanopores that consist of just a single protein (e.g. porins such as OmpF, OmpG, FhuA, etc) multiple mutations can be made along the sequence so as to place charged residues either in rings around the nanopore in plane with the membrane, and/or vertically up the nanopore channel perpendicular to plane of the membrane.

For nanopores with beta-barrel channels the charge mutations can be applied on both the “up” strands and the “down” of a beta-strand at positions that co-locate them approximately co-planar so as to further enhance the local electrostatic barrier.

Charged amino acids Asp or Glu, preferably Asp, can be readily introduced by single amino acid substitution(s). At least 3 negatively charged amino acids may be distributed evenly within the lumen. At least 1 negatively charged “flanking” residue may be positioned or introduced at pore entry and/or at least 1 negatively charged residue may be positioned or introduced at pore exit. In a specific aspect, the Spacing between Cα atom of the at least 1 internal negatively charged amino acid and the Cα atoms of the negatively charged flanking amino acid is in the range of 7-11 Angstrom, preferably more than 11 Angstrom.

In a specific embodiment, the nanopore is CytK or a genetically engineered mutant thereof. For example, the mutant CytK comprises one or more of the amino acid substitutions selected from the group consisting of K128D, K155Q, T116D, S120D, Q122D, S126D, T143D, Q145D, T147D and S1511D,

wherein the numbering corresponds to the CytK amino acid available under accession number A0A2S1A9G3_9BACI in UniProt.

In one embodiment, mutant CytK comprises K128D in combination with K155Q or K155D. Preferably, it further comprises an Asp (D) mutation at one or more of positions T116, S120, Q122, S126, T143, Q145, T147 or S151.

In a specific aspect, mutant CytK comprises mutations K128D, K155D and T116D, optionally further comprising T147D and/or S151D. In another specific aspect, mutant CytK comprises mutations K128D, K155D and S120D, optionally further comprising Q122D, T147D and/or S151D. Still further, the CytK mutant comprises mutations K128D, K155D, Q145D and S151D.

In some embodiments, EOF nanopores for use in the present invention include mutant nanopores include CytK-K128D/K155D, CytK-K128D/K155Q/Q122D, CytK-K128D/K155D/Q145D, CytK-K128D/K155D/T147D and CytK-K128D/K155D/Q145D/S151D.

See Table 1 herein below.

Also provided is an isolated nucleic acid molecule encoding a mutant CytK polypeptide as herein disclosed, and an expression vector comprising the isolated nucleic acid molecule. Further embodiments relate to a host cell comprising the mutant CytK expression vector. In a specific aspect, the invention provides a nanopore system comprising a mutant CytK nanopore as herein disclosed. A mutant CytK nanopore system or a mutant CytK polypeptide is advantageously used in (single-molecule) polymer analysis, preferably protein analysis, such as polypeptide mass detection and/or protein sequencing.

In some embodiments, the nanopore can comprise additional structures on the cis side of the membrane. In some embodiments, the nanopore can comprise additional structures on the trans side of the membrane. In some cases, the nanopore can comprise additional structures on the cis side of the membrane and on the trans side of the membrane. In some cases, the additional structures can comprise nucleic acid scaffold molecules. In some cases, the nucleic acid scaffold can be a DNA scaffold. In some cases, the nucleic acid scaffold can be an RNA scaffold. In some cases, the additional structures can comprise proteases. In some cases, the proteases can comprise serine proteases, thrombin, cysteine protease, metalloproteinase, chymotrypsin, trypsin, papain, subtilisin, or any combination thereof. In some cases, the additional structures can comprise docking proteins. In some cases, the docketing proteins can comprise ClpP, TatA, TatB, TatC, Tim50, Tim23, Tim17, or any combination thereof.

### Membrane

According to the invention, the nanopore is comprised in a membrane separating a fluidic chamber of a nanopore system into a cis side and a trans side. The term “membrane” is used herein in its conventional sense to refer to a thin, film-like structure that separates the chamber of the system into a cis side (or cis compartment) and a trans side (trans compartments). The membrane separating the cis and trans compartments comprises at least one nanopore or channel. Membranes can be generally classified into synthetic membranes and biological membranes. Any membrane may be used in accordance with the invention.

In some embodiments, the membrane is preferably an amphiphilic layer. An amphiphilic layer is a layer formed from amphiphilic molecules, such as phospholipids, which have both at least one hydrophilic portion and/or at least one lipophilic or hydrophobic portion. The amphiphilic layer may be a monolayer or a bilayer. The amphiphilic molecules may be synthetic or naturally occurring. Non-naturally occurring amphiphiles which form a monolayer are known in the art and include, for example, block copolymers (Gonzalez-Perez et al., Langmuir, 2009, 25, 10447-10450).

In some cases, the membranes made of a solid state material (eg. SiN). In this context, a nanopore system may comprise a solid state hole or a hybrid pore with a biological pore inserted.

### Polymer Analyte

In some embodiments, it is demonstrated herein that a cis to trans EOF resulting from a net cis-to-trans ion current flow cis to trans, in some examples, from a net ionic current flow cis to trans over total ionic current flow of greater than 0.2 or less than −0.2, is advantageously used to translocate a large polymer analyte across a nanopore. In some embodiments, a net ionic current flow cis to trans over a total ionic current flow can be at least about −0.99, at least about −0.95, at least about −0.9, at least about −0.8, at least about −0.7, at least about −0.6, at least about −0.5, at least about −0.4, at least about −0.3, at least about −0.2, at least about −0.1, at least about 0.0, at least about 0.1, at least about 0.2, at least about 0.3, at least about 0.4, at least about 0.5, at least about 0.6, at least about 0.7, at least about 0.8, at least about 0.9, at least about 0.95, about 0.99, or greater than about 0.99. In some embodiments, a net ionic current flow cis to trans over a total ionic current flow can be at most about 0.99, at most about 0.9, at most about 0.8, at most about 0.7, at most about 0.6, at most about 0.5, at most about 0.4, at most about 0.3, at most about 0.2, at most about 0.1, at most about 0.0, at most about −0.1, at most about −0.2, at most about −0.3, at most about −0.4, at most about −0.5, at most about −0.6, at most about −0.7, at most about −0.8, at most about −0.9, −0.95, at most about −0.99, or less than about −0.99.

In some embodiments, a net ionic current flow cis to trans over a total ionic current flow can be from about −0.99 to about 0.99. In some embodiments, a cis to trans EOF results from a net ionic current flow cis to trans over a total ionic current flow, also referred to as a relative net current flow cis to trans, from about −0.99 to about −0.9, about −0.99 to about −0.8, about −0.99 to about −0.6, about −0.99 to about −0.4, about −0.99 to about −0.2, about −0.99 to about 0, about −0.99 to about 0.2, about −0.99 to about 0.4, about −0.99 to about 0.6, about −0.99 to about 0.8, about −0.99 to about 0.99, about −0.9 to about −0.8, about −0.9 to about −0.6, about −0.9 to about −0.4, about −0.9 to about −0.2, about −0.9 to about 0, about −0.9 to about 0.2, about −0.9 to about 0.4, about −0.9 to about 0.6, about −0.9 to about 0.8, about −0.9 to about 0.99, about −0.8 to about −0.6, about −0.8 to about −0.4, about −0.8 to about −0.2, about −0.8 to about 0, about −0.8 to about 0.2, about −0.8 to about 0.4, about −0.8 to about 0.6, about −0.8 to about 0.8, about −0.8 to about 0.99, about −0.6 to about −0.4, about −0.6 to about −0.2, about −0.6 to about 0, about −0.6 to about 0.2, about −0.6 to about 0.4, about −0.6 to about 0.6, about −0.6 to about 0.8, about −0.6 to about 0.99, about −0.4 to about −0.2, about −0.4 to about 0, about −0.4 to about 0.2, about −0.4 to about 0.4, about −0.4 to about 0.6, about −0.4 to about 0.8, about −0.4 to about 0.99, about −0.2 to about 0, about −0.2 to about 0.2, about −0.2 to about 0.4, about −0.2 to about 0.6, about −0.2 to about 0.8, about −0.2 to about 0.99, about 0 to about 0.2, about 0 to about 0.4, about 0 to about 0.6, about 0 to about 0.8, about 0 to about 0.99, about 0.2 to about 0.4, about 0.2 to about 0.6, about 0.2 to about 0.8, about 0.2 to about 0.99, about 0.4 to about 0.6, about 0.4 to about 0.8, about 0.4 to about 0.99, about 0.6 to about 0.8, about 0.6 to about 0.99, or about 0.8 to about 0.99.

In some embodiments, a net ionic current flow cis to trans over a total ionic current flow can be about −0.99, about −0.95, about −0.9, about −0.8, about −0.7, about −0.6, about −0.5, about −0.4, about −0.3, about −0.2, about −0.1, about 0.0, about 0.1, about 0.2, about 0.3, about 0.4, about 0.5, about 0.6, about 0.7, about 0.8, about 0.9, about 0.95, or about 0.99.

The polymer analyte may be of heterogeneous charge. A heterogeneously charged polymer may comprises or consists of positively and negatively units charged repeating units and/or wherein charged units are unevenly distributed along the polymer, against an electrophoretic force acting on the analyte. This allows for nanopore-based detection of unmodified (label-free) polymer analyte. Hence, according to the invention a polymer analyte may be added to the nanopore system as such, or it may be provided with a label or tag.

The polymer analyte can be of synthetic, semi-synthetic or biological origin. In one aspect, it is a biopolymer, preferably comprising or consisting of peptide units, saccharide units and water-soluble plastic monomers, and any combination thereof. For example, the polymer analyte is a polypeptide, polysaccharide, or a water-soluble plastic, such as PEG, or PEGylated polypeptide.

A method or system of the invention allows for detection and analysis of non-nucleic acid based polymers. As used herein, “non-nucleic acid based” indicates that the polymer analyte does not consist entirely or predominantly of nucleic acid units, such as DNA or RNA analytes. However, the analyte may contain an occasional nucleic acid moiety as minor constituent e.g. less than 10%, preferably less than 5%, more preferably less than 3% of the polymer units is a nucleic acid.

At least the termini of the polymer may be unstructured to facilitate nanopore capture and/or entry. Preferably, at least a portion of the polymer is in an elongated or linearized state. For example, in case of a proteinaceous analyte it can be partially or fully denatured. In one embodiment, polypeptide analytes are pre-denatured prior to addition to the nanopore system.

In one embodiment, the length of the elongated polymer is larger than the than the longitudinal axis of the central channel of the nanopore in the direction perpendicular to the membrane, preferably wherein the length is >30, more preferably >40, most preferably >50 repeating (monomer) units.

In some embodiments, wherein the polymer analyte is a polypeptide of at least 30 peptide units and/or comprising positively and/or negatively charged residues, preferably wherein the polypeptide is in a denatured/unfolded state, more preferably wherein the polypeptide is added in a pre-denatured state. In some embodiments the polymer analyte is an unmodified protein or a portion thereof, or a naturally occurring polypeptide or a portion thereof. In some embodiments, the polymer analyte is a full length protein or naturally occurring polypeptide. In some embodiments polypeptides or polypeptide fragments can be conjugated to form a longer polypeptide.

Any number of analytes (e.g., proteins) can be translocated or characterised in the disclosed methods. The methods described herein may comprise characterizing at least about 2 protein analytes, at least about 3 protein analytes, at least about 4 protein analytes, at least about 5 protein analytes, at least about 6 protein analytes, at least about 7 protein analytes, at least about 8 protein analytes, at least about 9 protein analytes, at least about 10 protein analytes, at least about 20 protein analytes, at least about 30 protein analytes, at least about 50 protein analytes, at least about 100 protein analytes, at least about 200 protein analytes, at least about 300 protein analytes, at least about 400 protein analytes, at least about 500 protein analytes, at least about 600 protein analytes, at least about 700 protein analytes, at least about 800 protein analytes, at least about 900 protein analytes, at least about 1000 protein analytes, at least about 1500 protein analytes, at least about 2000 protein analytes, at least about 2500 protein analytes, at least about 3500 protein analytes, at least about 4000 protein analytes, at least about 4500 protein analytes, at least about 5000 protein analytes, at least about 5500 protein analytes, at least about 6000 protein analytes, at least about 6500 protein analytes, at least about 7000 protein analytes, at least about 7500 protein analytes, at least about 8000 protein analytes, at least about 8500 protein analytes, at least about 9000 protein analytes, at least about 9500 protein analytes, at least about 10000 protein analytes, or greater than about 10000 protein analytes.

The methods described herein may comprise characterizing at most about 10000 protein analytes protein analytes, at most about 9500 protein analytes, at most about 8500 protein analytes, at most about 8000 protein analytes, at most about 7500 protein analytes, at most about 7000 protein analytes, at most about 6500 protein analytes, at most about 6000 protein analytes, at most about 5500 protein analytes, at most about 5000 protein analytes, at most about 4500 protein analytes, at most about 4000 protein analytes, at most about 3500 protein analytes, at most about 3000 protein analytes, at most about 2500 protein analytes, at most about 2000 protein analytes, at most about 1500 protein analytes, at most about 1000 protein analytes, at most about 900 protein analytes, at most about 800 protein analytes, at most about 700 protein analytes, at most about 600 protein analytes, at most about 500 protein analytes, at most about 400 protein analytes, at most about 300 protein analytes, at most about 200 protein analytes, at most about 100 protein analytes, at most about 50 protein analytes, at most about 30 protein analytes, at most about 20 protein analytes, at most about 10 protein analytes, at most about 9 protein analytes, at most about 8 protein analytes, at most about 7 protein analytes, at most about 6 protein analytes, at most about 5 protein analytes, at most about 4 protein analytes, at most about 3 protein analytes, at most about or less than about 3 protein analytes.

The methods described herein may comprise characterizing from about 3 protein analytes to about 100 protein analytes. The methods described herein may comprise characterizing from about 3 protein analytes to about 5 protein analytes, about 3 protein analytes to about 10 protein analytes, about 3 protein analytes to about 20 protein analytes, about 3 protein analytes to about 30 protein analytes, about 3 protein analytes to about 40 protein analytes, about 3 protein analytes to about 50 protein analytes, about 3 protein analytes to about 60 protein analytes, about 3 protein analytes to about 70 protein analytes, about 3 protein analytes to about 80 protein analytes, about 3 protein analytes to about 90 protein analytes, about 3 protein analytes to about 100 protein analytes, about 5 protein analytes to about 10 protein analytes, about 5 protein analytes to about 20 protein analytes, about 5 protein analytes to about 30 protein analytes, about 5 protein analytes to about 40 protein analytes, about 5 protein analytes to about 50 protein analytes, about 5 protein analytes to about 60 protein analytes, about 5 protein analytes to about 70 protein analytes, about 5 protein analytes to about 80 protein analytes, about 5 protein analytes to about 90 protein analytes, about 5 protein analytes to about 100 protein analytes, about 10 protein analytes to about 20 protein analytes, about 10 protein analytes to about 30 protein analytes, about 10 protein analytes to about 40 protein analytes, about 10 protein analytes to about 50 protein analytes, about 10 protein analytes to about 60 protein analytes, about 10 protein analytes to about 70 protein analytes, about 10 protein analytes to about 80 protein analytes, about 10 protein analytes to about 90 protein analytes, about 10 protein analytes to about 100 protein analytes, about 20 protein analytes to about 30 protein analytes, about 20 protein analytes to about 40 protein analytes, about 20 protein analytes to about 50 protein analytes, about 20 protein analytes to about 60 protein analytes, about 20 protein analytes to about 70 protein analytes, about 20 protein analytes to about 80 protein analytes, about 20 protein analytes to about 90 protein analytes, about 20 protein analytes to about 100 protein analytes, about 30 protein analytes to about 40 protein analytes, about 30 protein analytes to about 50 protein analytes, about 30 protein analytes to about 60 protein analytes, about 30 protein analytes to about 70 protein analytes, about 30 protein analytes to about 80 protein analytes, about 30 protein analytes to about 90 protein analytes, about 30 protein analytes to about 100 protein analytes, about 40 protein analytes to about 50 protein analytes, about 40 protein analytes to about 60 protein analytes, about 40 protein analytes to about 70 protein analytes, about 40 protein analytes to about 80 protein analytes, about 40 protein analytes to about 90 protein analytes, about 40 protein analytes to about 100 protein analytes, about 50 protein analytes to about 60 protein analytes, about 50 protein analytes to about 70 protein analytes, about 50 protein analytes to about 80 protein analytes, about 50 protein analytes to about 90 protein analytes, about 50 protein analytes to about 100 protein analytes, about 60 protein analytes to about 70 protein analytes, about 60 protein analytes to about 80 protein analytes, about 60 protein analytes to about 90 protein analytes, about 60 protein analytes to about 100 protein analytes, about 70 protein analytes to about 80 protein analytes, about 70 protein analytes to about 90 protein analytes, about 70 protein analytes to about 100 protein analytes, about 80 protein analytes to about 90 protein analytes, about 80 protein analytes to about 100 protein analytes, or about 90 protein analytes to about 100 protein analytes.

The methods described herein may comprise characterizing from about 100 protein analytes to about 10,000 protein analytes. The methods described herein may comprise characterizing from about 100 protein analytes to about 250 protein analytes, about 100 protein analytes to about 500 protein analytes, about 100 protein analytes to about 750 protein analytes, about 100 protein analytes to about 1,000 protein analytes, about 100 protein analytes to about 2,000 protein analytes, about 100 protein analytes to about 3,000 protein analytes, about 100 protein analytes to about 4,000 protein analytes, about 100 protein analytes to about 5,000 protein analytes, about 100 protein analytes to about 7,500 protein analytes, about 100 protein analytes to about 10,000 protein analytes, about 250 protein analytes to about 500 protein analytes, about 250 protein analytes to about 750 protein analytes, about 250 protein analytes to about 1,000 protein analytes, about 250 protein analytes to about 2,000 protein analytes, about 250 protein analytes to about 3,000 protein analytes, about 250 protein analytes to about 4,000 protein analytes, about 250 protein analytes to about 5,000 protein analytes, about 250 protein analytes to about 7,500 protein analytes, about 250 protein analytes to about 10,000 protein analytes, about 500 protein analytes to about 750 protein analytes, about 500 protein analytes to about 1,000 protein analytes, about 500 protein analytes to about 2,000 protein analytes, about 500 protein analytes to about 3,000 protein analytes, about 500 protein analytes to about 4,000 protein analytes, about 500 protein analytes to about 5,000 protein analytes, about 500 protein analytes to about 7,500 protein analytes, about 500 protein analytes to about 10,000 protein analytes, about 750 protein analytes to about 1,000 protein analytes, about 750 protein analytes to about 2,000 protein analytes, about 750 protein analytes to about 3,000 protein analytes, about 750 protein analytes to about 4,000 protein analytes, about 750 protein analytes to about 5,000 protein analytes, about 750 protein analytes to about 7,500 protein analytes, about 750 protein analytes to about 10,000 protein analytes, about 1,000 protein analytes to about 2,000 protein analytes, about 1,000 protein analytes to about 3,000 protein analytes, about 1,000 protein analytes to about 4,000 protein analytes, about 1,000 protein analytes to about 5,000 protein analytes, about 1,000 protein analytes to about 7,500 protein analytes, about 1,000 protein analytes to about 10,000 protein analytes, about 2,000 protein analytes to about 3,000 protein analytes, about 2,000 protein analytes to about 4,000 protein analytes, about 2,000 protein analytes to about 5,000 protein analytes, about 2,000 protein analytes to about 7,500 protein analytes, about 2,000 protein analytes to about 10,000 protein analytes, about 3,000 protein analytes to about 4,000 protein analytes, about 3,000 protein analytes to about 5,000 protein analytes, about 3,000 protein analytes to about 7,500 protein analytes, about 3,000 protein analytes to about 10,000 protein analytes, about 4,000 protein analytes to about 5,000 protein analytes, about 4,000 protein analytes to about 7,500 protein analytes, about 4,000 protein analytes to about 10,000 protein analytes, about 5,000 protein analytes to about 7,500 protein analytes, about 5,000 protein analytes to about 10,000 protein analytes, or about 7,500 protein analytes to about 10,000 protein analytes.

The methods described herein may comprise characterizing about 2 protein analytes, about 3 protein analytes, about 4 protein analytes, about 5 protein analytes, about 6 protein analytes, about 7 protein analytes, about 8 protein analytes, about 9 protein analytes, about 10 protein analytes, about 20 protein analytes, about 30 protein analytes, about 50 protein analytes, about 100 protein analytes, about 200 protein analytes, about 300 protein analytes, about 400 protein analytes, about 500 protein analytes, about 600 protein analytes, about 700 protein analytes, about 800 protein analytes, about 900 protein analytes, about 1000 protein analytes, about 1500 protein analytes, about 2000 protein analytes, about 2500 protein analytes, about 3500 protein analytes, about 4000 protein analytes, about 4500 protein analytes, about 5000 protein analytes, about 5500 protein analytes, about 6000 protein analytes, about 6500 protein analytes, about 7000 protein analytes, about 7500 protein analytes, about 8000 protein analytes, about 8500 protein analytes, about 9000 protein analytes, about 9500 protein analytes, or about 10000 protein analytes. If two or more proteins are used, they may be different proteins or two or more copies of the same proteins.

In some embodiments, multiple analytes may be measured. The signal or signals from the multiple analytes may be used to characterize them. In some embodiments, at least about 2 analytes, at least about 3 analytes, at least about 4 analytes, at least about 5 analytes, at least about 6 analytes, at least about 7 analytes, at least about 8 analytes, at least about 9 analytes, at least about 10 analytes, at least about 20 analytes, at least about 30 analytes, at least about 40 analytes, at least about 50 analytes, at least about 100 analytes, at least about 200 analytes, at least about 300 analytes, at least about 400 analytes, at least about 500 analytes, at least about 600 analytes, at least about 700 analytes, at least about 800 analytes, at least about 900 analytes, at least about 1000 analytes, at least about 1500 analytes, at least about 2000 analytes, at least about 2500 analytes, at least about 3000 analytes, at least about 3500 analytes, at least about 4000 analytes, at least about 4500 analytes, at least about 5000 analytes, at least about 5500 analytes, at least about 6000 analytes, at least about 6500 analytes, at least about 7000 analytes, at least about 7500 analytes, at least about 8000 analytes, at least about 8500 analytes, at least about 9000 analytes, at least about 9500 analytes, at least about 10000 analytes, or greater than about 10000 analytes may be characterized. In some embodiments analytes, at most about 10000 analytes, at most about 9500 analytes, at most about 9000 analytes, at most about 8500 analytes, at most about 8000 analytes, at most about 7500 analytes, at most about 7000 analytes, at most about 6500 analytes, at most about 6000 analytes, at most about 5500 analytes, at most about 5000 analytes, at most about 4500 analytes, at most about 4000 analytes, at most about 3500 analytes, at most about 3000 analytes, at most about 2500 analytes, at most about 2000 analytes, at most about 1500 analytes, at most about 1000 analytes, at most about 900 analytes, at most about 800 analytes, at most about 700 analytes, at most about 600 analytes, at most about 500 analytes, at most about 400 analytes, at most about 300 analytes, at most about 200 analytes, at most about 100 analytes, at most about 90 analytes, at most about 80 analytes, at most about 70 analytes, at most about 60 analytes, at most about 50 analytes, at most about 40 analytes, at most about 30 analytes, at most about 20 analytes, at most about 10 analytes, at most about 9 analytes, at most about 8 analytes, at most about 7 analytes, at most about 6 analytes, at most about 5 analytes, at most about 4 analytes, at most about 3 analytes, at most about 2 analytes, or less than about 2 analytes may be characterized.

In some embodiments, from about 2 analytes to about 100 analytes may be characterized. In some embodiments, from about 2 analytes to about 5 analytes, about 2 analytes to about 10 analytes, about 2 analytes to about 20 analytes, about 2 analytes to about 30 analytes, about 2 analytes to about 40 analytes, about 2 analytes to about 50 analytes, about 2 analytes to about 60 analytes, about 2 analytes to about 70 analytes, about 2 analytes to about 80 analytes, about 2 analytes to about 90 analytes, about 2 analytes to about 100 analytes, about 5 analytes to about 10 analytes, about 5 analytes to about 20 analytes, about 5 analytes to about 30 analytes, about 5 analytes to about 40 analytes, about 5 analytes to about 50 analytes, about 5 analytes to about 60 analytes, about 5 analytes to about 70 analytes, about 5 analytes to about 80 analytes, about 5 analytes to about 90 analytes, about 5 analytes to about 100 analytes, about 10 analytes to about 20 analytes, about 10 analytes to about 30 analytes, about 10 analytes to about 40 analytes, about 10 analytes to about 50 analytes, about 10 analytes to about 60 analytes, about 10 analytes to about 70 analytes, about 10 analytes to about 80 analytes, about 10 analytes to about 90 analytes, about 10 analytes to about 100 analytes, about 20 analytes to about 30 analytes, about 20 analytes to about 40 analytes, about 20 analytes to about 50 analytes, about 20 analytes to about 60 analytes, about 20 analytes to about 70 analytes, about 20 analytes to about 80 analytes, about 20 analytes to about 90 analytes, about 20 analytes to about 100 analytes, about 30 analytes to about 40 analytes, about 30 analytes to about 50 analytes, about 30 analytes to about 60 analytes, about 30 analytes to about 70 analytes, about 30 analytes to about 80 analytes, about 30 analytes to about 90 analytes, about 30 analytes to about 100 analytes, about 40 analytes to about 50 analytes, about 40 analytes to about 60 analytes, about 40 analytes to about 70 analytes, about 40 analytes to about 80 analytes, about 40 analytes to about 90 analytes, about 40 analytes to about 100 analytes, about 50 analytes to about 60 analytes, about 50 analytes to about 70 analytes, about 50 analytes to about 80 analytes, about 50 analytes to about 90 analytes, about 50 analytes to about 100 analytes, about 60 analytes to about 70 analytes, about 60 analytes to about 80 analytes, about 60 analytes to about 90 analytes, about 60 analytes to about 100 analytes, about 70 analytes to about 80 analytes, about 70 analytes to about 90 analytes, about 70 analytes to about 100 analytes, about 80 analytes to about 90 analytes, about 80 analytes to about 100 analytes, or about 90 analytes to about 100 analytes may be characterized.

In some embodiments, from about 100 analytes to about 10,000 analytes may be characterized. In some embodiments, from about 100 analytes to about 200 analytes, about 100 analytes to about 300 analytes, about 100 analytes to about 400 analytes, about 100 analytes to about 500 analytes, about 100 analytes to about 750 analytes, about 100 analytes to about 1,000 analytes, about 100 analytes to about 2,500 analytes, about 100 analytes to about 5,000 analytes, about 100 analytes to about 7,500 analytes, about 100 analytes to about 10,000 analytes, about 200 analytes to about 300 analytes, about 200 analytes to about 400 analytes, about 200 analytes to about 500 analytes, about 200 analytes to about 750 analytes, about 200 analytes to about 1,000 analytes, about 200 analytes to about 2,500 analytes, about 200 analytes to about 5,000 analytes, about 200 analytes to about 7,500 analytes, about 200 analytes to about 10,000 analytes, about 300 analytes to about 400 analytes, about 300 analytes to about 500 analytes, about 300 analytes to about 750 analytes, about 300 analytes to about 1,000 analytes, about 300 analytes to about 2,500 analytes, about 300 analytes to about 5,000 analytes, about 300 analytes to about 7,500 analytes, about 300 analytes to about 10,000 analytes, about 400 analytes to about 500 analytes, about 400 analytes to about 750 analytes, about 400 analytes to about 1,000 analytes, about 400 analytes to about 2,500 analytes, about 400 analytes to about 5,000 analytes, about 400 analytes to about 7,500 analytes, about 400 analytes to about 10,000 analytes, about 500 analytes to about 750 analytes, about 500 analytes to about 1,000 analytes, about 500 analytes to about 2,500 analytes, about 500 analytes to about 5,000 analytes, about 500 analytes to about 7,500 analytes, about 500 analytes to about 10,000 analytes, about 750 analytes to about 1,000 analytes, about 750 analytes to about 2,500 analytes, about 750 analytes to about 5,000 analytes, about 750 analytes to about 7,500 analytes, about 750 analytes to about 10,000 analytes, about 1,000 analytes to about 2,500 analytes, about 1,000 analytes to about 5,000 analytes, about 1,000 analytes to about 7,500 analytes, about 1,000 analytes to about 10,000 analytes, about 2,500 analytes to about 5,000 analytes, about 2,500 analytes to about 7,500 analytes, about 2,500 analytes to about 10,000 analytes, about 5,000 analytes to about 7,500 analytes, about 5,000 analytes to about 10,000 analytes, or about 7,500 analytes to about 10,000 analytes may be characterized.

In some embodiments, about 2 analytes, about 3 analytes, about 4 analytes, about 5 analytes, about 6 analytes, about 7 analytes, about 8 analytes, about 9 analytes, about 10 analytes, about 20 analytes, about 30 analytes, about 40 analytes, about 50 analytes, about 100 analytes, about 200 analytes, about 300 analytes, about 400 analytes, about 500 analytes, about 600 analytes, about 700 analytes, about 800 analytes, about 900 analytes, about 1000 analytes, about 1500 analytes, about 2000 analytes, about 2500 analytes, about 3000 analytes, about 3500 analytes, about 4000 analytes, about 4500 analytes, about 5000 analytes, about 5500 analytes, about 6000 analytes, about 6500 analytes, about 7000 analytes, about 7500 analytes, about 8000 analytes, about 8500 analytes, about 9000 analytes, about 9500 analytes, or about 10000 analytes may be characterized.

In some embodiments, at least about 2 types of analytes, at least about 3 types of analytes, at least about 4 types of analytes, at least about 5 types of analytes, at least about 6 types of analytes, at least about 7 types of analytes, at least about 8 types of analytes, at least about 9 types of analytes, at least about 10 types of analytes, at least about 20 types of analytes, at least about 30 types of analytes, at least about 40 types of analytes, at least about 50 types of analytes, at least about 100 types of analytes, at least about 200 types of analytes, at least about 300 types of analytes, at least about 400 types of analytes, at least about 500 types of analytes, at least about 600 types of analytes, at least about 700 types of analytes, at least about 800 types of analytes, at least about 900 types of analytes, at least about 1000 types of analytes, at least about 1500 types of analytes, at least about 2000 types of analytes, at least about 2500 types of analytes, at least about 3000 types of analytes, at least about 3500 types of analytes, at least about 4000 types of analytes, at least about 4500 types of analytes, at least about 5000 types of analytes, at least about 5500 types of analytes, at least about 6000 types of analytes, at least about 6500 types of analytes, at least about 7000 types of analytes, at least about 7500 types of analytes, at least about 8000 types of analytes, at least about 8500 types of analytes, at least about 9000 types of analytes, at least about 9500 types of analytes, at least about 10000 types of analytes, or greater than about 10000 types of analytes may be characterized. In some embodiments types of analytes, at most about 10000 types of analytes, at most about 9500 types of analytes, at most about 9000 types of analytes, at most about 8500 types of analytes, at most about 8000 types of analytes, at most about 7500 types of analytes, at most about 7000 types of analytes, at most about 6500 types of analytes, at most about 6000 types of analytes, at most about 5500 types of analytes, at most about 5000 types of analytes, at most about 4500 types of analytes, at most about 4000 types of analytes, at most about 3500 types of analytes, at most about 3000 types of analytes, at most about 2500 types of analytes, at most about 2000 types of analytes, at most about 1500 types of analytes, at most about 1000 types of analytes, at most about 900 types of analytes, at most about 800 types of analytes, at most about 700 types of analytes, at most about 600 types of analytes, at most about 500 types of analytes, at most about 400 types of analytes, at most about 300 types of analytes, at most about 200 types of analytes, at most about 100 types of analytes, at most about 90 types of analytes, at most about 80 types of analytes, at most about 70 types of analytes, at most about 60 types of analytes, at most about 50 types of analytes, at most about 40 types of analytes, at most about 30 types of analytes, at most about 20 types of analytes, at most about 10 types of analytes, at most about 9 types of analytes, at most about 8 types of analytes, at most about 7 types of analytes, at most about 6 types of analytes, at most about 5 types of analytes, at most about 4 types of analytes, at most about 3 types of analytes, at most about 2 types of analytes, or less than about 2 types of analytes may be characterized.

In some embodiments, from about 2 types of analytes to about 100 types of analytes may be characterized. In some embodiments, from about 2 types of analytes to about 5 types of analytes, about 2 types of analytes to about 10 types of analytes, about 2 types of analytes to about 20 types of analytes, about 2 types of analytes to about 30 types of analytes, about 2 types of analytes to about 40 types of analytes, about 2 types of analytes to about 50 types of analytes, about 2 types of analytes to about 60 types of analytes, about 2 types of analytes to about 70 types of analytes, about 2 types of analytes to about 80 types of analytes, about 2 types of analytes to about 90 types of analytes, about 2 types of analytes to about 100 types of analytes, about 5 types of analytes to about 10 types of analytes, about 5 types of analytes to about 20 types of analytes, about 5 types of analytes to about 30 types of analytes, about 5 types of analytes to about 40 types of analytes, about 5 types of analytes to about 50 types of analytes, about 5 types of analytes to about 60 types of analytes, about 5 types of analytes to about 70 types of analytes, about 5 types of analytes to about 80 types of analytes, about 5 types of analytes to about 90 types of analytes, about 5 types of analytes to about 100 types of analytes, about 10 types of analytes to about 20 types of analytes, about 10 types of analytes to about 30 types of analytes, about 10 types of analytes to about 40 types of analytes, about 10 types of analytes to about 50 types of analytes, about 10 types of analytes to about 60 types of analytes, about 10 types of analytes to about 70 types of analytes, about 10 types of analytes to about 80 types of analytes, about 10 types of analytes to about 90 types of analytes, about 10 types of analytes to about 100 types of analytes, about 20 types of analytes to about 30 types of analytes, about 20 types of analytes to about 40 types of analytes, about 20 types of analytes to about 50 types of analytes, about 20 types of analytes to about 60 types of analytes, about 20 types of analytes to about 70 types of analytes, about 20 types of analytes to about 80 types of analytes, about 20 types of analytes to about 90 types of analytes, about 20 types of analytes to about 100 types of analytes, about 30 types of analytes to about 40 types of analytes, about 30 types of analytes to about 50 types of analytes, about 30 types of analytes to about 60 types of analytes, about 30 types of analytes to about 70 types of analytes, about 30 types of analytes to about 80 types of analytes, about 30 types of analytes to about 90 types of analytes, about 30 types of analytes to about 100 types of analytes, about 40 types of analytes to about 50 types of analytes, about 40 types of analytes to about 60 types of analytes, about 40 types of analytes to about 70 types of analytes, about 40 types of analytes to about 80 types of analytes, about 40 types of analytes to about 90 types of analytes, about 40 types of analytes to about 100 types of analytes, about 50 types of analytes to about 60 types of analytes, about 50 types of analytes to about 70 types of analytes, about 50 types of analytes to about 80 types of analytes, about 50 types of analytes to about 90 types of analytes, about 50 types of analytes to about 100 types of analytes, about 60 types of analytes to about 70 types of analytes, about 60 types of analytes to about 80 types of analytes, about 60 types of analytes to about 90 types of analytes, about 60 types of analytes to about 100 types of analytes, about 70 types of analytes to about 80 types of analytes, about 70 types of analytes to about 90 types of analytes, about 70 types of analytes to about 100 types of analytes, about 80 types of analytes to about 90 types of analytes, about 80 types of analytes to about 100 types of analytes, or about 90 types of analytes to about 100 types of analytes may be characterized.

In some embodiments, from about 100 types of analytes to about 10,000 types of analytes may be characterized. In some embodiments, from about 100 types of analytes to about 200 types of analytes, about 100 types of analytes to about 300 types of analytes, about 100 types of analytes to about 400 types of analytes, about 100 types of analytes to about 500 types of analytes, about 100 types of analytes to about 750 types of analytes, about 100 types of analytes to about 1,000 types of analytes, about 100 types of analytes to about 2,500 types of analytes, about 100 types of analytes to about 5,000 types of analytes, about 100 types of analytes to about 7,500 types of analytes, about 100 types of analytes to about 10,000 types of analytes, about 200 types of analytes to about 300 types of analytes, about 200 types of analytes to about 400 types of analytes, about 200 types of analytes to about 500 types of analytes, about 200 types of analytes to about 750 types of analytes, about 200 types of analytes to about 1,000 types of analytes, about 200 types of analytes to about 2,500 types of analytes, about 200 types of analytes to about 5,000 types of analytes, about 200 types of analytes to about 7,500 types of analytes, about 200 types of analytes to about 10,000 types of analytes, about 300 types of analytes to about 400 types of analytes, about 300 types of analytes to about 500 types of analytes, about 300 types of analytes to about 750 types of analytes, about 300 types of analytes to about 1,000 types of analytes, about 300 types of analytes to about 2,500 types of analytes, about 300 types of analytes to about 5,000 types of analytes, about 300 types of analytes to about 7,500 types of analytes, about 300 types of analytes to about 10,000 types of analytes, about 400 types of analytes to about 500 types of analytes, about 400 types of analytes to about 750 types of analytes, about 400 types of analytes to about 1,000 types of analytes, about 400 types of analytes to about 2,500 types of analytes, about 400 types of analytes to about 5,000 types of analytes, about 400 types of analytes to about 7,500 types of analytes, about 400 types of analytes to about 10,000 types of analytes, about 500 types of analytes to about 750 types of analytes, about 500 types of analytes to about 1,000 types of analytes, about 500 types of analytes to about 2,500 types of analytes, about 500 types of analytes to about 5,000 types of analytes, about 500 types of analytes to about 7,500 types of analytes, about 500 types of analytes to about 10,000 types of analytes, about 750 types of analytes to about 1,000 types of analytes, about 750 types of analytes to about 2,500 types of analytes, about 750 types of analytes to about 5,000 types of analytes, about 750 types of analytes to about 7,500 types of analytes, about 750 types of analytes to about 10,000 types of analytes, about 1,000 types of analytes to about 2,500 types of analytes, about 1,000 types of analytes to about 5,000 types of analytes, about 1,000 types of analytes to about 7,500 types of analytes, about 1,000 types of analytes to about 10,000 types of analytes, about 2,500 types of analytes to about 5,000 types of analytes, about 2,500 types of analytes to about 7,500 types of analytes, about 2,500 types of analytes to about 10,000 types of analytes, about 5,000 types of analytes to about 7,500 types of analytes, about 5,000 types of analytes to about 10,000 types of analytes, or about 7,500 types of analytes to about 10,000 types of analytes may be characterized.

In some embodiments, about 2 types of analytes, about 3 types of analytes, about 4 types of analytes, about 5 types of analytes, about 6 types of analytes, about 7 types of analytes, about 8 types of analytes, about 9 types of analytes, about 10 types of analytes, about 20 types of analytes, about 30 types of analytes, about 40 types of analytes, about 50 types of analytes, about 100 types of analytes, about 200 types of analytes, about 300 types of analytes, about 400 types of analytes, about 500 types of analytes, about 600 types of analytes, about 700 types of analytes, about 800 types of analytes, about 900 types of analytes, about 1000 types of analytes, about 1500 types of analytes, about 2000 types of analytes, about 2500 types of analytes, about 3000 types of analytes, about 3500 types of analytes, about 4000 types of analytes, about 4500 types of analytes, about 5000 types of analytes, about 5500 types of analytes, about 6000 types of analytes, about 6500 types of analytes, about 7000 types of analytes, about 7500 types of analytes, about 8000 types of analytes, about 8500 types of analytes, about 9000 types of analytes, about 9500 types of analytes, or about 10000 types of analytes may be characterized.

In some embodiments an electrophoretic force can act in a cis to trans direction or a trans to cis direction. An electrophoretic force can act in the same direction as an electro-osmotic force or in an opposing direction to an electro-osmotic force. An electrophoretic force can exert a greater or lesser force on an analyte than an electro-osmotic force. The electrophoretic force can assist or oppose a translocation of an analyte.

In some embodiments, an analyte can comprise a nucleic acid moiety. In some embodiments, the nucleic acid moiety may comprise at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or greater than about 95% of the polymer units. In some embodiments, the nucleic acid moiety may comprise at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 9%, at most about 8%, at most about 7%, at most about 6%, at most about 5%, at most about 4%, at most about 3%, at most about 2%, at most about 1%, or less than about 1% of the polymer units.

In some embodiments, the nucleic acid moiety may comprise from about 1% to about 95% of the polymer units. In some embodiments, the low mobility ions may comprise from about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 95%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 95%, about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 20% to about 30%, about 20% to about 40%, about 20% to about 50%, about 20% to about 60%, about 20% to about 70%, about 20% to about 80%, about 20% to about 90%, about 20% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95% of the polymer units.

In some embodiments, the nucleic acid moiety may comprise about 1%, about 2%, about 3%, about 4%, about 5%, about 6%, about 7%, about 8%, about 9%, about 10%, about 15%, about 20%, about 25%, about 35%, about 40%, about 45%, about 50%, about 55%, about 60%, about 65%, about 70%, about 75%, about 80%, about 85%, about 90%, or about 95% of the polymer units.

In some embodiments, the analyte can be at least about 1 kDa, at least about 2 kDa, at least about 3 kDa, at least about 4 kDa, at least about 5 kDa, at least about 6 kDa, at least about 7 kDa, at least about 8 kDa, at least about 9 kDa, at least about 10 kDa, at least about 15 kDa, at least about 20 kDa, at least about 25 kDa, at least about 30 kDa, at least about 35 kDa, at least about 40 kDa, at least about 45 kDa, at least about 50 kDa, at least about 55 kDa, at least about 60 kDa, at least about 65 kDa, at least about 70 kDa, at least about 75 kDa, at least about 80 kDa, at least about 85 kDa, at least about 90 kDa, at least about 95 kDa, at least about 100 kDa, at least about 125 kDa, at least about 150 kDa, at least about 175 kDa, at least about 200 kDa, at least about 250 kDa, at least about 300 kDa, at least about 350 kDa, at least about 400 kDa, at least about 450 kDa, at least about 500 kDa, at least about 550 kDa, at least about 600 kDa, at least about 650 kDa, at least about 700 kDa, at least about 750 kDa, at least about 800 kDa, at least about 850 kDa, at least about 900 kDa, at least about 950 kDa, at least about 1000 kDa, at least about 1500 kDa, at least about 2000 kDa, at least about 2500 kDa, at least about 3000 kDa, at least about 3500 kDa, at least about 4000 kDa, or greater than about 4000 kDa.

In some embodiments, the analyte can be at most about 4000 kDa, at most about 3500 kDa, at most about 3000 kDa, at most about 2500 kDa, at most about 2000 kDa, at most about 1500 kDa, at most about 1000 kDa, at most about 950 kDa, at most about 900 kDa, at most about 850 kDa, at most about 800 kDa, at most about 750 kDa, at most about 700 kDa, at most about 650 kDa, at most about 600 kDa, at most about 550 kDa, at most about 500 kDa, at most about 450 kDa, at most about 400 kDa, at most about 350 kDa, at most about 300 kDa, at most about 250 kDa, at most about 200 kDa, at most about 175 kDa, at most about 150 kDa, at most about 125 kDa, at most about 100 kDa, at most about 95 kDa, at most about 90 kDa, at most about 85 kDa, at most about 80 kDa, at most about 75 kDa, at most about 70 kDa, at most about 65 kDa, at most about 60 kDa, at most about 55 kDa, at most about 50 kDa, at most about 45 kDa, at most about 40 kDa, at most about 35 kDa, at most about 30 kDa, at most about 25 kDa, at most about 20 kDa, at most about 15 kDa, at most about 10 kDa, at most about 9 kDa, at most about 8 kDa, at most about 7 kDa, at most about 6 kDa, at most about 5 kDa, at most about 4 kDa, at most about 3 kDa, at most about 2 kDa, at most about 1 kDa, or less than about 1 kDa.

In some embodiments, the analyte can be from about 1 kDa to about 100 kDa. In some embodiments, the analyte can be from about 1 kDa to about 5 kDa, about 1 kDa to about 10 kDa, about 1 kDa to about 20 kDa, about 1 kDa to about 30 kDa, about 1 kDa to about 40 kDa, about 1 kDa to about 50 kDa, about 1 kDa to about 60 kDa, about 1 kDa to about 70 kDa, about 1 kDa to about 80 kDa, about 1 kDa to about 90 kDa, about 1 kDa to about 100 kDa, about 5 kDa to about 10 kDa, about 5 kDa to about 20 kDa, about 5 kDa to about 30 kDa, about 5 kDa to about 40 kDa, about 5 kDa to about 50 kDa, about 5 kDa to about 60 kDa, about 5 kDa to about 70 kDa, about 5 kDa to about 80 kDa, about 5 kDa to about 90 kDa, about 5 kDa to about 100 kDa, about 10 kDa to about 20 kDa, about 10 kDa to about 30 kDa, about 10 kDa to about 40 kDa, about 10 kDa to about 50 kDa, about 10 kDa to about 60 kDa, about 10 kDa to about 70 kDa, about 10 kDa to about 80 kDa, about 10 kDa to about 90 kDa, about 10 kDa to about 100 kDa, about 20 kDa to about 30 kDa, about 20 kDa to about 40 kDa, about 20 kDa to about 50 kDa, about 20 kDa to about 60 kDa, about 20 kDa to about 70 kDa, about 20 kDa to about 80 kDa, about 20 kDa to about 90 kDa, about 20 kDa to about 100 kDa, about 30 kDa to about 40 kDa, about 30 kDa to about 50 kDa, about 30 kDa to about 60 kDa, about 30 kDa to about 70 kDa, about 30 kDa to about 80 kDa, about 30 kDa to about 90 kDa, about 30 kDa to about 100 kDa, about 40 kDa to about 50 kDa, about 40 kDa to about 60 kDa, about 40 kDa to about 70 kDa, about 40 kDa to about 80 kDa, about 40 kDa to about 90 kDa, about 40 kDa to about 100 kDa, about 50 kDa to about 60 kDa, about 50 kDa to about 70 kDa, about 50 kDa to about 80 kDa, about 50 kDa to about 90 kDa, about 50 kDa to about 100 kDa, about 60 kDa to about 70 kDa, about 60 kDa to about 80 kDa, about 60 kDa to about 90 kDa, about 60 kDa to about 100 kDa, about 70 kDa to about 80 kDa, about 70 kDa to about 90 kDa, about 70 kDa to about 100 kDa, about 80 kDa to about 90 kDa, about 80 kDa to about 100 kDa, or about 90 kDa to about 100 kDa.

In some embodiments, the analyte can be from about 100 kDa to about 4,000 kDa. In some embodiments, the analyte can be from about 100 kDa to about 250 kDa, about 100 kDa to about 500 kDa, about 100 kDa to about 1,000 kDa, about 100 kDa to about 1,500 kDa, about 100 kDa to about 2,000 kDa, about 100 kDa to about 2,500 kDa, about 100 kDa to about 3,000 kDa, about 100 kDa to about 3,500 kDa, about 100 kDa to about 4,000 kDa, about 250 kDa to about 500 kDa, about 250 kDa to about 1,000 kDa, about 250 kDa to about 1,500 kDa, about 250 kDa to about 2,000 kDa, about 250 kDa to about 2,500 kDa, about 250 kDa to about 3,000 kDa, about 250 kDa to about 3,500 kDa, about 250 kDa to about 4,000 kDa, about 500 kDa to about 1,000 kDa, about 500 kDa to about 1,500 kDa, about 500 kDa to about 2,000 kDa, about 500 kDa to about 2,500 kDa, about 500 kDa to about 3,000 kDa, about 500 kDa to about 3,500 kDa, about 500 kDa to about 4,000 kDa, about 1,000 kDa to about 1,500 kDa, about 1,000 kDa to about 2,000 kDa, about 1,000 kDa to about 2,500 kDa, about 1,000 kDa to about 3,000 kDa, about 1,000 kDa to about 3,500 kDa, about 1,000 kDa to about 4,000 kDa, about 1,500 kDa to about 2,000 kDa, about 1,500 kDa to about 2,500 kDa, about 1,500 kDa to about 3,000 kDa, about 1,500 kDa to about 3,500 kDa, about 1,500 kDa to about 4,000 kDa, about 2,000 kDa to about 2,500 kDa, about 2,000 kDa to about 3,000 kDa, about 2,000 kDa to about 3,500 kDa, about 2,000 kDa to about 4,000 kDa, about 2,500 kDa to about 3,000 kDa, about 2,500 kDa to about 3,500 kDa, about 2,500 kDa to about 4,000 kDa, about 3,000 kDa to about 3,500 kDa, about 3,000 kDa to about 4,000 kDa, or about 3,500 kDa to about 4,000 kDa.

In some embodiments, the analyte can be about 1 kDa, about 2 kDa, about 3 kDa, about 4 kDa, about 5 kDa, about 6 kDa, about 7 kDa, about 8 kDa, about 9 kDa, about 10 kDa, about 15 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 125 kDa, about 150 kDa, about 175 kDa, about 200 kDa, about 250 kDa, about 300 kDa, about 350 kDa, about 400 kDa, about 450 kDa, about 500 kDa, about 550 kDa, about 600 kDa, about 650 kDa, about 700 kDa, about 750 kDa, about 800 kDa, about 850 kDa, about 900 kDa, about 950 kDa, about 1000 kDa, about 1500 kDa, about 2000 kDa, about 2500 kDa, about 3000 kDa, about 3500 kDa, or about 4000 kDa.

In some embodiments, the analyte comprises a polypeptide. In some embodiments, the analyte can be at least about 2 amino acids, at least about 5 amino acids, at least about 10 amino acids, at least about 15 amino acids, at least about 20 amino acids, at least about 30 amino acids, at least about 40 amino acids, at least about 50 amino acids, at least about 60 amino acids, at least about 70 amino acids, at least about 80 amino acids, at least about 90 amino acids, at least about 100 amino acids, at least about 150 amino acids, at least about 200 amino acids, at least about 250 amino acids, at least about 300 amino acids, at least about 350 amino acids, at least about 400 amino acids, at least about 450 amino acids, at least about 500 amino acids, at least about 600 amino acids, at least about 700 amino acids, at least about 800 amino acids, at least about 900 amino acids, at least about 1000 amino acids, at least about 2000 amino acids, at least about 3000 amino acids, at least about 4000 amino acids, at least about 5000 amino acids, at least about 6000 amino acids, at least about 7000 amino acids, at least about 8000 amino acids, at least about 9000 amino acids, at least about 10000 amino acids, at least about 20000 amino acids, at least about 30000, at least about 34000 amino acids, or greater than about 34000 amino acids in length. In some embodiments, the analyte can be at most about 34000 amino acids, at most about 30000 amino acids, at most about 20000 amino acids, at most about 10000 amino acids, at most about 9000 amino acids, at most about 8000 amino acids, at most about 7000 amino acids, at most about 6000 amino acids, at most about 5000 amino acids, at most about 4000 amino acids, at most about 3000 amino acids, at most about 2000 amino acids, at most about 1000 amino acids, at most about 900 amino acids, at most about 800 amino acids, at most about 700 amino acids, at most about 600 amino acids, at most about 500 amino acids, at most about 450 amino acids, at most about 400 amino acids, at most about 350 amino acids, at most about 300 amino acids, at most about 250 amino acids, at most about 30000 amino acids, at most about 30000 amino acids, at most about 200 amino acids, at most about 150 amino acids, at most about 100 amino acids, at most about 90 amino acids, at most about 80 amino acids, at most about 70 amino acids, at most about 60 amino acids, at most about 50 amino acids, at most about 40 amino acids, at most about 30 amino acids, at most about 20 amino acids, at most about 15 amino acids, at most about 10 amino acids, at most about 5 amino acids, at most about 2 amino acids, or less than about 2 amino acids in length.

In some embodiments, the analyte can be from about 2 amino acids to about 1,000 amino acids in length. In some embodiments, the analyte can be from about 2 amino acids to about 10 amino acids, about 2 amino acids to about 100 amino acids, about 2 amino acids to about 200 amino acids, about 2 amino acids to about 300 amino acids, about 2 amino acids to about 400 amino acids, about 2 amino acids to about 500 amino acids, about 2 amino acids to about 600 amino acids, about 2 amino acids to about 700 amino acids, about 2 amino acids to about 800 amino acids, about 2 amino acids to about 900 amino acids, about 2 amino acids to about 1,000 amino acids, about 10 amino acids to about 100 amino acids, about 10 amino acids to about 200 amino acids, about 10 amino acids to about 300 amino acids, about 10 amino acids to about 400 amino acids, about 10 amino acids to about 500 amino acids, about 10 amino acids to about 600 amino acids, about 10 amino acids to about 700 amino acids, about 10 amino acids to about 800 amino acids, about 10 amino acids to about 900 amino acids, about 10 amino acids to about 1,000 amino acids, about 100 amino acids to about 200 amino acids, about 100 amino acids to about 300 amino acids, about 100 amino acids to about 400 amino acids, about 100 amino acids to about 500 amino acids, about 100 amino acids to about 600 amino acids, about 100 amino acids to about 700 amino acids, about 100 amino acids to about 800 amino acids, about 100 amino acids to about 900 amino acids, about 100 amino acids to about 1,000 amino acids, about 200 amino acids to about 300 amino acids, about 200 amino acids to about 400 amino acids, about 200 amino acids to about 500 amino acids, about 200 amino acids to about 600 amino acids, about 200 amino acids to about 700 amino acids, about 200 amino acids to about 800 amino acids, about 200 amino acids to about 900 amino acids, about 200 amino acids to about 1,000 amino acids, about 300 amino acids to about 400 amino acids, about 300 amino acids to about 500 amino acids, about 300 amino acids to about 600 amino acids, about 300 amino acids to about 700 amino acids, about 300 amino acids to about 800 amino acids, about 300 amino acids to about 900 amino acids, about 300 amino acids to about 1,000 amino acids, about 400 amino acids to about 500 amino acids, about 400 amino acids to about 600 amino acids, about 400 amino acids to about 700 amino acids, about 400 amino acids to about 800 amino acids, about 400 amino acids to about 900 amino acids, about 400 amino acids to about 1,000 amino acids, about 500 amino acids to about 600 amino acids, about 500 amino acids to about 700 amino acids, about 500 amino acids to about 800 amino acids, about 500 amino acids to about 900 amino acids, about 500 amino acids to about 1,000 amino acids, about 600 amino acids to about 700 amino acids, about 600 amino acids to about 800 amino acids, about 600 amino acids to about 900 amino acids, about 600 amino acids to about 1,000 amino acids, about 700 amino acids to about 800 amino acids, about 700 amino acids to about 900 amino acids, about 700 amino acids to about 1,000 amino acids, about 800 amino acids to about 900 amino acids, about 800 amino acids to about 1,000 amino acids, or about 900 amino acids to about 1,000 amino acids in length.

In some embodiments, the analyte can be from about 1,000 amino acids to about 34,000 amino acids in length. In some embodiments, the analyte can be from about 1,000 amino acids to about 2,500 amino acids, about 1,000 amino acids to about 5,000 amino acids, about 1,000 amino acids to about 7,500 amino acids, about 1,000 amino acids to about 10,000 amino acids, about 1,000 amino acids to about 15,000 amino acids, about 1,000 amino acids to about 20,000 amino acids, about 1,000 amino acids to about 25,000 amino acids, about 1,000 amino acids to about 30,000 amino acids, about 1,000 amino acids to about 34,000 amino acids, about 2,500 amino acids to about 5,000 amino acids, about 2,500 amino acids to about 7,500 amino acids, about 2,500 amino acids to about 10,000 amino acids, about 2,500 amino acids to about 15,000 amino acids, about 2,500 amino acids to about 20,000 amino acids, about 2,500 amino acids to about 25,000 amino acids, about 2,500 amino acids to about 30,000 amino acids, about 2,500 amino acids to about 34,000 amino acids, about 5,000 amino acids to about 7,500 amino acids, about 5,000 amino acids to about 10,000 amino acids, about 5,000 amino acids to about 15,000 amino acids, about 5,000 amino acids to about 20,000 amino acids, about 5,000 amino acids to about 25,000 amino acids, about 5,000 amino acids to about 30,000 amino acids, about 5,000 amino acids to about 34,000 amino acids, about 7,500 amino acids to about 10,000 amino acids, about 7,500 amino acids to about 15,000 amino acids, about 7,500 amino acids to about 20,000 amino acids, about 7,500 amino acids to about 25,000 amino acids, about 7,500 amino acids to about 30,000 amino acids, about 7,500 amino acids to about 34,000 amino acids, about 10,000 amino acids to about 15,000 amino acids, about 10,000 amino acids to about 20,000 amino acids, about 10,000 amino acids to about 25,000 amino acids, about 10,000 amino acids to about 30,000 amino acids, about 10,000 amino acids to about 34,000 amino acids, about 15,000 amino acids to about 20,000 amino acids, about 15,000 amino acids to about 25,000 amino acids, about 15,000 amino acids to about 30,000 amino acids, about 15,000 amino acids to about 34,000 amino acids, about 20,000 amino acids to about 25,000 amino acids, about 20,000 amino acids to about 30,000 amino acids, about 20,000 amino acids to about 34,000 amino acids, about 25,000 amino acids to about 30,000 amino acids, about 25,000 amino acids to about 34,000 amino acids, or about 30,000 amino acids to about 34,000 amino acids in length.

In some embodiments, the analyte can be about 2 amino acids, about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, about 100 amino acids, about 150 amino acids, about 200 amino acids, about 250 amino acids, about 300 amino acids, about 350 amino acids, about 400 amino acids, about 450 amino acids, about 500 amino acids, about 600 amino acids, about 700 amino acids, about 800 amino acids, about 900 amino acids, about 1000 amino acids, about 2000 amino acids, about 3000 amino acids, about 4000 amino acids, about 5000 amino acids, about 6000 amino acids, about 7000 amino acids, about 8000 amino acids, about 9000 amino acids, about 10000 amino acids, about 20000 amino acids, about 30000, or about 34000 amino acids in length.

In some embodiments, the analyte can be at least about 2, at least about 5, at least about 10, at least about 15, at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500, at least about 600, at least about 700, at least about 800, at least about 900, at least about 1000, at least about 2000, at least about 3000, at least about 4000, at least about 5000, at least about 6000, at least about 7000, at least about 8000, at least about 9000, at least about 10000, at least about 20000, at least about 30000, at least about 34000, or greater than about 34000 repeated units, monomers, or groups. In some embodiments, the analyte can be at most about 34000, at most about 30000, at most about 20000, at most about 10000, at most about 9000, at most about 8000, at most about 7000, at most about 6000, at most about 5000, at most about 4000, at most about 3000, at most about 2000, at most about 1000, at most about 900, at most about 800, at most about 700, at most about 600, at most about 500, at most about 450, at most about 400, at most about 350, at most about 300, at most about 250, at most about 30000, at most about 30000, at most about 200, at most about 150, at most about 100, at most about 90, at most about 80, at most about 70, at most about 60, at most about 50, at most about 40, at most about 30, at most about 20, at most about 15, at most about 10, at most about 5, at most about 2, or less than about 2 repeated units, monomers, or groups.

In some embodiments, the analyte can be from about 2 to about 1,000 repeated units, monomers, or groups. In some embodiments, the analyte can be from about 2 to about 10, about 2 to about 100, about 2 to about 200, about 2 to about 300, about 2 to about 400, about 2 to about 500, about 2 to about 600, about 2 to about 700, about 2 to about 800, about 2 to about 900, about 2 to about 1,000, about 10 to about 100, about 10 to about 200, about 10 to about 300, about 10 to about 400, about 10 to about 500, about 10 to about 600, about 10 to about 700, about 10 to about 800, about 10 to about 900, about 10 to about 1,000, about 100 to about 200, about 100 to about 300, about 100 to about 400, about 100 to about 500, about 100 to about 600, about 100 to about 700, about 100 to about 800, about 100 to about 900, about 100 to about 1,000, about 200 to about 300, about 200 to about 400, about 200 to about 500, about 200 to about 600, about 200 to about 700, about 200 to about 800, about 200 to about 900, about 200 to about 1,000, about 300 to about 400, about 300 to about 500, about 300 to about 600, about 300 to about 700, about 300 to about 800, about 300 to about 900, about 300 to about 1,000, about 400 to about 500, about 400 to about 600, about 400 to about 700, about 400 to about 800, about 400 to about 900, about 400 to about 1,000, about 500 to about 600, about 500 to about 700, about 500 to about 800, about 500 to about 900, about 500 to about 1,000, about 600 to about 700, about 600 to about 800, about 600 to about 900, about 600 to about 1,000, about 700 to about 800, about 700 to about 900, about 700 to about 1,000, about 800 to about 900, about 800 to about 1,000, or about 900 to about 1,000 repeated units, monomers, or groups.

In some embodiments, the analyte can be from about 1,000 to about 34,000 in length. In some embodiments, the analyte can be from about 1,000 to about 2,500, about 1,000 to about 5,000, about 1,000 to about 7,500, about 1,000 to about 10,000, about 1,000 to about 15,000, about 1,000 to about 20,000, about 1,000 to about 25,000, about 1,000 to about 30,000, about 1,000 to about 34,000, about 2,500 to about 5,000, about 2,500 to about 7,500, about 2,500 to about 10,000, about 2,500 to about 15,000, about 2,500 to about 20,000, about 2,500 to about 25,000, about 2,500 to about 30,000, about 2,500 to about 34,000, about 5,000 to about 7,500, about 5,000 to about 10,000, about 5,000 to about 15,000, about 5,000 to about 20,000, about 5,000 to about 25,000, about 5,000 to about 30,000, about 5,000 to about 34,000, about 7,500 to about 10,000, about 7,500 to about 15,000, about 7,500 to about 20,000, about 7,500 to about 25,000, about 7,500 to about 30,000, about 7,500 to about 34,000, about 10,000 to about 15,000, about 10,000 to about 20,000, about 10,000 to about 25,000, about 10,000 to about 30,000, about 10,000 to about 34,000, about 15,000 to about 20,000, about 15,000 to about 25,000, about 15,000 to about 30,000, about 15,000 to about 34,000, about 20,000 to about 25,000, about 20,000 to about 30,000, about 20,000 to about 34,000, about 25,000 to about 30,000, about 25,000 to about 34,000, or about 30,000 to about 34,000 repeated units, monomers, or groups.

In some embodiments, the analyte can be about 2, about 5, about 10, about 15, about 20, about 30, about 40, about 50, about 60, about 70, about 80, about 90, about 100, about 150, about 200, about 250, about 300, about 350, about 400, about 450, about 500, about 600, about 700, about 800, about 900, about 1000, about 2000, about 3000, about 4000, about 5000, about 6000, about 7000, about 8000, about 9000, about 10000, about 20000, about 30000, or about 34000 repeated units, monomers, or groups.

In some embodiments, the polymer analyte comprises a repeated unit, monomer, or group. In some cases, a repeated unit can comprise an amino acid, a nucleotide, a saccharide, a monosaccharide, a plastic unit, glycerol, fatty acid, or any combinations thereof.

Protein analyte(s) can be denatured, partially denatured or non-denatured target protein(s) or any mixture thereof. In one embodiment, the target protein(s) is/are in a native form.

In one embodiment, a method of the invention comprises adding to the cis side of the nanopore system a partially denatured or non-denatured (label-free) non-nucleic acid based polymer analyte (e.g., target protein) or a mixture of partially denatured and/or non-denatured (label-free) target proteins. In one embodiment, the target protein(s) is/are all in their native form. In another embodiment, the protein analytes are denatured using common means known in the art, for example heating, extreme pH, or chemical denaturants (eg. urea, guanidinium hydrochloride, detergents, etc.) or a combination thereof. Denatured protein(s) might then be diluted and added to the cis compartment of the nanopore system under non-denaturing run conditions but remain denatured and unable to refold. In another embodiment, the pre-denatured target proteins are added to the nanopore system that retain mild or moderate denaturing conditions to prevent any refolding of the target protein. It is well known that nanopores can tolerate some level of denaturing conditions, so that some level of denaturing conditions might be employed in the running system to maintain the denatured or partially denatured state of the target proteins while retaining the integrity of the nanopore. For example, nanopores can withstand temperatures up to about 100° C. or urea concentrations up to 4 M.

The protein may be obtained, isolated or extracted from any organism or microorganism. For instance, it is obtained from a human or animal, e.g. from a bodily fluid, such as urine, lymph, saliva, mucus, seminal fluid or amniotic fluid, or from whole blood, plasma or serum. The protein may be obtained from a plant e.g. a cereal, legume, ornamental plant, fruit or vegetable, or part thereof including tubers, roots and bulbs.

The protein analyte can be produced inside (animal) cells such that it must be extracted from cells for characterisation by the disclosed methods. The protein may comprise the products of cellular expression of a plasmid in a (microbial) host cell. In some embodiments the protein analyte is secreted from cells.

The analyte (e.g., protein analyte) can be provided as an impure mixture of one or more proteins and/or one or more (proteinaceous) impurities. For example, the polypeptide may be a full length protein and/or impurities may comprise fragments of the protein. Impurities may comprise truncated forms of the target polypeptide which are distinct from the “polypeptide analyte(s)” for characterisation in the disclosed methods. Impurities may also comprise proteins other than the protein analyte e.g. which may be co-purified from a cell culture or obtained from a sample.

A protein may comprise any combination of any amino acids, amino acid analogs and/or modified amino acids (i.e. amino acid derivatives). Amino acids (and derivatives, analogs etc) in the polypeptide can be distinguished by their physical size and/or charge. The amino acids/derivatives/analogs can be naturally occurring or artificial.

In some embodiments, the non-nucleic acid based polymer analyte (e.g., target protein) is modified. In some embodiments, the analyte (e.g., polypeptide) is modified by a leader construct according to embodiments involving a translocase as disclosed herein below. In some embodiments, the disclosed methods are for characterising modifications in the target protein. In one aspect, one or more of the amino acids/derivatives/analogs in the target protein is post-translationally modified. Any one or more post-translational modifications may be present in the target protein.

In some embodiments, post-translational modifications include modification with a hydrophobic group, modification with a cofactor, addition of a chemical group, glycation (the non-enzymatic attachment of a sugar), biotinylation and/or PEGylation. Post-translational modifications can also be non-natural, for instance are chemical modifications introduced in a laboratory for biotechnological or biomedical purposes. This allows for monitoring the levels of post-translational modifications of the laboratory-derived peptide, polypeptide or protein as compared to the natural counterpart. As such, the methods disclosed herein can be used to detect the presence, absence, extent or number of positions of post-translational modifications in a polypeptide.

Examples of post-translational modification with a hydrophobic group can include myristoylation, palmitoylation, isoprenylation or prenylation, the attachment of an isoprenoid group; farnesylation, the attachment of a farnesol group; geranylgeranylation, the attachment of a geranylgeraniol group; and glypiation, and glycosylphosphatidylinositol (GPI) anchor formation via an amide bond. Examples of post-translational modification with a cofactor include lipoylation, attachment of a lipoate (Cs) functional group; flavination, attachment of a flavin moiety (e.g. flavin mononucleotide (FMN) or flavin adenine dinucleotide (FAD)); attachment of heme C, for instance via a thioether bond with cysteine; phosphopantetheinylation, the attachment of a 4′-phosphopantetheinyl group; and retinylidene Schiff base formation.

Examples of post-translational modification by addition of a chemical group can include acylation, e.g. O-acylation (esters), N-acylation (amides) or S-acylation (thioesters); acetylation, the attachment of an acetyl group for instance to the N-terminus or to lysine; formylation; alkylation, the addition of an alkyl group, such as methyl or ethyl; methylation, the addition of a methyl group for instance to lysine or arginine; amidation; butyrylation; gamma-carboxylation; glycosylation, the enzymatic attachment of a glycosyl group for instance to arginine, asparagine, cysteine, hydroxylysine, serine, threonine, tyrosine or tryptophan; polysialylation, the attachment of polysialic acid; malonylation; hydroxylation; iodination; bromination; citrulination; nucleotide addition, the attachment of any nucleotide such as any of those discussed above, ADP ribosylation; oxidation; phosphorylation, the attachment of a phosphate group for instance to serine, threonine or tyrosine (O-linked) or histidine (N-linked); adenylylation, the attachment of an adenylyl moiety for instance to tyrosine (O-linked) or to histidine or lysine (N-linked); propionylation; pyroglutamate formation; S-glutathionylation; Sumoylation; S— nitrosylation; succinylation, the attachment of a succinyl group for instance to lysine; selenoylation, the incorporation of selenium; and ubiquitinilation, the addition of ubiquitin subunits (N-linked).

In some embodiments, a system provided herein is particularly suitable for the analysis of an analyte (e.g., proteinaceous target), such as a polypeptide, greater than 30 amino acids in length. More in particular, a system of the invention provides for capture and translocation of peptides of 30, 50, 100, 300, 500 or greater than 1000 amino acids in length. In one aspect, the analyte is a protein having a mass in the range of between 200 and 5000 Da, preferably 500 and 1700 Da.

In a further embodiment, the polymer analyte is a polysaccharide. Polysaccharides (known in the art as glycans or polycarbohydrates), are long chain polymeric carbohydrates composed of monosaccharide repeating units bound together by glycosidic linkages. They range in structure from linear to highly branched. Examples include storage polysaccharides such as starch, glycogen and galactogen and structural polysaccharides such as cellulose and chitin.

Polysaccharides can be heterogeneous, containing slight modifications of the repeating unit. Depending on the structure, these macromolecules can have distinct properties from their monosaccharide building blocks. They may be amorphous or even insoluble in water.

A polysaccharide analyte may be a homopolysaccharide (or homoglycan) consisting of monosaccharides of the same type, or it is a heteropolysaccharide (or heteroglycan) comprising more than one type of monosaccharide repeating units.

### Protein Translocase

A method or system as herein disclosed for the translocation of a proteinaceous analyte may benefit from the presence of a protein translocase on at least one of the cis and trans side of the system, preferably at least on the cis side.

As used herein, the term “protein translocase” (also known in the art as motor protein, unfoldase) refers to a protein which can bind, and/or translocate along a target non-nucleic acid based polymer analyte (e.g., polypeptide) through the chemical energy provided by NTP (nucleoside triphosphate) hydrolysis, optionally being able to unfold protein structure in the process. Used herein it is able to move along a polypeptide to feed the polypeptide through the nanopore in sequential order. For example, the protein translocase is an NTP-driven unfoldase, preferably an AAA+ unfoldase. See for example US2016/0032235 and Dougan et al. (FEBS Letters 529 (2002) 1873-3468).

The protein translocase may be active or inactive, and the method or system may be run in the presence or absence of NTP. In some embodiments, the translocase can specifically translocate peptide, proteins, polypeptides, or any combination thereof. In some embodiments, the translocase can specifically translocate nucleic acid molecules.

In one aspect, the translocase-target protein complex is formed in solution in the cis side of the fluidic chamber. In another aspect, the translocase-target protein complex is formed in solution during a separate operation prior to adding the complex to the cis side of the fluidic chamber to contact the nanopore. This approach allows for the use of optimal conditions for binding (complex formation) of translocase and analyte (e.g., target protein). For example, in this pre-mixing (preloading) operation a higher concentration of both components, different salt conditions, temperature, pH, co-factors etc, may be chosen than what is typically used in (the cis chamber of) a nanopore sensor system. The premixture may be part of a kit that can be coupled to target proteins of interest. The premixture may be added in a diluted form into the cis chamber.

In another embodiment the translocase is coupled to the nanopore. In the system of the invention there is no need to couple the translocase tightly to the top of the nanopore to optimally feed the non-nucleic acid based polymer analyte (e.g., polypeptide) into the nanopore. Instead the strong cis-to-trans EOF of the invention enables the extruded polypeptide of a target protein to be captured into the nanopore and/or translocated, which will in turn pull the translocase atop the pore, whereupon it will continue to control the movement of the extruded polypeptide. In this embodiment the target protein would not require stall or capture motifs due to the proximity of the extruded polypeptide to the nanopore entrance.

Members of the AAA+ superfamily have been identified in all organisms studied to date. They can be involved in a wide range of cellular events. In bacteria, representatives of this superfamily are involved in functions as diverse as transcription and protein degradation and play an important role in the protein quality control network. Often they employ a common mechanism to mediate an ATP-dependent unfolding/disassembly of protein-protein or DNA-protein complexes. In an increasing number of examples, it appears that the activities of these AAA+ proteins may be modulated by a group of otherwise unrelated proteins, called adaptor proteins.

In some cases, protein translocases for use in the present invention include ClpX, ClpA, ClpC, ClpE, Mpa, Pan, LON, VAT, Cpa, Msp1, HslU/ClpY, AMA, 854, MBA, SAMP, CDC48, FtsH, SecA, or functional homologs or fragments thereof. Preferably, for translocases that contain a protease domain, the proteolytic activity of the protease components are disabled by mutagenesis, or the entire domain is removed.

In some cases, the translocase is the prokaryotic AAA+ unfoldase ClpX. ClpX unfolds substrate analytes (e.g., proteins) by ATP-driven translocation of the non-nucleic acid based polymer analyte (e.g., polypeptide) chain through the central pore of its hexameric assembly. In complex with the ClpP peptidase, ClpX carries out protein degradation by translocating unfolded substrates directly into the ClpP proteolytic chamber (Sauer et al., 2004).

In another embodiment, the protein translocase is the Thermoplasma VCP-like ATPase from Thermoplasma acidophilum (VAT), a member of the two-domain AAA ATPases and homologous to the mammalian p97/VCP and NSF proteins. In another embodiment, the proteasome-activating nucleotidase (PAN) from Methanococcus jannaschii is used, which is a complex of relative molecular mass 650,000 that is homologous to the ATPases in the eukaryotic 26S proteasome. Other examples include AMA, an AAA protein from Archaeoglobus and methanogenic archaea. In a still further embodiment, the translocase is the open reading frame number 854 in the M. mazei genome (Forouzan, Dara, et al. “The archaeal proteasome is regulated by a network of AAA ATPases.” J. Biological Chemistry 287.46 (2012): 39254-39262). Other suitable translocases for use in the present invention include MBA (membrane-bound AAA; Serek-Heuberger, Justyna, et al. “Two unique membrane-bound AAA proteins from Sulfolobus solfataricus.” (2009): 118-122) and SAMPs (Humbard, Matthew A., et al. “Ubiquitin-like small archaeal modifier proteins (SAMPs) in Haloferax volcanii.” Nature 463.7277 (2010): 54). Other examples include ClpA, a member of the two-domain AAA ATPases, from Escherichia coli (Effantin et al., 2010, J Biol Chem., 285, 19, pg. 14834-40). In another embodiment, the protein translocase is ClpC, an AAA protein from Staphylococcus Aureus (Frees et al., 2004, Mol Microbiol., 54, 5, pg. 1445-62). Other examples include CipE, a member of the two-domain AAA ATPases from Bacillus subtilis (Miethke et al., 2004, J Bacteriol., 188, 13, pg. 4610-9). Other examples include HslU/ClpY, an AAA protein from Escherichia coli (Baytshtok et al., 2016, Structure., 24, 10, pg. 1766-1777). In another embodiment, the protein translocase is Lon, a protease from Escherichia coli containing an AAA ATPase domain (Suzuki et al., 2008, Biochim Biophys Acta., 1784(5): pg. 727-35). In another embodiment, the protein translocase is FtsH, a membrane bound protease from Escherichia coli containing an AAA ATPase domain (Langklotz et al., 2012, Biochim Biophys Acta., 1823, 1, pg. 40-8). Other examples include ARC/Mpa (Kavalchuk et al., 2022, Nat Commun., 13, 1, pg. 276), an AAA ATPase from Mycobacterium tuberculosis. Other examples include Msp1, a membrane associated AAA ATPase from Saccharomyces cerevisiae (Castanzo, 2020, Proc Natl Acad Sci USA., 117, 26, pg. 14970-14977). Other examples include CDC48, an AAA ATPase from Saccharomyces cerevisiae (Buchberger, 2013, Subcell Biochem, 66, pg. 195-222). In another embodiment, the protein translocase is Cpa, a CDC48 homologue from actinobacteria (Ziemsky et al., 2018, Elife, pg. 34055). Other examples include SecA, a protein translocase from Escherichia coli.

A method of the invention comprises allowing a protein translocase in solution to capture and form a complex with the target analyte (e.g., target protein) to be translocated. The ratio translocase to target protein is not critical but it may have some effect on efficiency. Typically, a ˜1:1 or slightly greater ratio between translocase and target protein is desired to load one protein translocase per target substrate. Too much translocase may lead to the loading of more than one enzyme onto substrate, whereas too little may lead to large amount of free target protein to capture and block in pore.

In certain aspects of the invention, a protein translocase, and optionally its accessory protease such as ClpP, is present on both the cis and trans side of the nanopore. In this way the target protein added to the cis compartment is first fed through the nanopore via the translocase on the cis side, and the protein translocase on the trans side aids the progression of the target protein through the nanopore and/or prevents the folding of protein (the latter in particular when combined with an accessory protease unit in the trans that degrades the target protein once it reaches the trans side).

In an alternative embodiment, a protein translocase such as ClpX, and optionally its accessory protease such as ClpP, is only present on the trans side of the nanopore, in combination with the defined dominant EOF that is set up to act trans-to-cis according to the present invention, wherein the ClpX translocase binds the target protein added to cis compartment after it has been captured and/or partially translocated through the nanopore cis-to-trans via a polyionic tag that enables electrophoretic capture, and/or wherein the ClpX translocase then acts to pull the protein through the nanopore from cis-to-trans. This approach addresses the situation that, while the cis-to-trans EPF can aid the initial movement of the protein cis-to-trans, it can also be counterproductive, eg. pulling charged and/or unstructured parts of the protein through the nanopore too quickly. Here the EOF acting against the direction of analyte (e.g., polypeptide) translocation acts to keep the polypeptide within the nanopore and/or stretched as the translocase pulls it out under controlled, thus preventing uncontrolled EPF related slips.

In another embodiment of the invention the translocase is physically attached to the nanopore at either the cis or trans entrance (with respect to the nanopore geometry). The translocase may be coupled tightly to the top of the nanopore so that the translocase assembly is co-planar with the nanopore assembly, such that the central cavity of the translocase is aligned with the entrance to the nanopore channel to optimally feed the analyte (e.g., polypeptide) into the nanopore. One means of achieving this is to genetically fuse the translocase protomers to the protomers that form the pore. Preferably, the translocase is tethered to the top of the nanopore via a single anchor point that keeps the translocase in the vicinity of the nanopore entrance. Means of attaching analytes (e.g., proteins) to the top of nanopores are known in the art, including but not limited to chemical attachment (e.g. via cysteines), enzymatic attachment (e.g. Stranges et al. 2016, Proc Natl Acad Sci USA, 113 (44), pg. 6749-E6756), or via hybridization of complementary DNA tags that are attached to the top of the nanopore and/or the translocase, respectively.

### Leader Construct

According to the invention, a target analyte (e.g., target protein) may be coupled to a leader construct, for example to create substrates that can preload and/or stall translocases.

In some embodiments, it is not a pre-requisite to add leaders to make the system work. It is also possible to use unstalled translocase motors and then when target substrate is captured, there is a good chance of capturing the translocase at some point along the protein. On the other hand, an advantage of having it stalled is that all the proteins start at the start.

Various leader construct designs are possible.
The leader may comprise some or all of the following elements:
   1. A recognition motif
   2. A capture motif
   3. A stall motif
   4. A block motif
   5. A coupling motif

The individual elements may be arranged in any different order. The elements might be combined in one motif, so that one sequence performs multiple functions.

In some embodiments, the coupling motif is positioned at the end, preferably preceded by a block motif Variations may include:
Recognition-capture-stall-block-coupling
Capture-stall-block-coupling
Recognition-stall-block-coupling

Translocases can be capable of moving through a wide variety of chemical composition polymers, structures and/or cross-links, so the various motifs may fully or only partially consist of amino acids. They might contain no amino acids. The amino acids can be natural, non-natural, or a combination thereof. Since translocases are capable of moving along analytes (e.g., proteins) either in the C to N direction or the N to C direction the components of the leader may be arranged with either C>N or N>C orientation, or different combinations thereof in a single construct. The leader construct may contain stretches of other polymeric molecules, such as PEG. The overall composition of the construct may be optimised for good water solubility. The composition may be optimised for low structural propensity in regions of capture to enable more efficient pore capture.

Leaders may be added to the N- or C-terminus of a target protein, or to both ends. Since translocases can run either N>C or C>N, adding a leader (e.g. of the same design) to both ends of a target protein can enable capture and/or threading in both the C>N and N>C directions, which can provide different information that can be combined informatically to improve accuracy of the analysis for example. Alternatively, both termini of the target protein are coupled to different leader construct designs, creating separate “leader” and “tail” ends. Leader-tail constructs can be used to control which behaviours occur at which end, for example to control the orientation of the capture of the target protein, the loading and relative direction of the translocase, etc. For example, in combination with a leader that directs binding of translocase and/or capture in the nanopore (e.g. leader contains translocase recognition motif/capture motif/stall motif/block motif), a simple tail sequence (e.g. simple unstructured amino acid sequence) can be added to the opposite end to add additional sequence for translocase to travel along during nanopore translocation so that the entirety of the target protein sequence passes through the nanopore before the translocase encounters the end of the molecule and unbinds.

### Recognition Motif

In some embodiments, the recognition motif can enable optimal binding of the leader-conjugated target analyte (e.g., peptide) to the translocase/unfoldase. Suitable motifs are known in the art or can be discovered by known means, and include the peptide tag ssrA (AANDENYALAA (SEQ ID NO: 1)) to enhance binding of the tagged analyte (e.g., tagged protein) to ClpX, ClpA, ClpC, ClpE, PAN, FtsH, VAT etc, pup tag (Prokaryotic Ubiquitin-like Protein, MAQEQTKRGGGGGDDDDIAGSTAAGQERREKLTEETDDLLDEIDDVLEENAEDFVRAYVQ KGGQ (SEQ ID NO: 2)) to enhance binding to Mpa etc, C-terminal residues from SulA (SASSHATRQLSGLKIHSNLYH (SEQ ID NO: 3)) to enhance binding to HslU, Lon, etc, and portions of Pex15 (Pex15²⁵⁴⁻³⁰⁹ AKSKGKQRGVKQKIHHFHEPMLHNSSEEQVKVEDAFNQRTSTDSRLQSTGTAPRKK, (SEQ ID NO: 4) Pex15⁴³⁻³⁰⁹ SEVFQECVNLFIKRDIKDCLEKMSEVGFIDITVFKSNPMILDLFVSACDIMPSFTKLGLTLQSE ILNIFTLDTPQCIETRKIILGDLSKLLVINKFFRCCIKVIQFNLTDHTEQEEKTLELESIMSDFIFV YITKMRTTIDVVGLQELIEIFIFQVKVKLHHKKPSPNMYWALCKTLPKLSPTLKGLYLSKDV SIEDAILNSIDNKIQKDKAKSKGKQRGVKQKIHIFHEPMLHNSSEEQVKVEDAFNQRTSTDS RLQSTGTAPRKK (SEQ ID NO: 5), Pex15¹⁻³⁰⁹ MAASEIMNNLPMHSLDSSLRDLLNDDLFIESDESTKSVNDQRSEVFQECVNLFIKRDIKDCL EKMSEVGFIDITVFKSNPMILDLFVSACDIMPSFTKLGLTLQSEILNIFTLDTPQCIETRKIILGD LSKLLVINKFFRCCIKVIQFNLTDHTEQEEKTLELESIMSDFIFVYITKMRTTIDVVGLQELIEIF IFQVKVKLHHKKPSPNMYWALCKTLPKLSPTLKGLYLSKDVSIEDAILNSIDNKIQKDKAKS KGKQRGVKQKIHHFHEPMLHNSSEEQVKVEDAFNQRTSTDSRLQSTGTAPRKK (SEQ ID NO: 6)) to enhance binding to Msp1 etc.

Also encompassed are genetically engineered (mutated) variants of (known) recognition elements, e.g. those resulting from a screening to discover what binds (such as described in Flynn et al., 2001, Proc Natl Acad Sci USA, 98, 19, pg. 10584-9), or where translocase is evolved to recognise a chosen sequence.

### Capture Motif

In some embodiments, the capture motif can be optimised for capture in the nanopore. The recognition motif and pore-capture motif may be combined in one, bifunctional motif. Preferably, the capture motif comprises amino acids with a net charge to facilitate capture in a nanopore under the appropriate applied voltage by means of electrophoretic attraction. For example, the motif may be comprised of some or all positively charged amino acids, where capture is improved in conditions where a negative applied voltage is applied to the opposite side of the membrane in which the substrate is contained so that it is attracted into the pore (or vice versa for a negatively charged capture motif).

The capture motif unstructured to enable efficient capture in the nanopore. The capture motif is ideally designed to be long enough to aid efficient capture in the pore and in combination with other motifs is designed so that when captured in the nanopore from the cis side it reaches to the trans exit of the nanopore or vice versa when the bound translocase contacts the top of the nanopore and prevents further uncontrolled translocation.

For example, a polyanion tag might be wholly or partially created from n repeats of (Serine-Glycine-Aspartic Acid)n, (Serine-Aspartic Acid)n, (Aspartic Acid)n or various combinations thereof. Alternatively, a polycation tag might be wholly or partially created from n repeats of (Serine-Glycine-Arginine)n, (Serine-Arginine)n, (Arginine)n or various combinations thereof. In some embodiments, n can be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 40, at least about 45, at least about 50, or greater than about 50. In some embodiments, n can be at most about 50, at most about 45, at most about 40, at most about 35, at most about 30, at most about 25, at most about 20, at most about 19, at most about 18, at most about 17, at most about 16, at most about 15, at most about 14, at most about 13, at most about 12, at most about 11, at most about 10, at most about 9, at most about 8, at most about 7, at most about 6, at most about 5, at most about 4, at most about 3, at most about 2, at most about 1, or less than about 1.

In some embodiments, n can be from about 1 to about 50. In some embodiments, n can be from about 1 to about 2, about 1 to about 3, about 1 to about 4, about 1 to about 5, about 1 to about 10, about 1 to about 15, about 1 to about 20, about 1 to about 25, about 1 to about 30, about 1 to about 40, about 1 to about 50, about 2 to about 3, about 2 to about 4, about 2 to about 5, about 2 to about 10, about 2 to about 15, about 2 to about 20, about 2 to about 25, about 2 to about 30, about 2 to about 40, about 2 to about 50, about 3 to about 4, about 3 to about 5, about 3 to about 10, about 3 to about 15, about 3 to about 20, about 3 to about 25, about 3 to about 30, about 3 to about 40, about 3 to about 50, about 4 to about 5, about 4 to about 10, about 4 to about 15, about 4 to about 20, about 4 to about 25, about 4 to about 30, about 4 to about 40, about 4 to about 50, about 5 to about 10, about 5 to about 15, about 5 to about 20, about 5 to about 25, about 5 to about 30, about 5 to about 40, about 5 to about 50, about 10 to about 15, about 10 to about 20, about 10 to about 25, about 10 to about 30, about 10 to about 40, about 10 to about 50, about 15 to about 20, about 15 to about 25, about 15 to about 30, about 15 to about 40, about 15 to about 50, about 20 to about 25, about 20 to about 30, about 20 to about 40, about 20 to about 50, about 25 to about 30, about 25 to about 40, about 25 to about 50, about 30 to about 40, about 30 to about 50, or about 40 to about 50.

In some embodiments, n can be about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 40, about 45, or about 50. These are not intended to be limiting, and a person of skill in the art would understand that many more combinations of charged residues are possible and there are straightforward means of assessing that they enable capture by the nanopore under the chosen conditions (salt type, salt concentration, pH, orientation of applied voltage, polarity of net charge inside nanopore, etc) using the experimental systems described herein. For example, it is preferable to use a polyanion capture tag in combination with a nanopore with net positive internal charges in a system with a positive voltage applied to the trans compartment with a net EOF is cis-to-trans, wherein the tagged target protein is added to the cis side. Most preferably, the capture tag is a polycation tag, used in a system with a nanopore with net negative internal charge where a negative voltage is applied to the trans compartment to create a strong net EOF cis-to-trans, wherein the tagged target protein is added to the cis side.

### Stall Motif

In some embodiments, the stall motif is a region of low traction for the translocase protein. That is a region upon which the enzyme cannot easily gain traction, e.g. due to a lack of side chains or small side chains, or side chains that do not interact favourably with the “paddles” through which the translocase exerts its NTP driven powerstroke. When encountering the stall region the translocase will struggle to proceed and will undergo futile NTP turnover, most likely undergoing progressive periods of moving, slipping, back sliding, rebinding, moving etc. Without wishing to be limited, the stall motif preferably comprises a suitably long stretch amino acid residues such as glycines, alanines etc. and other sequences based on those described in Hiu-Mei Too et al., 2013, J Biol Chem, 288, 19, pg. 13243-57, for example 3 glycines, or 6 glycines, or 9 glycines or more. Optionally, the glycines are interspersed with other residues, such as serine, to improve non-nucleic acid based polymer analyte (e.g., polypeptide) properties such as structure and/or solubility.

In some embodiments, the Stall motif is alternatively comprised of non-amino acid polymeric linker chemistry, such as for example, polyethylene glycol. Many suitable linker chemistries may be used.

### Block Motif

In some embodiments, the block motif is a region positioned after the stall region through which the translocase cannot easily progress. A block region after a stall region is preferable to prevent the translocase diffusing past the stall region and regaining traction on the substrate beyond the stall. Alternatively, the block motif can replace the stall region if it is sufficiently robust and which the translocase is unable to overcome on its own.

The block motif is designed to provide some type of steric blockade. For example, the block may be formed by a folded analyte structure (e.g., folded protein structure) that is part of the analyte (e.g., polypeptide) backbone of the translocated substrate that the protein cannot easily unfold. The block motif can be a protein such as Maltose Binding Protein (MBP), Titin etc. Specific exemplary block motifs include proteins (enzymes) that are resistant to unfolding by ClpX, for example dihydrofolate reductase or barnase in presence of stabilizing ligands.

Alternatively, in some embodiments, the block motif is a large bulky side chain. The large bulky side-chain might for example be a covalently bound large molecule, such as a carbohydrate, a multi-ring molecule, or a branched dextran. Alternatively, the side chain might have a relatively small binder to which a larger species is bound non-covalently (and which is displaced when the motor proceeds through the block), such as for example a biotin to which a streptavidin protein is bound, or a small antigen element to which a nanobody or antibody is bound.

The leader construct may have stall and/or block motif(s) depending on the relative combined ability to limit progression of the translocase through the protein substrate.

### Coupling Motif

In some embodiments, the coupling motif allows for conjugation of the leader construct to the target analyte (e.g., target protein). For example, it provides the chemistry for chemically attaching the leader to the analyte. Chemical coupling systems and coupling motifs are known in the art. They include coupling chemistries for attaching to target proteins with cysteines or lysines. Preferably the motif comprises a tag for specifically coupling to either the N- or C-termini of any target protein. For example, N-terminal targeting chemistries are known (Rosen et al. 2017, Nature Chemical Biology Vol. 13, pg. 697-705).

Alternatively, the coupling motif may be a comprised of sequence that enables an enzymatic means of coupling the leader to the analyte. For example, it comprises a motif for binding/loading of an enzyme having peptide ligase activity, such as a broad spectrum peptiligase (e.g. Toplak et al., 2016, Water. Adv Synth Catal, 358, pg. 2140-7) e.g. omniligase (Nuijens et al. (2016) Adv Synth Catal 358: 4041-4048) or a similar enzyme. The coupling motif may employ recognition sequences that enable enzymes such as Sortases to couple either to the N- or C-terminus (Guimaraes et al. 2013, Nature Prot. Vol. 8, pg. 1787-1799).

In one embodiment, the target protein is conjugated at its N-terminus, or its C-terminus or both its N- and C-terminus to a leader construct of similar composition, so that when added to the nanopore system capture and/or translocation is enabled both N-to-C and C-to-N depending on what end the captured leader was attached. This enables target proteins to be characterized both in an N-to-C direction and a C-to-N direction. This can be advantageous to improve the accuracy of characterization, for example to capture information that is different in the each orientation.

According to the invention, a target protein may be conjugated to the leader construct using chemical or enzymatic means. The method may comprise the operation of conjugating target protein to a leader construct. In one embodiment, protein conjugation to a leader construct is performed prior to contacting a target protein with protein translocase. In another embodiment, the leader construct is pre-loaded with protein translocase prior to conjugation to the protein.

### Barcode Motif

The leader construct may also contain a barcode motif/sequence that produces a unique signal when passing through the nanopore so as to uniquely identify the Leader from a mixture of other barcoded leader constructs. Barcodes can be used to label multiple separate samples that are then combined and run as a single pool in nanopore system, enabling the separate barcoded samples to be separated again informatically based on their unique signals. Barcode motifs can be created from different amino-acid sequences, non-natural amino acids, modified amino acids, or other polymer moieties or combinations thereof to create unique signals when passed through a nanopore.

### Membrane Binding Motif

The leader construct may also contain a membrane- or nanopore-binding motif to direct the binding of the Leader construct and attached target analyte (e.g., target protein) either to a membrane or a nanopore, respectively. Membrane binding molecules are known in the art, and include for example hydrophobic or amphipathic molecules such as cholesterol that can be attached to the Leader via a side chain. Alternatively, the Leader construct may comprise a sequence that forms an amphipathic structure, e.g. an amphipathic alpha helix (Manuel Gimenez-Andres et al., Biomolecules. 2018 September; 8(3): 45 to enable membrane association.

### Binding and Preloading

In one embodiment one or more translocases are bound and/or loaded onto the target analyte (e.g., target protein) prior to addition to the nanopore system of the invention, which we term “preloading” herein. Preferably the preloading is performed under conditions that favour high efficiency of binding and/or loading, and/or optimal translocase movement along the target protein (whether modified or unmodified). For example, in one embodiment the translocase and one or more target proteins are incubated at relatively higher concentration, and then diluted when added to the nanopore system of the invention. Preferably the preloading is performed in conditions closer to the optimal binding conditions than employed in the nanopore system of the invention. For example, preloading is preferably performed in solutions that are closer to the optimal salt concentration and salt types, the optimal pH, the optimal temperature, and in the presence of optimal co-factors (e.g. NTP, M²⁺ ions, etc). Preloading may also be performed in combination with accessory cofactors that aid in binding. These include but are not limited to proteins derived from naturally occurring substrate adaptors such as those described in Bouchnak et al., 2021, J Biol Chem., 296, pg. 100338 (e.g., NblA/B, ClpS, ClpF), or engineered binding cofactors such as ones derived from antibodies, nanobodies, affimers etc.

In other embodiments the preloading is performed under conditions that enables multiple translocases to bind to a single target protein. This can be advantageous to provide better controlled movement of the polynucleotide through the nanopore in the methods of the invention, and/or to improve the ability of the translocase to progress through more problematic regions of proteins (e.g. regions of very stable structure, regions with bulky modifications of the side chains, or regions of low traction such those composed of a high percentage of glycines). In one aspect, preloading to load multiple translocases is performed under relatively high ratio of translocase to target protein. Preferably this is performed in combination with target proteins that are modified to optimally bind multiple translocases, for example through attachment of sufficiently long leaders to the terminus(i) of the target proteins. For example, leaders are designed to have sufficiently long binding and/or stall motifs to accommodate the footprint of the multiple translocases and/or stall the multiple translocases respectively.

In another embodiment the translocases are topologically closed around the leader and/or target protein to reduce or prevent unbinding, for example especially when the translocase:target-protein is added to nanopore systems that employ conditions relatively unfavourable to binding (e.g. high salt concentration). Means of fusing multiple units of oligomeric AAA+ translocases together by genetic fusion are known. Alternatively, oligomers can be connected by covalent coupling, e.g. by cross reaction between suitably placed cysteines between subunits. Genetic fusion and chemical coupling can be used in combination.

### Nanopore System

A further embodiment relates to a nanopore system for translocating a target polymer through a nanopore, the system comprising a membrane having nanopore therein, said membrane separating a chamber into a cis side and a trans side, and means for providing a voltage difference between the cis side and the trans side of the membrane, wherein the nanopore system has a cis to trans electro-osmotic force (EOF) resulting from a net ionic current flow cis to trans, preferably wherein the cis to trans EOF results from a net ionic current flow cis to trans over total ionic current flow of greater than 0.2 or less than −0.2. Preferably, the nanopore system has a cis to trans EOF resulting from a net ionic current flow cis-to-trans over total ionic current flow of greater than 0.3 or less than −0.3, more preferably greater than 0.35 or less than −0.35.

In some embodiments, the nanopore system has an ion-selectivity P₍₊₎/P₍₋₎ of greater than 2.0 or less than 0.5, preferably greater than 2.5 or less than 0.4, most preferably greater than 3.0 or less than 0.33. In one aspect, the nanopore system has an ion selectivity P+/P− of greater than 3.0 or less than 0.3, preferably greater than 3.5 or less than 0.2, under an applied voltage difference across the membrane.

As described herein above, the large and/or dominant EOF by inducing a strong ion-selectivity current bias through the nanopore can be achieved in various ways. These include modification engineering of the nanopore, applying specific system conditions, and any combination thereof.

In one embodiment, the system contains cation salts of K+, Na+, NH4+ or other suitably small and highly mobile cation salts on the cis side of the system to provide the majority of the ionic flux through the nanopore from cis to trans under negative applied voltage to trans. In a further aspect, the system preferably contains anionic salts of glutamate, acetate or other suitably large and less mobile anion salts on the trans side of the system to further limit to anionic flux through the nanopore from trans to cis under negative applied voltage to trans.

In certain aspects, a negative voltage is applied to the trans side of the device when target polymer(s) are added to the cis side of the nanopore. In this embodiment, the negative voltage drives a large excess of cations from cis to trans, creating a large cation biased cis-to-trans EOF acting on the system.

The system may further comprise means for measuring a signal based on ionic current flowing through the nanopore during a period of time of translocation. For example, the measuring means are set up to detect changes in the signal that reflect characteristics of the polymer as it is translocated. Other measuring means include those involving tunneling and/or plasmonics methods.

The system may comprise a circuit that can both apply the voltage and measure the current. Alternatively, it comprises one circuit to apply the voltage difference and another to measure the current. It is also possible to create the voltage difference with an asymmetric salt across the membrane.

Mechanisms for detecting the current between the cis and trans chambers were described in WO 00/79257 U.S. Pat. Nos. 6,46,594, 6,673, 6,673,615, 6,627,067, 6,464,842, 6,362,002, 6,267,872, 6,015,714, 6,428,959, 6,617,113 and 5,795,782 and US Publications Nos. 2004/0121525, 2003/0104428 and 2003/0104428. They may include electrodes directly associated with the channel or pore at or near the porous opening, electrodes placed within the cis and trans chambers, and insulated glass microelectrodes. Electrodes may be capable of, detecting differences in ionic current around two chambers or tunneling electrical current around the porous opening. In another configuration, the transport property is the flow of electrons around the diameter of the aperture which can be monitored by electrodes placed adjacent to or touching the circumference of the nanopore. Said electrodes can be attached to an Axopatch 200B amplifier to amplify a signal.

It is understood that acquisition systems described herein is not limited and that other systems for acquiring or measuring nanopore signals can be employed. Alternative electrical schemes can also be employed, on arrayed chip platforms for example, to achieve an equivalent voltage drop across the nanopore and/or membrane.

A further aspect of the invention relates to an analytical device comprising one or more nanopore systems according to the invention.

Also provided herein is the use of a system or analytical device according to the invention for single non-nucleic acid based polymer analysis, preferably for identification and/or sequencing of one or more (label-free) polypeptide(s). A person skilled in the art will recognize and appreciate that the invention provides a unique addition to the landscape of emerging (bio)polymer technologies. For example, it finds its application in a broad area of analyte (e.g., protein) analysis, including next-generation single-molecule protein sequencing and identification technologies and single-cell profiling of proteomes.

Other important areas of applications include glycomics and glycoproteomics. Because the glycome is influenced by both genetic and environmental factors, the information contained therein sheds light on intra- and interspecies variations, including providing indicators of disease that can be used for diagnosis and/or for monitoring the efficacy of drugs. Comparative glycomics, the comparison of glycome profiles obtained from two or more individuals, tissues, or conditions of interest, is therefore an exciting frontier in biology and medicine The numerous factors that influence the glycome (the transcriptome, the proteome, environmental nutrients, the secretory machinery, pH, and many other determinants) create a glycome that is highly diverse, adaptable, and dynamic. Thus, the glycome of a cell can change dramatically over time. It is this enormous structural plasticity in response to cellular and environmental states that underlies the essential roles of glycans in development, communication, and disease processes.

### Kit

In one aspect, the disclosure provides kits of analysis of one or more characteristics of one or more analytes. Kits can comprise one or more elements disclosed herein in relation to any of the various aspects, in any combination. In some embodiments, the kit can comprise a nanopore system. In some cases, the nanopore system can comprise a fluidic chamber with a membrane. In some cases, the membrane can split the fluidic chamber into a cis side and a trans side. In some cases, the fluidic chamber can further comprise a nanopore. In some cases, the nanopore is embedded into the membrane. In some embodiments, the kit can further comprise a first solution. In some cases, the first solution can be added to the cis side of the fluidic chamber. In some embodiments, the kit can comprise a second solution. In some cases, the second solution can be added to the trans side of the fluidic chamber. In some cases, the first solution and the second solution can be the same solution. In some cases, the first solution and the second solution can be different solutions. In some embodiments, the kit can further comprise a sample analyte for testing. In some cases, the sample analyte can be used to set up the system of the present disclosure.

In one aspect, the present disclosure provides a device comprising an array of the system. In some embodiments, the system can comprise any one of the systems disclosed herein.

In one aspect, the present disclosure provides a method of characterizing at least one structural feature of the non-nucleic acid based polymer analyte. In some embodiments, the method can comprise any one of the methods disclosed herein.

In one aspect, the present disclosure provides a method for analysis of an amino acid sequence or amino acid composition of one or more non-nucleic acid based polymer analytes. In some embodiments, the analysis can be performed at a single molecule level. In some embodiments, the method can comprise any one of the methods disclosed herein.

In one aspect, the present disclosure provides a system for characterizing at least one structural feature of the non-nucleic acid based polymer analyte. In some embodiments, the system can comprise any one of the systems disclosed herein.

In one aspect, the present disclosure provides a system for analysis of an amino acid sequence or amino acid composition of one or more non-nucleic acid based polymer analytes. In some embodiments, the analysis can be performed at a single molecule level. In some embodiments, the system can comprise any one of the systems disclosed herein.

### Computer Systems

The present disclosure provides computer systems that are programmed to implement methods of determining one or more characteristics of an analyte. FIG. 16 shows a computer system 701 that is programmed or otherwise configured to determine one or more characteristics of an analyte. The computer system 1601 can regulate various aspects of detecting presence or absence of one or more characteristics of the analyte, such as, for example, determining the sequence of the analyte. The computer system 1601 can be an electronic device of a user or a computer system that is remotely located with respect to the electronic device. The electronic device can be a mobile electronic device.

The computer system 1601 includes a central processing unit (CPU, also “processor” and “computer processor” herein) 1605, which can be a single core or multi core processor, or a plurality of processors for parallel processing. The computer system 1601 also includes memory or memory location 1610 (e.g., random-access memory, read-only memory, flash memory), electronic storage unit 1615 (e.g., hard disk), communication interface 1620 (e.g., network adapter) for communicating with one or more other systems, and peripheral devices 1625, such as cache, other memory, data storage and/or electronic display adapters. The memory 1610, storage unit 1615, interface 1620 and peripheral devices 1625 are in communication with the CPU 1605 through a communication bus (solid lines), such as a motherboard. The storage unit 1615 can be a data storage unit (or data repository) for storing data. The computer system 1601 can be operatively coupled to a computer network (“network”) 1630 with the aid of the communication interface 1620. The network 1630 can be the Internet, an internet and/or extranet, or an intranet and/or extranet that is in communication with the Internet. The network 1630 in some cases is a telecommunication and/or data network. The network 1630 can include one or more computer servers, which can enable distributed computing, such as cloud computing. The network 1630, in some cases with the aid of the computer system 1601, can implement a peer-to-peer network, which may enable devices coupled to the computer system 701 to behave as a client or a server.

The CPU 1605 can execute a sequence of machine-readable instructions, which can be embodied in a program or software. The instructions may be stored in a memory location, such as the memory 1610. The instructions can be directed to the CPU 1605, which can subsequently program or otherwise configure the CPU 1605 to implement methods of the present disclosure. Examples of operations performed by the CPU 1605 can include fetch, decode, execute, and writeback.

The CPU 1605 can be part of a circuit, such as an integrated circuit. One or more other components of the system 1601 can be included in the circuit. In some cases, the circuit is an application specific integrated circuit (ASIC).

The storage unit 1615 can store files, such as drivers, libraries and saved programs. The storage unit 1615 can store user data, e.g., user preferences and user programs. The computer system 1601 in some cases can include one or more additional data storage units that are external to the computer system 1601, such as located on a remote server that is in communication with the computer system 1601 through an intranet or the Internet.

The computer system 1601 can communicate with one or more remote computer systems through the network 1630. For instance, the computer system 1601 can communicate with a remote computer system of a user (e.g., a personal computer). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1601 via the network 1630.

Methods as described herein can be implemented by way of machine (e.g., computer processor) executable code stored on an electronic storage location of the computer system 1601, such as, for example, on the memory 1610 or electronic storage unit 1615. The machine executable or machine readable code can be provided in the form of software. During use, the code can be executed by the processor 1605. In some cases, the code can be retrieved from the storage unit 1615 and stored on the memory 1610 for ready access by the processor 1605. In some situations, the electronic storage unit 1615 can be precluded, and machine-executable instructions are stored on memory 1610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1601 can include or be in communication with an electronic display 1635 that comprises a user interface (UI) 1640 for providing, for example,

[symbol]

the identification of the target nucleic acid sequence. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1605.

Another aspect of the present disclosure provides a non-transitory computer readable medium comprising machine executable code that, upon execution by one or more computer processors, implements any of the methods above or elsewhere herein. Another aspect of the present disclosure provides a system comprising one or more computer processors and the computer memory coupled thereto. The computer memory comprises machine executable code that, upon execution by the one or more computer processors, implements any of the methods above or elsewhere herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1: Schematic drawing of strong electro-osmotic nanopore systems for improving the translocation and characterisation of polymer analytes such as polypeptides through the nanopores. Figure illustrates nanopores with a strong net Electro-Osmotic Force (EOF) in the direction cis-to-trans across a membrane as indicated by the arrow. The Electrophoretic Forces (EPF) acting on the analyte will depend on the composition of charges on the target analyte in the sections in and near the nanopore channel, and therefore can sometimes act in the net direction cis-to-trans or trans-to-cis. A strong and dominant cis-to-trans EOF enables capture, stretching and efficient translocation of long polymer analytes from the cis compartment to the trans compartment regardless of the net direction of the EPF. A) Schematic of the general case, illustrating a strong cis-to-trans EOF across the system for enabling capture and translocation of a polymer analyte in the cis to trans direction. Arrows through the pore schematically indicate the magnitude of the ion flow in each direction, showing that the EOF is generated by a large net flow of ions from cis to trans. The net flow arises from a large cis-to-trans ion flow dominating over any trans-to-cis ion flows (e.g a lower flow or counter-charged ions under an applied potential). B) A strong cis-to-trans EOF can be established in a system with positive voltage applied to the trans compartment across the membrane using nanopores with net positive internal charge to limit the flow of cations from trans to cis. Panel C) A strong cis-to-trans EOF can be established in a system with negative voltage applied to the trans compartment across the membrane using nanopores with net negative internal charge to limit the flow of anions from trans to cis.

FIG. 2: Exemplary nanopore-based systems for characterising and/or translocating polymer analytes, for example mixed amino-acid composition proteins. System comprising a nanopore in a membrane, where a polymer analyte is translocated through the nanopore from the cis compartment to the trans compartment with the aid of a translocase motor that progresses along the polymer analyte in the direction of the subset arrow (moving away from termini PA towards termini PB of polymer analyte). Depending on the charge composition of the portion of the polymer analyte within or near the nanopore central channel at any one time, and on the direction of the applied voltage, the net direction of the EPF acting on the polymer may be either cis-to-trans or trans-to-cis or effectively zero as the polymer progresses through the nanopore (as indicated by the dotted arrow labelled EPF).

FIG. 3. CytK nanopores. A) Cut-through of a surface representation of WT-CytK nanopores in 1 M KCl, pH 7.5. The nanopore was made by homology modelling from the alpha-hemolysin nanopore. B) Cartoon representation of WT-CytK β-barrel region. The N-terminal strand is in green and the C-terminal strand in orange. The charged residues are underlined. C) A schematic of the residues in each beta strand of the transmembrane beta-barrel region of wild-type CytK, marking water-facing residues of the down- and up-strands most suitable for mutagenesis. D) Cut-through of a surface (left) and cartoon (right) representation of a high ion selectivity mutant, CytK-2E-4D, in 1 M KCl, pH 7.5 [(p(K)/p(Cl) of 4.04±0.07, and p(K)/p(Cl) of 1.3 at pH 3.8].

FIG. 4. Amino acid sequence and corresponding schematic representation of the three designed unstructured model polypeptide substrates used herein (referred to as S1; tzatziki and mujdei). Red dots (solid circles) indicate negatively charged amino acids, and open circles indicate positively charged amino acids. FIG. 4 discloses SEQ ID NOS 11, 9 and 10, respectively, in order of appearance.

FIG. 5. Translocation of S1 through WT-CytK nanopores. A) Schematic representation of the translocation of S1 through 2E-4D-CytK. B) Voltage dependency of translocation rates for type 1 and type 2 blockades. C) Voltage dependency of the excluded current (Iex %) for type 1 and type 2 blockades. D) Representative traces at −160 mV bias. E) Dwell time versus current amplitude at −160 mV bias. Experiments were performed at pH 7.5 and under 1 M KCl. Traces were collected at 50 KHz sampling rates and filtered at 10 KHz using a Bessel filter.

FIG. 6. Translocation of S1 through 2E-4D-CytK nanopores. A) Schematic representation of the translocation of S1 through 2E-4D-CytK. B) Voltage dependency of translocation rate C) Voltage dependency of the excluded current (Iex %) D) Representative traces at −40 mV bias. E) Dwell time versus current amplitude at −40 mV bias. Experiments were performed at pH 7.5 and under 1 M KCl. Traces were collected at 50 KHz sampling rates and filtered at 10 KHz using a Bessel filter.

FIG. 7. Translocation of tzatziki through 2E-4D-CytK nanopores. A) Schematic representation of the translocation of S1 through 2E-4D-CytK. B) Voltage dependency of translocation rate C) Voltage dependency of the excluded current (Iex %) D) Representative traces at −160 mV bias. E) Dwell time versus current amplitude at −160 mV bias. Experiments were performed at pH 7.5 and under 1 M KCl. Traces were collected at 50 KHz sampling rates and filtered at 10 KHz using a Bessel filter.

FIG. 8. Translocation of mujdei through 2E-4D-CytK nanopores. A) Schematic representation of the translocation of S1 through 2E-4D-CytK. B) Voltage dependency of translocation rate C) Voltage dependency of the excluded current (Iex %) D) Representative traces at −160 mV bias. E) Dwell time versus current amplitude at −160 mV bias. Experiments were performed at pH 7.5 and under 1 M KCl. Traces were collected at 50 KHz sampling rates and filtered at 10 KHz using a Bessel filter.

FIG. 9. Translocation of model substrates through nanopores. Throughout the figure: panel i shows the cut-through of a surface representation of the nanopores (left) and a cartoon representation of its β-barrel region (right). Panel ii indicates the entry or translocation of S1, panel iii indicates the entry or translocation of tzatziki and panel iv shows the entry or translocation of mudjei. A) Representative current traces of substrates through WT-CytK, B) Representative current traces of substrates through K128D-CytK, C) Representative current traces of substrates through K128-K155Q-CytK, D) Representative current traces of K128D-K155D-CytK, E) Representative current traces of K128D-K155Q-Q122D-CytK, F) Representative current traces of K128D-K155D-Q145D-CytK, G) Representative current traces of substrates through K128D-K155D-T147D-CytK and H) K128D-K155D-Q145D-S151D-CytK. Experiments were performed at pH 7.5 and under 1 M KCl. Traces were collected at 50 KHz sampling rates and filtered at 10 KHz using a Bessel filter.

FIG. 10. Translocation of model substrates through nanopores. Throughout the figure: panel i shows the cut-through of a surface representation of the nanopores (left) and a cartoon representation of its β-barrel region (right). Panel ii indicates the dwell time and excluded current dependence on the bias in the case of the translocation of S1, panel iii indicates the dwell time and excluded current dependence on the bias in the case of the translocation of tzatziki and panel iv shows the dwell time and excluded current dependence on the bias in the case of the translocation of mudjei. A) Voltage dependency of translocation rates for type 1 and type 2 blockades (right) and voltage dependency of the excluded current (Iex %) for type 1 and type 2 blockades (left) for the translocation of substrates through WT-CytK, B) Voltage dependency of translocation rates (right) and voltage dependency of excluded current (Iex %) (left) for the translocation of substrates through K128D K128-K155Q-CytK. C) Voltage dependency of translocation rates (top) for the two types of events (type I on the left and type II on the right), and voltage dependency of excluded current (Iex %) of substrates through K128D-K155D-CytK (bottom), D) Voltage dependency of translocation rates (right), and voltage dependency of excluded current (Iex %)(left) of substrates through K128D-K155Q-Q122D-CytK, E) Voltage dependency of translocation rates (right), and voltage dependency of excluded current (Iex %)(left) of substrates through K128D-K155D-Q145D-CytK, F) Voltage dependency of translocation rates (right), and voltage dependency of excluded current (Iex %)(left) of substrates through K128D-K155D-T147D-CytK and G) Voltage dependency of translocation rates (right), and voltage dependency of excluded current (Iex %)(left) of substrates through K128D-K155D-Q145D-S151D-CytK. Experiments were performed at pH 7.5 and under 1 M KCl. Traces were collected at 50 KHz sampling rates and filtered at 10 KHz using a Bessel filter.

FIG. 11. Translocation of unfolded MalE219a across 2E-4D-CytK nanopores. A) representative traces of the translocation of MalE219a across 2E-4D-CytK in 2 M urea. B) dwell time versus amplitude of current blockades under −100 mV. C) cut-through of a surface representation of the nanopores (left) and a cartoon representation of its β-barrel region (right). D) cartoon representation of MalE219a. E) Voltage dependency of the translocation speed F) Voltage dependency of the excluded current. Traces were collected at 50 KHz sampling rates and filtered at 10 KHz using a Bessel filter.

FIG. 12. Translocation of unfolded H152A-GBP across 2E-4D-CytK nanopores. A) representative traces of the translocation of H152A-GBP across 2E-4D-CytK in 2.4 M urea. B) dwell time versus amplitude of current blockades under −100 mV. C) Cut-through of a surface representation of the nanopores (left) and a cartoon representation of its β-barrel region (right). D) cartoon representation of MalE219a. E) Voltage dependency of the translocation speed F) Voltage dependency of the excluded current for L1 and L2 levels as indicated in panel A. Traces were collected at 50 KHz sampling rates and filtered at 10 KHz using a Bessel filter.

FIG. 13. malE219a translocation through the 2E-2D CytK mutant in the presence of 1 M and 1.8 M GuHCl. A) Cut-through of a surface representation of the nanopores (left) and a cartoon representation of its β-barrel region (right). B) Voltage dependency of the dwell time of malE219a in 1 M (black squares) and 1.8 M (red circles) GuHCl. C) Voltage dependency of the excluded current of malE219a in 1 M (black squares) and 1.8 M (red circles) GuHCl. D) Dwell time versus amplitude dependence at various voltages as indicated.

FIG. 14. Characterization of the 2E-4D-CytK nanopore in the two denaturants. IV curves for 2E-4D-CytK nanopores in the urea (A) and GuHCl (B). Numerical values of the asymmetry using the ratio of the ionic current at −100 mV and +100 mV in different concentrations of urea (C) and GuHCl (D).

FIG. 15. MalE219a transport across WT-CytK. A) translocation events of 100 nM of malE219a-D10 unfolded by 2 M urea. B) 100 nM of malE219a added in the cis chamber did not induce events. C) sequence of malE219a-D10. FIG. 15C discloses SEQ ID NO: 33.

FIG. 16 shows a computer system that is programmed or otherwise configured to implement methods provided herein.

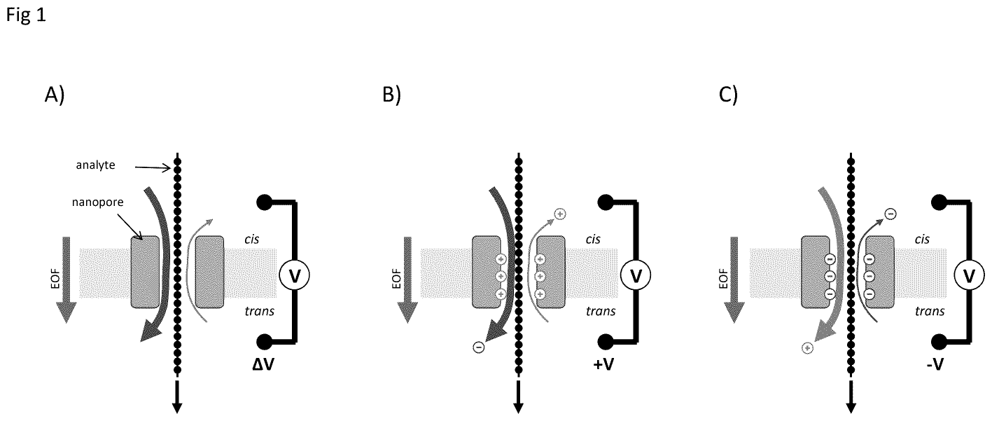

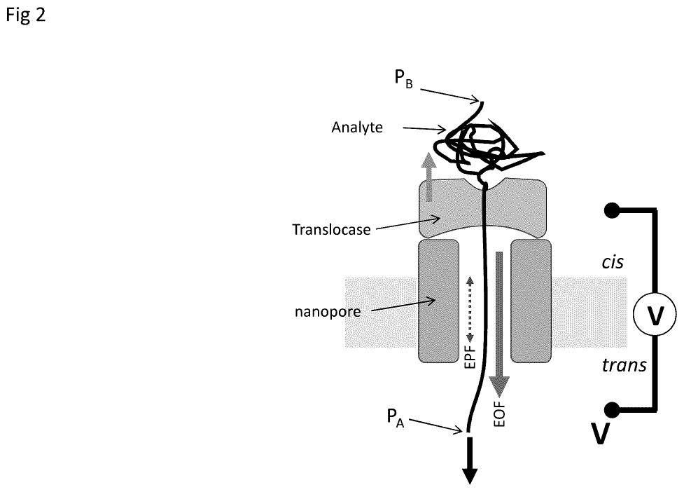

### EXPERIMENTAL SECTION

### Materials and Methods

### Chemicals and Reagents

Ampicillin sodium salt (Fisher Bio Reagents), chloramphenicol (≥98.0% (TLC), Sigma Life Science), LB medium (Roth), 2×YT medium (Roth), NaCl (≥99.5%, p.a., ACS, ISO, Roth), HEPES (PUFFERAN® CELLPURE® ≥99.5%, Roth), imidazole (≥99%, Roth), guanidinium chloride (≥99.5%, biochemistry, Roth), citric acid (≥99.6%, ACS reagent, anhydrous, Acros Organics), BIS-TRIS propane (BTP) (≥99.0%, Sigma Life Sciences), urea (≥99.5%, cryst., Roth), KCl (≥99.5%, p.a., ACS, ISO, Roth), isopropylthio-β-galactoside (IPTG) (≥99.0%, bioscience-grade, dioxin-free, animal-free, Roth), protease inhibitors (Pierce™ Protease inhibitor Mini tablets, EDTA-free, Thermo Scientific), Ni-NTA agarose (Qiagen), Strep Tactin® Sepharose® (IBA Lifesciences), D-desthiobiotin (IBA Lifesciences), mPEG-mal 5 k (Laysan Bio, Inc.), Tris(2-carboxyethyl)phosphine hydrochloride (TCEP) (≥98.0%, biochemistry, Roth), DPhPC (Avanti lipids), n-pentane (Sigma-Aldrich), n-hexadecane (99% pure, Acros organics).

### Methods

### Cloning and Production of the CytK Mutants

Plasmids containing the mutant CytK nanopores were constructed by means of USER cloning using uracil-containing primers (Integrated DNA Technologies). The mutation was included in the homology region and the upstream and downstream fragments would be rejoined together with the empty vector in the USER reaction. The two gene fragments and the linearised pT7-sc1 (AmpR) were generated using in-house made PfuX7 DNA polymerase (Norholm paper), with the mention that the extension time was lowered to 30 s/kb. The PCR products were either gel extracted using a GeneJET gel extraction kit (Thermo scientific), or directly cleaned up from the PCR mix using the GeneJET PCR purification kit (Thermo scientific), depending on the presence of by-products. The gene fragments and the linearised vector were mixed in a molar ratio F1:F2:V of 3:3:1 (recent USER paper) and the USER reaction was performed 25 min at 37 C, followed by a 60 C incubation operation of 10 min and lastly, the mixture was cooled down to r.t. (22 or 20 C) for 15 min and subsequently stored on ice/at 4 C until the transformation operation. The circularised plasmids were transformed into chemically competent E. coli cells using the heat shock procedure and the cells were selected on a LB-agar plate supplemented with 100 μg/mL ampicillin. Plasmids from individual colonies were isolated using a GeneJET Plasmid Miniprep kit (Thermo scientific) and the introduction of the mutations was confirmed by Sanger sequencing (Macrogen).

The plasmids encoding for the CytK mutants were electroporated into BL21(DE3) electrocompetent cells using a Bio Rad Micro Pulser (bacterial setting), and the cells were selected on plates containing 100 μg/mL ampicillin. Next day, several transformants were resuspended in LB medium supplemented with 100 μg/mL ampicillin and added to 200 mL LB medium containing 100 μg/mL ampicillin such that the starting optical density at 600 nm (OD600) was 0.05-0.1. Cells were grown at 37 C, 180 rpm until an OD600 of 0.6-0.8, when the culture was chilled on ice for 5-10 min, followed protein expression being induced with 0.5 mM IPTG. After an incubation of 19-21 h at 25 C, 180 rpm, the cells were harvested (7500 rpm, 5 min). Following a 1 h incubation at −80 C, the cell pellets (100 mL culture) were resuspended in 20-25 mL ice-cold lysis buffer (50 mM HEPES, 150 mM NaCl, 10 mM imidazole, pH 7.4+0.02% DDM) supplemented with ¼ tablet cocktail protease inhibitors per 100 mL cell culture and subsequent operations were performed at 4 C degrees unless stated otherwise. The cell suspension was sonicated (Branson sonifier 450) at 25% duty cycle, 2.5 output control for 2-3 min and the cellular debris was removed (8000 rpm, 20 min). The supernatant was incubated (with shaking) for 20-30 min with 200 μL Ni²⁺-NTA slurry, pre-equilibrated and prewashed with 1 mL lysis buffer. The beads were briefly pelleted (3000 rpm, 1 min) and transferred to the column (1.2 mL bed volume bio-spin chromatography, BioRad) while allowing the flow through pass, at r.t. The column was washed in operations with 10 mL wash buffer (50 mM HEPES, 150 mM NaCl, 30 mM imidazole, pH 7.4+0.02% DDM). The protein was eluted with 200 μL elution buffer (50 mM HEPES, 150 mM NaCl, 250 mM imidazole, pH 7.4+0.02% DDM)( ) in three elution fractions. The presence of the SDS-stable CytK mutant oligomers was confirmed by SDS-PAGE, omitting the heating operation in the sample preparation.

### Preparation of the Target Polymers

S1 substrate was as shown (Zhang et al., 2021, Nat Chem., 13(12):1192-1199). The plasmid bearing tzatziki was prepared from a gblock (Integrated DNA Technologies) introduced in the linearised pT7-sc1 using USER cloning as previously described. The DNA for mujdei was generated by using tzatziki as template. Fusion PCR was used to obtain the full mujdei insert, which would be introduced into pT7-sc1 also by USER cloning. The malE219a, His6 (SEQ ID NO: 34)-malE219 and strep-malE219 substrates were prepared in a similar fashion. All substrates' sequences were confirmed by Sanger sequencing (Macrogen). The substrates were expressed in the SG1146a strain (reference) was used, in order to limit protein degradation. The plasmids containing the S1 and malE219a DNA were transformed into chemically competent SG1146a cells and transformants were selected on ampicillin-containing plates. Next day, several colonies were resuspended in LB medium with ampicillin and added to the culture medium (LB supplemented with 100 μg/mL Amp and 25 μg/mL Chloramphenicol) such that the starting OD600 was 0.05-0.1. Cells were grown until OD600 0.6-0.8, when the culture was chilled (5-10 min on ice) followed by induction with 0.5 mM IPTG at 25 C, 180 rpm for 18-22 h. The cells were harvested (7500 rpm, 5 min) and stored at −80 C for 1 h, followed by resuspension in 20 mL lysis buffer (50 mM HEPES, 150 mM NaCl, 10 mM imidazole, 6 M GuHCl, pH 7.4) at r.t. and all the subsequent operations were performed at r.t. The cell suspension was sonicated (25% duty cycle, 2.5 output control for 2-3 min). The cellular debris was removed (8000 rpm, 20 min) and the resulting supernatant was incubated with 200 μL Ni²⁺-NTA slurry per 100 mL culture with shaking for 30 min. The resin was transferred to the column (2 mL, biorad) and washed with 10 mL wash buffer (50 mM HEPES, 150 mM NaCl, 30 mM imidazole, 1.5 M GuHCl, pH 7.4. Lastly, the protein was eluted four times in 100 μL elution buffer (50 mM HEPES, 150 mM NaCl, 250 mM imidazole, 1.5 M GuHCl, pH 7.4). The presence of the protein was confirmed by SDS-PAGE, followed by aliquoting of the elution fractions and storage at −20 C.

The plasmids containing the tzatziki and mujdei plasmids were transformed into electrocompetent SG1146a cells, which were plated on Amp plates. Several transformants were resuspended in 2YT medium and diluted into 2YT medium supplemented with 100 μg/mL Ampicillin and 20 μg/mL Chloramphenicol to a final OD600 of 0.05-0.1. When OD600 reached 0.6, protein expression was induced with 0.5 mM IPTG at 25 C, 180 rpm for 18-20 h. The proteins were purified as described for S1 and malE219, although under native conditions (thus omitting the GuHCl in the buffers). The presence of the protein was confirmed by SDS-PAGE and using a PEG-maleimide reaction as an additional check.

### Electrophysiology Measurements

Recordings in planar lipid bilayers were performed using a chamber consisting of two compartments, delimitated by a 25 μm thick Teflon membrane which an aperture of approximately 100 m was sparked into. A droplet (half of a 10 μL capillary) consisting of n-hexadecane dissolved in n-pentane (6.25%) was applied on the Teflon membrane, followed by the addition of 500 μL buffer and two droplets of DPhPC lipids in n-pentane (5 mg/mL) in each compartment. Ag/AgCl electrodes were connected to each chamber via agarose bridges (2.5% agarose, 3M KCl solution), grounding the cis compartment. Measurements were performed using an Axon™ Digidata® 1550B digitizer and an Axopatch 200B amplifier (Molecular Devices) and recorded with the Clampex 11.1 software.

### Ion Selectivity

Buffers: Buffer A 2 M KCl, 15 mM HEPES, pH 7.5, Buffer B 0.5 M KCl, 15 mM HEPES, pH 7.5, Buffer C 0 M KCl, 15 mM HEPES, pH 7.5, Buffer D 2 M KCl, 50 mM citric acid, BTP, pH 3.8, Buffer E 0.5 M KCl, 50 mM citric acid, BTP, pH 3.8, Buffer F 0 M KCl, 50 mM citric acid, BTP, pH 3.8

The ion selectivity of the CytK mutants was determined in buffers of either pH 7.5 or 3.8. Firstly, both the cis and trans compartments were filled with 2 M buffer (Buffer A/Buffer D) and a single nanopore was isolated, followed by the pipet-offset adjustment of the current at 0 mV bias to 0 pA. The I/V curve was determined between −140 mV and +140 mV, in operations of 20 mV, using a 10 kHz sampling rate coupled with a 2 kHz Bessel filter. Next, the concentration of KCl in the trans compartment was lowered to approximately 0.5 M by flushing with 0 M buffer (Buffer C/Buffer F) and repeated flushing with 0.5 M buffer (Buffer B/Buffer E) ensured that the final concentration of KCl in the trans compartment was correctly fine-tuned to 0.5 M. Similarly, the I/V curve of the nanopore was measured. The reversal potential was determined from the second I/V curve from the linear function fitting the data points between −20 and +20 mV. The ion selectivity was established from triplicate experiments and expressed as the fraction p_{K+}/p_{Cl-} was calculated using the formula below

$$\frac{{p}_{K +}}{{p}_{Cl -}} = \frac{{[ {a}_{{Cl}^{-}} ]}_{trans} - {[ {a}_{{Cl}^{-}} ]}_{cis} \times {e}^{{V}_{r} F / RT}}{{[ {a}_{{K}^{+}} ]}_{trans} \times {e}^{{V}_{r} F / RT} - {[ {a}_{{K}^{+}} ]}_{cis}}$$

where [a] is the activity of the K⁺ or Cl⁻ in the cis or trans compartment, Vᵣ was the reversal potential, which is obtained from the experiments, F corresponds to the Faraday constant (96 485 C/mol), R the gas constant (8.3145 J mol⁻¹ K⁻¹) and T the temperature (298 K).

### Translocation Experiments

The same setup as described above was employed, with the only difference being the KCl concentration in the buffer, namely 1 M. Single pores of the CytK mutants were isolated and the pore orientation was determined from the I/V curve. Provided the pore vestibule was located in cis, the model substrates were added in the cis compartment (1-2 μL S1 elution, or 5-7 μL tzatziki or mujdei elution sample) and translocation was induced by applying a negative bias. These recordings were collected at 50 kHz sampling rate and a Bessel filter of 10 kHz, using a sweep protocol where the first 200-500 ms were used to unclog the pore by applying a positive bias, followed by approximately 2 s of recording at negative bias. In the case of native substrates, urea was introduced into the system after determining the pore orientation and prior to substrate addition, by flushing both cis and trans compartments with 1 M KCl, 4 M urea, 15 mM HEPES, pH 7.5 buffer in 100 μL operations until the aimed concentration was reached (e.g. 2 M urea). After addition of urea, the substrate (1-2 μL malE219 or 3 μL GBP H152A sample, pre-unfolded in 1.5 M GuHCl) was added in the cis compartment and translocation was followed like described for the other substrates. Each individual set of conditions was tested in triplicate.

### Data Analysis

In the case of the translocation experiments, the files containing the recorded sweeps were analysed using the Clampfit 11.1 software. Firstly, the open pore current (level 0, L0) and the corresponding noise, σ, was determined from the conventional histogram. Secondly, the detection limit, L1, was set at 10σ, which in practise is 5σ (Clampfit sets it half-way, at 5σ). Event detection with the set L0 and L1 was done on those approximately 2 s of recording at negative bias, with a dwell time cut-off of 0.08 ms. The resulting L1 data points were used to construct the log(dwell time) vs amplitude scatter plot, from which the amplitude boundaries of the cluster are defined. Next, using the amplitude boundaries, the logarithmic histogram of the dwell time and the conventional histogram of either the amplitude or the I_{ex %} were constructed. In both cases, the bin value was set such that the distribution within the histogram would resemble as much as possible a Gaussian shape. The values for the log (dwell time) and either the amplitude or the Iex were established by fitting a Gaussian function to the histogram, whose μ is either log (dwell time) or the amplitude/I_{ex %}.

### Example 1: Engineering the Electro-Osmotic Flow in CytK Nanopores

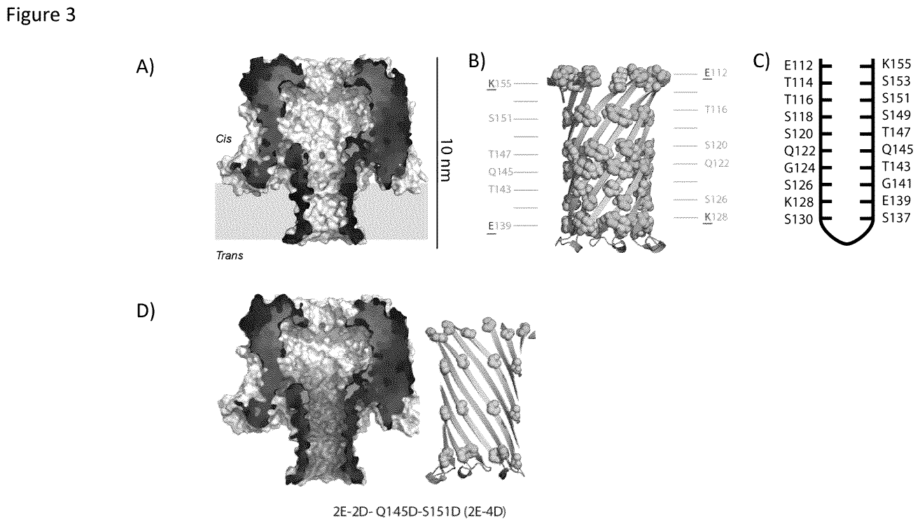

To establish whether an electro-osmotic flow (EOF) can be engineered to translocate and stretch polypeptides against an electrophoretic force (EPF), we used CytK nanopores (FIG. 3). The nanopore was formed by a spherical vestibule ˜5 nm in diameter connected to a ˜5 nm long by 2 nm diameter cylindrical β-barrel region. The latter dominated the resistance of the nanopore, and the wild-type nanopore had no overall charge (the β-barrel region contained two pairs of opposite charge residues: K128-E139 and K155-E112). Consequently, the ion selectivity [(p(K)/p(Cl)] of WT-CytK was nearly one (0.99±0.079), indicating that the WT pore was non-selective and thus showed no electroosmotic flow.

An EOF was induced in WT-CytK by lowering the pH to 3.8, which in turn increased the overall positive charge of the nanopore by protonation of the acidic residues (E112 and E139) making the nanopore anion selective[(p(K)/p(Cl)=0.600±0.000]. Alternatively, an EOF at physiological pH was introduced by removing a positively charged residues near the cis [K128D-CytK, (p(K)/p(Cl)=2.63±0.02] and trans (K128D-K155Q-CytK and K128D-K155D-CytK) entries of the β-barrel region. These modifications increased the ion selectivity, but to [(p(K)/p(Cl)=2.96±0.12 and 3.10±0.08, respectively]. At pH 3.8, the nanopores remained cation selective, but only weakly [(p(K)/p(Cl)=1.36±0.10 for K128D-K155D-CytK].

To test how much the ion selectivity could be increased in CytK, we introduced an additional negative charge at different positions within the β-barrel of K128D-K155D-CytK (2E-2D-CytK). We found that the greatest effect was obtained when the three rings of charges were distributed more evenly along the length of the beta barrel [i.e. the (p(K)/p(Cl) was 3.7 or 3.8 when the charges were placed at ˜1.5 nm apart from the charges at the cis and trans nanopore entry, Table 1, FIG. 3]. The addition of a fourth charge increased the ion selectivity further (Table 1). Overall, the pore with the highest ion selectivity was K128D-Q145D-S151D-K155D-CytK (2E-4D-CytK, Table 1), which showed a (p(K)/p(Cl) of 4.04±0.07. At pH 3.8 the effect of the additional aspartate residues was minimal (Table 1). An anion selective nanopore could also be made by replacing the negative charges for positive charges, resulting in the E112K-E139K-Q145K-S151K-CytK mutant (CytK-6K), with an anion selectivity of 0.207±0.008 (pH 7.5), which remained virtually the same at pH 3.8 (0.213±0.012).

TABLE 1

| | pH 7.5 | | | | pH 3.8 | | | |
|---|---|---|---|---|---|---|---|---|
| | R.P. | R.P. | P_{K}/P_{CI} | P_{K}/P_{CI} | R.P. | R.P. | P_{K}/P_{CI} | P_{K}/P_{CI} |
| Mutant | avg | stdev | avg | stdev | avg | stdev | avg | stdev |
| 6K | −19.3082 | 0.295947 | 0.20764 | 0.008135 | −19.0541 | 0.430281 | 0.213452 | 0.012095 |
| WT | −0.14499 | 0.93088 | 0.991853 | 0.078977 | −6.97445 | 0.008046 | 0.603839 | 0.000512 |
| K128D | 12.90689 | 0.110296 | 2.637693 | 0.02426 | 2.1993 | 1.201764 | 1.174445 | 0.097804 |
| K128D K155Q | 14.24263 | 0.452406 | 2.959448 | 0.118611 | 4.082796 | 0.168512 | 1.339853 | 0.016365 |
| K128D K155D | 14.77041 | 0.276684 | 3.097926 | 0.075129 | 4.333412 | 0.4065 | 1.364928 | 0.039817 |
| K128D K155D T116D | 15.80865 | 0.829636 | 3.414994 | 0.2565 | 6.874349 | 0.674812 | 1.645894 | 0.099883 |
| K128D K155D S120D | 16.73486 | 0.17531 | 3.710497 | 0.061888 | 4.402462 | 0.094033 | 1.37116 | 0.009331 |
| K128D K155D Q122D | 16.69164 | 0.331709 | 3.697167 | 0.144758 | 4.722214 | 0.275372 | 1.403646 | 0.03974 |
| K128D K155D S126D | 13.41838 | 0.276886 | 2.75487 | 0.079607 | 3.729563 | 0.070704 | 1.306001 | 0.008162 |
| K128D K155D T143D | 14.66842 | 0.115902 | 3.069036 | 0.038374 | 3.465806 | 0.2951 | 1.281696 | 0.033533 |
| K128D K155D Q145D | 15.69622 | 0.133073 | 3.36655 | 0.050095 | 3.905657 | 0.158716 | 1.32279 | 0.018525 |
| K128D K155D T147D | 16.50404 | 0.137101 | 3.629647 | 0.046791 | 3.700666 | 0.071011 | 1.303281 | 0.006666 |
| K128D K155D S151D | 17.17087 | 0.673547 | 3.880897 | 0.247497 | 4.346843 | 0.218138 | 1.365795 | 0.021517 |
| K128D K155D T116D T147D | 16.70064 | 0.180842 | 3.698435 | 0.077535 | 3.520646 | 0.075161 | 1.286474 | 0.008517 |
| K128D K155D T116D S151D | 16.10496 | 0.535072 | 3.501811 | 0.209061 | 5.44246 | 0.335793 | 1.479538 | 0.044699 |
| K128D K155D S120D Q122D | 17.26572 | 0.094966 | 3.905306 | 0.044557 | 5.022353 | 0.692365 | 1.436375 | 0.088142 |
| K128D K155D S120D T147D | 16.07314 | 0.08806 | 3.485588 | 0.035042 | 5.92812 | 0.24134 | 1.53296 | 0.033551 |
| K128D K155D S120D S151D | 17.2489 | 0.529757 | 3.906139 | 0.198708 | 4.18201 | 0.379784 | 1.349959 | 0.045391 |
| K128D K155D Q145D S151D | 17.60126 | 0.139616 | 4.036893 | 0.068788 | 4.313304 | 0.014851 | 1.362292 | 0.001796 |

### Example 2: Translocation of Model Substrates Across CytK Nanopores

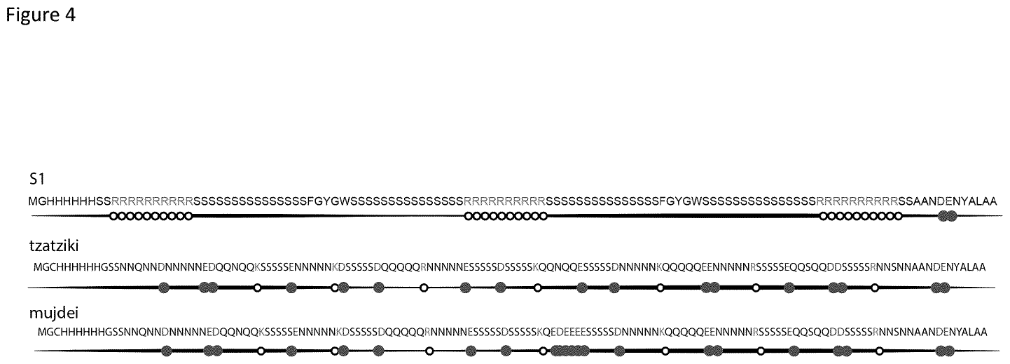

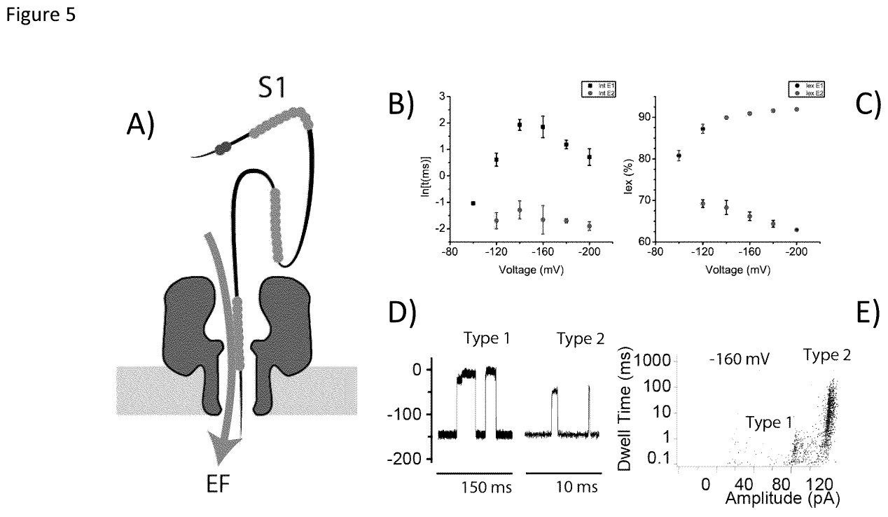

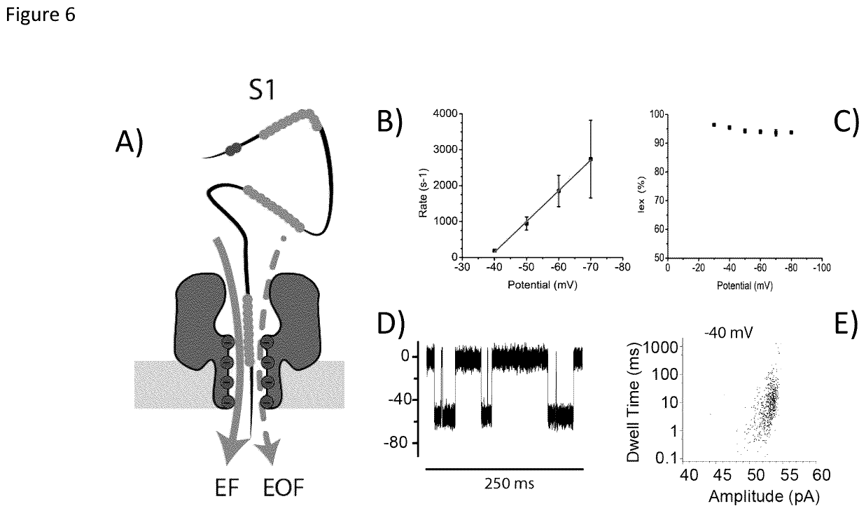

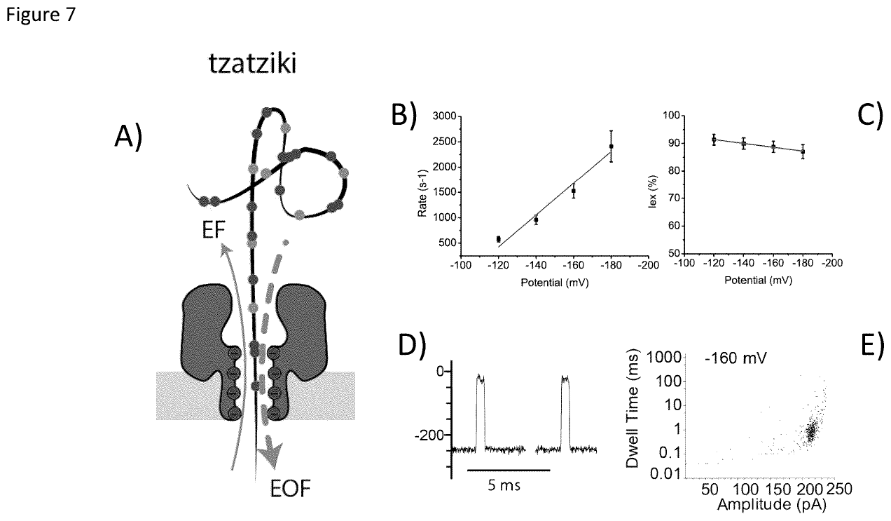

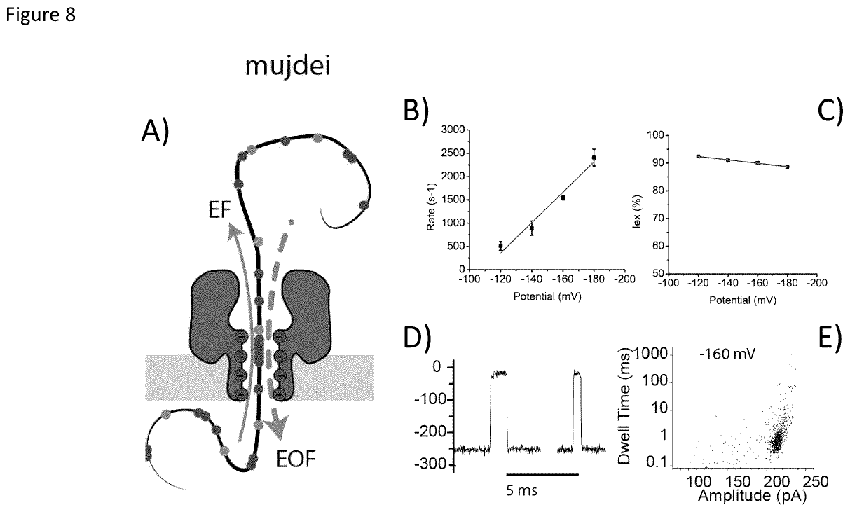

The linearised translocation of a protein across a nanopore requires unfolding the polypeptide and overcoming steric, entropic and electrostatic energy barriers. Unfolded polypeptide translocation was initially tested with S1, a 123 amino acid polypeptide designed to be unstructured and to carry large stretches of positive charges (net charge +28, or +23 net charge over 100 amino acids, +23₁₀₀ at pH 7.5, FIG. 4). The addition of S1 to the cis side of WT-CytK induced current blockades of two types, and showed different dwell times and ionic current during the peptide block I_{b} (here blockades are indicated as the excluded current I_{ex %}=(Iₒ−I_{b})/Iₒ×100, where Iₒ is the open pore current). Type I blockades showed an I_{ex %} that increased from 80.78±1.25% to 91.93±0.22% from −100 mV to −200 mV. The dwell time increased from 0.35±0.02 ms at −100 mV to 7.00±1.41 ms at −140 mV, stayed rather similar at −160 mV (6.74±2.88 ms), then decreased to 2.11±0.71 ms at −200 mV. Type II showed shallower and shorter blockades which I_{ex %} decreased from 69.24±0.94% at −120 mV to 62.92±0.06% at −200 mV. As observed for type I the duration of type II blockades first increased (from 0.19±0.05 ms to 0.28±0.09 ms from −120 mV to −140 mV) and then decreased with the applied bias (0.15±0.02 ms at −200 mV). Possibly, the type of events reflected the orientation of the polypeptide during translocation, capturing first from either the N- or C-terminus. The voltage dependency of the blockades indicates that the electrophoretic force drove S1 translocation across a non-selective nanopore (WT-CytK) above a threshold potential of ˜−160 mV and −140 mV for type I and type II events, respectively.

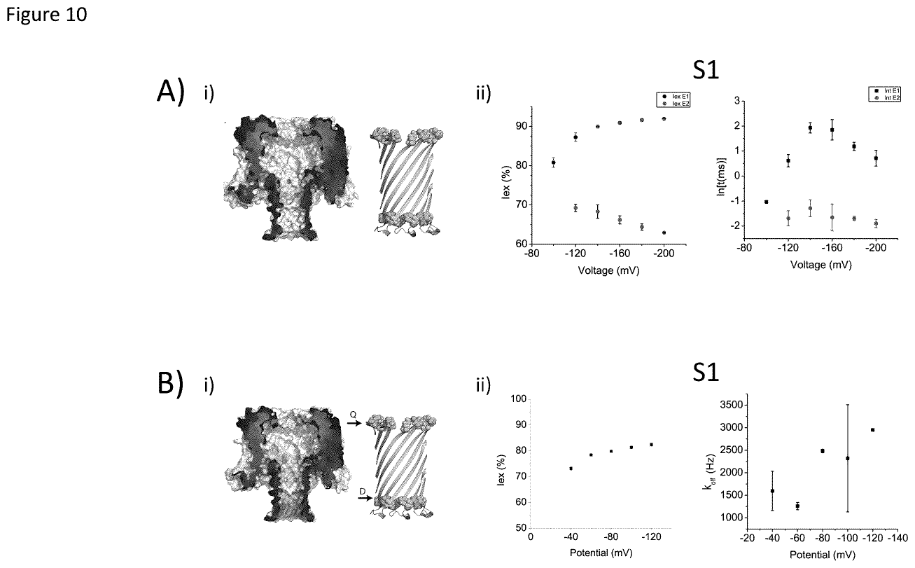

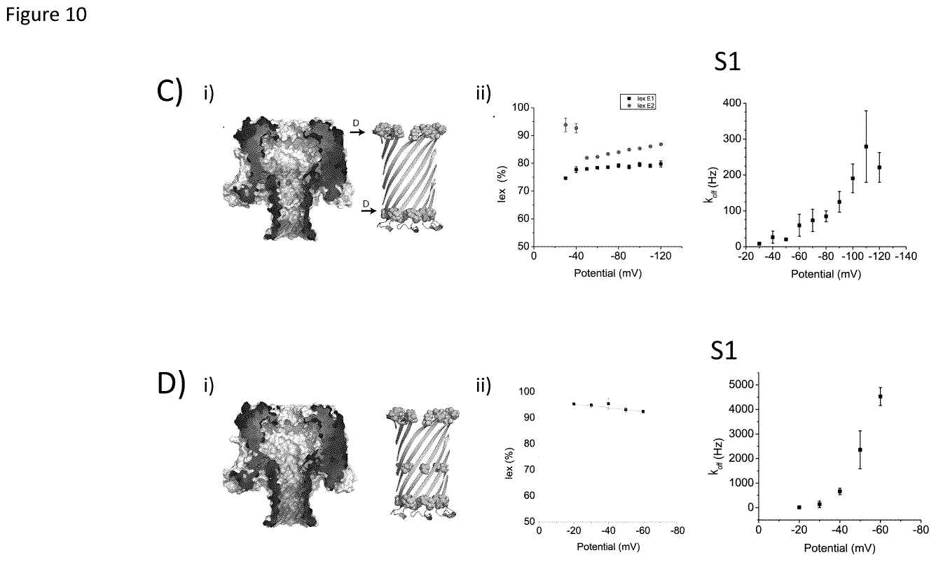

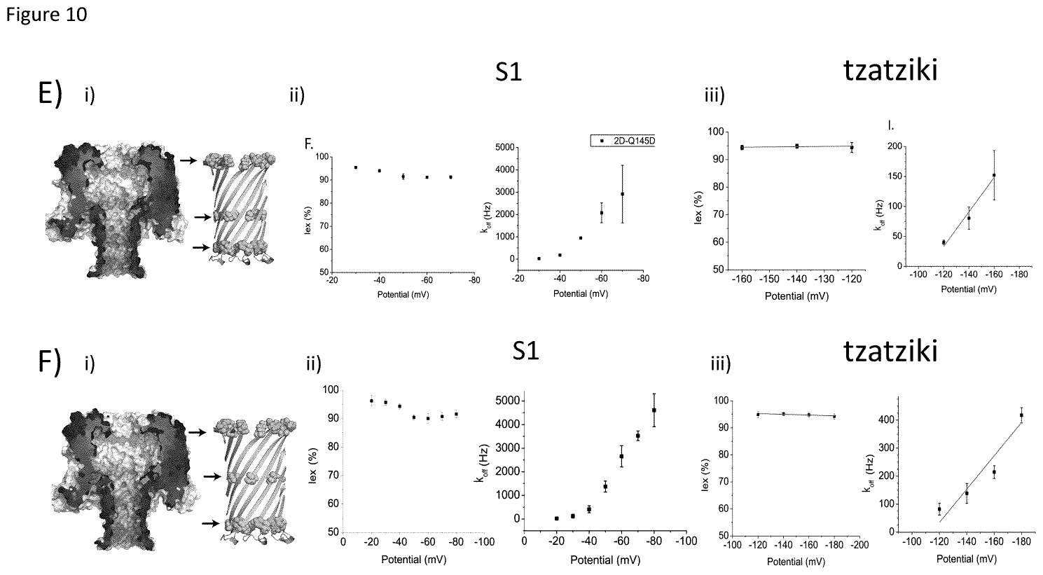

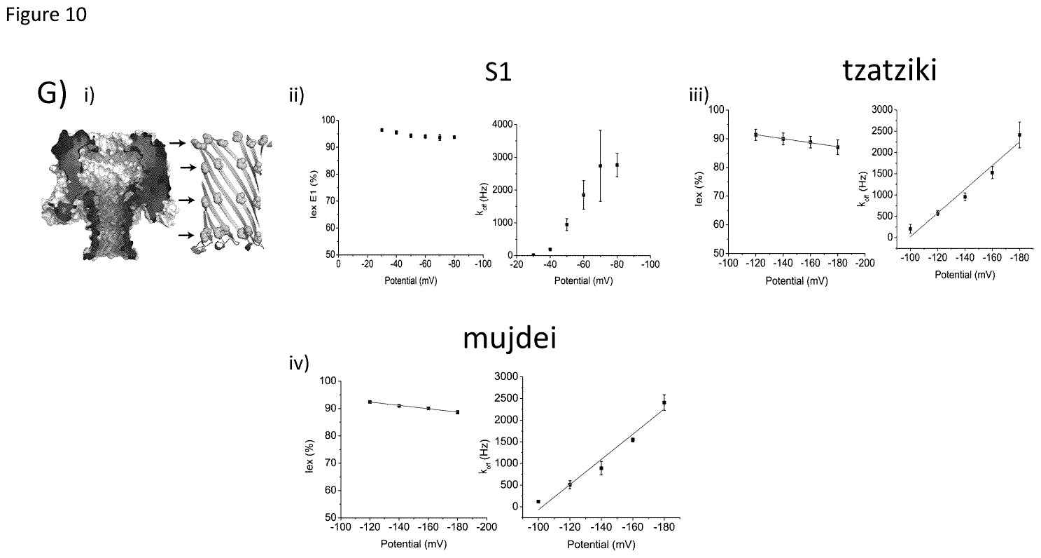

Using the nanopores having an enhanced EOF, the threshold bias to obtain translocation was strongly reduced (e.g. to ˜−30 mV for 2E-2D-CytK),), indicating that the EOF can further drive the translocation of the polypeptide in combination with the net EPF acting in the same direction. In general, however, above the threshold potential, the dwell time was strongly reduced by the additional EOF (FIG. 10).

S1 is a highly positively charged model polypeptide, with an amino acid composition and distribution not found in native proteins.

In order to test a native-like substrate and establish whether the engineered EOF strength can be used to translocate unfolded polypeptides against an EPF, which is a requirement if proteins are to be characterized or sequenced with nanopores, we prepared a new model polypeptide 140 amino acid long named tzatziki, illustrated in FIG. 4. Tzatziki was designed to be unstructured and to carry a relatively large negative charge density of −7.0₁₀₀.

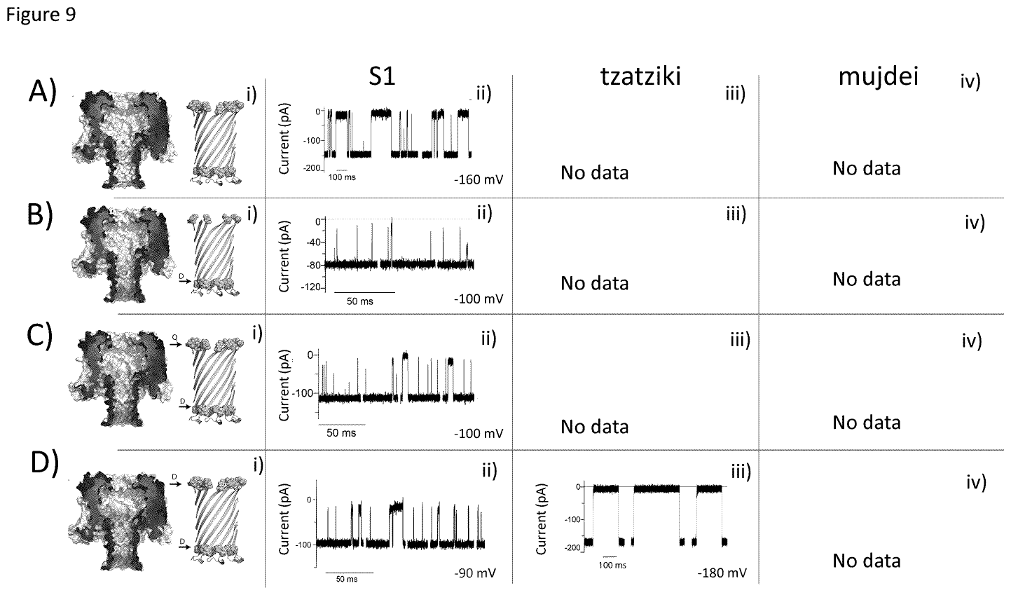

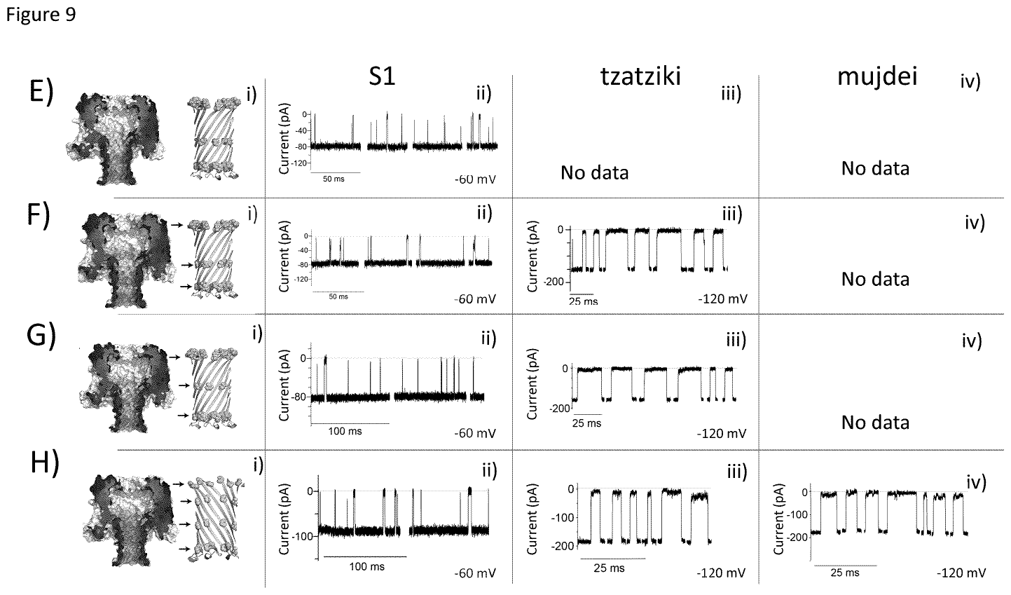

When Tzatziki was added to the cis sides, blockades were not observed with the WT-CytK nanopores at up to +200 mV, indicating that the EPF was too weak to induce polypeptide translocation. In the case of 2E-2D-CytK, capture events were observed above −180 mV against the direction of the EPF that is acting to repel and prevent translocation, although substrate translocation is inconclusive. Tzatziki translocation events were observed in mutant pores with at least three rings of negative residues (cation selectivity >3.5, Table 1) at a bias above −120 mV (FIGS. 9 and 10). Translocation events were observed in direction of the EOF and against the average EPF force acting on the polypeptide. The dwell time of tzatziki decreased with the EOF of the nanopore (FIGS. 9 and 10), further indicating that the EOF drove the translocation of the polypeptide. Hence, a relatively highly negatively charged polypeptide can be translocated against the EPF.

Proteins found in nature might contain stretches with a highly focused charge density. Since the EPF drops very steeply within the nanopore and in particular near the constriction, we investigated whether a substrate with several consecutive negative charges could be translocated across 2E-4D-CytK nanopores. We designed mujdei, identical to tzatziki with the exception of a stretch of five negatively charged residues (EDEEE) in the middle region of the polypeptide (FIG. 4). The overall net charge density of this substrate increased to −9.9₁₀₀. The 2E-4D-CytK nanopore could capture and translocate mujdei showing a slight increase in dwell time compared to tzatziki. Interestingly, the difference in the dwell time between the two substrates decreased with the potential (from 45% at −100 mV to 9% at −140 mV), suggesting that at high bias the electrophoretic effect on the polypeptide becomes almost negligible compared to the EOF.

### Example 3: Stretching the Polypeptides Inside the Nanopore

In nanopore protein sequencing it is important that the polymer is linearised during translocation and that there is enough current associated with the polypeptide blockade to identify individual amino acids. The I_{ex %}is typically dominated by the excluded volume of the polymer inside the nanopore. Therefore, if the polypeptide translocates as a linear polypeptide, the I_{ex %}is expected to be low, while if the polypeptide is folded inside the nanopore the I_{ex %}is expected to be high. However, the I_{ex %}might also be influenced by the charges of the analyte and the nanopore, which might create additional energy barriers for the translocation of ions from solution. For example, in a nanopore with a highly positively charged lumen the translocation of a polymer with high negative charge density such as DNA increased the I_{ex %}to almost hundred percent, most likely because the transport of anions is blocked by the charge in the DNA and that of cations by the charge of the nanopore.

For most substrates, we observed a reduced I_{ex %}with the increasing of the applied voltage, suggesting that the polypeptide was stretched as the electroosmotic pulling force is increased. A notable exception was the translocation of S1 through 2E-2D-CytK nanopores. Possibly, this effect was due to the charge distribution of S1 which might specifically interact with the fixed charges in the nanopore. Interestingly, when comparing the two protein-like substrates the I_{ex %} of mujdei was ˜1% higher than in tzatziki for all nanopores, suggesting that the increased EPF opposing translocation on the more negatively charged mujdei slightly reduced the stretching of the substrate inside the nanopore.

### Example 4: Non-Enzymatic Translocation of Native Substrates Across the 2E-4D Mutant

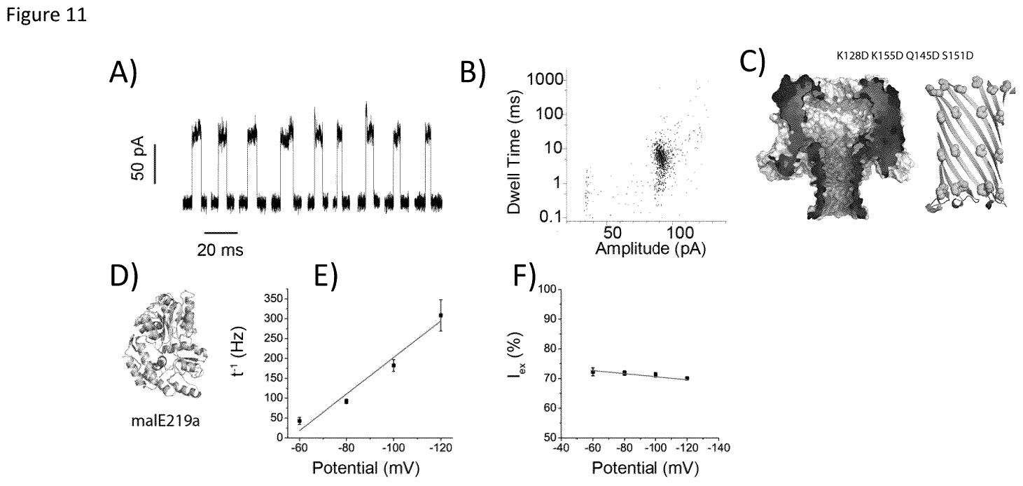

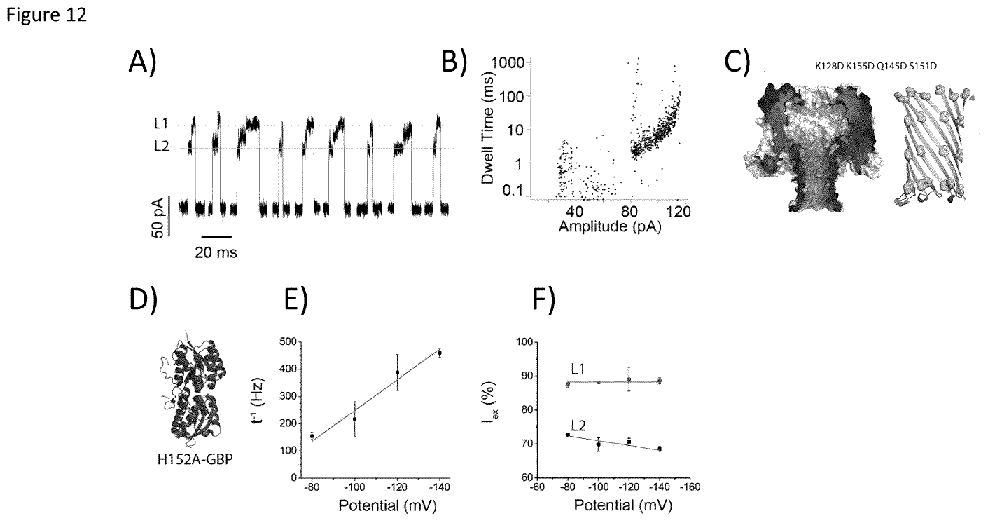

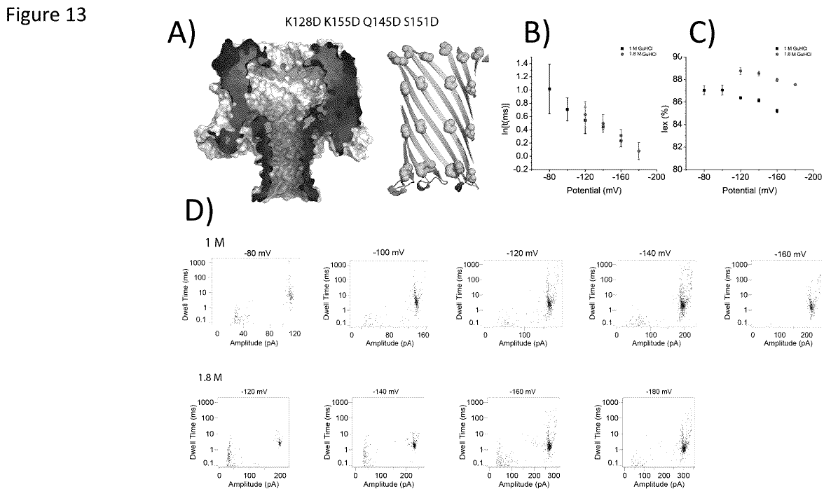

S1, tzatziki and mujdei were designed to contain only disorder-promoting hydrophilic amino acids to minimize the folding of the polymer. In order to test the non-enzymatic translocation of native proteins, we chose two proteins. A maltose-binding-protein variant, malE219a (containing the G220D and E221P destabilising mutations and a total of 412 amino acids, charge density of −2.3₁₀₀ at pH 7.5, FIG. 11), and a glucose binding protein H152A-GBP (bearing a destabilising mutation H152A, 341 amino acids, charge density −1.1₁₀₀ at pH 7.5, FIG. 12). As previously reported, malE219 fully unfolds in 0.7 M GuHCl, while H152A-GBP is unfolded in 1 M GuHCl.

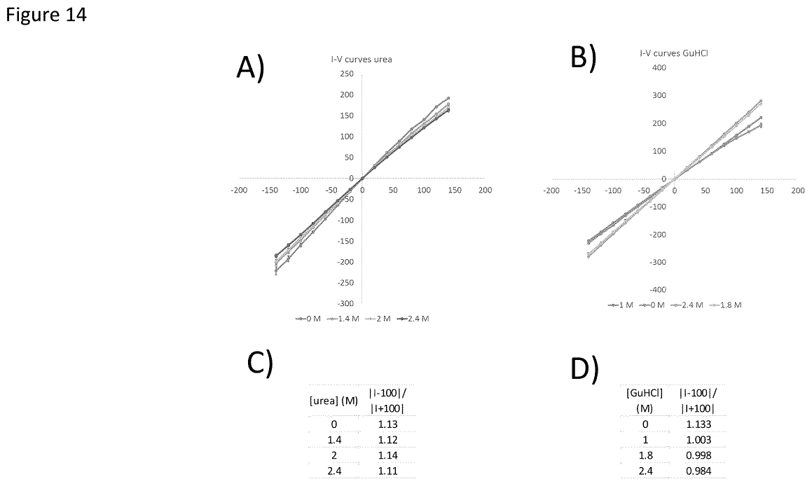

The presence of urea (a neutral denaturant) decreased the open pore current of 2E-4D-CytK while retaining the current asymmetry under opposite bias (FIG. 14), suggesting that it did not change the ionic properties of the nanopore. By contrast, in the presence of Gu.HCl the current symmetry was lost and even reverted at higher concentrations (FIG. 14), suggesting that, as previously shown by MD simulations, the Gu.H⁺ binds to the negative charges within the lumen of the nanopore reducing the ion selectivity.

When adding (pre-unfolded) malE219a substrate to the cis side of 2E-4D-CytK in the presence of 2 M urea, we observed translocation events, which were significantly different from the translocation of the model substrates (FIG. 11). The threshold translocation of MalE219a was ˜−60 mV compared to ˜−120 mV for tzatziki, possibly reflected the lower negative charge density of the protein (−2.3₁₀₀ vs −7.0₁₀₀). The dwell times, on the other hand, was about two-fold longer compared to the dwell time of the model substrates adjusted for their length, possibly reflecting a stronger interaction between the hydrophobic amino acids and the lumen of the nanopore. Importantly, during the blockade, a substantial larger residual current was observed during the translocation of the protein (I_{ex %}70.5±0.57% at −120 mV vs 88.79%±2.03 (tzatziki) or 90.05±0.50 (mujdei) at −160 mV, FIGS. 9 and 10), suggesting a more stretched polypeptide. Furthermore, the current signature showed rather distinctive patterns, which could be related to the sequence and/or structure of the translocating proteins.

Pre-unfolded H152A-GBP (added to the cis side) also translocated through 2E-4D-CytK nanopores in the presence of urea (2.44 M), although the threshold for translocation was higher than for malE219a (−80 mV, FIG. 4B) despite having a lower charge density (−1.1₁₀₀ vs −2.3₁₀₀). Interestingly, the I_{ex %} showed two levels: level 1 defined by an I_{ex %}, of 70.62±1.08% (at −120 mV), which was similar to the I_{ex %} measured for malE219a, and Level 2 with an I_{ex %}, of 89.11±3.50% (at −120 mV, FIGS. 9 and 10), which was similar to the model substrates. This behaviour may be explained by the two-operation unfolding of H152A-GBP, as previously observed for thioredoxin, or by the translocation of a partially folded structure. H152A-GBP translocation threshold was similar to that of malE219a (FIG. 10).

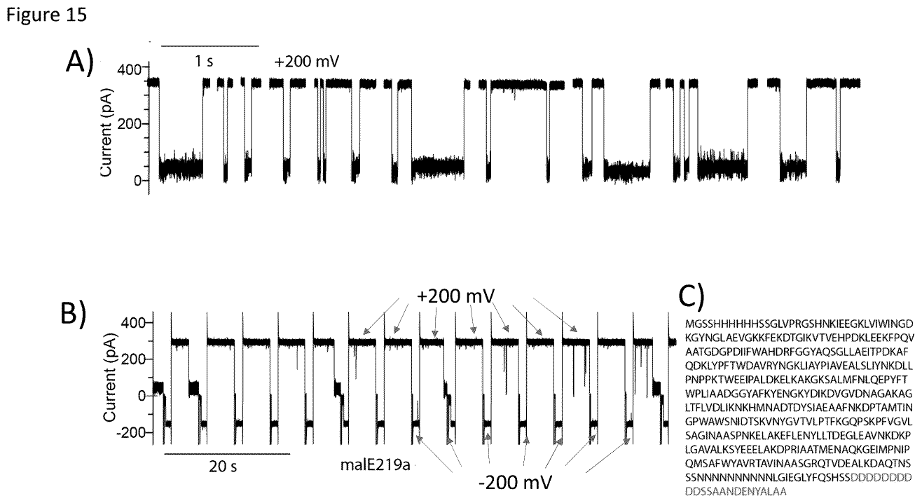

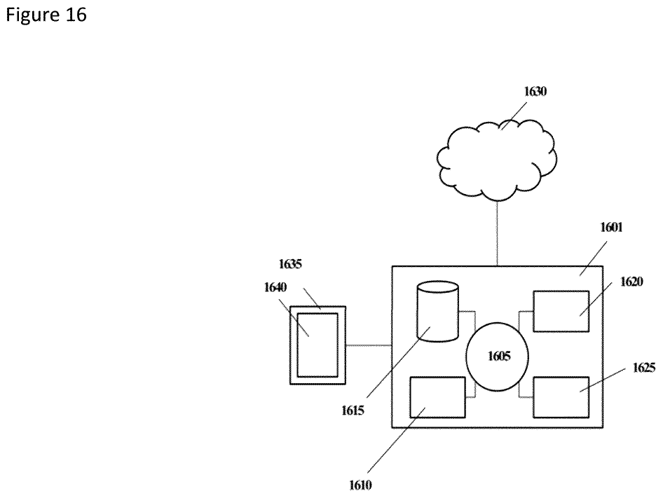

Previous work showed that unfolded model proteins might be translocated across β-barrel nanopores, such as wild type α-hemolysin, which was homologous in structure to CytK, in the presence of Gu.HCl. However, a 10-residue aspartate tag at the N- or C-termini was added to drive the transport across nanopores in this case. We found that malE219a could not be transported using WT-CytK in the presence of 1.5 M GuHCl at any tested bias (up to +200 mV). However, the addition of a D10 tag at the C-terminus (malE219a-D10) allowed translocation at positive applied potentials (FIG. 15). Therefore, the EOF induced by Gu.HCl alone cannot drive polypeptide translocation, and an additional EPF force on the D10 tag must be applied. By contrast, the urea-promoted translocation of substrates across 2E-4D-CytK nanopores is only induced by the engineered EOF, which can overcome the opposing EPF exerted on the negatively charged native proteins during translocation.

When GuHCl (1 M or 1.8 M) was used with 2E-4D-CytK, we observed translocation events at negative bias but with a reduced frequency and the threshold potential shifted towards higher values with more GuHCl. Hence, the EOF induced by the nanopore is reduced due to the binding of GuH⁺ ions to the nanopore, but still allows polypeptide translocation against the EPF. In addition, the I_{ex %}was substantially lower in 2 M urea (70.5±0.57% at −120 mV) compared to 1.8M GuHCl (88.75±0.31%, at −120 mV) and the dwell time was longer (3.28±0.45 ms vs 1.90±1.02 ms, FIG. 10). A possible explanation is that the polypeptides in the presence of urea are more stretched (higher I_{ex %}) and weaker interactions occur between the GuH⁺-coated nanopore and the negative substrates (faster translocation in urea compared to GuHCl).

LISTING OF SEQUENCES USED
Protein Analytes
1. malE219a
MGSSHHHHHHSSGLVPRGSHNKIEEGKLVIWINGDKGYNGLAEVGKKFEKDTGIKV
TVEH
PDKLEEKFPQVAATGDGPDIIFWAHDRFGGYAQSGLLAEITPDKAFQDKLYPFTWDA
VRY
NGKLIAYPIAVEALSLIYNKDLLPNPPKTWEEIPALDKELKAKGKSALMFNLQEPYFT
WP
LIAADGGYAFKYENGKYDIKDVGVDNAGAKAGLTFLVDLIKNKHMNADTDYSIAE
AAFNK
DPTAMTINGPWAWSNIDTSKVNYGVTVLPTFKGQPSKPFVGVLSAGINAASPNKELA
KEF
LENYLLTDEGLEAVNKDKPLGAVALKSYEEELAKDPRIAATMENAQKGEIMPNIPQ
MSAF
WYAVRTAVINAASGRQTVDEALKDAQTNSSSNNNNNNNNNNLGIEGLYFQSH (SEQ
ID NO: 7)
2. malE219
MGSSHHHHHHSSGLVPRGSHNKIEEGKLVIWINGDKGYNGLAEVGKKFEKDTGIKV
TVEH
PDKLEEKFPQVAATGDGPDIIFWAHDRFGGYAQSGLLAEITPDKAFQDKLYPFTWDA
VRY
NGKLIAYPIAVEALSLIYNKDLLPNPPKTWEEIPALDKELKAKGKSALMFNLQEPYFT
WP
LIAADGGYAFKYENGKYDIKDVGVDNAGAKAGLTFLVDLIKNKHMNADTDYSIAE
AAFNK
DPTAMTINGPWAWSNIDTSKVNYGVTVLPTFKGQPSKPFVGVLSAGINAASPNKELA
KEF
LENYLLTDEGLEAVNKDKPLGAVALKSYEEELAKDPRIAATMENAQKGEIMPNIPQ
MSAF
WYAVRTAVINAASGRQTVDEALKDAQT (SEQ ID NO: 8)
3. tzatziki
MGCHHHHHHGSSNNQNNDNNNNNEDQQNQQKSSSSSENNNNNKDSSSSSDQQQQ
QRNNNN
NESSSSSDSSSSSKQQNQQESSSSSDNNNNNKQQQQQEENNNNNRSSSSSEQQSQQD
DSS
SSSRNNSNNAANDENYALAA (SEQ ID NO: 9)
4. mujdei
MGCHHHHHHGSSNNQNNDNNNNNEDQQNQQKSSSSSENNNNNKDSSSSSDQQQQ
QRNNNN
NESSSSSDSSSSSKQEDEEESSSSSDNNNNNKQQQQQEENNNNNRSSSSSEQQSQQDD
SS
SSSRNNSNNAANDENYALAA (SEQ ID NO: 10)
5. S1
MGHHHHHHSSRRRRRRRRRRSSSSSSSSSSSSSSSFGYGWSSSSSSSSSSSSSSSRRRRR
RRRRRSSSSSSSSSSSSSSSFGYGWSSSSSSSSSSSSSSSRRRRRRRRRRSSAANDENYA
LAA (SEQ ID NO: 11)
6. GBP-H152A
MANKKVITLSAVMASMLFGAAAHAADTRIGVTIYKYDDNFMSVVRKAIEQDAKAA
PDVQL
LMNDSQNDQSKQNDQIDVLLAKGVKALAINLVDPAAAGTVIEKARGQNVPVVFFN
KEPSR
KALDSYDKAYYVGTDSKESGIIQGDLIAKHWAANQGWDLNKDGQIQFVLLKGEPG
APDAE
ARTTYVIKELNDKGIKTEQLQLDTAMWDTAQAKDKMDAWLSGPNANKIEVVIANN
DAMAM
GAVEALKAHNKSSIPVFGVDALPEALALVKSGALAGTVLNDANNQAKATFDLAKN
LADGK
GAADGTNWKIDNKVVRVPYVGVDKDNLAEFSKKGSSHHHHH (SEQ ID NO: 12)
CytK nanopores
1. 6K
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEKTT
VTSS
VSYQLGGSIKASVTPSGPSGKSGATGKVTWSDKVSYKQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 13)
2. WT
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYQLGGSIKASVTPSGPSGESGATGQVTWSDSVSYKQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 14)
3. K128D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYQLGGSIDASVTPSGPSGESGATGQVTWSDSVSYKQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 15)
4. K128D K155Q
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYQLGGSIDASVTPSGPSGESGATGQVTWSDSVSYQQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 16)
5. K128D K155D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYQLGGSIDASVTPSGPSGESGATGQVTWSDSVSYDQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 17)
6. K128D K155D T116D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
DSS
VSYQLGGSIDASVTPSGPSGESGATGQVTWSDSVSYDQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 18)
7. K128D K155D S120D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VDYQLGGSIDASVTPSGPSGESGATGQVTWSDSVSYDQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 19)
8. K128D K155D Q122D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYDLGGSIDASVTPSGPSGESGATGQVTWSDSVSYDQTSYKTNLIDQTNKHVKWN
VFFN
GYNNQNWGIYTRDSYHALYGNQLFMYSRTYPHETDARGNLVPMNDLPTLINSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 20)
9. K128D K155D S126D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYQLGGDIDASVTPSGPSGESGATGQVTWSDSVSYDQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 21)
10. K128D K155D T143D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYQLGGSIDASVTPSGPSGESGADGQVTWSDSVSYDQTSYKTNLIDQTNKHVKWN
VFFN
GYNNQNWGIYTRDSYHALYGNQLFMYSRTYPHETDARGNLVPMNDLPTLINSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 22)
11. K128D K155D Q145D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYQLGGSIDASVTPSGPSGESGATGDVTWSDSVSYDQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 23)
12. K128D K155D T147D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYQLGGSIDASVTPSGPSGESGATGQVDWSDSVSYDQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 24)
13. K128D K155D S151D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYQLGGSIDASVTPSGPSGESGATGQVTWSDDVSYDQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTENKSFVL
DWKN
KKLVEKKGSAHHHHHH (SEQ ID NO: 25)
14. K128D K155D T116D T147D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
DSS
VSYQLGGSIDASVTPSGPSGESGATGQVDWSDSVSYDQTSYKTNLIDQTNKHVKWN
VFFN
GYNNQNWGIYTRDSYHALYGNQLFMYSRTYPHETDARGNLVPMNDLPTLINSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 26)
15. K128D K155D T116D S151D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
DSS
VSYQLGGSIDASVTPSGPSGESGATGQVTWSDDVSYDQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 27)
16. K128D K155D S120D Q122D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VDYDLGGSIDASVTPSGPSGESGATGQVTWSDSVSYDQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 28)
17. K128D K155D S120D T147D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VDYQLGGSIDASVTPSGPSGESGATGQVDWSDSVSYDQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 29)
18. K128D K155D S120D S151D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VDYQLGGSIDASVTPSGPSGESGATGQVTWSDDVSYDQTSYKTNLIDQTNKHVKWN
VFFN
GYNNQNWGIYTRDSYHALYGNQLFMYSRTYPHETDARGNLVPMNDLPTLINSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 30)
19. K128D K155D Q145D S151D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYQLGGSIDASVTPSGPSGESGATGDVTWSDDVSYDQTSYKTNLIDQTNKHVKWN
VFFN
GYNNQNWGIYTRDSYHALYGNQLFMYSRTYPHETDARGNLVPMNDLPTLTNSGFS
PGMIA
VVISEKDTEQSSIQVAYTKHADDYTLRPGFTFGTGNWVGNNIKDVDQKTFNKSFVL
DWKN
KKLVEKKGSAHHHHHH (SEQ ID NO: 31)
20. K128D K155Q Q122D
MAQTTSQVVTDIGQNAKTHTSYNTFNNEQADNMTMSLKVTFIDDPSADKQIAVINT
TGSF
MKANPTLSDAPVDGYPIPGASVTLRYPSQYDIAMNLQDNTSRFFHVAPTNAVEETTV
TSS
VSYDLGGSIDASVTPSGPSGESGATGQVTWSDSVSYQQTSYKTNLIDQTNKHVKWN
VFFNGYNNQNWGIYTRDSYHALYGNQLFMYSRTYPHETDARGNLVPMNDLPTLTN
SGFSPGMIA
VVISEKDTEQSSIQVAYTKHADDYTLRPGFTFGTGNWVGNNIKDVDQKTFNKSFVL
DWKNKKLVEKKGSAHHHHHH (SEQ ID NO: 32)

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1.-138. (canceled)

139. A method comprising:
(a) providing:
(i) a nanopore system, wherein the nanopore system comprises (1) a fluidic chamber and; (2) a membrane that separates the fluidic chamber into a first side and a second side; and (3) at least a portion of a nanopore disposed in the membrane; and
(ii) a non-nucleic acid based polymer analyte, wherein the non-nucleic acid based polymer analyte comprises a linear length greater than a channel length of the nanopore and an elongated structure;
(b) translocating the non-nucleic acid based polymer analyte from the first side toward the second side of the fluidic chamber, wherein the nanopore system has an electro-osmotic force resulting from a net ionic current flow from the first side to the second side, wherein the electro-osmotic force translocates the non-nucleic acid based polymer analyte against an electrophoretic force acting in a direction opposite the electro-osmotic force.

140. The method of claim 139, wherein the electro-osmotic force is at least 10% greater than the electrophoretic force.

141. The method of claim 139, further comprising measuring a signal generated by the translocating of (b).

142. The method of claim 141, wherein the measuring comprises: measuring a signal for a state of (a) an open channel of the nanopore; (b) capture of the non-nucleic acid based polymer analyte by the nanopore; or (c) passage of the non-nucleic acid based polymer analyte through the nanopore.

143. The method of claim 141, wherein the signal comprises an ionic current, a change in ionic current, or derivations thereof.

144. The method of claim 139, wherein the electro-osmotic force comprises a net ionic current flow from the first side to second side.

145. The method of claim 139, wherein the electro-osmotic force is modulated by a pH, a type of a salt, a concentration of a salt, an osmotic pressure across the membrane of the system, a modification of the nanopore, or any combination thereof.

146. The method of claim 139, wherein the electro-osmotic force is modulated by an asymmetric salt distribution between the first side of the membrane and the second side of the membrane.

147. The method of claim 139, wherein the nanopore system further comprises a pair of electrodes configured to provide an applied voltage to generate the electrophoretic force.

148. The method of claim 147, wherein the applied voltage is a negative voltage on the second side.

149. The method of claim 147, wherein the applied voltage is a positive voltage on the second side.

150. The method of claim 147, wherein an absolute relative net electro-osmotic current over the applied voltage (I_{reIV}) of the nanopore system is greater than 0.1 pA/mV.

151. The method of claim 139, wherein the linear length of the non-nucleic acid based polymer analyte is at least 30 monomeric units.

152. The method of claim 139, wherein the nanopore has an ion-selectivity P(+)/P(−) of greater than 2.0.

153. The method of claim 139, wherein the nanopore has an ion-selectivity P(+)/P(−) of less than 0.50.

154. The method of claim 139, wherein the nanopore is an alpha-helical oligomeric pore forming protein or fragment thereof.

155. The method of claim 139, wherein the nanopore is a beta-barrel oligomeric pore forming protein or fragment thereof.

156. The method of claim 139, wherein the nanopore comprises a de novo nanopore.

157. The method of claim 139, wherein the nanopore comprises one or more monomers of an Aerolysin (Aer) pore, a Cytolysin K (CytK) pore, a Mycobacterium smegmatis (Msp) pore, an alpha-hemolysin (aHL) pore, a Curli production assembly/transport component CsgG pore, a Fragaceatoxin C (FraC) pore, a Lysenin pore, an outer membrane porin F (OmpF) pore, an outer membrane porin G (OmpG) pore, or a ferric hydroxamate uptake component A (FhuA) pore, or homolog, paralog, ortholog thereof, or phage derived portal proteins, or modified variants thereof, or ion-selective mutants thereof.

158. The method of claim 139, wherein the non-nucleic acid based polymer analyte comprises a peptide, a polypeptide, a protein, a polysaccharide, a lipid, a water-soluble plastic, or combination thereof.


SEQUENCE LISTING (34 sequences)

SEQ ID NO: 1 - protein, 11 aa, synthetic construct
AANDENYALAA

SEQ ID NO: 2 - protein, 64 aa, synthetic construct
MAQEQTKRGGGGGDDDDIAGSTAAGQERREKLTEETDDLLDEIDDVLEENAEDFVRAYVQKGGQ

SEQ ID NO: 3 - protein, 21 aa, synthetic construct
SASSHATRQLSGLKIHSNLYH

SEQ ID NO: 4 - protein, 56 aa, synthetic construct
AKSKGKQRGVKQKIHHFHEPMLHNSSEEQVKVEDAFNQRTSTDSRLQSTGTAPRKK

SEQ ID NO: 5 - protein, 267 aa, synthetic construct
SEVFQECVNLFIKRDIKDCLEKMSEVGFIDITVFKSNPMILDLFVSACDIMPSFTKLGLTLQSEILNIFTLDTPQCIETRKIILGDLSKLLVINKFFRCCIKVIQFNLTDHTEQEEKTLELESIMSDFIFVYITKMRTTIDVVGLQELIEIFIFQVKVKLHHKKPSPNMYWALCKTLPKLSPTLKGLYLSKDVSIEDAILNSIDNKIQKDKAKSKGKQRGVKQKIHHFHEPMLHNSSEEQVKVEDAFNQRTSTDSRLQSTGTAPRKK

SEQ ID NO: 6 - protein, 309 aa, synthetic construct
MAASEIMNNLPMHSLDSSLRDLLNDDLFIESDESTKSVNDQRSEVFQECVNLFIKRDIKDCLEKMSEVGFIDITVFKSNPMILDLFVSACDIMPSFTKLGLTLQSEILNIFTLDTPQCIETRKIILGDLSKLLVINKFFRCCIKVIQFNLTDHTEQEEKTLELESIMSDFIFVYITKMRTTIDVVGLQELIEIFIFQVKVKLHHKKPSPNMYWALCKTLPKLSPTLKGLYLSKDVSIEDAILNSIDNKIQKDKAKSKGKQRGVKQKIHHFHEPMLHNSSEEQVKVEDAFNQRTSTDSRLQSTGTAPRKK

SEQ ID NO: 7 - protein, 412 aa, synthetic construct
MGSSHHHHHHSSGLVPRGSHNKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKDPTAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGLYFQSH

SEQ ID NO: 8 - protein, 387 aa, synthetic construct
MGSSHHHHHHSSGLVPRGSHNKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKDPTAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 9 - protein, 140 aa, synthetic construct
MGCHHHHHHGSSNNQNNDNNNNNEDQQNQQKSSSSSENNNNNKDSSSSSDQQQQQRNNNNNESSSSSDSSSSSKQQNQQESSSSSDNNNNNKQQQQQEENNNNNRSSSSSEQQSQQDDSSSSSRNNSNNAANDENYALAA

SEQ ID NO: 10 - protein, 140 aa, synthetic construct
MGCHHHHHHGSSNNQNNDNNNNNEDQQNQQKSSSSSENNNNNKDSSSSSDQQQQQRNNNNNESSSSSDSSSSSKQEDEEESSSSSDNNNNNKQQQQQEENNNNNRSSSSSEQQSQQDDSSSSSRNNSNNAANDENYALAA

SEQ ID NO: 11 - protein, 123 aa, synthetic construct
MGHHHHHHSSRRRRRRRRRRSSSSSSSSSSSSSSSFGYGWSSSSSSSSSSSSSSSRRRRRRRRRRSSSSSSSSSSSSSSSFGYGWSSSSSSSSSSSSSSSRRRRRRRRRRSSAANDENYALAA

SEQ ID NO: 12 - protein, 341 aa, synthetic construct
MANKKVITLSAVMASMLFGAAAHAADTRIGVTIYKYDDNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGAPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNLADGKGAADGTNWKIDNKVVRVPYVGVDKDNLAEFSKKGSSHHHHH

SEQ ID NO: 13 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEKTTVTSSVSYQLGGSIKASVTPSGPSGKSGATGKVTWSDKVSYKQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 14 - protein, 316 aa, unidentified
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGSIKASVTPSGPSGESGATGQVTWSDSVSYKQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 15 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGSIDASVTPSGPSGESGATGQVTWSDSVSYKQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 16 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGSIDASVTPSGPSGESGATGQVTWSDSVSYQQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 17 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGSIDASVTPSGPSGESGATGQVTWSDSVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 18 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVDSSVSYQLGGSIDASVTPSGPSGESGATGQVTWSDSVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 19 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVDYQLGGSIDASVTPSGPSGESGATGQVTWSDSVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 20 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYDLGGSIDASVTPSGPSGESGATGQVTWSDSVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 21 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGDIDASVTPSGPSGESGATGQVTWSDSVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 22 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGSIDASVTPSGPSGESGADGQVTWSDSVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 23 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGSIDASVTPSGPSGESGATGDVTWSDSVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 24 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGSIDASVTPSGPSGESGATGQVDWSDSVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 25 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGSIDASVTPSGPSGESGATGQVTWSDDVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 26 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVDSSVSYQLGGSIDASVTPSGPSGESGATGQVDWSDSVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 27 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVDSSVSYQLGGSIDASVTPSGPSGESGATGQVTWSDDVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 28 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVDYDLGGSIDASVTPSGPSGESGATGQVTWSDSVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 29 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVDYQLGGSIDASVTPSGPSGESGATGQVDWSDSVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 30 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVDYQLGGSIDASVTPSGPSGESGATGQVTWSDDVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 31 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGSIDASVTPSGPSGESGATGDVTWSDDVSYDQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 32 - protein, 316 aa, synthetic construct
MAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYDLGGSIDASVTPSGPSGESGATGQVTWSDSVSYQQTSYKTNLIDQTNKHVKWNVFFNGYNNQNWGIYTRDSYHALYGNQLFMYSRTYPHETDARGNLVPMNDLPTLTNSGFSPGMIAVVISEKDTEQSSIQVAYTKHADDYTLRPGFTFGTGNWVGNNIKDVDQKTFNKSFVLDWKNKKLVEKKGSAHHHHHH

SEQ ID NO: 33 - protein, 437 aa, synthetic construct
MGSSHHHHHHSSGLVPRGSHNKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKDPTAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGLYFQSHSSDDDDDDDDDDSSAANDENYALAA

SEQ ID NO: 34 - protein, 6 aa, synthetic construct
HHHHHH